(19) United States
(12) Patent Application Publication
(10) Pub. No.: US 2025/0049961 A1
(43) Pub. Date: Feb. 13, 2025

(54) SCALABLE AND HIGH-PURITY CELL-FREE SYNTHESIS OF CLOSED-ENDED DNA VECTORS

(71) Applicant: Generation Bio Co., Cambridge, MA (US)

(72) Inventors: Russell Monds, Cambridge, MA (US); Joris Cipi, Cambridge, MA (US); Daniel Jason Blackstock, Walpole, MA (US); John Chester Durant, Cambridge, MA (US)

(21) Appl. No.: 18/722,701

(22) PCT Filed: Dec. 22, 2022
(86) PCT No.: PCT/US2022/053868

§ 371 Date: Jun. 21, 2024

Related U.S. Application Data
(60) Provisional application No. 63/293,337, filed on Dec. 23, 2021.

(51) Int. Cl. A61K 48/00; C07K 14/755; C12N 15/85

(52) U.S. Cl. CPC A61K 48/0066; C07K 14/755; C12N 15/85; C12N 2800/107

(57) ABSTRACT

This disclosure provides methods for scalable and high-purity cell-free synthesis of DNA vectors, particularly closed-ended DNA vectors (e.g., ceDNA vectors) having linear and continuous structure for delivery and expression of a transgene. The cell-free synthesis includes digesting a double-stranded DNA construct with at least one restriction endonuclease that is capable of cleaving the construct at cleavage sites, which are distinct from the recognition sites, to release an insert having unique overhangs that regulate the high specificity of the subsequent ligation reaction. The insert is then ligated with inverted terminal repeat (ITR) oligonucleotides to form the closed-ended DNA vector. Corresponding DNA vectors prepared by these methods and related products as well other base and intermediate vectors and constructs associated with the methods are also provided in this disclosure.

### RELATED APPLICATIONS

This application claims priority to U.S. Provisional Application No. 63/293,337, filed on Dec. 23, 2021. The entire contents of the foregoing application are expressly incorporated herein by reference.

### TECHNICAL FIELD

The present disclosure relates to the field of gene therapy, including high throughput and high purity production of non-viral vectors for the purpose of expressing a transgene in a subject or cell. For example, the present disclosure provides cell-free methods of synthesizing non-viral DNA vectors. The disclosure also relates to the nucleic acid constructs produced thereby and methods of their use.

### BACKGROUND

Gene therapy aims to improve clinical outcomes for patients suffering from either genetic mutations or acquired diseases caused by an aberration in the gene expression profile. Gene therapy includes the treatment or prevention of medical conditions resulting from defective genes or abnormal regulation or expression, e.g., underexpression or overexpression, that can result in a disorder, disease, malignancy, etc. For example, a disease or disorder caused by a defective gene might be treated, prevented or ameliorated by delivery of a corrective genetic material to a patient, or might be treated, prevented or ameliorated by altering or silencing a defective gene, e.g., with a corrective genetic material to a patient resulting in the therapeutic expression of the genetic material within the patient.

The basis of gene therapy is to supply a transcription cassette with an active gene product (sometimes referred to as a transgene), e.g., that can result in a positive gain-of-function effect, a negative loss-of-function effect, or another outcome. Gene therapy can also be used to treat a disease or malignancy caused by other factors. Human monogenic disorders can be treated by the delivery and expression of a normal gene to the target cells. Delivery and expression of a corrective gene in the patient's target cells can be carried out via numerous methods, including the use of engineered viruses and viral gene delivery vectors. Among the many virus-derived vectors available (e.g., recombinant retrovirus, recombinant lentivirus, recombinant adenovirus, and the like), recombinant adeno-associated virus (rAAV) is gaining popularity as a versatile vector in gene therapy.

Adeno-associated viruses (AAV) belong to the Parvoviridae family and more specifically constitute the dependoparvovirus genus. Vectors derived from AAV (i.e., recombinant AAV (rAVV) or AAV vectors) are attractive for delivering genetic material because (i) they are able to infect (transduce) a wide variety of non-dividing and dividing cell types including myocytes and neurons; (ii) they are devoid of the virus structural genes, thereby diminishing the host cell responses to virus infection, e.g., interferon-mediated responses; (iii) wild-type viruses are considered non-pathologic in humans; (iv) in contrast to wild type AAV, which are capable of integrating into the host cell genome, replication-deficient AAV vectors lack the rep gene and generally persist as episomes, thus limiting the risk of insertional mutagenesis or genotoxicity; and (v) in comparison to other vector systems, AAV vectors are generally considered to be relatively poor immunogens and therefore do not trigger a significant immune response (see ii), thus gaining persistence of the vector DNA and potentially, long-term expression of the therapeutic transgenes.

However, there are several major deficiencies in using AAV particles as a gene delivery vector. One major drawback associated with rAAV is its limited viral packaging capacity of about 4.5 kb of heterologous DNA (Dong et al., 1996; Athanasopoulos et al., 2004; Lai et al., 2010), and as a result, use of AAV vectors has been limited to less than 150,000 Da protein coding capacity. The second drawback is that as a result of the prevalence of wild-type AAV infection in the population, candidates for rAAV gene therapy have to be screened for the presence of neutralizing antibodies that eliminate the vector from the patient. A third drawback is related to the capsid immunogenicity that prevents re-administration to patients that were not excluded from an initial treatment. The immune system in the patient can respond to the vector which effectively acts as a “booster” shot to stimulate the immune system generating high titer anti-AAV antibodies that preclude future treatments. Some recent reports indicate concerns with immunogenicity in high dose situations. Another notable drawback is that the onset of AAV-mediated gene expression is relatively slow, given that single-stranded AAV DNA must be converted to double-stranded DNA prior to heterologous gene expression.

Additionally, conventional AAV virions with capsids are produced by introducing a plasmid or plasmids containing the AAV genome, rep genes, and cap genes (Grimm et al., 1998). However, such encapsidated AAV virus vectors were found to inefficiently transduce certain cell and tissue types and the capsids also induce an immune response.

Accordingly, use of adeno-associated virus (AAV) vectors for gene therapy is limited due to the single administration to patients (owing to the patient immune response), the limited range of transgene genetic material suitable for delivery in AAV vectors due to minimal viral packaging capacity (about 4.5 kb), and slow AAV-mediated gene expression.

Closed-ended DNA vectors have been developed that are capable of delivering one or more desired transgenes in vivo for therapeutic or other purposes, and which avoid the above-described liabilities of AAV and other virus vector systems. However, methods of producing such ceDNA vectors have relied upon traditional bacterial or insect cell production methods. Such methods can result in contaminants (e.g., nucleic acid contaminants) from the cells used to produce the vectors (e.g., Sf9 cells) that are inconvenient or costly to remove and which may have undesirable side effects if included in ceDNA therapeutic formulation. Accordingly, there is need in the field for a technology that allows for the generation of recombinant vectors to be used in methods of controlling gene expression with minimal off-target effects such as those introduced by such contaminants or other artifacts of the purification method. It would also be advantageous that the technology is suitable for and supports large-scale manufacturing of the recombinant vectors. The methods provided herein addresses these unmet needs.

### SUMMARY

Conventional methods for production of viral and virally-derived DNA typically use eukaryotic cells, e.g., mammalian or insect cells. One commonly used insect cell line is Sf9. However, not only do these cells both contain enzymes and other proteins which may have a deleterious effect on the DNA to be replicated, but the process of purifying the desired DNA from cell lysates introduces cellular nucleic acids whose presence can make purification of the desired DNA product more difficult. Furthermore, such impurities or contaminants can have a range of deleterious and/or unwanted effects in the subject to which the desired DNA is administered. Additionally, such traditional cell-based production methods can have issues with respect to the quantity of DNA vector product produced, and it is not uncommon for significant engineering of the cell line itself or the production technology to be required to produce desirable yields.

The technical solution to these vector production challenges, as provided herein, relates to a cell-free synthetic production method that can readily produce closed circle hairpin loop-containing DNA vectors such as, but not limited to, close-ended DNA vectors (ceDNA vectors) in higher purities than by conventional means using eukaryotic cells (see, e.g., FIG. 13).

Accordingly, provided herein is a method of producing a closed-ended DNA (ceDNA) vector, the method comprising: contacting a double-stranded DNA construct having a sense strand and an antisense strand with at least a first restriction endonuclease and at least a second restriction endonuclease, wherein the construct comprises a transgene expression cassette, a first non-palindromic restriction endonuclease recognition site and a corresponding first cleavage site upstream of the transgene expression cassette, and a second non-palindromic restriction endonuclease recognition site and a corresponding second cleavage site downstream of the transgene expression cassette; and wherein the first restriction endonuclease is capable of cleaving the double-stranded DNA construct at the first cleavage site, and wherein the second restriction endonuclease is capable of cleaving the double-stranded DNA construct at the second cleavage site, and wherein contacting the double-stranded DNA construct with the first restriction endonuclease and the second restriction endonuclease releases an insert having a first end comprising a first single-stranded overhang and a second end comprising a second single-stranded overhang; ligating the first end to a first oligonucleotide comprising one or more hairpin structures; and ligating the second end to a second oligonucleotide comprising one or more hairpin structures; thereby producing a ceDNA vector.

In one embodiment, the first and/or second oligonucleotides comprise inverted terminal repeats (ITRs). In one embodiment, the first oligonucleotide and the second oligonucleotide are different. In another embodiment, the first oligonucleotide and the second oligonucleotide are the same. In one embodiment, each of oligonucleotides independently includes 1, 2, 3, 4, or more stem-loop regions. In another embodiment, each of the oligonucleotides independently includes 2 or 3 stem-loop regions.

In one embodiment, the first restriction endonuclease and the second restriction endonuclease are different restriction endonucleases. In another embodiment, the first restriction endonuclease and the second restriction endonuclease are the same restriction endonuclease.

In one embodiment, the first non-palindromic restriction endonuclease recognition site and the corresponding first cleavage site are separate and distinct sites from each other, and wherein both sites are located upstream of the transgene expression cassette. In another embodiment, the first cleavage site is about 1 to about 22 nucleotides away from the first non-palindromic restriction endonuclease recognition site in at least one of the sense strand and the antisense strand of the construct. In another embodiment, the first cleavage site is about 1 to about 8 nucleotides away from the first non-palindromic restriction endonuclease recognition site in at least one of the sense strand and the antisense strand of the construct.

In one embodiment, the second non-palindromic restriction endonuclease recognition site and the corresponding second cleavage site are separate and distinct sites from each other, and wherein both sites are located downstream of the expression cassette. In another embodiment, the second cleavage site is about 1 to about 22 nucleotides away (e.g., about 2 to about 22, about 2 to about 20, about 5 to about 22, about 5 to about 20, about 10 to about 22, about 10 to about 20, about 15 to about 22, about 15 to about 20, about 2 to about 15, about 5 to about 15, about 10 to about 15 about 2 to about 10, about 5 to about 10, about 2 to about 10, about 2 to about 10 nucleotides away) of the second non-palindromic restriction endonuclease recognition site in at least one of the sense strand and the antisense strand of the construct. In another embodiment, the second cleavage site is about 1 to about 8 nucleotides away from the second non-palindromic restriction endonuclease recognition site in at least one of the sense and the antisense strand of the construct.

In one embodiment, the first non-palindromic restriction endonuclease recognition site and the second non-palindromic restriction endonuclease recognition site are each a double-stranded polynucleotide having different 5′ to 3′ nucleotide sequences in each of the sense strand and the antisense strand.

In one embodiment, one or both of the single-stranded overhangs at the ends of the insert are 5′ overhangs. In another embodiment, one or both of the single-stranded overhangs at the ends of the insert are 3′ overhangs.

In one embodiment, the first oligonucleotide comprising one or more hairpin structures and the second oligonucleotide comprising one or more hairpin structures are each a single-stranded oligonucleotide that self-anneals to form a three-dimensional configuration. In one embodiment, the three-dimensional configuration is a T- or Y-shaped stem-loop structure. In another embodiment, the first oligonucleotide comprising one or more hairpin structures and the second oligonucleotide comprising one or more hairpin structures each self-anneal to further form a single-stranded overhang at either the 5′ end or the 3′ end of each oligonucleotide.

In one embodiment, the first oligonucleotide and the second oligonucleotide each self-anneal to further form a single-stranded overhang at the 5′ end of each oligonucleotide. In another embodiment, the first oligonucleotide and the second oligonucleotide each self-anneal to further form a single-stranded overhang at the 3′ end of each oligonucleotide. In one embodiment, the single-stranded overhang of the first oligonucleotide and the single-stranded overhang of the second oligonucleotide are each about 1 to about 12 nucleotides in length, about 1 to about 8 nucleotides in length, about 2 to about 6 nucleotides in length, or about 3, about 4, about 5, or about 6 nucleotides in length.

In one embodiment, the 5′ end of each oligonucleotide is phosphorylated.

In one embodiment, the 5′ to 3′ nucleotide sequences of the single-stranded overhang of the first oligonucleotide and the single-stranded overhang of the second oligonucleotide are non-complementary to each other. In another embodiment, the 5′ to 3′ nucleotide sequences of the single-stranded overhang of the first oligonucleotide and the single-stranded overhang of the second oligonucleotide are the same. In another embodiment, the first oligonucleotide and the second oligonucleotide have the same nucleotide sequence. In another embodiment, the single-stranded overhangs at each end of the insert comprise the same 5′ to 3′ nucleotide sequence. In another embodiment, the single-stranded overhang of the first oligonucleotide and the single-stranded overhang of the second oligonucleotide are each complementary to both of the single-stranded overhangs at the ends of the insert. In another embodiment, the 5′ to 3′ nucleotide sequences of the single-stranded overhang of the first oligonucleotide and the single-stranded overhang of the second oligonucleotide are different. In another embodiment, he first oligonucleotide and the second oligonucleotide comprise different nucleotide sequences. In another embodiment, the 5′ to 3′ nucleotide sequences of the single-stranded overhangs at each end of the insert are different.

In one embodiment, the single-stranded overhang of the first oligonucleotide and the single-stranded overhang of the second oligonucleotide are each complementary to only one of the single-stranded overhangs at the ends of the insert.

In one embodiment, the single-stranded overhang of the first oligonucleotide and/or the single-stranded overhang of the second oligonucleotide comprises a 5′ to 3′ nucleotide sequence selected from the group consisting of CTCT, CTCA, CACT, CTC, and GCT. In another embodiment, one or both of the first oligonucleotide and the second oligonucleotide is synthetic.

In one embodiment, the first oligonucleotide and the second oligonucleotide are each about 40 nucleotides to about 75 nucleotides in length, or about 45 to about 65 nucleotides in length. In another embodiment, the first oligonucleotide and the second oligonucleotide each independently comprise a nucleotide sequence selected from the group consisting of SEQ ID NO: 1; SEQ ID NO: 2; SEQ ID NO: 3; SEQ ID NO: 4; SEQ ID NO: 5; SEQ ID NO: 6; SEQ ID NO:7; and SEQ ID NO:8. In another embodiment, the first oligonucleotide and the second oligonucleotide each independently comprise a nucleotide sequence at least 95%, at least 96%, at least 97%, at least 98% or at least 99% identical to a sequence to a nucleotide sequence selected from the group consisting of SEQ ID NO: 1; SEQ ID NO: 2; SEQ ID NO: 3; SEQ ID NO: 4; SEQ ID NO: 5; SEQ ID NO: 6; SEQ ID NO:7; and SEQ ID NO: 8. In another embodiment, the first oligonucleotide and the second oligonucleotide each independently consists of a nucleotide sequence selected from the group consisting of SEQ ID NO: 1; SEQ ID NO: 2; SEQ ID NO: 3; SEQ ID NO: 4; SEQ ID NO: 5; SEQ ID NO: 6; SEQ ID NO:7; and SEQ ID NO: 8.

In one embodiment, each hairpin structure and/or each T- or Y-shaped stem-loop structure of the first oligonucleotide and each hairpin structure and/or each T- or Y-shaped stem-loop structure of the second oligonucleotide comprises a stem region that is at least about 4 base pairs in length, about 4 base pairs to about 20 base pairs in length, about 4 base pairs to about 15 base pairs in length, about 4 base pairs to about 6 base pairs in length, or about 6 base pairs to about 8 base pairs in length. In another embodiment, the stem region length does not include any single-stranded overhang.

In one embodiment, at least one or both of the restriction endonucleases is a Type IIS restriction endonuclease. In another embodiment, the first restriction endonuclease and the second restriction endonuclease are the same restriction endonuclease. In another embodiment, the first restriction endonuclease and the second restriction endonuclease are different restriction endonucleases. In another embodiment, the Type IIS restriction endonuclease is selected from group consisting of AcuI, AlwI, Alw26I, BasI, BbsI, BbvI, BceAI, BcgI, BCiVI, BcoDI, BruAI, BmrI, BpiI, BpuEI, BsaI, BsaXI, BseGI, BseRI, BsgI, BsmAI, BsmBI, BsmFI, BsmI, BspCNI, BspMI, BspQI, BsrDI, BsrI, BtgZI, BtsCI, BtsI, MutI, CspCI, Earl, EciI, Eco31I, Esp3I, FauI, FokI, HgaI, HphI, HpyAV, LguI, MboII, MlyI, MmeI, MnlI, Mva1269I, NmeAIII, PaqCI, PleI, SapI, SfaNI, and an isoschizomer thereof. In another embodiment, the each of the first and second restriction endonucleases is a Type IIS restriction endonuclease independently selected from the group consisting of AcuI, AlwI, Alw26I, BasI, BbsI, BbvI, BceAI, BcgI, BCVI, BcoDI, BruAI, BmrI, BpiI, BpuEI, BsaI, BsaXI, BseGI, BseRI, BsgI, BsmAI, BsmBI, BsmFI, BsmI, BspCNI, BspMI, BspQI, BsrDI, BsrI, BtgZI, BtsCI, BtsI, MutI, CspCI, Earl, EciI, Eco31I, Esp3I, FauI, FokI, HgaI, HphI, HpyAV, LguI, MboII, MlyI, MmeI, MnlI, Mva1269I, NmeAIII, PaqCI, PleI, SapI, SfaNI, and an isoschizomer thereof. In a further embodiment, the at least one Type IIS restriction endonuclease is selected from group consisting of BbsI, BsaI, Esp3I, and SapI, and an isoschizomer thereof. In a further embodiment, the at least one Type IIS restriction endonuclease is BsaI or an isoschizomer thereof.

In one embodiment, after the ligating, the first non-palindromic restriction endonuclease recognition site and the second non-palindromic restriction endonuclease recognition site are not regenerated in the resulting ceDNA vector.

In one embodiment, the double-stranded DNA construct further comprises a least a first partial ITR and a second partial ITR each flanking the transgene expression cassette. In another embodiment, the first partial ITR is upstream of the transgene expression cassette and downstream of the first non-palindromic restriction endonuclease recognition site and the corresponding first cleavage site. In another embodiment, the second partial ITR is downstream of the transgene expression cassette and upstream of the second non-palindromic restriction endonuclease recognition site and the corresponding second cleavage site. In another embodiment, the first cleavage site is adjacent to the first partial ITR and the second cleavage site is adjacent to the second partial ITR. In another embodiment, the double-stranded DNA construct further comprises a first spacer between the first partial ITR and the transgene expression cassette. In another embodiment, the double-stranded DNA construct further comprises a second spacer between the second partial ITR and the transgene expression cassette. In another embodiment, the double-stranded DNA construct is selected from the group consisting of a bacmid, a plasmid, a minicircle, and a linear double-stranded DNA molecule.

In one embodiment, the resulting ceDNA vector comprises the transgene expression cassette and at least a first ITR and a second ITR each flanking the transgene expression cassette. In another embodiment, the first ITR is upstream of the transgene expression cassette. In another embodiment, the second ITR is downstream of the transgene expression cassette. In another embodiment, the first ITR comprises nucleotide sequences from the first oligonucleotide and the first partial ITR. In another embodiment, the second ITR comprises nucleotide sequences from the second oligonucleotide and the second partial ITR. In another embodiment, the first ITR is devoid of the first non-palindromic restriction endonuclease recognition site. In another embodiment, the second ITR is devoid of the second non-palindromic restriction endonuclease recognition site.

In one embodiment, the first ITR and the second ITR each comprise a hairpin structure and/or a T- or Y-shaped stem-loop structure. In another embodiment, the first ITR and the second ITR each comprise a T- or Y-shaped stem-loop structure. In another embodiment, the T- or Y-shaped stem-loop structure comprises a stem comprising A-A′ and D-D′ stem regions and two B-B′ and C-C′ loops. In another embodiment, one or both of the first ITR and the second ITR is an adeno-associated virus (AAV) ITR or an AAV-derived ITR. In another embodiment, one or both of the first ITR and the second ITR is a wild-type ITR. In another embodiment, both the first ITR and the second ITR are wild-type ITRs. In another embodiment, one or both of the first ITR and the second ITR is a modified ITR. In another embodiment, the first ITR and the second ITR are symmetrical or substantially symmetrical to each other. In another embodiment, the first ITR and the second ITR are asymmetrical ITRs.

In one embodiment, one or both of the first ITR and the second ITR comprises one or more modifications selected from the group consisting of an addition, a deletion, a truncation, and a point mutation. In another embodiment, the one or more modifications are located in the A-A′ stem region, the B-B′ loop, the C-C′ loop, and/or D-D′ stem region of one or both of the first ITR and the second ITR. In another embodiment, the one or more modifications are located in the B-B′ loop and/or the C-C′ loop of one or both of the first ITR and the second ITR. In another embodiment, the B-B′ loop and the C-C′ loop of one of the first ITR and the second ITR are truncated.

In one embodiment, the transgene expression cassette further comprises a first spacer between the first ITR and the transgene expression cassette. In another embodiment, the transgene expression cassette further comprises a first spacer between the second ITR and the transgene expression cassette. In another embodiment, the transgene expression cassette further comprises a first spacer between the first ITR and the transgene expression cassette, and a second spacer between the second ITR and the transgene expression cassette.

In one embodiment, the transgene expression cassette comprises a transgene. In another embodiment, the transgene encodes a therapeutic protein. In another embodiment, the therapeutic protein is selected from the group consisting of an enzyme, a coagulation factor or co-factor, an antibody or an antigen-binding fragment thereof, an antigen, a gene-editing protein, and a cytotoxic protein.

In one embodiment, the transgene expression cassette further comprises a genetic element selected from the group consisting of a promoter, an enhancer, an intron, a posttranscriptional regulatory element, and a polyadenylation signal. In another embodiment, the posttranscriptional regulatory element comprises a WHP posttranscriptional regulatory element (WPRE).

In one embodiment the ligating is effected by a ligase or an AAV Rep protein. In another embodiment, the ligase is T4 ligase.

In one embodiment, the method further comprises isolating or purifying the resulting ceDNA vector. In another embodiment, the method further comprises isolating or purifying the insert prior to the ligating. In another embodiment, the method does not comprise isolating or purifying the insert prior to the ligating. In another embodiment, the contacting and ligating steps are performed in a single reaction vessel.

In one embodiment, the resulting ceDNA vector comprises at least about 85%, at least about 90%, at least about 95%, at least about 96%, at least about 97%, at least about 98%, or at least about 99% of a monomeric species of the ceDNA vector.

In one embodiment, provided herein is a closed-ended DNA (ceDNA) vector produced by any of the methods disclosed herein. In another embodiment, provided herein is a pharmaceutical composition comprising the closed-ended DNA (ceDNA) vector produced by any of the methods disclosed herein. In another embodiment, provided herein is a lipid nanoparticle composition comprising the closed-ended DNA (ceDNA) vector produced by any of the methods disclosed herein. In another embodiment, provided herein is an isolated host cell comprising the closed-ended DNA (ceDNA) vector produced by any of the methods disclosed herein. In another embodiment, provided herein is a transgenic animal comprising the closed-ended DNA (ceDNA) vector produced by any of the methods disclosed herein.

In one embodiment, provided herein is a method of treating a disorder, disease, or condition in a subject (e.g., a genetic disorder, disease, or condition), the method comprising administering to the subject a therapeutically effective amount of the closed-ended DNA (ceDNA) vector produced by any of the methods disclosed herein, or a pharmaceutical composition of comprising the ceDNA vector produced by any of the methods disclosed herein, or a lipid nanoparticle composition comprising the ceDNA vector produced by any of the methods disclosed herein.

In one embodiment, provided herein is a method of delivering a therapeutic protein to a subject, the method comprising administering to the subject a therapeutically effective amount of the closed-ended DNA (ceDNA) vector produced by any of the methods disclosed herein, or a pharmaceutical composition of comprising the ceDNA vector produced by any of the methods disclosed herein, or a lipid nanoparticle composition comprising the ceDNA vector produced by any of the methods disclosed herein.

In another embodiment, the therapeutic protein is selected from the group consisting of an enzyme, a coagulation factor or co-factor, an antibody or an antigen-binding fragment thereof, an antigen, a gene-editing protein, and a cytotoxic protein.

In another embodiment, provided herein is an inverted terminal repeat (ITR) nucleotide sequence selected from the group consisting of SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30 and SEQ ID NO: 31 as shown below:

| SEQ | Left | |
|---|---|---|
| ID | or | |
| NO: | Right | Sequence (5′ to 3′) |
| 18 | Left | CTCACTGAGGCCGCCCGGGCAAAGCCCGGGCGT |
| | | CGGGCGACCTTTGGTCGCCCGGCCTCAG |
| 19 | Left | CTCACTGAGGCCGCCCGGGCAAAGCCCGGGCGT |
| | | CGGGCGACCTTTGGTCGCCCGGCCTCAGTGAGC |
| | | GAGCGAGCGCGCAGAGAGGGAGTGGCCAACTCC |
| | | ATCACTAGGGGTTCCT |
| 20 | Left | CTCTCTGAGGCCGCCCGGGCAAAGCCCGGGCGT |
| | | CGGGCGACCTTTGGTCGCCCGGCCTCAG |
| 21 | Left | CTCTCTGAGGCCGCCCGGGCAAAGCCCGGGCGT |
| | | CGGGCGACCTTTGGTCGCCCGGCCTCAGTGAGC |
| | | GAGCGAGCGCGCAGAGAGGGAGTGGCCAACTCC |
| | | ATCACTAGGGGTTCCT |
| 22 | Left | CACTGAGGCCGCCCGGGCAAAGCCCGGGCGTCG |
| | | GGCGACCTTTGGTCGCCCGGCCTCAG |
| 23 | Left | CACTGAGGCCGCCCGGGCAAAGCCCGGGCGTCG |
| | | GGCGACCTTTGGTCGCCCGGCCTCAGTGAGCGA |
| | | GCGAGCGCGCAGAGAGGGAGTGGCCAACTCCAT |
| | | CACTAGGGGTTCCT |
| 24 | Left | CTCACTGAGGCCGCCCGGGCAAAGCCCGGGCGT |
| | | CGGGCGACCTTTGGTCGCCCGGCCTCAG |
| 25 | Left | CTCACTGAGGCCGCCCGGGCAAAGCCCGGGCGT |
| | | CGGGCGACCTTTGGTCGCCCGGCCTCAGTGAGC |
| | | GAGCGAGCGCGCAGAGAGGGAGTGGCCAACTCC |
| | | ATCACTAGGGGTTCCT |
| 26 | Right | CTGAGGCCGGGCGACCAAAGGTCGCCCGACGCC |
| | | CGGGCTTTGCCCGGGCGGCCTCAGTGAG |
| 27 | Right | AGGAACCCCTAGTGATGGAGTTGGCCACTCCCT |
| | | CTCTGCGCGCTCGCTCGCTCACTGAGGCCGGGC |
| | | GACCAAAGGTCGCCCGACGCCCGGGCTTTGCCC |
| | | GGGCGGCCTCAGTGAG |
| 28 | Right | CTGAGGCCGGGCGACCAAAGGTCGCCCGACGCC |
| | | CGGGCGGCCTC |
| 29 | Right | AGGAACCCCTAGTGATGGAGTTGGCCACTCCCT |
| | | CTCTGCGCGCTCGCTCGCTCACTGAGGCCGGGC |
| | | GACCAAAGGTCGCCCGACGCCCGGGCGGCCTC |
| 30 | Right | CTGAGGCCGGGCGACCAAAGGTCGCCCGACGCC |
| | | CGGGCTTTGCCCGGGCGGCCTCAGTGAG |
| 31 | Right | AGGAACCCCTAGTGATGGAGTTGGCCACTCCCT |
| | | CTCTGCGCGCTCGCTCGCTCACTGAGGCCGGGC |
| | | GACCAAAGGTCGCCCGACGCCCGGGCTTTGCCC |
| | | GGGCGGCCTCAGTGAG |

In another embodiment, provided herein is an inverted terminal repeat (ITR) nucleotide sequence comprising a sequence selected from the group consisting of: SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30 and SEQ ID NO: 31. In another embodiment, provided herein is an inverted terminal repeat (ITR) nucleotide sequence comprising a sequence at least 95%, at least 96%, at least 975, at least 98% or at least 99% identical to a sequence selected from the group consisting of: SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30 and SEQ ID NO: 31. In another embodiment, provided herein is an inverted terminal repeat (ITR) nucleotide sequence consisting of a sequence selected from the group consisting of: SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30 and SEQ ID NO: 31.

In another embodiment, the ITR nucleotide sequence further includes a spacer selected from the group consisting of SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, and SEQ ID NO: 40 as shown below:

| SEQ | Left | |
|---|---|---|
| ID | or | |
| NO: | Right | Sequence (5′ to 3′) |
| 32 | Left | TGTAGTTAATGATTAACCCGCCATGCTACTTAT |
| | | CGCGGCCGC |
| 33 | Left | TGTAGTTAATGATTAACCCGCCATGCTACTTAT |
| | | CTACGTAGCCATGCATATG |
| 34 | Left | TGTAGTTAATGATTAACCCGCCATGCTACTTAT |
| | | C |
| 35 | Left | TGTAGTTAATGATTAACCCACCATGCTACTTAT |
| | | GGCCTGCAGG |
| 36 | Left | TGTAGTTAATGATTAACCCACCATGCTACTTAT |
| | | CACCAATTG |
| 37 | Right | TCTAGAGCATGGCTACGTAGATAAGTAGCATGG |
| | | CGGGTTAATCATTAACTACACCTGCAGG |
| 38 | Right | GCTAGCCCACAATCTGCCTCCCAGTAGTACATG |
| | | ACATTAGTTTATTAATAGCCT |
| 39 | Left | TCCACAATCTGCCTCCCAGTAGTACATGACATT |
| | | AGTTTATTAATAGCCT |
| 40 | Right | CTTAAGCATGGCTACGTAGATAAGTAGCATGGC |
| | | GGGTTAATCATTAACTACACCTGCAGG |

In another embodiment, the ITR nucleotide sequence further includes a spacer sequence comprising a sequence selected from the group consisting of SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, and SEQ ID NO: 40. In another embodiment, the ITR nucleotide sequence further includes a spacer sequence comprising a sequence at least 95%, at least 96%, at least 975, at least 98% or at least 99% identical to a sequence selected from the group consisting of: SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, and SEQ ID NO: 40. In another embodiment, the ITR nucleotide sequence further includes a spacer sequence consisting of a sequence selected from the group consisting of: SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, and SEQ ID NO: 40.

In another embodiment, provided herein is a closed-ended DNA (ceDNA) vector comprising a transgene expression cassette and at least a first inverted terminal repeat (ITR) and a second ITR flanking the transgene expression cassette; wherein the first ITR and the second ITR each comprise a nucleotide sequence selected from the group consisting of the ITR nucleotide sequences disclosed herein. In another embodiment, the vector comprises double-stranded DNA.

In another embodiment, provided herein is pharmaceutical composition comprising a ceDNA vector comprising any of the ITR sequences disclosed herein and at least one pharmaceutically acceptable excipient. In another embodiment, provided herein is a lipid nanoparticle composition comprising a ceDNA vector comprising any of the ITR sequences disclosed herein. In another embodiment, provided herein is an isolated host cell comprising a ceDNA vector of any of the ITR sequences disclosed herein. In another embodiment, provided herein is a transgenic animal comprising a ceDNA vector comprising any of the ITR sequences disclosed herein.

In embodiment, provided herein is a method of treating a disorder, disease, or condition in a subject (e.g., a genetic disorder, disease, or condition), the method comprising administering to the subject a therapeutically effective amount of a ceDNA vector comprising any of the ITR sequences disclosed herein, or a pharmaceutical composition comprising a ceDNA vector comprising any of the ITR sequences disclosed herein, or a lipid nanoparticle composition comprising a ceDNA vector comprising any of the ITR sequences disclosed herein.

In another embodiment, provided herein is a method of delivering a therapeutic protein to a subject, the method comprising administering to the subject a therapeutically effective amount of a ceDNA vector comprising any of the ITR sequences disclosed herein, or a pharmaceutical composition comprising a ceDNA vector comprising any of the ITR sequences disclosed herein, or a lipid nanoparticle composition comprising a ceDNA vector comprising any of the ITR sequences disclosed herein. In another embodiment, the therapeutic protein is selected from the group consisting of an enzyme, an antibody or an antigen-binding fragment thereof, an antigen, a gene-editing protein, and a cytotoxic protein.

In one embodiment, provided herein is a DNA vector for use in synthetic production of a closed-ended DNA vector (ceDNA), comprising: a multiple cloning site capable of receiving a transgene; a first non-palindromic restriction endonuclease recognition site and a corresponding first cleavage site upstream of the multiple cloning site; a second non-palindromic restriction endonuclease recognition site and a corresponding second cleavage site downstream of the multiple cloning site; and a first partial ITR and a second partial ITR each flanking the multiple cloning site. In another embodiment, the first partial ITR is upstream of the multiple cloning site and downstream of the first non-palindromic restriction endonuclease recognition site and the corresponding first cleavage site. In another embodiment, the second partial ITR is downstream of the multiple cloning site and upstream of the second non-palindromic restriction endonuclease recognition site and the corresponding second cleavage site.

In another embodiment, the DNA vector comprises one or more spacers. In another embodiment the DNA vector comprises an origin of replication and a selectable marker gene. In another embodiment, the multiple cloning site is capable of receiving a transgene and one or more additional genetic elements selected from the group consisting of a promoter, an enhancer, an intron, a posttranscriptional regulatory element and a polyadenylation signal.

In one embodiment the first non-palindromic restriction endonuclease recognition site is specific for a first restriction endonuclease, and the second non-palindromic restriction endonuclease recognition site is specific for at least a second restriction endonuclease. In another embodiment, the first restriction endonuclease and the second restriction endonuclease are the same restriction endonuclease. In another embodiment, the first restriction endonuclease and the second restriction endonuclease are different restriction endonucleases.

In one embodiment, at least one of the restriction endonucleases is a Type IIS restriction endonuclease. In another embodiment, each of the first and second restriction endonucleases is a Type IIS restriction endonuclease. In another embodiment, the Type IIS restriction endonuclease is selected from group consisting of AcuI, AlwI, Alw26I, BasI, BbsI, BbvI, BceAI, BcgI, BCiVI, BcoDI, BruAI, BmrI, BpiI, BpuEI, BsaI, BsaXI, BseGI, BseRI, BsgI, BsmAI, BsmBI, BsmFI, BsmI, BspCNI, BspMI, BspQI, BsrDI, BsrI, BtgZI, BtsCI, BtsI, MutI, CspCI, Earl, EciI, Eco31I, Esp3I, FauI, FokI, HgaI, HphI, HpyAV, LguI, MboII, MlyI, MmeI, MnlI, Mva1269I, NmeAIII, PaqCI, PleI, SapI, SfaNI, and an isoschizomer thereof. In another embodiment, each Type IIS restriction endonuclease is independently selected from group consisting of AcuI, AlwI, Alw26I, BasI, BbsI, BbvI, BceAI, BcgI, BCVI, BcoDI, BruAI, BmrI, BpiI, BpuEI, BsaI, BsaXI, BseGI, BseRI, BsgI, BsmAI, BsmBI, BsmFI, BsmI, BspCNI, BspMI, BspQI, BsrDI, BsrI, BtgZI, BtsCI, BtsI, MutI, CspCI, Earl, EciI, Eco31I, Esp3I, FauI, FokI, HgaI, HphI, HpyAV, LguI, MboII, MlyI, MmeI, MnlI, Mva1269I, NmeAIII, PaqCI, PleI, SapI, SfaNI, and an isoschizomer thereof. In another embodiment, the Type IIS restriction endonuclease is selected from group consisting of BbsI, BsaI, Esp3I, and SapI, and an isoschizomer thereof.

In one embodiment, the DNA vector comprises a nucleotide sequence selected from the group consisting of SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; and SEQ ID NO: 17. In one embodiment, the DNA vector comprises a nucleotide sequence at least 95%, at least 96%, at least 97%, at least 98% or at least 99% identical to a sequence selected from the group consisting of SEQ ID NO: 9; SEQ ID NO: 10; SEQ ID NO:11; SEQ ID NO:12; SEQ ID NO:13; SEQ ID NO:14; SEQ ID NO:15; SEQ ID NO: 16; and SEQ ID NO: 17. In one embodiment, the DNA vector consists of a nucleotide sequence selected from the group consisting of SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; and SEQ ID NO: 17.

In one embodiment, provided herein is a kit for preparing a closed-ended DNA (ceDNA) vector comprising a transgene, the kit comprising: any DNA vector disclosed herein; at least one restriction endonuclease capable of cleaving the DNA vector at the multiple cloning site to allow the multiple cloning site to receive a transgene; at least one restriction endonuclease capable of cleaving at the first and second cleavage sites; and a ligase. In another embodiment, the kit further comprises at least one oligonucleotide comprising one or more hairpin structures.

In one embodiment, provided herein is a double-stranded circular DNA construct engineered to facilitate preparation of a closed-ended DNA (ceDNA) vector comprising a transgene expression cassette, the double-stranded circular DNA construct comprising: a transgene expression cassette; a first non-palindromic restriction endonuclease recognition site and a corresponding first cleavage site upstream of the transgene expression cassette; a second non-palindromic restriction endonuclease recognition site and a corresponding second cleavage site downstream of the transgene expression cassette; and a first partial ITR and a second partial ITR each flanking the transgene expression cassette.

In one embodiment, the first partial ITR is upstream of the transgene expression cassette and downstream of the first non-palindromic restriction endonuclease recognition site and the corresponding first cleavage site. In another embodiment, the second partial ITR is downstream of the transgene expression cassette and upstream of the second non-palindromic restriction endonuclease recognition site and the corresponding second cleavage site. In another embodiment, the first non-palindromic restriction endonuclease recognition site is specific for a first restriction endonuclease and the second non-palindromic restriction endonuclease recognition site are specific for at least a second restriction endonuclease.

In one embodiment, the first restriction endonuclease and the second restriction endonuclease are the same restriction endonuclease. In another embodiment, the first restriction endonuclease and the second restriction endonuclease are different restriction endonucleases.

In one embodiment, at least one of the restriction endonucleases is a Type IIS restriction endonuclease. In another embodiment, each of the first and second restriction endonucleases is a Type IIS restriction endonuclease. In another embodiment, the Type IIS restriction endonuclease is selected from group consisting of AcuI, AlwI, Alw26I, BasI, BbsI, BbvI, BceAI, BcgI, BCiVI, BcoDI, BruAI, BmrI, BpiI, BpuEI, BsaI, BsaXI, BseGI, BseRI, BsgI, BsmAI, BsmBI, BsmFI, BsmI, BspCNI, BspMI, BspQI, BsrDI, BsrI, BtgZI, BtsCI, BtsI, MutI, CspCI, Earl, EciI, Eco31I, Esp3I, FauI, FokI, HgaI, HphI, HpyAV, LguI, MboII, MlyI, MmeI, MnlI, Mva1269I, NmeAIII, PaqCI, PleI, SapI, SfaNI, and an isoschizomer thereof. In another embodiment, each Type IIS restriction endonuclease is independently selected from group consisting of AcuI, AlwI, Alw26I, BasI, BbsI, BbvI, BceAI, BcgI, BCVI, BcoDI, BruAI, BmrI, BpiI, BpuEI, BsaI, BsaXI, BseGI, BseRI, BsgI, BsmAI, BsmBI, BsmFI, BsmI, BspCNI, BspMI, BspQI, BsrDI, BsrI, BtgZI, BtsCI, BtsI, MutI, CspCI, Earl, EciI, Eco31I, Esp3I, FauI, FokI, HgaI, HphI, HpyAV, LguI, MboII, MlyI, MmeI, MnlI, Mva1269I, NmeAIII, PaqCI, PleI, SapI, SfaNI, and an isoschizomer thereof. In another embodiment, the at least one Type IIS restriction endonuclease is selected from group consisting of BbsI, BsaI, Esp3I, and SapI, and an isoschizomer thereof.

In one embodiment, provided herein is a kit for preparing a closed-ended DNA (ceDNA) vector comprising a transgene expression cassette, the kit comprising: any of the double-stranded DNA constructs disclosed herein; at least one restriction endonuclease capable of cleaving the double-stranded DNA construct at the first and second cleavage sites; a ligase; and instructions for use. In another embodiment, the kit further comprises at least one oligonucleotide comprising one or more hairpin structures.

In one embodiment, provided herein is a method of producing a double-stranded DNA construct from a plasmid template via rolling-circle amplification, comprising the steps of: contacting the plasmid template with a thermostable polymerase having strand-displacement activity, wherein the ratio of plasmid template concentration (in ng/μl) to polymerase concentration (in U/μl) is greater than about 1; contacting the plasmid template with an oligonucleotide primer and dNTPs; incubating the plasmid template, the polymerase, the oligonucleotide primer, and the dNTPs at a temperature of about 40° C. or less, for a time period of at least about 5 hours; thereby producing a double-stranded DNA construct.

In one embodiment, the ratio of plasmid template concentration (in ng/μl) to polymerase concentration (in U/μl) is greater than about 1, about 2, about 3, about 4, about 5, about 6, about 7, about 8, about 9, about 10, about 11, about 12, about 13, about 14, about 15, about 16, about 17, about 18, about 19, or about 20.

In one embodiment, the plasmid template concentration is about 0.01 ng/μl, about 0.05 ng/μl, about 0.1 ng/μl, about 0.15 ng/μl, about 0.2 ng/μl, about 0.21 ng/μl, about 0.22 ng/μl, about 0.23 ng/μl, about 0.24 ng/μl, about 0.2 ng/μl 5, about 0.26 ng/μl, about 0.27 ng/μl, about 0.28 ng/μl, about 0.29 ng/μl, about 0.3 ng/μl, about 0.35 ng/μl, about 0.4 ng/μl, about 0.45 ng/μl, about 0.5 ng/μl, about 0.6 ng/μl, about 0.7 ng/μl, about 0.8 ng/μl, about 0.9 ng/μl, or about 1.0 ng/μl.

In one embodiment the polymerase concentration is about 0.01 U/μl, about 0.02 U/μl, about 0.03 U/μl, about 0.04 U/μl, about 0.05 U/μl, about 0.06 U/μl, about 0.07 U/μl, about 0.08 U/μl, about 0.09 U/μl, about 0.1 U/μl, about 0.15 U/μl, about 0.2 U/μl, about 0.25 U/μl, about 0.3 U/μl, about 0.35 U/μl, about 0.4 U/μl, or about 0.45 U/μl.

In one embodiment, the temperature in step (c) is less than about 40° C., about 39° C., about 38° C., about 37° C., about 36° C., about 35° C., about 34° C., about 33° C., about 32° C., about 31° C., about 30° C., about 29° C., about 28° C., about 27° C., about 26° C., about 25° C., about 24° C., about 23° C., about 22° C., or about 21° C.

In one embodiment, the time period is at least about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 21 hours, about 33 hours, about 34 hours, about 35 hours, about 36 hours, about 37 hours, about 38 hours, about 39 hours, or about 40 hours.

In one embodiment, the time period is less than about 6 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 21 hours, about 33 hours, about 34 hours, about 35 hours, about 36 hours, about 37 hours, about 38 hours, about 39 hours, or about 40 hours.

In another embodiment the plasmid template concentration is about 0.25 ng/μl, the temperature is about 30° C., the polymerase concentration is about 0.05 U/l, and the time period is about 18-26 hours. In another embodiment, the oligonucleotide primer concentration is less than about 50 μM, at least about 10 μM, or at least about 10 μM and less than about 50 μM. 171. In another embodiment, the dNTP concentration is about 4 mM.

In one embodiment, the thermostable polymerase is Phi29 DNA polymerase or a derivative or variant thereof. In another embodiment, the thermostable polymerase is EquiPhi29™.

In one embodiment, the method is performed in a total reaction volume of at least about 100 μl. In another embodiment, the method is performed in a total reaction volume of at least about 100 μl, about 200 μl, about 300 μl, about 400 μl, about 500 μl, about 600 μl, about 700 μl, about 800 μl, about 900 μl, about 1 ml, about 2 ml, about 3 ml, about 4 ml, about 5 ml, about 6 ml, about 7 ml, about 8 ml, about 9 ml, about 10 ml, about 15 ml, about 20 ml, about 25 ml, about 30 ml, about 35 ml, about 40 ml, about 45 ml, about 50 ml, about 55 ml, about 60 ml, about 65 ml, about 70 ml, about 75 ml, about 80 ml, about 85 ml, about 90 ml, about 95 ml, about 100 ml, about 200 ml, about 300 ml, about 400 ml, about 500 ml, about 600 ml, about 700 ml, about 800 ml, about 900 ml, about 1 L, about 2 L, about 3 L, 4 L, about 5 L, about 6 L, about 7 L, about 8 L, about 9 L, about 10 L, about 20 L, about 30 L, about 40 L, about 50 L, about 60 L, about 70 L, about 80 L, about 90 L, about 100 L, about 200 L, about 300 L, about 400 L, about 500 L, about 600 L, about 700 L, about 800 L, about 900 L, or about 1000 L. In another embodiment, the method is performed in a reaction vessel that has a capacity of at least twice the total reaction volume.

In one embodiment, the oligonucleotide primer hybridizes to a backbone sequence in the plasmid template. In another embodiment, the oligonucleotide primer is a universal primer.

In one embodiment, provided herein is a double-stranded DNA construct produced by any of the methods disclosed herein.

In one embodiment, provided herein is a method of producing a closed-ended DNA (ceDNA) vector, the method comprising: producing a double-stranded DNA construct using any of the methods disclosed herein; and performing any of the methods disclosed herein to produce a ceDNA vector from the double-stranded DNA construct. In another embodiment, provided herein is a ceDNA vector produced by any of the above methods, a pharmaceutical composition comprising a ceDNA vector produced by any of the above methods and at least one pharmaceutically acceptable excipient, and a lipid nanoparticle composition comprising a vector produced by any of the above methods.

In another embodiment, provided herein is a method of preparing a closed-ended DNA (ceDNA) vector, the method comprising contacting a double-stranded DNA construct with at least one restriction endonuclease, wherein: the construct comprises: a transgene expression cassette; a first non-palindromic restriction endonuclease recognition site and a corresponding first cleavage site upstream of the transgene expression cassette, and a second non-palindromic restriction endonuclease recognition site and a corresponding second cleavage site downstream of the transgene expression cassette; and the at least one restriction endonuclease is capable of cleaving the construct at the first and second cleavage sites to release an insert having single-stranded overhangs at the 5′ and 3′ ends of the insert; and ligating the 5′ and 3′ ends of the insert to a first inverted terminal repeat (ITR) oligonucleotide and a second ITR oligonucleotide to form the ceDNA vector.

These and other aspects of the disclosure are described in further detail below.

### BRIEF DESCRIPTION OF DRAWINGS

The patent or application file contains at least one drawing executed in color. Copies of this patent or patent application publication with color drawing(s) will be provided by the Office upon request and payment of the necessary fee.

Embodiments of the present disclosure, briefly summarized above and discussed in greater detail below, can be understood by reference to the illustrative embodiments of the disclosure depicted in the appended drawings. However, the appended drawings illustrate only typical embodiments of the disclosure and are therefore not to be considered limiting of scope, for the disclosure may admit to other equally effective embodiments.

FIG. 1A and FIG. 1B show schematics of non-limiting exemplary ceDNA vectors having symmetrical or substantially symmetrical inverted terminal repeats (ITRs) or asymmetric ITRs, respectively, at both ends of the vector flanking the transgene expression cassette that includes the transgene and one or more regulatory sequences that allow and/or control the expression of the transgene.

FIG. 2 is map of an exemplary base vector Plasmid 11.

FIG. 3 is a map of an exemplary construct, namely Construct 1, generated by sub-cloning a Factor VIII (VIII)-expressing transgene into Plasmid 11.

FIG. 4 is a schematic representation of an exemplary cell-free synthesis of ceDNA using a single ITR oligonucleotide and the restriction endonuclease BsaI.

FIG. 5 is a schematic comparing the activity of conventional restriction endonucleases such as EcoRI and Type IIS restriction endonucleases, specifically how the enzymes recognize and cleave nucleotide sequences on a substrate DNA and the properties of the overhangs generated from the cleavage.

FIG. 6 is a schematic illustrating the mechanism of BsaI recognizing and cleaving a double-stranded DNA construct whereby only the fragment carrying the transgene expression cassette and having the appropriate overhang would ligate with ITR oligo 1 having the complementary overhang.

FIG. 7A is a schematic representation of exemplary ITR oligonucleotides that self-anneal to form the three-dimensional stem-loop structure, where the stem length is 7 bp or 3 bp. FIG. 7B is an agarose gel image analyzing the ligation reactions using Plasmid 20 insert and ITR oligonucleotides having different stem lengths: 14 bp, 7 bp, 5 bp, and 3 bp. FIG. 7C is a map of an exemplary base vector Plasmid 20.

FIG. 8 is a schematic illustrating the mechanism of generating a ceDNA vector having asymmetric ITRs.

FIG. 9A is an agarose gel image analyzing the multiple ligation reactions using different combinations of the base vector, ITR oligonucleotides, and Type IIS restriction endonuclease. FIG. 9B and FIG. 9C are agarose gel images confirming the ligation specificity at both the 5′ and 3′ ends of the insert using labeled ITR oligonucleotides.

FIG. 10 is a schematic showing the stem-loop structure of a wild-type ITR of AAV serotype 2 (AAV2) (SEQ ID NO: 52 of International Patent Application Publication No. WO2019/143885, the entire contents of which are expressly incorporated herein by reference) with the identification of the A-A′ and D-D′ stem regions and B-B′ and C-C′ loops.

FIG. 11 is an agarose gel image analyzing the uncut Construct 1 (Lane 1), digestion/ligation reaction of Construct 1 (Lane 2), and the reaction mixture after exonuclease digestion (Lane 3).

FIG. 12 illustrates: (i) the predicted sizes of FVIII-ceDNA produced from Construct 1, uncut or cut with BglII, as a closed-ended vector, as open-ended stranded DNA, or as a closed-ended vector containing one or more nicks or gaps; and (ii) denaturing gel analysis of FVIII-ceDNA, uncut or cut with BglII.

FIG. 13 is a chromatogram of FVIII-ceDNA drug substance eluting as a single, sharp peak from an ion-exchange chromatography column.

FIG. 14 is a schematic illustrating DNA sequence analysis comparing the DNA sequences of FVIII-ceDNA at the 5′ and 3′ ligation junctions against the DNA sequences of the ITR oligo 1 and Construct 1, thereby revealing unique junction sequences that are present only in the FVIII-ceDNA ligation product but not in ITR oligo 1 and Construct 1.

FIG. 15 is an agarose gel image showing the ceDNA generated using the cell-free synthetic method described herein, at small and medium scales.

FIG. 16A is a graph showing the in vivo FVIII-expressing levels of synthetic and Sf9-produced FVIII-ceDNA at increasing dose levels in hydrodynamic tail vein injection studies with mice. FIG. 16B is a graph showing the in vivo FVIII-expressing levels of synthetic and Sf9-produced FVIII-ceDNA formulated as lipid nanoparticle compositions in a 42-day intravenous injection study with mice.

FIG. 17 is a schematic depiction of rolling-circle plasmid amplification using primer-driven multiple strand displacement, followed by enzymatic conversion of the amplified product into ceDNA, both with (bottom) and without (top) an optional intermediate digestion step.

FIG. 18 depicts the evaluation of the effects of primer concentration on amplified plasmid product quality and DNA yield using agarose gel analysis of DNA banding profiles of an E. coli plasmid template and amplified plasmid. Lanes, in order from left to right: (1) size markers; (2) plasmid, no BsaI digestion; (3) plasmid, BsaI digestion; (4) amplified plasmid, BsaI digestion, 500 μM primer; (5) amplified plasmid, BsaI digestion, 100 μM primer; (6) amplified plasmid, BsaI digestion, 50 μM primer; (7) amplified plasmid, BsaI digestion, 10 μM primer; (8) amplified plasmid, BsaI digestion, 5 μM primer; (9) amplified plasmid, BsaI digestion, 1 μM primer; (10) amplified plasmid, BsaI digestion, 0 μM primer.

FIG. 19 depicts the effects of temperature and polymerase amount on amplified product quality. FIG. 19A shows agarose gel analysis of DNA banding and product quality for BsaI-treated amplified plasmid at different amplification temperatures, amounts of polymerase enzyme, and length of amplification time. For each amplification reaction temperature (from left to right: 40° C., 37° C., 33° C., 30° C., and 20° C.) results are shown for 50 Units EquiPhi29™ (top) and 5 Units EquiPhi39™ (bottom). Additionally, for each reaction temperature, the four lanes, from left to right, show results for amplification reaction times of 3 hours, 12 hours, 24 hours, and 36 hours. The left-most lane contains size markers. FIG. 19B shows an agarose gel comparing the results of amplification at 30° C. with different amounts of wild-type Phi29 polymerase enzyme (left) and engineered EquiPhi29™ polymerase enzyme (right). Lanes, for each gel, from left to right: (1) size markers; (2) 2 Units of enzyme; (3) 5 Units of enzyme.

FIG. 20A depicts an agarose gel comparison of BsaI-digested E. coli plasmid, plasmid amplified at 30° C., and plasmid amplified at 45° C. (left) and the corresponding ceDNA vectors produced from both E. coli plasmid and each amplified plasmid (right). Lanes, from left to right: (1) size markers; (2) BsaI-digested plasmid; (3) BsaI-digested 30° C.-amplified plasmid; (4) BsaI-digested 45° C.-amplified plasmid; (5) size markers; (6) purified ceDNA produced from plasmid; (7) purified ceDNA produced from 30° C.-amplified plasmid; (8) purified ceDNA produced from 45° C.-amplified plasmid; (9) size markers. FIG. 20B depicts an agarose gel comparison of ceDNA produced from five different amplified plasmid constructs. The expected ceDNA product sizes are, from left to right: 2.9 kb (construct 1); 3.8 kb (construct 2); 5.9 kb (construct 3); 4.7 kb (construct 4); and 6.2 kb (construct 5). The left-most lane contains size markers.

FIG. 21 depicts scaled plasmid amplification with increasing reaction volumes. FIG. 21A shows agarose gel analysis of BsaI-digested amplified plasmid products, along with the quantified amplified yield of DNA. Lanes, from left to right: (1) 100 μl volume, 1.5 mL tube: 25 ng plasmid input, 80 μg amplified DNA yield; (2) 1 mL volume, 1.5 mL tube: 250 ng plasmid input, 800 μg amplified DNA yield; (3) 25 mL volume, 50 mL tube: 6.3 ng plasmid input, 20 mg amplified DNA yield; (4) size markers. FIG. 21B depicts a fragment chromatogram and agarose gel analysis of 20 mg ceDNA product from 25 mL reaction volume (corresponding to lane 3 in FIG. 21A).

FIG. 22 depicts agarose gel analysis comparing plasmid amplification using different amounts of polymerase enzyme, reaction temperatures, and reaction time lengths. Shown are results for BsaI digestion of the crude reaction (left) and Zymo-purified ceDNA (right). Initial process: 0.25 ng/μl plasmid template, 10 μM annealing primer, 45° C., 0.5 U/μl EquiPhi29™, 5 mM dNTPs, 3 hours. Updated process: 0.25 ng/μl plasmid template, 25 μM annealing primer, 30° C., 0.05 U/μl EquiPhi29™, 4 mM dNTPs, 18-26 hours. Lanes, left to right: (1) size markers; (2) BsaI-digested plasmid; (3) BsaI-digested crude amplified plasmid, updated process; (4) BsaI-digested crude amplified plasmid, initial process; (5) size markers; (6) purified ceDNA produced from plasmid; (7) purified ceDNA produced from amplified plasmid, updated process; (8) purified plasmid produced from amplified plasmid, initial process; (9) size markers.

### DETAILED DESCRIPTION

The present disclosure describes the development of a new approach for more rapid and cost-effective plasmid or DNA production using cell-free DNA amplification methods. The methods and compositions provided herein are based, at least in part, on the discovery of a cell-free synthetic production method useful for generating DNA vectors, including but not limited to closed-ended DNA (ceDNA) vectors that have high purity and yield as compared to DNA vectors produced in an insect cell line, such as the Sf9 cell line, and/or where the production process is streamlined or made more efficient or cost-effective relative to traditional cell-based production methods. For example, in one embodiment, the high specificity in the restriction endonuclease digestion and ligation reactions allow both reactions to run simultaneously in a single reaction vessel. In some embodiments, the high specificity in the digestion and ligation reactions further eliminates the need for a purification procedure between the two reactions. This high specificity in the restriction endonuclease digestion and ligation reactions is facilitated by the design in the base materials that make the closed-ended vectors, i.e., the inverted terminal repeat (ITR) oligonucleotides and double-stranded DNA construct. Specifically, the ITR oligonucleotides and double-stranded DNA construct contain nucleotide sequences that leverage the unique activity of the restriction endonucleases used in these cell-free synthetic methods in recognizing, binding and cleaving DNA.

The results of the work described in embodiments herein are a scalable, robust, cell-free, enzymatic method that generated large quantities of completely synthetic closed-ended DNA molecules, representing a significant advantage over cell-based production methods.

Furthermore, leveraging the unique activity of the restriction endonucleases used in these cell-free synthetic methods allows the directionality of ligation reactions that utilize more than one ITR oligonucleotides to be controlled, thereby enabling preparation of ceDNA having asymmetric ITRs.

Another significant advantage offered by the cell-free synthetic methods provided herein over cell-based production methods is that, in addition to the higher yield, the methods described herein are readily scalable small reactions (˜1 mL) and up to at least medium (>40 mL) and further without compromising the purity.

The vectors synthesized as described herein can express any desired transgene, for example, a transgene to treat or cure a given disease. One of ordinary skill in the art will readily recognize that any transgene used in conventional gene therapy methods with conventional recombinant vectors can be adapted for expression by e.g., ceDNA vectors made by the synthetic methods described herein.

### I. Definitions

Unless otherwise defined herein, scientific and technical terms used in connection with the present application shall have the meanings that are commonly understood by those of ordinary skill in the art to which this disclosure belongs. It should be understood that this disclosure is not limited to the particular methodology, protocols, and reagents, etc., described herein and as such can vary. The terminology used herein is for the purpose of describing particular embodiments only, and is not intended to limit the scope of the present disclosure, which is defined solely by the claims. Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al., (eds.), Fields Virology, 6^{th} Edition, published by Lippincott Williams & Wilkins, Philadelphia, PA, USA (2013), Knipe, D. M. and Howley, P. M. (ed.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737).

As used herein, the terms “cell-free,” “cell-free synthesis,” “cell-free production,” “synthetic closed-ended DNA vector production” and “synthetic production” and all other related counterparts are used interchangeably and refer to the production of one or more molecules in a manner that does not involve replication or other multiplication of the molecule by or inside of a cell or using a cellular extract. Synthetic production avoids contamination of the produced molecule with cellular contaminants (e.g., cellular proteins or cellular nucleic acids) and further avoids unwanted cellular-specific modification of the molecule during the production process (e.g., methylation or glycosylation or other post-translational modification).

As used herein, the terms “heterologous nucleotide sequence” and “transgene” are used interchangeably and refer to a nucleic acid of interest (other than a nucleic acid encoding a capsid polypeptide) that is incorporated into and may be delivered and expressed by a ceDNA vector as disclosed herein.

As used herein, the terms “transgene expression cassette,” “expression cassette,” “transcription cassette” and “gene expression unit” are used interchangeably and refer to a linear stretch of nucleic acids that includes a transgene that is operably linked to one or more promoters or other regulatory sequences sufficient to direct transcription of the transgene, but which does not comprise capsid-encoding sequences, other vector sequences or inverted terminal repeat regions. An expression cassette may additionally comprise one or more regulatory genetic elements including cis-acting sequences (e.g., promoters, enhancers, or repressors), one or more introns, one or more polyadenylation signals and one or more post-transcriptional regulatory elements such as a WHP post-transcriptional regulatory element (WPRE).

The terms “polynucleotide” and “nucleic acid,” used interchangeably herein, refer to a polymeric form of nucleotides of any length, either ribonucleotides or deoxyribonucleotides. Thus, this term includes single, double, or multi-stranded DNA or RNA, genomic DNA, cDNA, DNA-RNA hybrids, or a polymer including purine and pyrimidine bases or other natural, chemically or biochemically modified, non-natural, or derivatized nucleotide bases. “Oligonucleotide” generally refers to polynucleotides of between about 5 and about 100 nucleotides of single- or double-stranded DNA. However, for the purposes of this disclosure, there is no upper limit to the length of an oligonucleotide. Oligonucleotides are also known as “oligomers” or “oligos” and may be isolated from genes, or chemically synthesized by methods known in the art. “Inverted terminal repeat oligonucleotides” or “ITR oligonucleotides” as used herein refer to single-stranded oligonucleotides containing at least partial sequences of a full ITR as defined herein, and are capable of self-annealing to form a three-dimensional configuration of an ITR having a hairpin structure or a T- or Y-shaped stem-loop structure. The terms “polynucleotide” and “nucleic acid” should be understood to include, as applicable to the embodiments being described, single-stranded (such as sense or antisense) and double-stranded polynucleotides.

The term “nucleic acid construct” as used herein refers to a nucleic acid molecule (e.g., DNA construct”, either single- or double-stranded, which is isolated from a naturally occurring gene or which is modified to contain segments of nucleic acids in a manner that would not otherwise exist in nature or which is synthetic.

By “hybridizable” or “complementary” or “substantially complementary” it is meant that a nucleic acid (e.g., single-stranded DNA) includes a sequence of nucleotides that enables it to non-covalently bind, i.e. form Watson-Crick base pairs and/or G/U base pairs, “anneal”, or “hybridize,” to another nucleic acid (e.g., another single-stranded DNA) in a sequence-specific, antiparallel, manner (i.e., a nucleic acid specifically binds to a complementary nucleic acid) under the appropriate in vitro and/or in vivo conditions of temperature and solution ionic strength. As is known in the art, standard Watson-Crick base-pairing includes: adenine (A) pairing with thymidine (T), adenine (A) pairing with uracil (U), and guanine (G) pairing with cytosine (C).

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones. In some embodiments, the transgene in the transgene expression cassette as defined encodes a therapeutic protein. In some embodiments, the therapeutic protein is selected from an enzyme, a coagulation factor or co-factor, an antibody or an antigen-binding fragment thereof, an antigen, a gene-editing protein, and a cytotoxic protein.

A DNA sequence, such as a transgene, that “encodes” a particular RNA or protein gene product is a DNA nucleic acid sequence that is transcribed into the particular RNA and/or protein. A DNA polynucleotide may encode an RNA (mRNA) that is translated into protein, or a DNA polynucleotide may encode an RNA that is not translated into protein (e.g., tRNA, rRNA, or a DNA-targeting RNA; also called “non-coding” RNA or “ncRNA”).

As used herein, the term “gene delivery” means a process by which foreign DNA is transferred to host cells for applications of gene therapy.

As used herein, the term “terminal repeat” or “TR” may include any viral terminal repeat or synthetic sequence that comprises at least one minimal required origin of replication and a region comprising a palindromic hairpin structure. A Rep-binding sequence (“RBS”) (also referred to as RBE (Rep-binding element)) and a terminal resolution site (“TRS”) together constitute a “minimal required origin of replication” and thus the TR comprises at least one RBS and at least one TRS. TRs that are the inverse complement of one another within a given stretch of polynucleotide sequence are typically each referred to as an “inverted terminal repeat” or “ITR”. In the context of a virus, ITRs mediate replication, virus packaging, integration and provirus rescue. As was unexpectedly found in the disclosure herein, TRs that are not inverse complements across their full length can still perform the traditional functions of ITRs, and thus the term ITR is used herein to refer to a TR in a ceDNA genome or ceDNA vector that is capable of mediating replication of ceDNA vector. It will be understood by one of ordinary skill in the art that in complex ceDNA vector configurations more than two ITRs or asymmetric ITR pairs may be present. The ITR can be an AAV ITR or a non-AAV ITR, or can be derived from an AAV ITR or a non-AAV ITR. For example, the ITR can be derived from the family Parvoviridae, which encompasses parvoviruses and dependoviruses (e.g., canine parvovirus, bovine parvovirus, mouse parvovirus, porcine parvovirus, human parvovirus B-19), or the SV40 hairpin that serves as the origin of SV40 replication can be used as an ITR, which can further be modified by truncation, substitution, deletion, insertion and/or addition. Parvoviridae family viruses consist of two subfamilies: Parvovirinae, which infect vertebrates, and Densovirinae, which infect invertebrates. Dependoparvoviruses include the viral family of the adeno-associated viruses (AAV) which are capable of replication in vertebrate hosts including, but not limited to, human, primate, bovine, canine, equine and ovine species. For convenience herein, an ITR located 5′ to (upstream of) an expression cassette in a ceDNA vector is referred to as a “5′ ITR” or a “left ITR”, and an ITR located 3′ to (downstream of) an expression cassette in a ceDNA vector is referred to as a “3′ ITR” or a “right ITR”.

A “wild-type ITR” or “WT-ITR” refers to the sequence of a naturally occurring ITR sequence in an AAV or other dependovirus that retains, e.g., Rep binding activity and Rep nicking ability. The nucleotide sequence of a WT-ITR from any AAV serotype may slightly vary from the canonical naturally occurring sequence due to degeneracy of the genetic code or drift, and therefore WT-ITR sequences encompassed for use herein include WT-ITR sequences as result of naturally occurring changes taking place during the production process (e.g., a replication error).

As used herein, the term “substantially symmetric WT-ITRs” or a “substantially symmetrical WT-ITRs” refers to a pair of WT-ITRs within a single ceDNA genome or ceDNA vector that are both wild type ITRs that have an inverse complement sequence across their entire length. For example, an ITR can be considered to be a wild-type sequence, even if it has one or more nucleotides that deviate from the canonical naturally occurring sequence, so long as the changes do not affect the properties and overall three-dimensional structure of the sequence. In some aspects, the deviating nucleotides represent conservative sequence changes. As one non-limiting example, a sequence that has at least 95%, 96%, 97%, 98%, or 99% sequence identity to the canonical sequence (as measured, e.g., using BLAST at default settings), and also has a symmetrical three-dimensional spatial organization to the other WT-ITR such that their 3D structures are the same shape in geometrical space. The substantially symmetrical WT-ITR has the same A, C-C′ and B-B′ loops in 3D space. A substantially symmetrical WT-ITR can be functionally confirmed as WT by determining that it has an operable Rep binding site (RBE or RBE′) and terminal resolution site (trs) that pairs with the appropriate Rep protein. One can optionally test other functions, including transgene expression under permissive conditions.

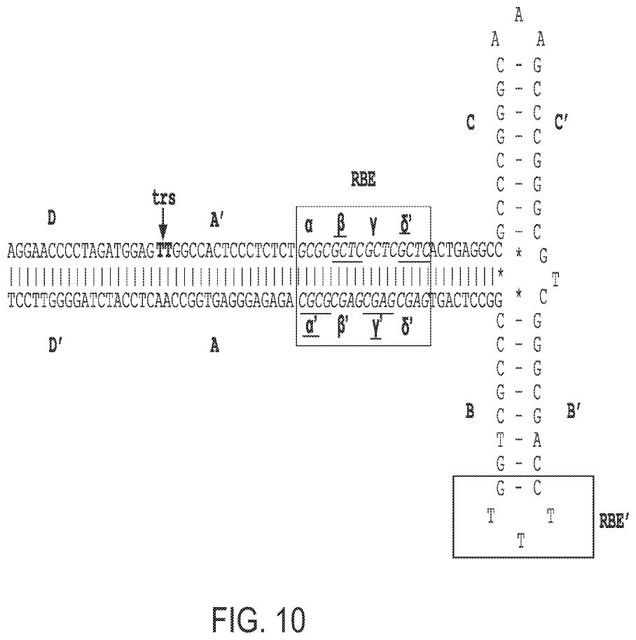

As used herein, the terms “modified ITR,” or “mod-ITR,” or “mutant ITR” are used interchangeably herein and refer to an ITR that has a mutation in at least one or more nucleotides as compared to the WT-ITR from the same serotype. The mutation can result in a change in one or more of A-A′ and D-D′ stem regions and B-B′ and C-C′ loops in the ITR (see FIG. 10), and can result in a change in the three-dimensional spatial organization (i.e. its 3D structure in geometric space) as compared to the 3D spatial organization of a WT-ITR of the same serotype.

As used herein, the term “asymmetric ITRs” also referred to as “asymmetric ITR pairs” refers to a pair of ITRs within a single ceDNA genome or ceDNA vector that are not inverse complements across their full length. As one non-limiting example, an asymmetric ITR pair does not have a symmetrical three-dimensional spatial organization to their cognate ITR such that their 3D structures are different shapes in geometrical space. Stated differently, an asymmetric ITR pair have the different overall geometric structure, i.e., they have different organization of their A-A′ and D-D′ stem regions and B-B′ and C-C′ loops in 3D space (e.g., one ITR may have a short C-C′ loop and/or short B-B′ loop as compared to the cognate ITR). The difference in sequence between the two ITRs may be due to one or more nucleotide addition, deletion, truncation, or point mutation. In one embodiment, one ITR of the asymmetric ITR pair may be a wild-type AAV ITR and the other ITR a modified ITR as defined herein (e.g., a non-wild-type or synthetic ITR). In another embodiment, neither ITRs of the asymmetric ITR pair is a wild-type AAV ITR and the two ITRs are modified ITRs that have different shapes in geometrical space (i.e., a different overall geometric structure). In some embodiments, one mod-ITRs of an asymmetric ITR pair can have a short C-C′ loop and the other ITR can have a different modification (e.g., a single loop, or a short B-B′ loop etc.) such that they have different three-dimensional spatial organization as compared to the cognate asymmetric mod-ITR.

As used herein, the term “symmetric ITRs” refers to a pair of ITRs within a single ceDNA genome or ceDNA vector that are mutated or modified relative to wild-type dependoviral ITR sequences and are inverse complements across their full length. Neither ITRs are wild-type ITR (i.e., they are a modified ITR, also referred to as a mutant ITR), and can have a difference in sequence from the wild-type ITR due to nucleotide addition, deletion, substitution, truncation, or point mutation. For convenience herein, an ITR located 5′ to (upstream of) an expression cassette in a ceDNA vector is referred to as a “5′ ITR” or a “left ITR”, and an ITR located 3′ to (downstream of) an expression cassette in a ceDNA vector is referred to as a “3′ ITR” or a “right ITR”.

As used herein, the terms “substantially symmetrical modified-ITRs” or a “substantially symmetrical mod-ITR pair” refers to a pair of modified-ITRs within a single ceDNA genome or ceDNA vector that are both that have an inverse complement sequence across their entire length. For example, a modified ITR can be considered substantially symmetrical, even if it has some nucleotide sequences that deviate from the inverse complement sequence so long as the changes do not affect the properties and overall shape. As one non-limiting example, a sequence that has at least 85%, 90%, 95%, 96%, 97%, 98%, or 99% sequence identity to the canonical sequence (as measured using BLAST at default settings), and also has a symmetrical three-dimensional spatial organization to their cognate modified ITR such that their 3D structures are the same shape in geometrical space. Stated differently, a substantially symmetrical modified-ITR pair have the same A-A′ and D-D′ stem regions and B-B′ and C-C′ loops organized in 3D space. In some embodiments, the ITRs from a mod-ITR pair may have different reverse complement nucleotide sequences but still have the same symmetrical three-dimensional spatial organization—that is both ITRs have mutations that result in the same overall 3D shape. For example, one ITR (e.g., 5′ ITR) in a mod-ITR pair can be from one serotype, and the other ITR (e.g., 3′ ITR) can be from a different serotype, however, both can have the same corresponding mutation (e.g., if the 5′ITR has a deletion in the C region, the cognate modified 3′ITR from a different serotype has a deletion at the corresponding position in the C′ region), such that the modified ITR pair has the same symmetrical three-dimensional spatial organization. In such embodiments, each ITR in a modified ITR pair can be from different serotypes (e.g. AAV1, 2, 3, 4, 5, 6, 7, 8, 9, 10, 11, and 12) such as the combination of AAV2 and AAV6, with the modification in one ITR reflected in the corresponding position in the cognate ITR from a different serotype. In one embodiment, a substantially symmetrical modified ITR pair refers to a pair of modified ITRs (mod-ITRs) so long as the difference in nucleotide sequences between the ITRs does not affect the properties or overall shape and they have substantially the same shape in 3D space. As a non-limiting example, a mod-ITR that has at least 95%, 96%, 97%, 98% or 99% sequence identity to the canonical mod-ITR as determined by standard means well known in the art such as BLAST (Basic Local Alignment Search Tool), or BLASTN at default settings, and also has a symmetrical three-dimensional spatial organization such that their 3D structure is the same shape in geometric space. A substantially symmetrical mod-ITR pair has the same A-A′ and D-D′ stem regions and B-B′ and C-C′ loops in 3D space, e.g., if a modified ITR in a substantially symmetrical mod-ITR pair has a deletion of a C-C′ arm, then the cognate mod-ITR has the corresponding deletion of the C-C′ loop and also has a similar 3D structure of the remaining A-A stem region and B-B′ loop in the same shape in geometric space of its cognate mod-ITR.

The term “flanking” refers to a relative position of one nucleic acid sequence with respect to another nucleic acid sequence. Generally, in the sequence ABC, B is flanked by A and C. The same is true for the arrangement A×B×C. Thus, a flanking sequence precedes or follows a flanked sequence but need not be contiguous with, or immediately adjacent to the flanked sequence. In one embodiment, the term flanking refers to terminal repeats at each end of the linear duplex ceDNA vector.

As used herein, the term “ceDNA genome” refers to an expression cassette that further incorporates at least one inverted terminal repeat region. A ceDNA genome may further comprise one or more spacer regions. In some embodiments the ceDNA genome is incorporated as an intermolecular duplex polynucleotide of DNA into a plasmid or viral genome.

As used herein, the term “ceDNA spacer region” refers to an intervening sequence that separates functional elements in the ceDNA vector or ceDNA genome. In some embodiments, ceDNA spacer regions keep two functional elements at a desired distance for optimal functionality. In some embodiments, ceDNA spacer regions provide or add to the genetic stability of the ceDNA genome within e.g., a plasmid or baculovirus. In some embodiments, ceDNA spacer regions facilitate ready genetic manipulation of the ceDNA genome by providing a convenient location for cloning sites and the like. For example, in certain aspects, an oligonucleotide “polylinker” containing several restriction endonuclease sites, or a non-open reading frame sequence designed to have no known protein (e.g., transcription factor) binding sites can be positioned in the ceDNA genome to separate the cis-acting factors, e.g., inserting a 6mer, 12mer, 18mer, 24mer, 48mer, 86mer, 176mer, etc. between the terminal resolution site and the upstream transcriptional regulatory element. Similarly, the spacer may be incorporated between the polyadenylation signal sequence and the 3′-terminal resolution site.

As used herein, the terms “Rep binding site, “Rep binding element, “RBE” and “RBS” are used interchangeably and refer to a binding site for Rep protein (e.g., AAV Rep 78 or AAV Rep 68) which upon binding by a Rep protein permits the Rep protein to perform its site-specific endonuclease activity on the sequence incorporating the RBS. An RBS sequence and its inverse complement together form a single RBS. RBS sequences are known in the art, and include, for example, SEQ ID NO: 60 of International Patent Application Publication No. WO2019/143885), an RBS sequence identified in AAV2. Any known RBS sequence may be used in the embodiments of the disclosure, including other known AAV RBS sequences and other naturally known or synthetic RBS sequences. Without being bound by theory it is thought that he nuclease domain of a Rep protein binds to the duplex nucleotide sequence GCTC (see SEQ ID NO: 60 of WO2019/143885), and thus the two known AAV Rep proteins bind directly to and stably assemble on the duplex oligonucleotide. In addition, soluble aggregated conformers (i.e., undefined number of inter-associated Rep proteins) dissociate and bind to oligonucleotides that contain Rep binding sites. Each Rep protein interacts with both the nitrogenous bases and phosphodiester backbone on each strand. The interactions with the nitrogenous bases provide sequence specificity whereas the interactions with the phosphodiester backbone are non- or less-sequence specific and stabilize the protein-DNA complex.

As used herein, the terms “terminal resolution site” and “TRS” are used interchangeably herein and refer to a region at which Rep forms a tyrosine-phosphodiester bond with the 5′ thymidine generating a 3′ OH that serves as a substrate for DNA extension via a cellular DNA polymerase, e.g., DNA pol delta or DNA pol epsilon. Alternatively, the Rep-thymidine complex may participate in a coordinated ligation reaction. In some embodiments, a TRS minimally encompasses a non-base-paired thymidine. In some embodiments, the nicking efficiency of the TRS can be controlled at least in part by its distance within the same molecule from the RBS. When the acceptor substrate is the complementary ITR, then the resulting product is an intramolecular duplex. TRS sequences are known in the art, and include, for example, 5′-GGTTGA-3′ (SEQ ID NO: 61 of WO2019/143885), the hexanucleotide sequence identified in AAV2. Any known TRS sequence may be used in the embodiments of the disclosure, including other known AAV TRS sequences and other naturally known or synthetic TRS sequences such as AGTT, GGTTGG (SEQ ID NO: 63 of WO2019/143885), AGTTGG (SEQ ID NO: 64 of WO2019/143885), AGTTGA (SEQ ID NO: 65 of WO2019/143885), and other motifs such as RRTTRR (SEQ ID NO: 66 of WO2019/143885).

As used herein, the term “ceDNA-plasmid” refers to a plasmid that comprises a ceDNA genome as an intermolecular duplex.

As used herein, the term “ceDNA-bacmid” refers to an infectious baculovirus genome comprising a ceDNA genome as an intermolecular duplex that is capable of propagating in E. coli as a plasmid, and so can operate as a shuttle vector for baculovirus.

As used herein, the term “ceDNA-baculovirus” refers to a baculovirus that comprises a ceDNA genome as an intermolecular duplex within the baculovirus genome.

As used herein, the terms “ceDNA-baculovirus infected insect cell” and “ceDNA-BIIC” are used interchangeably, and refer to an invertebrate host cell (including, but not limited to an insect cell (e.g., an Sf9 cell)) infected with a ceDNA-baculovirus.

As used herein, the term “closed-ended DNA vector” refers to a capsid-free DNA vector with at least one covalently closed end and where at least part of the vector has an intramolecular duplex structure.

As used herein, the terms “ceDNA vector” and “ceDNA” are used interchangeably and refer to a closed-ended DNA vector comprising at least one terminal covalently-closed end. In some embodiments, the ceDNA comprises two covalently-closed ends.

As defined herein, “reporters” refer to proteins that can be used to provide detectable read-outs. Reporters generally produce a measurable signal such as fluorescence, color, or luminescence. Reporter protein coding sequences encode proteins whose presence in the cell or organism is readily observed. For example, fluorescent proteins cause a cell to fluoresce when excited with light of a particular wavelength, luciferases cause a cell to catalyze a reaction that produces light, and enzymes such as β-galactosidase convert a substrate to a colored product. Exemplary reporter polypeptides useful for experimental or diagnostic purposes include, but are not limited to β-lactamase, β-galactosidase (LacZ), alkaline phosphatase (AP), thymidine kinase (TK), green fluorescent protein (GFP) and other fluorescent proteins, chloramphenicol acetyltransferase (CAT), luciferase, and others well known in the art.

As used herein, the term “effector protein” refers to a polypeptide that provides a detectable read-out, either as, for example, a reporter polypeptide, or more appropriately, as a polypeptide that kills a cell, e.g., a toxin, or an agent that renders a cell susceptible to killing with a chosen agent or lack thereof. Effector proteins include any protein or peptide that directly targets or damages the host cell's DNA and/or RNA. For example, effector proteins can include, but are not limited to, a restriction endonuclease that targets a host cell DNA sequence (whether genomic or on an extrachromosomal element), a protease that degrades a polypeptide target necessary for cell survival, a DNA gyrase inhibitor, and a ribonuclease-type toxin. In some embodiments, the expression of an effector protein controlled by a synthetic biological circuit as described herein can participate as a factor in another synthetic biological circuit to thereby expand the range and complexity of a biological circuit system's responsiveness.

Transcriptional regulators refer to transcriptional activators and repressors, including induce and repressor proteins, that either activate or repress transcription of a gene of interest. Promoters are regions of nucleic acid that initiate transcription of a particular gene Transcriptional activators typically bind nearby to transcriptional promoters and recruit RNA polymerase to directly initiate transcription. Repressors bind to transcriptional promoters and sterically hinder transcriptional initiation by RNA polymerase. Other transcriptional regulators may serve as either an activator or a repressor depending on where they bind and cellular and environmental conditions. Non-limiting examples of transcriptional regulator classes include, but are not limited to homeodomain proteins, zinc-finger proteins, winged-helix (forkhead) proteins, and leucine-zipper proteins.

As used herein, “carrier” or “excipient includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutically active substances is well known in the art. Supplementary active ingredients can also be incorporated into the compositions. The phrase “pharmaceutically-acceptable” refers to molecular entities and compositions that do not produce a toxic, an allergic, or similar untoward reaction when administered to a host.

The term “in vivo” refers to assays or processes that occur in or within an organism, such as a multicellular animal. In some of the aspects described herein, a method or use can be said to occur “in vivo” when a unicellular organism, such as a bacterium, is used. The term “ex vivo” refers to methods and uses that are performed using a living cell with an intact membrane that is outside of the body of a multicellular animal or plant, e.g., explants, cultured cells, including primary cells and cell lines, transformed cell lines, and extracted tissue or cells, including blood cells, among others. The term “in vitro” refers to assays and methods that do not require the presence of a cell with an intact membrane, such as cellular extracts, and can refer to the introducing of a programmable synthetic biological circuit in a non-cellular system, such as a medium not comprising cells or cellular systems, such as cellular extracts.

The term “promoter,” as used herein, refers to any nucleic acid sequence that regulates the expression of another nucleic acid sequence by driving transcription of the nucleic acid sequence, which can be a heterologous target gene encoding a protein or an RNA. Promoters can be constitutive, inducible, repressible, tissue-specific, or any combination thereof. A promoter is a control region of a nucleic acid sequence at which initiation and rate of transcription of the remainder of a nucleic acid sequence are controlled. A promoter can also contain genetic elements at which regulatory proteins and molecules can bind, such as RNA polymerase and other transcription factors. In some embodiments of the aspects described herein, a promoter can drive the expression of a transcription factor that regulates the expression of the promoter itself. Within the promoter sequence will be found a transcription initiation site, as well as protein binding domains responsible for the binding of RNA polymerase. Eukaryotic promoters will often, but not always, contain “TATA” boxes and “CAT” boxes. Various promoters, including inducible promoters, may be used to drive the expression of transgenes in the ceDNA vectors disclosed herein. A promoter sequence may be bounded at its 3′ terminus by the transcription initiation site and extends upstream (5′ direction) to include the minimum number of bases or elements necessary to initiate transcription at levels detectable above background.

The term “enhancer” as used herein refers to a cis-acting regulatory sequence (e.g., 50-1,500 base pairs) that binds one or more proteins (e.g., activator proteins, or transcription factor) to increase transcriptional activation of a nucleic acid sequence. Enhancers can be positioned up to 1,000,000 base pars upstream of the gene start site or downstream of the gene start site that they regulate. An enhancer can be positioned within an intronic region, or in the exonic region of an unrelated gene.

A promoter can be said to drive expression or drive transcription of the nucleic acid sequence that it regulates. The phrases “operably linked,” “operatively positioned,” “operatively linked,” “under control,” and “under transcriptional control” indicate that a promoter is in a correct functional location and/or orientation in relation to a nucleic acid sequence it regulates to control transcriptional initiation and/or expression of that sequence. An “inverted promoter,” as used herein, refers to a promoter in which the nucleic acid sequence is in the reverse orientation, such that what was the coding strand is now the non-coding strand, and vice versa. Inverted promoter sequences can be used in various embodiments to regulate the state of a switch. In addition, in various embodiments, a promoter can be used in conjunction with an enhancer.

A promoter can be one naturally associated with a gene or sequence, as can be obtained by isolating the 5′ non-coding sequences located upstream of the coding segment and/or exon of a given gene or sequence. Such a promoter can be referred to as “endogenous.” Similarly, in some embodiments, an enhancer can be one naturally associated with a nucleic acid sequence, located either downstream or upstream of that sequence.

In some embodiments, a coding nucleic acid segment is positioned under the control of a “recombinant promoter” or “heterologous promoter,” both of which refer to a promoter that is not normally associated with the encoded nucleic acid sequence it is operably linked to in its natural environment. A recombinant or heterologous enhancer refers to an enhancer not normally associated with a given nucleic acid sequence in its natural environment. Such promoters or enhancers can include promoters or enhancers of other genes; promoters or enhancers isolated from any other prokaryotic, viral, or eukaryotic cell; and synthetic promoters or enhancers that are not “naturally occurring,” i.e., comprise different elements of different transcriptional regulatory regions, and/or mutations that alter expression through methods of genetic engineering that are known in the art. In addition to producing nucleic acid sequences of promoters and enhancers synthetically, promoter sequences can be produced using recombinant cloning and/or nucleic acid amplification technology, including PCR, in connection with the synthetic biological circuits and modules disclosed herein (see, e.g., U.S. Pat. Nos. 4,683,202, 5,928,906). Furthermore, it is contemplated that control sequences that direct transcription and/or expression of sequences within non-nuclear organelles such as mitochondria, chloroplasts, and the like, can be employed as well.

As described herein, an “inducible promoter” is one that is characterized by initiating or enhancing transcriptional activity when in the presence of, influenced by, or contacted by an inducer or inducing agent. An “inducer” or “inducing agent,” as defined herein, can be endogenous, or a normally exogenous compound or protein that is administered in such a way as to be active in inducing transcriptional activity from the inducible promoter. In some embodiments, the inducer or inducing agent, i.e., a chemical, a compound or a protein, can itself be the result of transcription or expression of a nucleic acid sequence (i.e., an inducer can be an inducer protein expressed by another component or module), which itself can be under the control or an inducible promoter. In some embodiments, an inducible promoter is induced in the absence of certain agents, such as a repressor. Examples of inducible promoters include but are not limited to, tetracycline, metallothionine, ecdysone, mammalian viruses (e.g., the adenovirus late promoter; and the mouse mammary tumor virus long terminal repeat (MMTV-LTR)) and other steroid-responsive promoters, rapamycin responsive promoters and the like.

The term “promoter” as contemplated herein encompasses promoter sets whereby refers to a system comprising one or more promoters (or promoter sequences) as defined herein and one or more enhancers (or enhancer sequences) as defined herein. The term “promoter set” as used herein encompasses sequences whereby the promoter and enhancer elements or sequences are separated by spacer regions or sequences that are about 1-50 nucleotides in length, e.g., about 2, 5, 7, 8, 10, 11, 12, 13, 15, 17, 18, 20, 22, 23, 25, 27, 28, 30, 32, 33, 35, 37, 38, 40, 42, 43, 45, 47, 48, or 50 nucleotides.

The terms “DNA regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate transcription of a non-coding sequence (e.g., DNA-targeting RNA) or a coding sequence (e.g., site-directed modifying polypeptide, or Cas9/Csn1 polypeptide) and/or regulate translation of an encoded polypeptide.

“Operably linked” refers to a juxtaposition wherein the components so described are in a relationship permitting them to function in their intended manner. For instance, a promoter is operably linked to a coding sequence if the promoter affects its transcription or expression. An “expression cassette” includes a heterologous DNA sequence that is operably linked to a promoter or other regulatory sequence sufficient to direct transcription of the transgene in the ceDNA vector. Suitable promoters include, for example, tissue specific promoters. Promoters can also be of AAV origin.

The term “subject” as used herein refers to a human or animal, to whom treatment, including prophylactic treatment, with the ceDNA vector according to the present disclosure, is provided. Usually the animal is a vertebrate such as, but not limited to a primate, rodent, domestic animal or game animal. Primates include but are not limited to, chimpanzees, cynomolgus monkeys, spider monkeys, and macaques, e.g., Rhesus. Rodents include mice, rats, woodchucks, ferrets, rabbits and hamsters. Domestic and game animals include, but are not limited to, cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In certain embodiments of the aspects described herein, the subject is a mammal, e.g., a primate or a human. A subject can be male or female. Additionally, a subject can be an infant or a child. In some embodiments, the subject can be a neonate or an unborn subject, e.g., the subject is in utero. Preferably, the subject is a mammal. The mammal can be a human, non-human primate, mouse, rat, dog, cat, horse, or cow, but is not limited to these examples. Mammals other than humans can be advantageously used as subjects that represent animal models of diseases and disorders. In addition, the methods and compositions described herein can be used for domesticated animals and/or pets. A human subject can be of any age, gender, race or ethnic group, e.g., Caucasian (white), Asian, African, black, African American, African European, Hispanic, Mideastern, etc. In some embodiments, the subject can be a patient or other subject in a clinical setting. In some embodiments, the subject is already undergoing treatment. In some embodiments, the subject is an embryo, a fetus, neonate, infant, child, adolescent, or adult. In some embodiments, the subject is a human fetus, human neonate, human infant, human child, human adolescent, or human adult. In some embodiments, the subject is an animal embryo, or non-human embryo or non-human primate embryo. In some embodiments, the subject is a human embryo.

As used herein, the term “host cell,” includes any cell type that is susceptible to transformation, transfection, transduction, and the like with a nucleic acid construct or ceDNA expression vector of the present disclosure. As non-limiting examples, a host cell can be an isolated primary cell, pluripotent stem cells, CD34⁺ cells), induced pluripotent stem cells, or any of a number of immortalized cell lines (e.g., HepG2 cells). Alternatively, a host cell can be an in situ or in vivo cell in a tissue, organ or organism.

The term “exogenous” refers to a substance present in a cell other than its native source. The term “exogenous” when used herein can refer to a nucleic acid (e.g., a nucleic acid encoding a polypeptide) or a polypeptide that has been introduced by a process involving the hand of man into a biological system such as a cell or organism in which it is not normally found and one wishes to introduce the nucleic acid or polypeptide into such a cell or organism. Alternatively, “exogenous” can refer to a nucleic acid or a polypeptide that has been introduced by a process involving the hand of man into a biological system such as a cell or organism in which it is found in relatively low amounts and one wishes to increase the amount of the nucleic acid or polypeptide in the cell or organism, e.g., to create ectopic expression or levels. In contrast, the term “endogenous” refers to a substance that is native to the biological system or cell.

The term “sequence identity” refers to the relatedness between two nucleotide sequences. For purposes of the present disclosure, the degree of sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the—nobrief option) is used as the percent identity and is calculated as follows: (Identical Deoxyribonucleotides.times.100)/(Length of Alignment-Total Number of Gaps in Alignment). The length of the alignment is preferably at least 10 nucleotides, preferably at least 25 nucleotides more preferred at least 50 nucleotides and most preferred at least 100 nucleotides.

The term “homology” or “homologous” as used herein is defined as the percentage of nucleotide residues in the homology arm that are identical to the nucleotide residues in the corresponding sequence on the target chromosome, after aligning the sequences and introducing gaps, if necessary, to achieve the maximum percent sequence identity. Alignment for purposes of determining percent nucleotide sequence homology can be achieved in various ways that are within the skill in the art, for instance, using publicly available computer software such as BLAST, BLAST-2, ALIGN, ClustalW2 or Megalign (DNASTAR) software. Those skilled in the art can determine appropriate parameters for aligning sequences, including any algorithms needed to achieve maximal alignment over the full length of the sequences being compared. In some embodiments, a nucleic acid sequence (e.g., DNA sequence), for example of a homology arm of a repair template, is considered “homologous” when the sequence is at least 70%, at least 75%, at least 80%, at least 85%, at least 90%, at least 91%, at least 92%, at least 93%, at least 94%, at least 95%, at least 96%, at least 97%, at least 98%, at least 99%, or more, identical to the corresponding native or unedited nucleic acid sequence (e.g., genomic sequence) of the host cell.

The term “heterologous,” as used herein, means a nucleotide or polypeptide sequence that is not found in the native nucleic acid or protein, respectively. A heterologous nucleic acid sequence may be linked to a naturally-occurring nucleic acid sequence (or a variant thereof) (e.g., by genetic engineering) to generate a chimeric nucleotide sequence encoding a chimeric polypeptide. A heterologous nucleic acid sequence may be linked to a variant polypeptide (e.g., by genetic engineering) to generate a nucleotide sequence encoding a fusion variant polypeptide.

A “vector” or “expression vector” is a replicon, such as plasmid, bacmid, phage, virus, virion, or cosmid, to which another DNA segment, i.e., an “insert”, may be attached so as to bring about the replication of the attached segment in a cell. A vector can be a nucleic acid construct designed for delivery to a host cell or for transfer between different host cells. As used herein, a vector can be viral or non-viral in origin and/or in final form, however for the purpose of the present disclosure, a “vector” generally refers to a ceDNA vector, as that term is used herein. The term “vector” encompasses any genetic element that is capable of replication when associated with the proper control elements and that can transfer gene sequences to cells. In some embodiments, a vector can be an expression vector or recombinant vector.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g., 5′ untranslated (5′UTR) or “leader” sequences and 3′ UTR or “trailer” sequences, as well as intervening sequences (introns) between individual coding segments (exons).

By “recombinant vector” is meant a vector that includes a heterologous nucleic acid sequence, or “transgene” that is capable of expression in vivo. It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

The phrase “genetic disease” as used herein refers to a disease, partially or completely, directly or indirectly, caused by one or more abnormalities in the genome, especially a condition that is present from birth. The abnormality may be a mutation, an insertion or a deletion. The abnormality may affect the coding sequence of the gene or its regulatory sequence. The genetic disease may be, but not limited to DMD, hemophilia, cystic fibrosis, Huntington's chorea, familial hypercholesterolemia (LDL receptor defect), hepatoblastoma, Wilson's disease, congenital hepatic porphyria, inherited disorders of hepatic metabolism, Lesch Nyhan syndrome, sickle cell anemia, thalassaemias, xeroderma pigmentosum, Fanconi's anemia, retinitis pigmentosa, ataxia telangiectasia, Bloom's syndrome, retinoblastoma, and Tay-Sachs disease.

As used herein the term “comprising” or “comprises” is used in reference to compositions, methods, and respective component(s) thereof, that are essential to the method or composition, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the disclosure.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Other than in the operating examples, or where otherwise indicated, all numbers expressing quantities of ingredients or reaction conditions used herein should be understood as modified in all instances by the term “about.” The term “about” when used in connection with percentages can mean±1%. The present disclosure is further explained in detail by the following examples, but the scope of the disclosure should not be limited thereto.

Groupings of alternative elements or embodiments of the disclosure disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the disclosure.

All patents and other publications; including literature references, issued patents, published patent applications, unpublished patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior disclosure or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting. It should be understood that this disclosure is not limited to the particular methodology, protocols, and reagents, etc., described herein and as such can vary. The terminology used herein is for the purpose of describing particular embodiments only, and is not intended to limit the scope of the present disclosure, which is defined solely by the claims.

### II. Cell-Free Synthesis of DNA Vectors

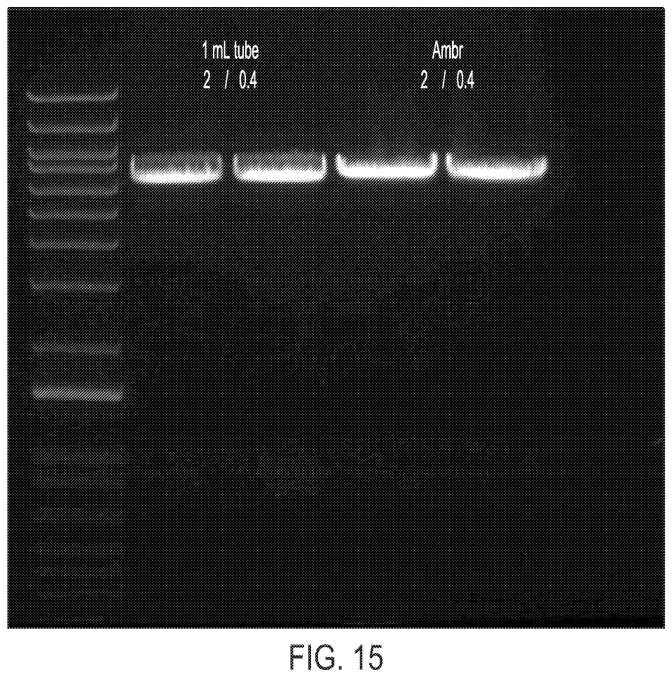

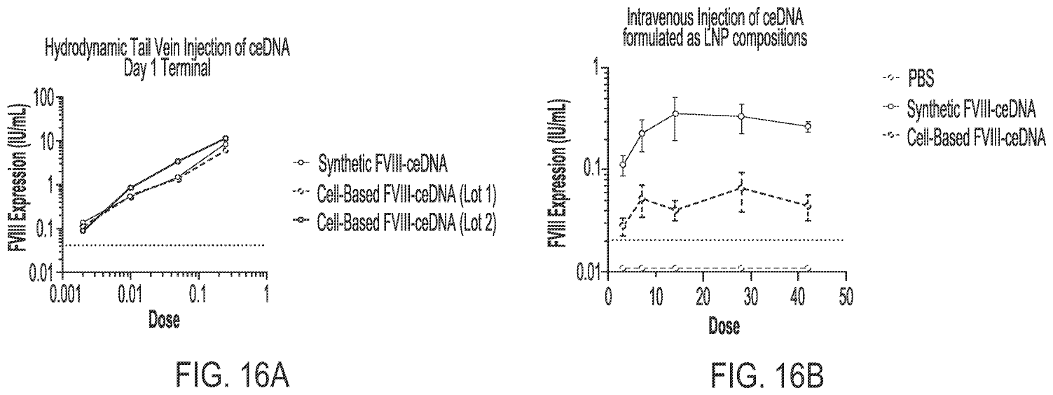

The technology described herein is directed in general to methods for generating DNA vectors in the absence of cells or cell lines. As such, the resulting vectors have fewer impurities than comparable vectors made using conventional cell-based production methodologies, which may translate into better in vivo expression that is sustained a longer duration of time after administration (see e.g., FIG. 16B). As exemplified here, this cell-free synthesis is also scalable, e.g., from small reactions (˜1 mL) and up to a large scale (>40, 100, 200, 500, 1,000 mL) and further without compromising the purity (see e.g., FIG. 15), therefore allowing the vectors to prepared in large quantities for therapeutic uses. Moreover, while the cell-based methods could take up to weeks for the vectors to be produced, the cell-free methods described herein produce vectors in less than a week, such as 2-4 days depending on the scale.

According to some embodiments, the cell-free method described herein involves rolling circle and multiple strand displacement (MSD) amplification of DNA plasmid template by >1000 fold and subsequent conversion of the resultant products into ceDNA molecules using type II endonuclease, ligase, ITR oligos, and exonuclease enzymes.

According to one aspect, the disclosure provides a method of producing a closed-ended DNA (ceDNA) vector, the method comprising (a) contacting a double-stranded DNA construct having a sense strand and an antisense strand with at least a first restriction endonuclease and at least a second restriction endonuclease, wherein the construct comprises a transgene expression cassette, a first non-palindromic restriction endonuclease recognition site and a corresponding first cleavage site upstream of the transgene expression cassette, and a second non-palindromic restriction endonuclease recognition site and a corresponding second cleavage site downstream of the transgene expression cassette; and wherein the first restriction endonuclease is capable of cleaving the double-stranded DNA construct at the first cleavage site, and wherein the second restriction endonuclease is capable of cleaving the double-stranded DNA construct at the second cleavage site, and wherein contacting the double-stranded DNA construct with the first restriction endonuclease and the second restriction endonuclease releases an insert having a first end comprising a first single-stranded overhang and a second end comprising a second single-stranded overhang; (b) ligating the first end to a first oligonucleotide comprising one or more hairpin structures; and (c) ligating the second end to a second oligonucleotide comprising one or more hairpin structures; thereby producing a ceDNA vector.

According to some embodiments, the first oligonucleotide comprises an inverted terminal repeat (ITR). According to further embodiments, the second oligonucleotide comprises an ITR. According to other embodiments, the first oligonucleotide and the second oligonucleotide are different. According to some other embodiments, the first oligonucleotide and the second oligonucleotide are the same.

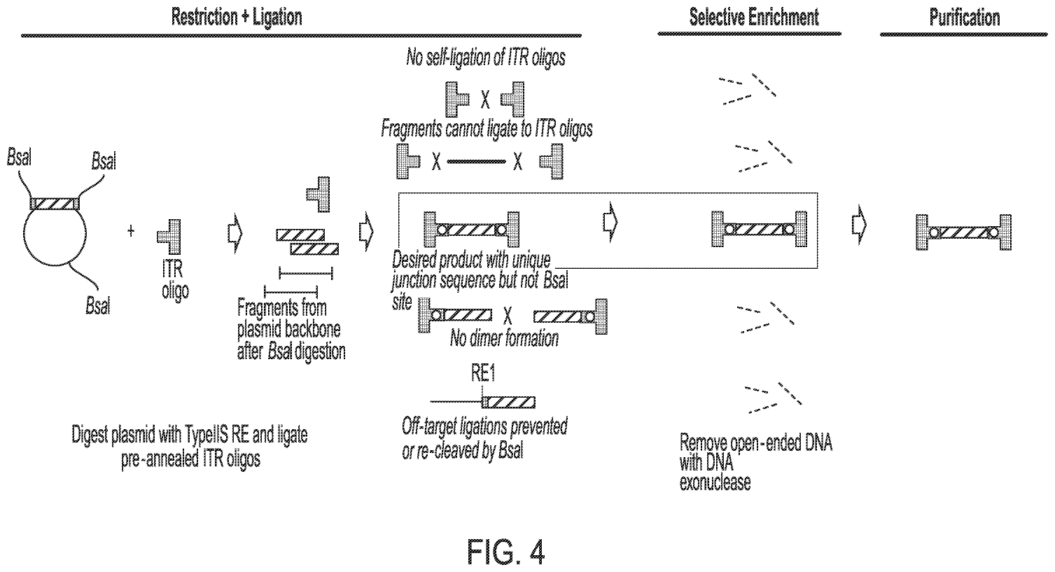

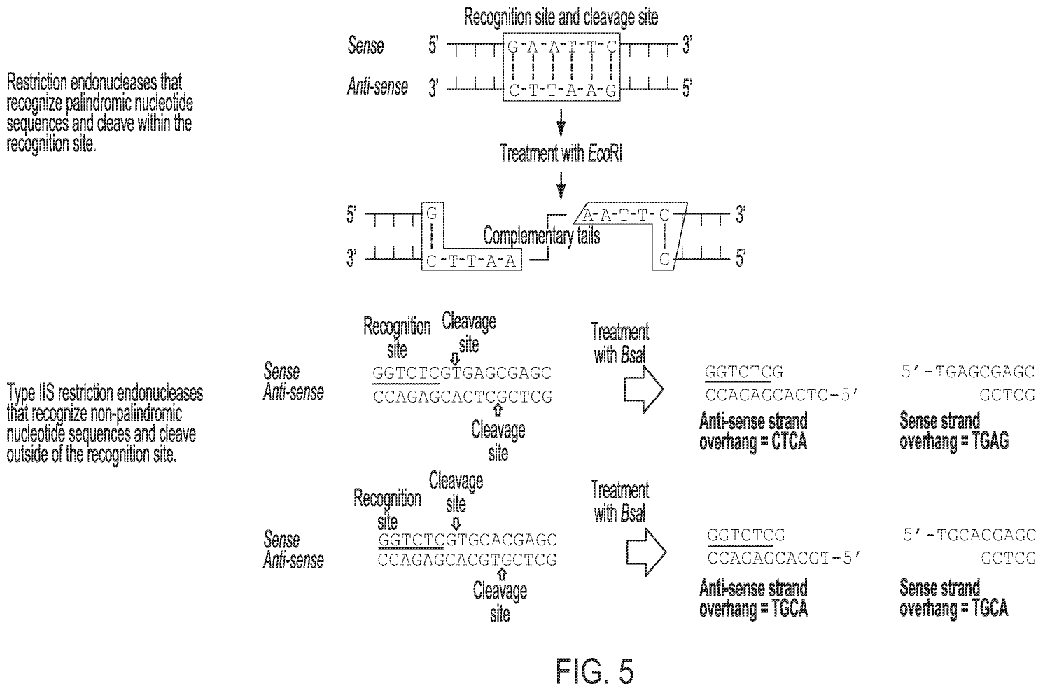

An overview of an exemplary embodiment of cell-free synthetic method of preparing a ceDNA vector is illustrated in FIG. 4. Briefly, the transgene expression cassette (in diagonal stripes) is excised from a double-stranded DNA construct using with at least one restriction endonuclease, followed up by ligation of the insert with inverted terminal repeat (ITR) oligonucleotides to form ceDNA. ITR oligonucleotides are single-stranded oligonucleotides that self-anneal to form an ITR-like three-dimensional configuration. Restriction endonucleases used in the methods described herein, such as but not necessarily limited to Type IIS restriction endonucleases, cleave the DNA at a site that is distinct and not within the recognition site. These restriction endonucleases used in the cell-free synthetic methods disclosed herein also recognize non-palindromic nucleotide sequences such that the recognition sequence (which is also the binding site) for the enzyme is only encoded on one strand (see e.g., FIG. 5). Therefore, cleavage by this class of restriction endonucleases is directional, occurring either upstream or downstream of the recognition site, but not within the recognition site itself, unlike other restriction endonucleases that are most heavily used in molecular biology such as EcoRI (see FIG. 5). The strand that encodes the recognition sequence dictates which side (i.e., downstream or upstream) of the sequence is cleaved. Taken together, the unique activity of the restriction endonucleases used in the methods described herein allows any sequence within a pre-determined distance from a specific recognition site to be cleaved by the restriction endonuclease and consequently, any overhang sequence to be generated. Digestion with the special restriction endonuclease(s) creates cohesive overhangs at both 5′ and 3′ ends of the excised insert that are compatible with the overhangs of the ITR oligonucleotide. In other words, the design of the ITR oligonucleotide and insert overhangs drives the high specificity of the ligation process such that the ITR oligonucleotide overhangs and the insert overhangs are compatible with each other. Once ligated, the desired ceDNA product is not susceptible to digestion with the restriction endonuclease because the recognition site is not re-generated. However, in situations where the excised insert and the plasmid fragments re-ligate into the original construct, the recognition sites are re-generated and therefore allow the construct to be cleaved.

Due to the unique activity of the restriction endonucleases used in the cell-free synthetic methods described herein, the generation of unique overhangs on the insert ends and the design of unique overhangs on the ITR oligonucleotides that drive the high specificity of the sub-cloning process, the digestion and ligation can take place in a single reaction vessel without a need to purify the digestion products prior to ligation. In some embodiments where the digestion/ligation is ensued by treatment with an exonuclease to degrade open-ended DNA fragments and intermediates, as shown in FIG. 4, the restriction endonuclease digestion, ligation, and exonuclease degradation take place in a single reaction vessel and all the reactions can occur simultaneously.

In addition, the unique activity of the restriction endonucleases used in the cell-free synthetic methods described herein allow the directionality of ligation reactions that utilize more than one ITR oligonucleotides, thereby enabling preparation of ceDNA having asymmetric ITRs.

### Cell-Free Synthetic Production Method in General

Disclosed herein is a process for synthesis of closed-ended DNA vectors which does not require use of any microbiological steps. In some embodiments, the process allows for synthesis of closed-ended DNA vectors in a system using enzymatic cleavage steps using restriction endonucleases and ligation steps to generate the closed-ended DNA vectors. In nearly all embodiments, the synthetic system for DNA vector production is a cell-free system.

It will be appreciated by one of ordinary skill in the art that one or more enzymes used in the synthetic production method or one or more of the oligonucleotide components can be produced from a cell and used in the methods of the disclosure in purified form. Accordingly, in some embodiments, the procedures themselves in synthetic production method are cell-free. However, the base materials such as the double-stranded DNA construct and ITR oligonucleotides as well as enzymes such as restriction endonucleases and ligases may have been produced using methods and techniques utilizing cells.

In one embodiment, a restriction endonuclease and/or a ligation-competent protein can be expressed or provided from an expression vector in a cell, e.g., bacterial cell. In one embodiment, a cell, such as a bacterial cell, comprising an expression vector expressing one or more of the restriction endonucleases or the ligase enzymes can be present. Therefore, while the methods disclosed herein are primarily directed to cell-free synthetic methods to generate the DNA vectors disclosed herein, also encompassed in one embodiment are synthetic production methods where a cell, e.g., bacterial cell but not an insect cell is present and can be used to express one or more of the enzymes required in the method. In such embodiments, the cell expressing a restriction endonuclease and/or ligation-competent protein is not an insect cell. In all embodiments where a cell is present and expresses one or more restriction endonucleases or ligation-competent proteins, the cell does not replicate the close-ended DNA vector. Stated differently, the intracellular machinery of the cell does not replicate, or is not involved in the replication of the DNA vector.

In some embodiments, synthesis of DNA vectors (e.g., ceDNA vectors) described herein is carried out in an in vitro cell-free process starting from either a double-stranded DNA construct or one or more oligonucleotides. The double-stranded DNA construct or one or more oligonucleotides are cleaved with restriction endonucleases and ligated to form the DNA molecules. In some embodiments, the oligonucleotides which can be synthesized chemically, thus avoiding use of large starting templates encoding the entirety of the desired sequence which would typically need to be propagated in bacteria. Once a desired DNA sequence is synthesized, it can be cleaved and ligated with other oligonucleotides as disclosed herein. The use of multiple oligonucleotides in the generation of closed-ended DNA vectors using the methods disclosed herein allows for a modular approach to DNA vector generation, enabling tailoring and/or specific selection of the terminal repeats, e.g., ITRs, as well as the spacing of the terminal repeats, and also selection of the heterologous nucleic acid sequence in the synthetically produced closed-ended DNA vectors.

### Cell-Free Synthetic Production of DNA Vectors

Certain methods for the production of a ceDNA vector comprising various ITR configurations using cell-based methods are described in Example 1 of International Patent Application Publication Nos. WO2019/051255 and WO2019/113310, the contents of which are incorporated by reference in their entireties herewith.

In contrast, the methods provided herein relate to a synthetic production method, e.g., in some embodiments, a cell-free production method, and is also referred to herein as “synthetic closed-ended DNA vector production” or “synthetic production”.

In one aspect, a closed-ended DNA vector is generated by excising a transgene expression cassette from a double-stranded DNA construct, followed by ligation of the ends of the insert to a first oligonucleotide comprising one or more hairpin structures and a second oligonucleotide comprising one or more hairpin structures to form the ceDNA. In some embodiments, each of oligonucleotides independently includes 1, 2, 3, 4, or more stem-loop regions. In some embodiments, each of the oligonucleotides independently includes 2 or 3 stem-loop regions. In some embodiments, the first oligonucleotide comprising one or more hairpin structures and the second oligonucleotide comprising one or more hairpin structures are each a single-stranded oligonucleotide that self-anneals to form a three-dimensional configuration. In further embodiments, the three-dimensional configuration is a T- or Y-shaped stem-loop structure.

In another aspect, a closed-ended DNA vector is generated by excising a transgene expression cassette from a double-stranded DNA construct, followed by ligation of the ends of the insert to ITR oligonucleotides to form the ceDNA. The ligation may be effected by a ligase (e.g., T4 ligase) or an AAV Rep protein. In one embodiment, the reaction mixture is not purified prior to ligation. In such an embodiment, the excision of the transgene expression cassette (e.g., with one or more restriction endonucleases) and ligation take place simultaneously in a single reaction vessel. In an alternative embodiment, the reaction mixture is purified prior to ligation.

The resultant closed-ended DNA vector as prepared by the cell-free synthetic methods described herein comprises at least about 85%, at least about 90%, at least about 95%, at least about 96%, at least about 97%, at least about 98%, or at least about 99% of the monomeric species of the vector. The resultant closed-ended DNA vector as prepared by the cell-free synthetic methods described herein comprises less than about 15%, less than about 10%, less than about 5%, less than about 4%, less than about 3%, less than about 2%, or less than about 1% of the sub-monomeric species of the vector.

As used herein, the term “sub-monomeric species” is meant to refer to a complex of nucleic acid that is generally smaller than the monomeric form of a therapeutic nucleic acid as defined herein, such as a ceDNA genome, ceDNA vector, AAV genome, or AAV vector as determined for example, by ion exchange chromatography (IEX). The terms “sub-monomeric species” and “sub-monomeric DNA” also encompass dimers formed by two sub-monomer units and multimers formed by three or more sub-monomer units. The formation of dimers and multimers may be unstable and therefore transient, where the dimers and multimers may eventually disintegrate into their sub-monomeric forms. Amounts and concentrations of sub-monomeric DNA can be quantitated and expressed in mass units (e.g., μg, ng, pg) or mass/volume units, for example, using ion exchange high-performance liquid chromatography (IEX-HPLC) for peak quantitation, and/or capillary electrophoresis, such as using a chip-based capillary electrophoresis machine such as Bioanalyzer.

In one embodiment, the double-stranded DNA construct is selected from a bacmid, a plasmid, a minicircle, and a linear double-stranded DNA molecule. In such an embodiment, the double-stranded DNA construct is provided with at least, in 5′ to 3′ order: a first non-palindromic restriction endonuclease recognition site and a corresponding first cleavage site upstream of the transgene expression cassette; a transgene expression cassette; and a second non-palindromic restriction endonuclease recognition site and a corresponding second cleavage site downstream of the transgene expression cassette. In one embodiment, the first restriction endonuclease is capable of cleaving the double-stranded DNA construct at the first cleavage site, and wherein the second restriction endonuclease is capable of cleaving the double-stranded DNA construct at the second cleavage site, and wherein contacting the double-stranded DNA construct with the first restriction endonuclease and the second restriction endonuclease releases an insert having a first end comprising a first single-stranded overhang and a second end comprising a second single-stranded overhang. In further embodiments, the first restriction endonuclease and the second restriction endonuclease are different restriction endonucleases. In other embodiments, the first restriction endonuclease and the second restriction endonuclease are the same restriction endonuclease. In another embodiment, the double-stranded DNA construct is provided with at least, in 5′ to 3′ order: a first non-palindromic restriction endonuclease recognition site and a corresponding first cleavage site upstream of the transgene expression cassette; a first partial ITR; a transgene expression cassette; a second partial ITR; and a second non-palindromic restriction endonuclease recognition site and a corresponding second cleavage site downstream of the transgene expression cassette. The double-stranded DNA construct is contracted with at least one restriction endonuclease is capable of cleaving the construct at the first and second cleavage sites to release an insert having single-stranded overhangs at the 5′ and 3′ ends (i.e., cohesive ends) of the insert. These ends of the insert are then ligated to a first inverted terminal repeat (ITR) oligonucleotide and a second ITR oligonucleotide to form the ceDNA vector. In one embodiment, one or both of the single-stranded overhangs at the 5′ and 3′ ends of the inserts is a 5′ overhang. In one embodiment, one or both of the single-stranded overhangs at the 5′ and 3′ ends of the inserts is a 3′ overhang. In one embodiment, these overhangs are about 1 to about 30 nucleotides in length, e.g. about 1 to about 25 nucleotides, or about 1 to about 20 nucleotides, or about 1 to about 18 nucleotides, or about 1 to about 15 nucleotides, or about 1 to about 12 nucleotides, or about 1 to about 10 nucleotides, or about 1 to about 8 nucleotides, or about 2 to about 8 nucleotides, or about 2 to about 7 nucleotides, or about 2 to about 6 nucleotides, or about 1 nucleotide, or about 2 nucleotides, or about 3 nucleotides, or about 4 nucleotides, or about 5 nucleotides, or about 6 nucleotides, or about 7 nucleotides, or about 8 nucleotides, or about 9 nucleotides, or about 10 nucleotides in length.

Use of Restriction Endonucleases that Recognize Non-Palindromic Nucleotide Sequences and have Cleavage Sites Distinct from their Recognition and Binding Sites

Of note, the restriction endonuclease(s) used in the synthetic methods provided herein recognizes non-palindromic nucleotide sequences. As used herein, the term “non-palindromic” when referring to a double-stranded polynucleotide or oligonucleotide having different 5′→3′ nucleotide sequences between the sense strand and the anti-sense strand; whereas the term “palindromic” when referring to a double-stranded polynucleotide or oligonucleotide having identical 5′→3′ nucleotide sequences between the sense strand and the anti-sense strand.

Accordingly, as illustrated in FIG. 5, such a restriction endonuclease recognizes a double-stranded polynucleotide or oligonucleotide having different 5′→3′ nucleotide sequences between the sense strand and the anti-sense strand. This means that the recognition sequence for the restriction endonuclease, such as 5′-GGTCTC-3′ for the example illustrated in FIG. 5, is encoded only one of the strands. Another prominent feature about restriction endonuclease(s) used in the synthetic methods described herein is that the enzyme cleaves the DNA at a cleavage site that is either upstream or downstream of the recognition sequence, but not within the recognition site itself. The strand that encodes the recognition site dictates which side (i.e., downstream or upstream) of the recognition sequence is cleaved.

In certain embodiments, the first non-palindromic restriction endonuclease recognition site and the corresponding first cleavage site are separate and distinct sites from each other that are located upstream of the transgene expression cassette. The first cleavage site is about 1 to 35 nucleotides away from the first non-palindromic restriction endonuclease recognition site in at least one of the sense and the antisense strands of the construct, e.g., about 1 to about 22 nucleotides away, or about 1 to about 20 nucleotides away, or about 1 to about 15 nucleotides away, or about 1 to about 12 nucleotides away, or about 1 to about 10 nucleotides away, or about 1 to about 8 nucleotides away, or about 1, or about 2, or about 3, or about 4, or about 5, or about 6, or about 7, or about 8, or about 9, or about 10 nucleotides away from the first non-palindromic restriction endonuclease recognition site in at least one of the sense and the antisense strands of the construct.

In certain embodiments, the second non-palindromic restriction endonuclease recognition site and the corresponding second cleavage site are separate and distinct sites from each other that are located downstream of the expression cassette. The second cleavage site is about 1 to 35 nucleotides away from the second non-palindromic restriction endonuclease recognition site in at least one of the sense and the antisense strands of the construct, e.g., about 1 to about 22 nucleotides away, or about 1 to about 20 nucleotides away, or about 1 to about 15 nucleotides away, or about 1 to about 12 nucleotides away, or about 1 to about 10 nucleotides away, or about 1 to about 8 nucleotides away, or about 1, or about 2, or about 3, or about 4, or about 5, or about 6, or about 7, or about 8, or about 9, or about 10 nucleotides away from the second non-palindromic restriction endonuclease recognition site in at least one of the sense and the antisense strands of the construct.

A single restriction endonuclease can target both the first and second non-palindromic restriction endonuclease recognition sites and their corresponding cleavage sites. Alternatively, two different restriction endonucleases target both the first and second non-palindromic restriction endonuclease recognition sites and their corresponding cleavage sites.

### Type IIS Restriction Endonucleases

In one embodiment, the restriction endonuclease(s) used in the synthetic methods provided herein, which recognizes non-palindromic nucleotide sequences and cleaves a DNA outside of the recognition site is a Type IIS restriction endonuclease. Non-limiting examples of Type IIS restriction endonucleases include AcuI, AlwI, Alw26I, BasI, BbsI, BbvI, BceAI, BcgI, BCiVI, BcoDI, BruAI, BmrI, BpiI, BpuEI, BsaI, BsaXI, BseGI, BseRI, BsgI, BsmAI, BsmBI, BsmFI, BsmI, BspCNI, BspMI, BspQI, BsrDI, BsrI, BtgZI, BtsCI, BtsI, MutI, CspCI, Earl, EciI, Eco31I, Esp3I, FauI, FokI, HgaI, HphI, HpyAV, LguI, MboII, MlyI, MmeI, MnlI, Mva1269I, NmeAIII, PaqCI, PleI, SapI, SfaNI, and an isoschizomer of any of the foregoing. Isoschizomers are pairs of restriction endonucleases that are specific to the same recognition sequence. For example, BcoDI and BsmAI are isoschizomers of each other, both being specific to the recognition sequence of 5′-GTCTC-3′. In one embodiment, the Type IIS endonuclease(s) is selected from BbsI, BsaI, Esp3I, and SapI, and an isoschizomer thereof. In one embodiment, the Type IIS endonuclease is BbsI or an isoschizomer thereof. In one embodiment, the Type IIS endonuclease is BsaI or an isoschizomer thereof. In one embodiment, the Type IIS endonuclease is BbsI or an isoschizomer thereof. In one embodiment, the Type IIS endonuclease is Esp3I or an isoschizomer thereof. In one embodiment, the Type IIS endonuclease is SapI or an isoschizomer thereof.

### ITR Oligonucleotides

The first and second ITR oligonucleotides to which the 5′ and 3′ ends of the insert are ligated to in the cell-free synthetic methods disclosed herein are each a single-stranded oligonucleotide that self-anneals to form a three-dimensional configuration, such has an ITR three-dimensional structure like a hairpin structure or a T- or Y-shaped stem-loop structure. In one embodiment, one or both of the ITR oligonucleotides are synthetic or synthesized.

In some embodiments, in addition to the three-dimensional ITR structure, the ITR oligonucleotides each self-anneal to further form a single-stranded overhang at either the 5′ or the 3′ end of the oligonucleotide. In such embodiments, the single-stranded overhangs of the insert are ligated to the single-stranded overhangs of the ITR oligonucleotides. The ITR oligonucleotide overhangs are about 1 to about 30 nucleotides in length, e.g. about 1 to about 25 nucleotides, or about 1 to about 20 nucleotides, or about 1 to about 18 nucleotides, or about 1 to about 15 nucleotides, or about 1 to about 12 nucleotides, or about 1 to about 10 nucleotides, or about 1 to about 8 nucleotides, or about 2 to about 8 nucleotides, or about 2 to about 7 nucleotides, or about 2 to about 6 nucleotides, or about 1 nucleotide, or about 2 nucleotides, or about 3 nucleotides, or about 4 nucleotides, or about 5 nucleotides, or about 6 nucleotides, or about 7 nucleotides, or about 8 nucleotides, or about 9 nucleotides, or about 10 nucleotides in length. In particular embodiment, the ITR oligonucleotide overhang comprises a 5′→3′ nucleotide sequence of CTCT, CTCA, CACT, CTC, or GCT.

The overhangs of the first and second ITR oligonucleotides comprise non-complementary 5′→3′ nucleotide sequences to each other. In one embodiment, overhangs of the first and second ITR oligonucleotides comprise or have identical sequences, i.e., the same 5′→3′ nucleotide sequence and in a further embodiment, the first and second ITR oligonucleotides are the same oligonucleotide. In such an embodiment, the overhangs at the 5′ and 3′ ends of the insert comprise or have the same 5′→3′ nucleotide sequence. The overhangs of the ITR oligonucleotides are complementary to either and both of the overhangs of the insert. Such an embodiment whereby a single ITR oligonucleotide is used in the cell-free synthesis of ceDNA is useful for preparing ceDNA having symmetric ITRs as defined herein.

In alternative embodiments, overhangs of the first and second ITR oligonucleotides comprise or have different sequences, i.e., different 5′→3′ nucleotide sequences and hence, the first and second ITR oligonucleotides are different oligonucleotides. In such an embodiment, the overhangs at the 5′ and 3′ ends of the insert comprise or have different 5′→3′ nucleotide sequences. The overhangs of the ITR oligonucleotides are each complementary to only one of the overhangs of the insert.

As for the length of the ITR oligonucleotides, in some embodiments, the ITR oligonucleotides are each about 40 nucleotides to about 75 nucleotides in length, e.g., about 40 nucleotides to about 72 nucleotides, or about 40 nucleotides to about 70 nucleotides, or about 40 nucleotides to about 68 nucleotides, or about 40 nucleotides to about 65 nucleotides, or about 40 nucleotides to about 75 nucleotides, or about 45 nucleotides to about 72 nucleotides, or about 45 nucleotides to about 70 nucleotides, or about 45 nucleotides to about 68 nucleotides, or about 45 nucleotides to about 65 nucleotides. In one embodiment, the first ITR oligonucleotide and the second ITR oligonucleotide each and independently comprise a nucleotide sequence selected from the group consisting of SEQ ID NO: 1; SEQ ID NO: 2; SEQ ID NO: 3; SEQ ID NO: 4; SEQ ID NO: 5; SEQ ID NO: 6; SEQ ID NO: 7; and SEQ ID NO:8 (see Table 1).

In one embodiment, the first ITR oligonucleotide and the second ITR oligonucleotide each and independently comprise a nucleotide sequence at least 95%, at least 96%, at least 97%, at least 98%, or at least 99% identical to selected from the group consisting of SEQ ID NO: 1; SEQ ID NO: 2; SEQ ID NO: 3; SEQ ID NO: 4; SEQ ID NO: 5; SEQ ID NO: 6; SEQ ID NO:7; and SEQ ID NO:8 (see Table 1). In one embodiment, the first ITR oligonucleotide and the second ITR oligonucleotide each and independently consist of a nucleotide sequence selected from the group consisting of SEQ ID NO: 1; SEQ ID NO: 2; SEQ ID NO: 3; SEQ ID NO: 4; SEQ ID NO: 5; SEQ ID NO: 6; SEQ ID NO: 7; and SEQ ID NO:8 (see Table 1).

TABLE 1
Exemplary ITR Oligonucleotides for Cell-Free
Synthesis of DNA Vectors Including ceDNA
Vectors

| | 5′ → 3′ | |
|---|---|---|
| ITR oligo- | sequence of ITR | |
| nucleotide | oligonucleotide | Description |
| ITR oligo 1 | CTCACTGAGGCCGCCC | AAV2 wt-ITR sequence; |
| (SEQ ID NO: | GGGCAAAGCCCGGGCG | generates 7-bp stem |
| 1) | TCGGGCGACCTTTGGT | with 5′-CTCA overhang. |
| | CGCCCGGCCTCAG | |
| ITR oligo 2 | CTCTCTGAGGCCGCCC | AAV2 wt-ITR sequence; |
| (SEQ ID NO: | GGGCAAAGCCCGGGCG | generates 7-bp stem |
| 2) | TCGGGCGACCTTTGGT | with 5′-CTCT overhang. |
| | CGCCCGGCCTCAG | |
| ITR oligo 3 | CACTGAGGCCGGGCGA | Mutant ITR sequence |
| (SEQ ID NO: | CCAAAGGTCGCCCGAC | with 9-bp deletion; |
| 3) | GCCCGGGCGGCCTC | generates 5-bp stem |
| | | with 5′-CACT overhang. |
| ITR oligo 4 | CACTGAGGCCGCCCGG | AAV2 wt-ITR sequence; |
| (SEQ ID NO: | GCAAAGCCCGGGCGTC | generates 5-bp stem |
| 4) | GGGCGACCTTTGGTCG | with 5′-CACT overhang. |
| | CCCGGCCTC | |
| ITR oligo 5 | CTCAGGCCGCCCGGGC | AAV2 wt-ITR sequence; |
| (SEQ ID NO: | AAAGCCCGGGCGTCGG | generates 3-bp stem |
| 5) | GCGACCTTTGGTCGCC | with 5′-CTCA overhang. |
| | CGGCC | |
| ITR oligo 6 | CTCACTGAGGCCGCCC | AAV2 wt-ITR sequence; |
| (SEQ ID NO: | GGGCAAAGCCCGGGCG | generates 8-bp stem |
| 6) | TCGGGCGACCTTTGGT | with 5′-CTC overhang. |
| | CGCCCGGCCTCAGT | |
| ITR oligo 7 | GCTCACTGAGGCCGCC | AAV2 wt-ITR sequence; |
| (SEQ ID NO: | CGGGCAAAGCCCGGGC | generates 9-bp stem |
| 7) | GTCGGGCGACCTTTGG | with 5′-GCT overhang. |
| | TCGCCCGGCCTCAGTG | |
| ITR oligo 8 | CTCAGAGTGAGGTGAG | AAV2 wt-ITR sequence; |
| (SEQ ID NO: | GCCGCCCGGGCAAAGC | generates 14-bp stem |
| 8) | CCGGGCGTCGGGCGAC | with 5′-CTCA overhang. |
| | CTTTGGTCGCCCGGCC | |
| | TCACCTCACTC | |

To enhance ligation efficiency, the 5′ ends of the ITR oligonucleotide, including but not limited to the ITR oligonucleotides in Table 1, may be phosphorylated.

In one embodiment, the first ITR and the second ITR each comprise a hairpin structure and/or a T- or Y-shaped stem-loop structure. In another embodiment, the first ITR and the second ITR each comprise a T- or Y-shaped stem-loop structure. In another embodiment, the T- or Y-shaped stem-loop structure comprises a stem comprising A-A′ and D-D′ stem regions and two B-B′ and C-C′ loops. In another embodiment, one or both of the first ITR and the second ITR is an adeno-associated virus (AAV) ITR or an AAV-derived ITR. In another embodiment, one or both of the first ITR and the second ITR is a wild-type ITR. In another embodiment, both the first ITR and the second ITR are wild-type ITRs. In another embodiment, one or both of the first ITR and the second ITR is a modified ITR. In another embodiment, the first ITR and the second ITR are symmetrical or substantially symmetrical to each other. In another embodiment, the first ITR and the second ITR are asymmetrical ITRs.

In some embodiments, the T- or Y-shaped stem-loop structure (e.g., single stem+two loops or single stem+two loops) of the first ITR oligonucleotide and the second ITR oligonucleotide comprises a stem region (i.e., A-A′ stem region, D-D′ stem region, or both A-A′ and D-D′ stem regions as indicated in FIG. 10) that is at least about 4 base pairs (nucleotides) in length, e.g., about 4 base pairs to about 30 base pairs, or 4 base pairs to about 25 base pairs, or 4 base pairs to about 22 base pairs, or 4 base pairs to about 20 base pairs, or 4 base pairs to about 18 base pairs, or 4 base pairs to about 15 base pairs, or 4 base pairs to about 12 base pairs, or 4 base pairs to about 10 base pairs, or 4 base pairs to about 8 base pairs, or 4 base pairs to about 7 base pairs, or 4 base pairs to about 6 base pairs or 6 base pairs to about 8 base pairs, or about 4 base pairs, or about 5 base pairs, or about 6 base pairs, or about 7 base pairs, or about 8 base pairs, or about 9 base pairs, or about 10 base pairs, or about 11 base pairs, or about 12 base pairs, or about 13 base pairs, or about 14 base pairs, or about 15 base pairs. In one embodiment, this stem region length does not include the overhang length.

### Partial ITRs and Spacers

In some embodiments, the ITR oligonucleotides form the full ITRs of a ceDNA product on both ends. In other embodiments the ITR oligonucleotides partially for the ITRs of the ceDNA, while the remaining and continuing sequence of the ITRs is found in partial ITRs on the insert. Accordingly, in some embodiments, the double-stranded DNA construct from which the transgene expression cassette is excised in the cell-free synthetic methods disclosed herein further comprises a least a first partial ITR and a second partial ITR each flanking the transgene expression cassette. In one embodiment, the first partial ITR is upstream of the transgene expression cassette and downstream of the first non-palindromic restriction endonuclease recognition site and the corresponding first cleavage site. The second partial ITR is downstream of the transgene expression cassette and upstream of the second non-palindromic restriction endonuclease recognition site and the corresponding second cleavage site. In one embodiment, the first cleavage site is adjacent the first partial ITR and the second cleavage site is adjacent to the second partial ITR (i.e., no spacer between the cleavage site and the partial ITR).

In certain embodiments, the double-stranded DNA construct or the excised insert further comprises one or more spacer regions. In one embodiment, the double-stranded DNA construct or the insert further comprises a first spacer between the first partial ITR and the transgene expression cassette. In another embodiment, the double-stranded DNA construct further comprises a second spacer between the second partial ITR and the transgene expression cassette. Each spacer region or sequence is about 1-50 nucleotides in length, e.g., about 2, 5, 7, 8, 10, 11, 12, 13, 15, 17, 18, 20, 22, 23, 25, 27, 28, 30, 32, 33, 35, 37, 38, 40, 42, 43, 45, 47, 48, or 50 nucleotides. In some embodiments, the spacer between a first (or left) partial ITR or the second (or right partial ITR) and the transgene expression cassette is selected from the spacers comprising the sequences as shown in Table 2. According to some embodiments, the spacer comprises a nucleic acid sequence that is at least 95%, 96%, 97%, 98% or 99% identical to a nucleic acid sequence selected from the group consisting of: SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, and SEQ ID NO: 40. According to some embodiments, the spacer consists of a nucleic acid sequence selected from the group consisting of: SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, and SEQ ID NO: 40.

TABLE 2
Exemplary spacer sequences

| SEQ | Left | |
|---|---|---|
| ID | or | |
| NO: | Right | Sequence (5′ → 3′) |
| 32 | Left | TGTAGTTAATGATTAACCCGCCATGCTACTTAT |
| | | CGCGGCCGC |
| 33 | Left | TGTAGTTAATGATTAACCCGCCATGCTACTTAT |
| | | CTACGTAGCCATGCATATG |
| 34 | Left | TGTAGTTAATGATTAACCCGCCATGCTACTTAT |
| | | C |
| 35 | Left | TGTAGTTAATGATTAACCCACCATGCTACTTAT |
| | | GGCCTGCAGG |
| 36 | Left | TGTAGTTAATGATTAACCCACCATGCTACTTAT |
| | | CACCAATTG |
| 37 | Right | TCTAGAGCATGGCTACGTAGATAAGTAGCATGG |
| | | CGGGTTAATCATTAACTACACCTGCAGG |
| 38 | Right | GCTAGCCCACAATCTGCCTCCCAGTAGTACATG |
| | | ACATTAGTTTATTAATAGCCT |
| 39 | Left | TCCACAATCTGCCTCCCAGTAGTACATGACATT |
| | | AGTTTATTAATAGCCT |
| 40 | Right | CTTAAGCATGGCTACGTAGATAAGTAGCATGGC |
| | | GGGTTAATCATTAACTACACCTGCAGG |

In some aspects, the disclosure provides a method of producing a double-stranded DNA construct from a plasmid template via rolling-circle amplification, comprising the steps of (a) contacting the plasmid template with a thermostable polymerase having strand-displacement activity, wherein the ratio of plasmid template concentration (in ng/μl) to polymerase concentration (in U/μl) is greater than about 1; (b) contacting the plasmid template with an oligonucleotide primer and dNTPs; (c) incubating the plasmid template, the polymerase, the oligonucleotide primer, and the dNTPs at a temperature of about 40° C. or less, for a time period of at least about 5 hours; thereby producing a double-stranded DNA construct. In some embodiments, the ratio of plasmid template concentration (in ng/μl) to polymerase concentration (in U/μl) is greater than about 1, about 2, about 3, about 4, about 5, about 6, about 7, about 8, about 9, about 10, about 11, about 12, about 13, about 14, about 15, about 16, about 17, about 18, about 19, or about 20. In some embodiments, the plasmid template concentration is about 0.01 ng/μl, about 0.05 ng/μl, about 0.1 ng/μl, about 0.15 ng/μl, about 0.2 ng/μl, about 0.21 ng/μl, about 0.22 ng/μl, about 0.23 ng/μl, about 0.24 ng/μl, about 0.2 ng/μl 5, about 0.26 ng/μl, about 0.27 ng/μl, about 0.28 ng/μl, about 0.29 ng/μl, about 0.3 ng/μl, about 0.35 ng/μl, about 0.4 ng/μl, about 0.45 ng/μl, about 0.5 ng/μl, about 0.6 ng/μl, about 0.7 ng/μl, about 0.8 ng/μl, about 0.9 ng/μl, or about 1.0 ng/μl. In other embodiments, the polymerase concentration is about 0.01 U/μl, about 0.02 U/μl, about 0.03 U/μl, about 0.04 U/μl, about 0.05 U/μl, about 0.06 U/μl, about 0.07 U/μl, about 0.08 U/μl, about 0.09 U/μl, about 0.1 U/μl, about 0.15 U/μl, about 0.2 U/μl, about 0.25 U/μl, about 0.3 U/μl, about 0.35 U/μl, about 0.4 U/μl, or about 0.45 U/μl. In further embodiments, the temperature in step (c) is less than about 40° C., about 39° C., about 38° C., about 37° C., about 36° C., about 35° C., about 34° C., about 33° C., about 32° C., about 31° C., about 30° C., about 29° C., about 28° C., about 27° C., about 26° C., about 25° C., about 24° C., about 23° C., about 22° C., or about 21° C. In some embodiments, the time period is at least about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 21 hours, about 33 hours, about 34 hours, about 35 hours, about 36 hours, about 37 hours, about 38 hours, about 39 hours, or about 40 hours. In some embodiments, the time period is less than about 6 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 21 hours, about 33 hours, about 34 hours, about 35 hours, about 36 hours, about 37 hours, about 38 hours, about 39 hours, or about 40 hours. In some embodiments, the plasmid template concentration is about 0.25 ng/l, the temperature is about 30° C., the polymerase concentration is about 0.05 U/μl, and the time period is about 18-26 hours. In further embodiments, the oligonucleotide primer concentration is less than about 50 μM. In other embodiments, the oligonucleotide primer concentration is at least about 10 μM. In further embodiments, the oligonucleotide primer concentration is at least about 10 μM and less than about 50 μM. In further embodiments, the thermostable polymerase is Phi29 DNA polymerase or a derivative or variant thereof. In other further embodiments, the thermostable polymerase is EQUIPHI29™. In other embodiments, the method is performed in a total reaction volume of at least about 100 μl. In other further embodiments, the method is performed in a total reaction volume of at least about 100 μl, about 200 μl, about 300 μl, about 400 μl, about 500 μl, about 600 μl, about 700 μl, about 800 μl, about 900 μl, about 1 ml, about 2 ml, about 3 ml, about 4 ml, about 5 ml, about 6 ml, about 7 ml, about 8 ml, about 9 ml, about 10 ml, about 15 ml, about 20 ml, about 25 ml, about 30 ml, about 35 ml, about 40 ml, about 45 ml, about 50 ml, about 55 ml, about 60 ml, about 65 ml, about 70 ml, about 75 ml, about 80 ml, about 85 ml, about 90 ml, about 95 ml, about 100 ml, about 200 ml, about 300 ml, about 400 ml, about 500 ml, about 600 ml, about 700 ml, about 800 ml, about 900 ml, about 1 L, about 2 L, about 3 L, 4 L, about 5 L, about 6 L, about 7 L, about 8 L, about 9 L, about 10 L, about 20 L, about 30 L, about 40 L, about 50 L, about 60 L, about 70 L, about 80 L, about 90 L, about 100 L, about 200 L, about 300 L, about 400 L, about 500 L, about 600 L, about 700 L, about 800 L, about 900 L, or about 1000 L.

In some embodiments, the method is performed in a reaction vessel that has a capacity of at least twice the total reaction volume. In some embodiments, the oligonucleotide primer hybridizes to a backbone sequence in the plasmid template. In some embodiments, the oligonucleotide primer is a universal primer.

In some embodiments, the dNTP concentration is about 4 mM.

### Isolation and Purification

Methods to generate and isolate a ceDNA vector, which is an exemplary closed-ended DNA vector, are described herein. For example, a closed-ended DNA vector, e.g., ceDNA vector produced by the synthetic methods described herein can be harvested or collected at an appropriate time after the last ligation reaction and can be optimized to achieve a high-yield production of the ceDNA vectors. The closed-ended DNA vector, e.g., ceDNA vectors can be purified by any means known to those of skill in the art for purification of DNA. In one embodiment, ceDNA vectors are purified as DNA molecules. Generally, any art-known nucleic acid purification methods can be adopted, as well as commercially available DNA extraction kits.

Alternatively, purification can be implemented by subjecting a reaction mixture to chromatographic separation. As one non-limiting example, the process can be performed by loading the reaction mixture on an ion exchange column (e.g. SARTOBIND Q®) which retains nucleic acids, and then eluting (e.g. with a 1.2 M NaCl solution) and performing a further chromatographic purification on a gel filtration column (e.g. 6 fast flow GE). The DNA vector, e.g., ceDNA vector is then recovered by, e.g., precipitation.

The presence of the ceDNA vector can be confirmed by digesting the vector DNA isolated from the cells with a restriction enzyme having a single recognition site on the DNA vector and analyzing both digested and undigested DNA material using gel electrophoresis to confirm the presence of characteristic bands of linear and continuous DNA as compared to linear and non-continuous DNA.

In some embodiments, the closed-ended DNA vectors produced by the synthetic production methods disclosed herein can be delivered to a target cell in vitro or in vivo by various suitable methods as discussed herein. Vectors alone can be applied or injected. Vectors can be delivered to a cell without the help of a transfection reagent or other physical means. Alternatively, vectors can be delivered using a transfection reagent or other physical means that facilitates entry of DNA into a cell, e.g., liposomes, alcohols, polylysine-rich compounds, arginine-rich compounds calcium phosphate, microvesicles, microinjection, and the like.

### Circular DNA Vectors Produced Using the Synthetic Production Method

Provided herein are various methods of in vitro production of DNA molecules and closed-ended DNA vectors. In some embodiments, the closed-ended DNA vector is a ceDNA vector, as described herein. In alternative embodiments, the closed-ended DNA vector is, e.g., a dumbbell DNA vector or a dog-bone DNA vector (see e.g., WO2010/0086626, incorporated by reference in its entirety herein).

### III. ceDNA Vectors in General

In some embodiments, a closed-ended DNA vector produced using the synthetic process as described herein is a ceDNA vector, including ceDNA vectors that can express a transgene. The ceDNA vectors described herein are not limited by size, thereby permitting, for example, expression of all of the components necessary for expression of a transgene from a single vector. The ceDNA vector is preferably duplex, e.g. self-complementary, over at least a portion of the molecule, such as the expression cassette (e.g. ceDNA is not a double stranded circular molecule). The ceDNA vector has covalently closed ends, and thus is resistant to exonuclease digestion (e.g. exonuclease I, exonuclease III), e.g. for over an hour at 37° C.

In general, a ceDNA vector produced using the synthetic process as described herein, comprises a transgene expression cassette, a first non-palindromic restriction endonuclease recognition site and a corresponding first cleavage site upstream of the transgene expression cassette, and a second non-palindromic restriction endonuclease recognition site and a corresponding second cleavage site downstream of the transgene expression cassette. In some embodiments, a ceDNA vector produced using the synthetic process as described herein, comprises in the 5′ to 3′ direction: a first adeno-associated virus (AAV) inverted terminal repeat (ITR), a nucleotide sequence of interest (for example a transgene expression cassette as described herein) and a second AAV ITR. The ITRs selected from any of: (i) at least one WT ITR and at least one modified AAV inverted terminal repeat (mod-ITR) (e.g., asymmetric modified ITRs); (ii) two modified ITRs where the mod-ITR pair have a different three-dimensional spatial organization with respect to each other (e.g., asymmetric modified ITRs), or (iii) symmetrical or substantially symmetrical WT-WT ITR pair, where each WT-ITR has the same three-dimensional spatial organization, or (iv) symmetrical or substantially symmetrical modified ITR pair, where each mod-ITR has the same three-dimensional spatial organization.

Encompassed herein are methods and compositions comprising the ceDNA vector produced using the synthetic process as described herein, which may further include a delivery system, such as but not limited to, a liposome nanoparticle delivery system. Non-limiting exemplary liposome nanoparticle systems encompassed for use are disclosed herein. In some aspects, the disclosure provides for a lipid nanoparticle comprising ceDNA and an ionizable lipid. For example, a lipid nanoparticle formulation that is made and loaded with a ceDNA vector obtained by the process is disclosed in International Patent Application Publication No. WO2019/051289, incorporated by reference in its entirety herein.

The ceDNA vectors produced using the synthetic process as described herein have no packaging constraints imposed by the limiting space within the viral capsid. This permits the insertion of control elements, e.g., regulatory switches as disclosed herein, large transgenes, multiple transgenes etc.

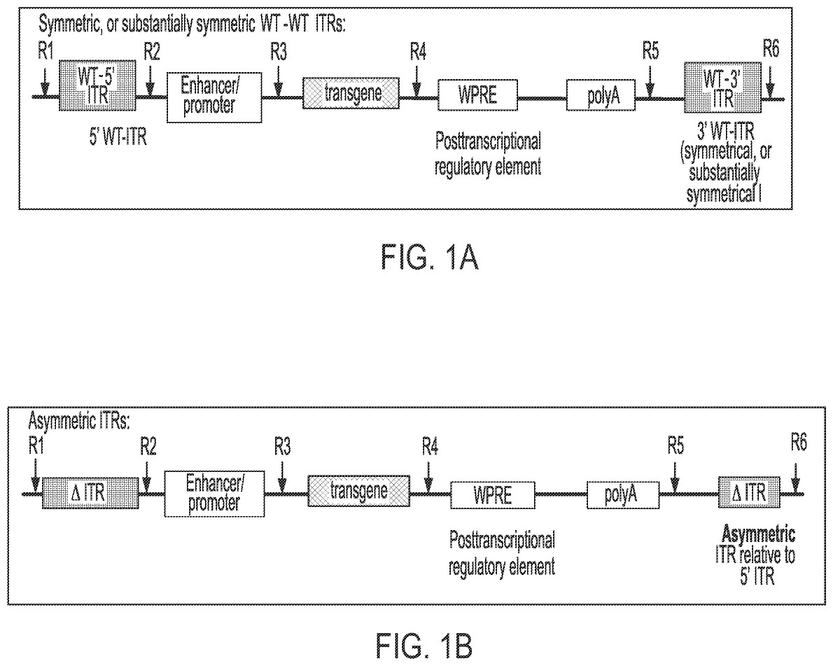

FIGS. 1A-1G of International Patent Application Publication No. WO2019/143885, show schematics of non-limiting, exemplary ceDNA vectors, or the corresponding sequence of ceDNA plasmids. ceDNA vectors are capsid-free and can be obtained from a plasmid encoding in this order: a first ITR, an expression cassette comprising a transgene and a second ITR. The expression cassette may include one or more regulatory sequences that allows and/or controls the expression of the transgene, e.g., where the expression cassette can comprise one or more of, in this order: an enhancer/promoter, an ORF reporter such as luciferase or a transgene, a post-transcription regulatory element (e.g., WPRE), and a polyadenylation and termination signal (e.g., BGH polyA).

The expression cassette can also comprise an internal ribosome entry site (IRES) and/or a 2A element. The cis-regulatory elements include, but are not limited to, a promoter, a riboswitch, an insulator, a mir-regulatable element, a post-transcriptional regulatory element, a tissue- and cell type-specific promoter and an enhancer. In some embodiments the ITR can act as the promoter for the transgene. In some embodiments, the ceDNA vector comprises additional components to regulate expression of the transgene, for example, a regulatory switch, which are described herein in the section entitled “Regulatory Switches” for controlling and regulating the expression of the transgene, and can include if desired, a regulatory switch which is a kill switch to enable controlled cell death of a cell comprising a ceDNA vector.

The expression cassette can comprise more than 4000 nucleotides, 5000 nucleotides, 10,000 nucleotides or 20,000 nucleotides, or 30,000 nucleotides, or 40,000 nucleotides or 50,000 nucleotides, or any range between about 4000-10,000 nucleotides or 10,000-50,000 nucleotides, or more than 50,000 nucleotides. In some embodiments, the expression cassette can comprise a transgene in the range of 500 to 50,000 nucleotides in length. In some embodiments, the expression cassette can comprise a transgene in the range of 500 to 75,000 nucleotides in length. In some embodiments, the expression cassette can comprise a transgene which is in the range of 500 to 10,000 nucleotides in length. In some embodiments, the expression cassette can comprise a transgene which is in the range of 1000 to 10,000 nucleotides in length. In some embodiments, the expression cassette can comprise a transgene which is in the range of 500 to 5,000 nucleotides in length. The ceDNA vectors do not have the size limitations of encapsidated AAV vectors, thus enable delivery of a large-size expression cassette to provide efficient transgene. In some embodiments, the ceDNA vector is devoid of prokaryote-specific methylation.

A ceDNA expression cassette can include, for example, an expressible exogenous sequence (e.g., open reading frame) or transgene that encodes a protein that is either absent, inactive, or insufficient activity in the recipient subject or a gene that encodes a protein having a desired biological or a therapeutic effect. The transgene can encode a gene product that can function to correct the expression of a defective gene or transcript. In principle, the expression cassette can include any gene that encodes a protein, polypeptide or RNA that is either reduced or absent due to a mutation or which conveys a therapeutic benefit when overexpressed is considered to be within the scope of the disclosure.

The expression cassette can comprise any transgene useful for treating a disease or disorder in a subject. A ceDNA vector produced using the synthetic process as described herein can be used to deliver and express any gene of interest in the subject, which includes but are not limited to, nucleic acids encoding polypeptides, or non-coding nucleic acids (e.g., RNAi, miRs etc.), as well as exogenous genes and nucleotide sequences, including virus sequences in a subjects' genome, e.g., HIV virus sequences and the like. Preferably a ceDNA vector disclosed herein is used for therapeutic purposes (e.g., for medical, diagnostic, or veterinary uses) or immunogenic polypeptides. In certain embodiments, a ceDNA vector is useful to express any gene of interest in the subject, which includes one or more polypeptides, peptides, ribozymes, peptide nucleic acids, siRNAs, RNAi's, antisense oligonucleotides, antisense polynucleotides, or RNAs (coding or non-coding; e.g., siRNAs, shRNAs, micro-RNAs, and their antisense counterparts (e.g., antagoMiR)), antibodies, antigen binding fragments, or any combination thereof.

The expression cassette can also encode polypeptides, sense or antisense oligonucleotides, or RNAs (coding or non-coding; e.g., siRNAs, shRNAs, micro-RNAs, and their antisense counterparts (e.g., antagoMiR)). Expression cassettes can include an exogenous sequence that encodes a reporter protein to be used for experimental or diagnostic purposes, such as β-lactamase, β-galactosidase (LacZ), alkaline phosphatase, thymidine kinase, green fluorescent protein (GFP), chloramphenicol acetyltransferase (CAT), luciferase, and others well known in the art.

Sequences provided in the expression cassette, expression construct of a ceDNA vector described herein can be codon optimized for the target host cell. As used herein, the term “codon optimized” or “codon optimization” refers to the process of modifying a nucleic acid sequence for enhanced expression in the cells of the vertebrate of interest, e.g., mouse or human, by replacing at least one, more than one, or a significant number of codons of the native sequence (e.g., a prokaryotic sequence) with codons that are more frequently or most frequently used in the genes of that vertebrate. Various species exhibit particular bias for certain codons of a particular amino acid. Typically, codon optimization does not alter the amino acid sequence of the original translated protein. Optimized codons can be determined using e.g., Aptagen's GENE FORGE® codon optimization and custom gene synthesis platform (Aptagen, Inc., 2190 Fox Mill Rd. Suite 300, Herndon, Va. 20171) or another publicly available database.

In some embodiments, a transgene expressed by the ceDNA vector is a therapeutic gene. In some embodiments, a therapeutic gene is an antibody, or antibody fragment, or antigen-binding fragment thereof, e.g., a neutralizing antibody or antibody fragment and the like.

In particular, a therapeutic gene is one or more therapeutic agent(s), including, but not limited to, for example, protein(s), polypeptide(s), peptide(s), enzyme(s), antibodies, antigen binding fragments, as well as variants, and/or active fragments thereof, for use in the treatment, prophylaxis, and/or amelioration of one or more symptoms of a disease, dysfunction, injury, and/or disorder. Exemplary therapeutic genes are described herein in the section entitled “Method of Treatment”.

There are many structural features of ceDNA vectors that differ from plasmid-based expression vectors. ceDNA vectors produced by the synthetic methods herein may possess one or more of the following features: the lack of original (i.e. not inserted) bacterial DNA, the lack of a prokaryotic origin of replication, being self-containing, i.e., they do not require any sequences other than the two ITRs, including the Rep binding and terminal resolution sites (RBS and TRS), and an exogenous sequence between the ITRs, the presence of ITR sequences that form hairpins, and the absence of bacterial-type DNA methylation or indeed any other methylation associated with production in a given cell type and considered abnormal by a mammalian host. In general, it is preferred for the present vectors not to contain any prokaryotic DNA but it is contemplated that some prokaryotic DNA may be inserted as an exogenous sequence, as a non-limiting example in a promoter or enhancer region. Another important feature distinguishing ceDNA vectors from plasmid expression vectors is that ceDNA vectors are single-stranded linear DNA having closed ends, while plasmids are always double-stranded DNA.

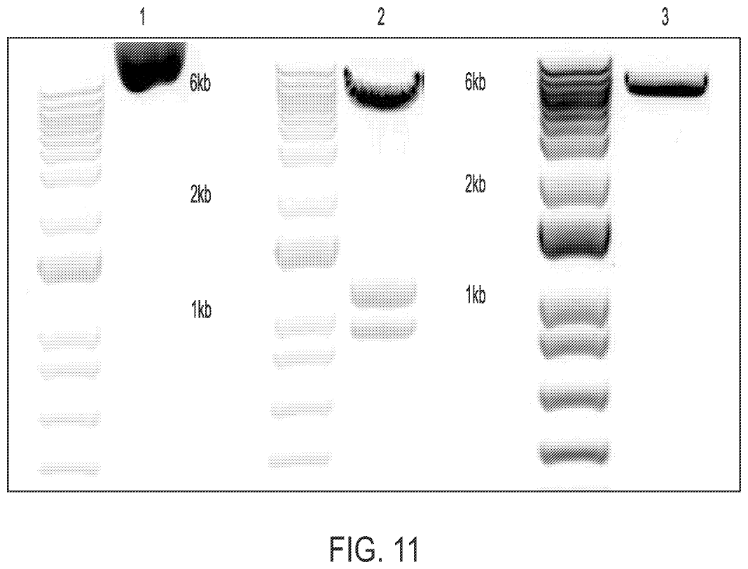

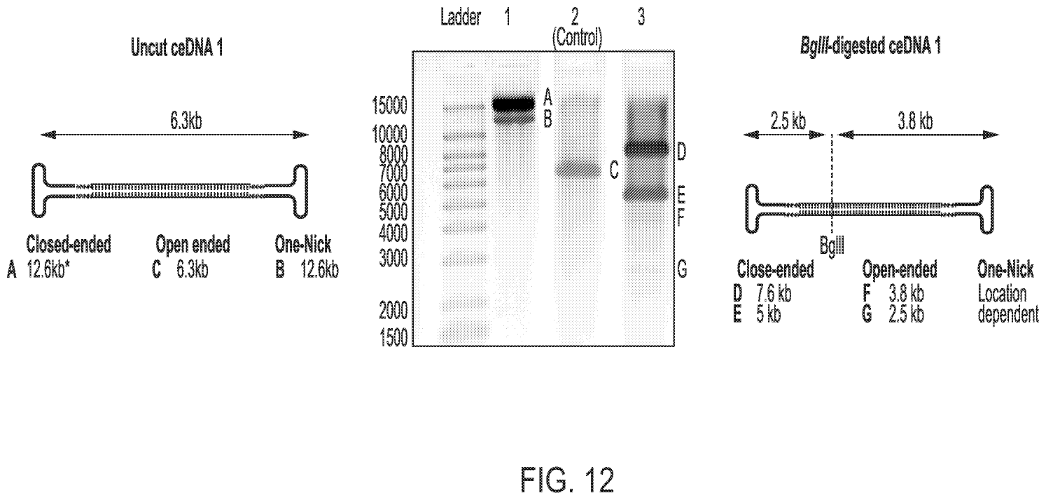

ceDNA vectors produced by the synthetic methods provided herein preferably have a linear and continuous structure rather than a non-continuous structure, as determined by restriction enzyme digestion assay (see e.g., FIG. 11 and FIG. 12). The linear and continuous structure is believed to be more stable from attack by cellular endonucleases, as well as less likely to be recombined and cause mutagenesis. Thus, a ceDNA vector in the linear and continuous structure is a preferred embodiment. The continuous, linear, single strand intramolecular duplex ceDNA vector can have covalently bound terminal ends, without sequences encoding AAV capsid proteins. These ceDNA vectors are structurally distinct from plasmids (including ceDNA plasmids described herein), which are circular duplex nucleic acid molecules of bacterial origin. The complimentary strands of plasmids may be separated following denaturation to produce two nucleic acid molecules, whereas in contrast, ceDNA vectors, while having complimentary strands, are a single DNA molecule and therefore even if denatured, remain a single molecule. In some embodiments, ceDNA vectors as described herein can be produced without DNA base methylation of prokaryotic type, unlike plasmids. Therefore, the ceDNA vectors and ceDNA-plasmids are different both in term of structure (in particular, linear versus circular) and also in view of the methods used for producing and purifying these different objects (see below), and also in view of their DNA methylation which is of prokaryotic type for ceDNA-plasmids and of eukaryotic type for the ceDNA vector.

There are several advantages of using a ceDNA vector as described herein over plasmid-based expression vectors, such advantages include, but are not limited to: 1) plasmids contain bacterial DNA sequences and are subjected to prokaryotic-specific methylation, e.g., 6-methyl adenosine and 5-methyl cytosine methylation, whereas capsid-free AAV vector sequences are of eukaryotic origin and do not undergo prokaryotic-specific methylation; as a result, capsid-free AAV vectors are less likely to induce inflammatory and immune responses compared to plasmids; 2) while plasmids require the presence of a resistance gene during the production process, ceDNA vectors do not; 3) while a circular plasmid is not delivered to the nucleus upon introduction into a cell and requires overloading to bypass degradation by cellular nucleases, ceDNA vectors contain viral cis-elements, i.e., ITRs, that confer resistance to nucleases and can be designed to be targeted and delivered to the nucleus. It is hypothesized that the minimal defining elements indispensable for ITR function are a Rep-binding site SEQ ID NO: 60 of International Patent Application Publication No. WO2019/143885) for AAV2) and a terminal resolution site (TRS; 5′-AGTTGG-3′ (SEQ ID NO: 64 of WO2019/143885) for AAV2) plus a variable palindromic sequence allowing for hairpin formation; and 4) ceDNA vectors do not have the over-representation of CpG dinucleotides often found in prokaryote-derived plasmids that reportedly binds a member of the Toll-like family of receptors, eliciting a T cell-mediated immune response. In contrast, transductions with capsid-free AAV vectors disclosed herein can efficiently target cell and tissue-types that are difficult to transduce with conventional AAV virions using various delivery reagent.

### Unique Junction Sequences

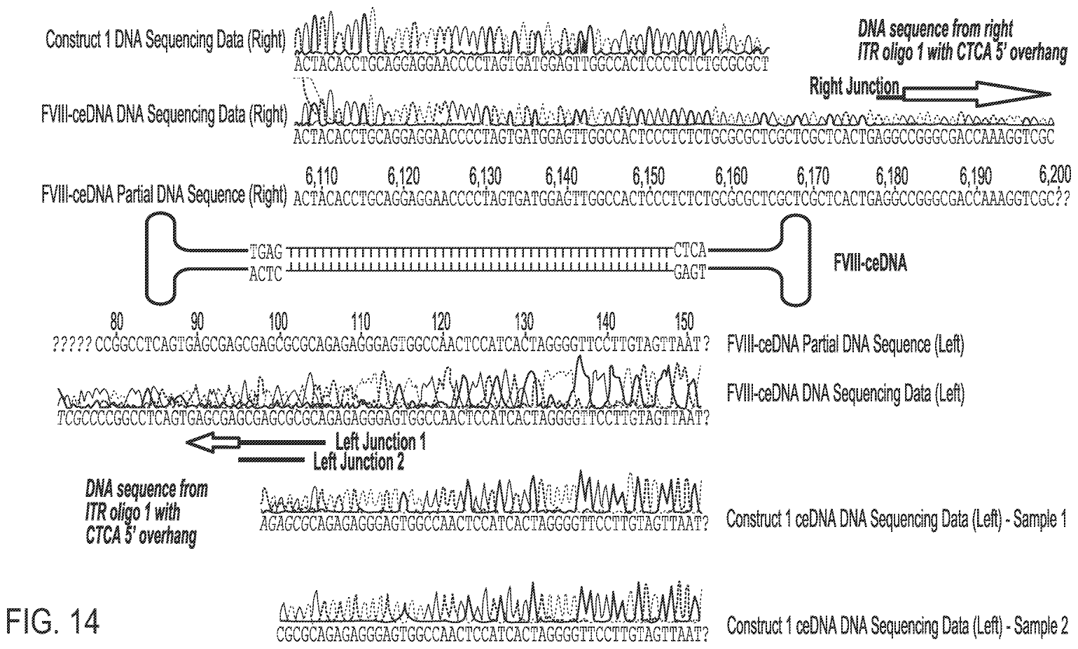

The ceDNA vectors prepared using the cell-free synthetic methods described herein include nucleotide sequences at the ligation junctions between the insert and the ITR oligonucleotides on both ends that unique to the ceDNA. Such junction sequences are not found in the ITR oligonucleotides nor in the double-stranded DNA construct from which the transgene expression cassette is excised. The presence of such junction sequences, as revealed in DNA sequencing analysis (see e.g., FIG. 14) is useful for confirming the success of the ligation reaction.

Accordingly, another aspect of this disclosure is directed to ITR nucleotide sequences found in ceDNA vectors, ceDNA vectors comprising these ITR nucleotide sequences, as well as compositions, host cells, and transgenic animals comprising such ceDNA vectors. A non-exhaustive list of examples of such ITR nucleotide sequences are provided below in Table 3.

TABLE 3
ITR nucleotide sequences in synthetically-
produced ceDNA vectors

| SEQ | Left | |
|---|---|---|
| ID | or | |
| NO: | Right | Sequence (5′ → 3′) |
| 18 | Left | CTCACTGAGGCCGCCCGGGCAAAGCCCGGGCGT |
| | | CGGGCGACCTTTGGTCGCCCGGCCTCAG |
| 19 | Left | CTCACTGAGGCCGCCCGGGCAAAGCCCGGGCGT |
| | | CGGGCGACCTTTGGTCGCCCGGCCTCAGTGAGC |
| | | GAGCGAGCGCGCAGAGAGGGAGTGGCCAACTC |
| | | CATCACTAGGGGTTCCT |
| 20 | Left | CTCTCTGAGGCCGCCCGGGCAAAGCCCGGGCGT |
| | | CGGGCGACCTTTGGTCGCCCGGCCTCAG |
| 21 | Left | CTCTCTGAGGCCGCCCGGGCAAAGCCCGGGCGT |
| | | CGGGCGACCTTTGGTCGCCCGGCCTCAGTGAGC |
| | | GAGCGAGCGCGCAGAGAGGGAGTGGCCAACTC |
| | | CATCACTAGGGGTTCCT |
| 22 | Left | CACTGAGGCCGCCCGGGCAAAGCCCGGGCGTC |
| | | GGGCGACCTTTGGTCGCCCGGCCTCAG |
| 23 | Left | CACTGAGGCCGCCCGGGCAAAGCCCGGGCGTC |
| | | GGGCGACCTTTGGTCGCCCGGCCTCAGTGAGC |
| | | GAGCGAGCGCGCAGAGAGGGAGTGGCCAACT |
| | | CCATCACTAGGGGTTCCT |
| 24 | Left | CTCACTGAGGCCGCCCGGGCAAAGCCCGGGCG |
| | | TCGGGCGACCTTTGGTCGCCCGGCCTCAG |
| 25 | Left | CTCACTGAGGCCGCCCGGGCAAAGCCCGGGCG |
| | | TCGGGCGACCTTTGGTCGCCCGGCCTCAGTGAG |
| | | CGAGCGAGCGCGCAGAGAGGGAGTGGCCAACT |
| | | CCATCACTAGGGGTTCCT |
| 26 | Right | CTGAGGCCGGGCGACCAAAGGTCGCCCGACGCC |
| | | CGGGCTTTGCCCGGGCGGCCTCAGTGAG |
| 27 | Right | AGGAACCCCTAGTGATGGAGTTGGCCACTCCCTC |
| | | TCTGCGCGCTCGCTCGCTCACTGAGGCCGGGCGA |
| | | CCAAAGGTCGCCCGACGCCCGGGCTTTGCCCGGG |
| | | CGGCCTCAGTGAG |
| 28 | Right | CTGAGGCCGGGCGACCAAAGGTCGCCCGACGCCC |
| | | GGGCGGCCTC |
| 29 | Right | AGGAACCCCTAGTGATGGAGTTGGCCACTCCCTCT |
| | | CTGCGCGCTCGCTCGCTCACTGAGGCCGGGCGACC |
| | | AAAGGTCGCCCGACGCCCGGGCGGCCTC |
| 30 | Right | CTGAGGCCGGGCGACCAAAGGTCGCCCGACGCCC |
| | | GGGCTTTGCCCGGGCGGCCTCAGTGAG |
| 31 | Right | AGGAACCCCTAGTGATGGAGTTGGCCACTCCCTCTC |
| | | TGCGCGCTCGCTCGCTCACTGAGGCCGGGCGACCAA |
| | | AGGTCGCCCGACGCCCGGGCTTTGCCCGGGCGGCCT |
| | | CAGTGAG |

In some embodiments, the ITR nucleotide sequences provided herein further include a spacer sequence. In one embodiment, the spacer is selected from any one of the spacer sequences provided in Table 2. In one embodiment, any one of the ITR nucleotide sequences of Table 3 may fuirther include any one of the spacer sequences provided in Table 2.

### IV. ITRs

As disclosed herein, ceDNA vectors contain a transgene or heterologous nucleic acid sequence positioned between at least two inverted terminal repeats (ITRs), where the ITRs can be an asymmetrical ITR pair or a symmetrical or substantially symmetrical ITR pair, as these terms are defined herein. A ceDNA vector as disclosed herein can comprise ITR sequences that are selected from any of: (i) at least one WT-ITR and at least one modified AAV inverted terminal repeat (mod-ITR) (e.g., asymmetric modified ITRs); (ii) two modified ITRs where the mod-ITR pair have a different three-dimensional spatial organization with respect to each other (e.g., asymmetric modified ITRs), or (iii) symmetrical or substantially symmetrical WT-WT ITR pair, where each WT-ITR has the same three-dimensional spatial organization, or (iv) symmetrical or substantially symmetrical modified ITR pair, where each mod-ITR has the same three-dimensional spatial organization, where the methods of the present disclosure may further include a delivery system, such as but not limited to a liposome nanoparticle delivery system.

In some embodiments, the ITR sequence can be from viruses of the Parvoviridae family, which includes two subfamilies: Parvovirinae, which infect vertebrates, and Densovirinae, which infect insects. The subfamily Parvovirinae (referred to as the parvoviruses) includes the genus Dependovirus, the members of which, under most conditions, require coinfection with a helper virus such as adenovirus or herpes virus for productive infection. The genus Dependovirus includes adeno-associated virus (AAV), which normally infects humans (e.g., serotypes 2, 3A, 3B, 5, and 6) or primates (e.g., serotypes 1 and 4), and related viruses that infect other warm-blooded animals (e.g., bovine, canine, equine, and ovine adeno-associated viruses). The parvoviruses and other members of the Parvoviridae family are generally described in Kenneth I. Berns, “Parvoviridae: The Viruses and Their Replication,” Chapter 69 in FIELDS VIROLOGY (3d Ed. 1996).

While the WT-ITRs exemplified in the specification and Examples herein are AAV2 WT-ITRs, one of ordinary skill in the art is aware that one can as stated above use ITRs from any known parvovirus, for example a dependovirus such as AAV (e.g., AAV1, AAV2, AAV3, AAV4, AAV5, AAV 5, AAV7, AAV8, AAV9, AAV10, AAV 11, AAV12, AAVrh8, AAVrh10, AAV-DJ, and AAV-DJ8 genome. E.g., NCBI: NC 002077; NC 001401; NC001729; NC001829; NC006152; NC 006260; NC 006261), chimeric ITRs, or ITRs from any synthetic AAV. In some embodiments, the AAV can infect warm-blooded animals, e.g., avian (AAAV), bovine (BAAV), canine, equine, and ovine adeno-associated viruses. In some embodiments the ITR is from B19 parvovirus (GenBank Accession No: NC 000883), Minute Virus from Mouse (MVM) (GenBank Accession No. NC 001510); goose parvovirus (GenBank Accession No. NC 001701); snake parvovirus 1 (GenBank Accession No. NC 006148). In some embodiments, the 5′ WT-ITR can be from one serotype and the 3′ WT-ITR from a different serotype, as discussed herein.

An ordinarily skilled artisan is aware that ITR sequences have a common structure of a double-stranded Holliday junction, which typically is a T-shaped or Y-shaped hairpin structure (see e.g., FIG. 10), where each WT-ITR is formed by two loops (B-B′ and C-C′, both of which comprising palindromic double-stranded DNA sequences) and a stem (A-A′, also comprising a palindromic double-stranded DNA sequence), and a single stranded D sequence, (where the order of these palindromic sequences defines the flip or flop orientation of the ITR). See, for example, structural analysis and sequence comparison of ITRs from different AAV serotypes (AAV1-AAV6) and described in Grimm et al., J. Virology, 2006; 80(1); 426-439; Yan et al., J. Virology, 2005; 364-379; Duan et al., Virology 1999; 261; 8-14. One of ordinary skill in the art can readily determine WT-ITR sequences from any AAV serotype for use in a ceDNA vector or ceDNA-plasmid based on the exemplary AAV2 ITR sequences provided herein. See, for example, the sequence comparison of ITRs from different AAV serotypes (AAV1-AAV6, and avian AAV (AAAV) and bovine AAV (BAAV)) described in Grimm et al., J. Virology, 2006; 80(1); 426-439; that show the % identity of the left ITR of AAV2 to the left ITR from other serotypes: AAV-1 (84%), AAV-3 (86%), AAV-4 (79%), AAV-5 (58%), AAV-6 (left ITR) (100%) and AAV-6 (right ITR) (82%).

### Symmetrical or Substantially Symmetrical ITR Pairs

In some embodiments, a ceDNA vector as described herein comprises, in the 5′ to 3′ direction: a first adeno-associated virus (AAV) inverted terminal repeat (ITR), a nucleotide sequence of interest (for example an expression cassette as described herein) and a second AAV ITR, where the first ITR (5′ ITR) and the second ITR (3′ ITR) are symmetrical, or substantially symmetrical with respect to each other—that is, a ceDNA vector can comprise ITRs that have a symmetrical three-dimensional spatial organization such that their structure is the same shape in geometrical space, or have the same A-A′ stem, C-C′ and B-B′ loops in 3D space. In such an embodiment, a symmetrical ITR pair, or substantially symmetrical ITR pair can be modified ITRs (e.g., mod-ITRs) that are not wild-type ITRs. A mod-ITR pair can have the same sequence which has one or more modifications from wild-type ITR and are reverse complements (inverted) of each other. In alternative embodiments, a modified ITR pair are substantially symmetrical as defined herein, that is, the modified ITR pair can have a different sequence but have corresponding or the same symmetrical three-dimensional shape.

### Wildtype ITRs (WT-ITRs)

In some embodiments, the symmetrical ITRs, or substantially symmetrical ITRs are wild type (WT-ITRs) as described herein. That is, both ITRs have a wild-type sequence, but do not necessarily have to be WT-ITRs from the same AAV serotype. That is, in some embodiments, one WT-ITR can be from one AAV serotype, and the other WT-ITR can be from a different AAV serotype. In such an embodiment, a WT-ITR pair are substantially symmetrical as defined herein, that is, they can have one or more conservative nucleotide modification while still retaining the symmetrical three-dimensional spatial organization.

Accordingly, as disclosed herein, ceDNA vectors contain a transgene or heterologous nucleic acid sequence positioned between at least two flanking wild-type inverted terminal repeats (WT-ITRs), that are either the reverse complement (inverted) of each other, or alternatively, are substantially symmetrical relative to each other—that is a WT-ITR pair having symmetrical three-dimensional spatial organization. In some embodiments, a wild-type ITR sequence (e.g. AAV WT-ITR) comprises a functional Rep binding site (RBS; e.g., SEQ ID NO: 60 of International Patent Application Publication No. WO2019/143885 for AAV2, incorporated by reference in its entirety herein) and a functional terminal resolution site (TRS; e.g. 5′-AGTT-3′, SEQ ID NO: 62 of WO2019/143885, incorporated by reference in its entirety herein).

In one aspect, ceDNA vectors are obtainable from a double-stranded DNA construct that encodes a transgene operatively positioned between at least two WT-ITRs (e.g. AAV WT-ITRs). That is, both ITRs have a wild-type sequence, but do not necessarily have to be WT-ITRs from the same AAV serotype. That is, in some embodiments, one WT-ITR can be from one AAV serotype, and the other WT-ITR can be from a different AAV serotype. In such an embodiment, the WT-ITR pair are substantially symmetrical as defined herein, that is, they can have one or more conservative nucleotide modification while still retaining the symmetrical three-dimensional spatial organization. In some embodiments, the 5′ WT-ITR is from one AAV serotype, and the 3′ WT-ITR is from the same or a different AAV serotype. In some embodiments, the 5′ WT-ITR and the 3′WT-ITR are mirror images of each other, that is they are symmetrical. In some embodiments, the 5′ WT-ITR and the 3′ WT-ITR are from the same AAV serotype.

WT-ITRs are well-known. In one embodiment the two ITRs are from the same AAV2 serotype. In certain embodiments one can use WT from other serotypes. There are a number of serotypes that are homologous, e.g. AAV2, AAV4, AAV6, AAV8. In one embodiment, closely homologous ITRs (e.g. ITRs with a similar loop structure) can be used. In another embodiment, one can use AAV WT ITRs that are more diverse, e.g., AAV2 and AAV5, and still another embodiment, one can use an ITR that is substantially WT—that is, it has the basic loop structure of the WT but some conservative nucleotide changes that do not alter or affect the properties. When using WT-ITRs from the same viral serotype, one or more regulatory sequences may further be used. In certain embodiments, the regulatory sequence is a regulatory switch that permits modulation of the activity of the ceDNA.

In some embodiments, one aspect of the technology described herein relates to a synthetically produced ceDNA vector, wherein the ceDNA vector comprises at least one heterologous nucleotide sequence, operably positioned between two wild-type inverted terminal repeat sequences (WT-ITRs), wherein the WT-ITRs can be from the same serotype, different serotypes or substantially symmetrical with respect to each other (i.e., have the symmetrical three-dimensional spatial organization such that their structure is the same shape in geometrical space, or have the same A, C-C′ and B-B′ loops in 3D space). In some embodiments, the symmetric WT-ITRs comprises a functional terminal resolution site and a Rep binding site. In some embodiments, the heterologous nucleic acid sequence encodes a transgene, and wherein the vector is not in a viral capsid.

In some embodiments, the WT-ITRs are the same but the reverse complement of each other. For example, the sequence AACG in the 5′ ITR may be CGTT (i.e., the reverse complement) in the 3′ ITR at the corresponding site. In one example, the 5′ WT-ITR sense strand comprises the sequence of ATCGATCG and the corresponding 3′ WT-ITR sense strand comprises CGATCGAT (i.e., the reverse complement of ATCGATCG). In some embodiments, the WT-ITRs ceDNA further comprises a terminal resolution site and a replication protein binding site (sometimes referred to as a replicative protein binding site), e.g. a Rep binding site (RBS).

Exemplary WT-ITR sequences for use in the ceDNA vectors comprising WT-ITRs are shown in Table 2 of International Patent Application Publication No. WO2019/143885, which shows pairs of WT-ITRs (5′ WT-ITR and the 3′ WT-ITR).

As an exemplary example, the present disclosure provides a synthetically produced ceDNA vector comprising a promoter operably linked to a transgene (e.g., heterologous nucleic acid sequence), with or without the regulatory switch, where the ceDNA is devoid of capsid proteins and is: (a) produced from a ceDNA-plasmid (e.g., see FIGS. 1F-1G of WO2019/143885) that encodes WT-ITRs, where each WT-ITR has the same number of intramolecularly duplexed base pairs in its hairpin secondary configuration (preferably excluding deletion of any AAA or TTT terminal loop in this configuration compared to these reference sequences), and (b) is identified as ceDNA using the assay for the identification of ceDNA by agarose gel electrophoresis under native gel and denaturing conditions.

In some embodiments, the flanking WT-ITRs are substantially symmetrical to each other. In this embodiment the 5′ WT-ITR can be from one serotype of AAV, and the 3′ WT-ITR from a different serotype of AAV, such that the WT-ITRs are not identical reverse complements. For example, the 5′ WT-ITR can be from AAV2, and the 3′ WT-ITR from a different serotype (e.g. AAV1, 3, 4, 5, 6, 7, 8, 9, 10, 11, and 12. In some embodiments, WT-ITRs can be selected from two different parvoviruses selected from any to of: AAV1, AAV2, AAV3, AAV4, AAV5, AAV6, AAV7, AAV8, AAV9, AAV10, AAV11, AAV12, AAV13, snake parvovirus (e.g., royal python parvovirus), bovine parvovirus, goat parvovirus, avian parvovirus, canine parvovirus, equine parvovirus, shrimp parvovirus, porcine parvovirus, or insect AAV. In some embodiments, such a combination of WT ITRs is the combination of WT-ITRs from AAV2 and AAV6. In one embodiment, the substantially symmetrical WT-ITRs are when one is inverted relative to the other ITR at least 90% identical, at least 95% identical, at least 96% . . . 97% . . . 98% . . . 99% . . . 99.5% and all points in between, and has the same symmetrical three-dimensional spatial organization. In some embodiments, a WT-ITR pair are substantially symmetrical as they have symmetrical three-dimensional spatial organization, e.g., have the same 3D organization of the A-A′ and D-D′ stem regions and B-B′ and C-C′ loops. In one embodiment, a substantially symmetrical WT-ITR pair are inverted relative to the other, and are at least 95% identical, at least 96% . . . 97% . . . 98% . . . 99% . . . 99.5% and all points in between, to each other, and one WT-ITR retains the Rep-binding site (RBS) (SEQ ID NO: 60 of International Patent Application Publication No. WO2019/143885 and a terminal resolution site (trs). In some embodiments, a substantially symmetrical WT-ITR pair are inverted relative to each other, and are at least 95% identical, at least 96% . . . 97% . . . 98% . . . 99% . . . 99.5% and all points in between, to each other, and one WT-ITR retains the Rep-binding site (RBS) (SEQ ID NO: 60 of WO2019/143885) and a terminal resolution site (trs) and in addition to a variable palindromic sequence allowing for hairpin secondary structure formation. Homology can be determined by standard means well known in the art such as BLAST (Basic Local Alignment Search Tool), BLASTN at default setting.

In some embodiments, the structural element of the ITR can be any structural element that is involved in the functional interaction of the ITR with a large Rep protein (e.g., Rep 78 or Rep 68). In certain embodiments, the structural element provides selectivity to the interaction of an ITR with a large Rep protein, i.e., determines at least in part which Rep protein functionally interacts with the ITR. In other embodiments, the structural element physically interacts with a large Rep protein when the Rep protein is bound to the ITR. Each structural element can be, e.g., a secondary structure of the ITR, a nucleotide sequence of the ITR, a spacing between two or more elements, or a combination of any of the above. In one embodiment, the structural elements are selected from the group consisting of an A and an A′ arm, a B and a B′ arm, a C and a C′ arm, a D arm, a Rep binding site (RBE) and an RBE′ (i.e., complementary RBE sequence), and a terminal resolution sire (trs).

Table 1 of International Patent Application Publication No. WO2019/143885, provides a non-exhaustive list of exemplary combinations of WT-ITRs. Table 2 of WO2019/143385 provides the sequences of a non-exhaustive list of exemplary WT-ITRs from different AAV serotypes.

In some embodiments, the nucleotide sequence of the WT-ITR sequence can be modified (e.g., by modifying 1, 2, 3, 4 or 5, or more nucleotides or any range therein), whereby the modification is a substitution for a complementary nucleotide, e.g., G for a C, and vice versa, and T for an A, and vice versa.

In certain embodiments of the present disclosure, the synthetically produced ceDNA vector does not have a WT-ITR consisting of the nucleotide sequence selected from any of: SEQ ID NOs: 1, 2, 5-14 of WO2019/143885. In alternative embodiments of the present disclosure, if a ceDNA vector has a WT-ITR comprising the nucleotide sequence selected from any of: SEQ ID NOs: 1, 2, 5-14 of WO2019/143885, then the flanking ITR is also WT and the ceDNA vector comprises a regulatory switch, e.g., as disclosed herein and in International Patent Application Publication No. WO2019/051255 (e.g., see Table 11 of WO2019/051255, incorporated by reference in its entirety herein). In some embodiments, the ceDNA vector comprises a regulatory switch as disclosed herein and a WT-ITR selected having the nucleotide sequence selected from any of the group consisting of: SEQ ID NO: 1, 2, 5-14 of WO2019/143885.

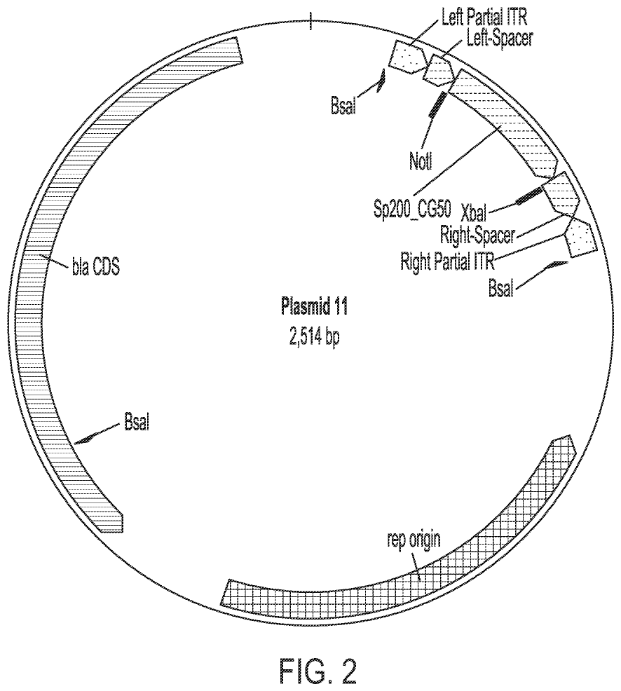

The ceDNA vector described herein can include WT-ITR structures that retains an operable RBE, trs and RBE′ portion. FIG. 2A and FIG. 2B of WO2019/143885, using wild-type ITRs for exemplary purposes, show one possible mechanism for the operation of a trs site within a wild type ITR structure portion of a ceDNA vector. In some embodiments, the ceDNA vector contains one or more functional WT-ITR polynucleotide sequences that comprise a Rep-binding site (SEQ ID NO: 60 of WO2019/143885) for AAV2) and a terminal resolution site (TRS; 5′-AGTT (SEQ ID NO: 62 of WO2019/143885)). In some embodiments, at least one WT-ITR is functional. In alternative embodiments, where a ceDNA vector comprises two WT-ITRs that are substantially symmetrical to each other, at least one WT-ITR is functional and at least one WT-ITR is non-functional.

Modified ITRs (Mod-ITRs) in General for ceDNA Vectors Comprising Asymmetric ITR Pairs or Symmetrical or ITR Pairs

As discussed herein, a synthetically produced ceDNA vector can comprise a symmetrical ITR pair or an asymmetrical ITR pair. In both instances, one or both of the ITRs can be modified ITRs—the difference being that in the first instance (i.e., symmetric mod-ITRs), the mod-ITRs have the same three-dimensional spatial organization (i.e., have the same A-A′ and D-D′ stem region and B-B′ and C-C′ loop configurations), whereas in the second instance (i.e., asymmetric mod-ITRs), the mod-ITRs have a different three-dimensional spatial organization (i.e., have a different configuration of A-A′ and D-D′ stem region and B-B′ and C-C′ loops).

In some embodiments, a modified ITR is an ITRs that is modified by deletion, insertion, and/or substitution as compared to a wild-type ITR sequence (e.g. AAV ITR). In some embodiments, at least one of the ITRs in the ceDNA vector comprises a functional Rep binding site (RBS; e.g. SEQ ID NO: 60 of WO2019/143885) and a functional terminal resolution site (TRS; e.g. 5′-AGTT-3′, SEQ ID NO: 62 of WO2019/143885) In one embodiment, at least one of the ITRs is a non-functional ITR. In one embodiment, the different or modified ITRs are not each wild type ITRs from different serotypes.

Specific alterations and mutations in the ITRs are described in detail herein, but in the context of ITRs, “altered” or “mutated” or “modified”, it indicates that nucleotides have been inserted, deleted, and/or substituted relative to the wild-type, reference, or original ITR sequence. The altered or mutated ITR can be an engineered ITR. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature.

In some embodiments, a mod-ITR may be synthetic. In one embodiment, a synthetic ITR is based on ITR sequences from more than one AAV serotype. In another embodiment, a synthetic ITR includes no AAV-based sequence. In yet another embodiment, a synthetic ITR preserves the ITR structure described above although having only some or no AAV-sourced sequence. In some aspects, a synthetic ITR may interact preferentially with a wild type Rep or a Rep of a specific serotype, or in some instances will not be recognized by a wild-type Rep and be recognized only by a mutated Rep.

The skilled artisan can determine the corresponding sequence in other serotypes by known means. For example, determining if the change is in the A, A′, B, B′, C, C′ or D-D′ region and determine the corresponding region in another serotype. One can use BLAST® (Basic Local Alignment Search Tool) or other homology alignment programs at default status to determine the corresponding sequence. The disclosure further provides populations and pluralities of ceDNA vectors comprising mod-ITRs from a combination of different AAV serotypes—that is, one mod-ITR can be from one AAV serotype and the other mod-ITR can be from a different serotype. Without wishing to be bound by theory, in one embodiment one ITR can be from or based on an AAV2 ITR sequence and the other ITR of the ceDNA vector can be from or be based on any one or more ITR sequence of AAV serotype 1 (AAV1), AAV serotype 4 (AAV4), AAV serotype 5 (AAV5), AAV serotype 6 (AAV6), AAV serotype 7 (AAV7), AAV serotype 8 (AAV8), AAV serotype 9 (AAV9), AAV serotype 10 (AAV10), AAV serotype 11 (AAV11), or AAV serotype 12 (AAV12).

Any parvovirus ITR can be used as an ITR or as a base ITR for modification. Preferably, the parvovirus is a dependovirus. More preferably AAV. The serotype chosen can be based upon the tissue tropism of the serotype. AAV2 has a broad tissue tropism, AAV1 preferentially targets to neuronal and skeletal muscle, and AAV5 preferentially targets neuronal, retinal pigmented epithelia, and photoreceptors. AAV6 preferentially targets skeletal muscle and lung. AAV8 preferentially targets liver, skeletal muscle, heart, and pancreatic tissues. AAV9 preferentially targets liver, skeletal and lung tissue. In one embodiment, the modified ITR is based on an AAV2 ITR.

More specifically, the ability of a structural element to functionally interact with a particular large Rep protein can be altered by modifying the structural element. For example, the nucleotide sequence of the structural element can be modified as compared to the wild-type sequence of the ITR. In one embodiment, the structural element (e.g., A arm, A′ arm, B arm, B′ arm, C arm, C′ arm, D arm, RBE, RBE′, and trs) of an ITR can be removed and replaced with a wild-type structural element from a different parvovirus. For example, the replacement structure can be from AAV1, AAV2, AAV3, AAV4, AAV5, AAV6, AAV7, AAV8, AAV9, AAV10, AAV11, AAV12, AAV13, snake parvovirus (e.g., royal python parvovirus), bovine parvovirus, goat parvovirus, avian parvovirus, canine parvovirus, equine parvovirus, shrimp parvovirus, porcine parvovirus, or insect AAV. For example, the ITR can be an AAV2 ITR and the A or A′ arm or RBE can be replaced with a structural element from AAV5. In another example, the ITR can be an AAV5 ITR and the C or C′ arms, the RBE, and the trs can be replaced with a structural element from AAV2. In another example, the AAV ITR can be an AAV5 ITR with the B and B′ arms replaced with the AAV2 ITR B and B′ arms.

Table 3 of International Patent Application Publication No. WO2019/143885, provides non-exhaustive examples of modifications of at least one nucleotide (e.g., a deletion, insertion and/or substitution) in regions of a modified ITR, where X is indicative of a modification of at least one nucleic acid (e.g., a deletion, insertion and/or substitution) in that section relative to the corresponding wild-type ITR. In some embodiments, any modification of at least one nucleotide (e.g., a deletion, insertion and/or substitution) in any of the regions of C and/or C′ and/or B and/or B′ retains three sequential T nucleotides (i.e., TTT) in at least one terminal loop. For example, if the modification results in any of: a single arm ITR (e.g., single C-C′ loop, or a single B-B′ loop), or a modified C-B′ arm or C′-B arm, or a two-loop ITR with at least one truncated loop (e.g., a truncated C-C′ loop and/or truncated B-B′ loop), at least the single loop, or at least one of the loops of a two-loop ITR (where one arm can be truncated) retains three sequential T nucleotides (i.e., TTT) in at least one terminal loop. In some embodiments, a truncated C-C′ arm and/or a truncated B-B′ arm has three sequential T nucleotides (i.e., TTT) in the terminal loop.

In some embodiments, mod-ITR for use in a synthetically produced ceDNA vector comprising an asymmetric ITR pair, or a symmetric mod-ITR pair as disclosed herein can comprise any one of the combinations of modifications shown in Table 3 of WO2019/143885, and also a modification of at least one nucleotide in any one or more of the regions selected from: between A′ and C, between C and C′, between C′ and B, between B and B′ and between B′ and A. In some embodiments, any modification of at least one nucleotide (e.g., a deletion, insertion and/or substitution) in the C or C′ or B or B′ regions, still preserves the terminal loop of the stem-loop. In some embodiments, any modification of at least one nucleotide (e.g., a deletion, insertion and/or substitution) between C and C′ and/or B and B′ retains three sequential T nucleotides (i.e., TTT) in at least one terminal loop. In alternative embodiments, any modification of at least one nucleotide (e.g., a deletion, insertion and/or substitution) between C and C′ and/or B and B′ retains three sequential A nucleotides (i.e., AAA) in at least one terminal loop In some embodiments, a modified ITR for use herein can comprise any one of the combinations of modifications shown in Table 3, and also a modification of at least one nucleotide (e.g., a deletion, insertion and/or substitution) in any one or more of the regions selected from: A′, A and/or D. For example, in some embodiments, a modified ITR for use herein can comprise any one of the combinations of modifications shown in Table 3 of WO2019/143885, and also a modification of at least one nucleotide (e.g., a deletion, insertion and/or substitution) in the A region. In some embodiments, a modified ITR for use herein can comprise any one of the combinations of modifications shown in Table 3 of WO2019/143885, and also a modification of at least one nucleotide (e.g., a deletion, insertion and/or substitution) in the A′ region. In some embodiments, a modified ITR for use herein can comprise any one of the combinations of modifications shown in Table 3 of WO2019/143885, and also a modification of at least one nucleotide (e.g., a deletion, insertion and/or substitution) in the A and/or A′ region. In some embodiments, a modified ITR for use herein can comprise any one of the combinations of modifications shown in Table 3 of WO2019/143885, and also a modification of at least one nucleotide (e.g., a deletion, insertion and/or substitution) in the D region.

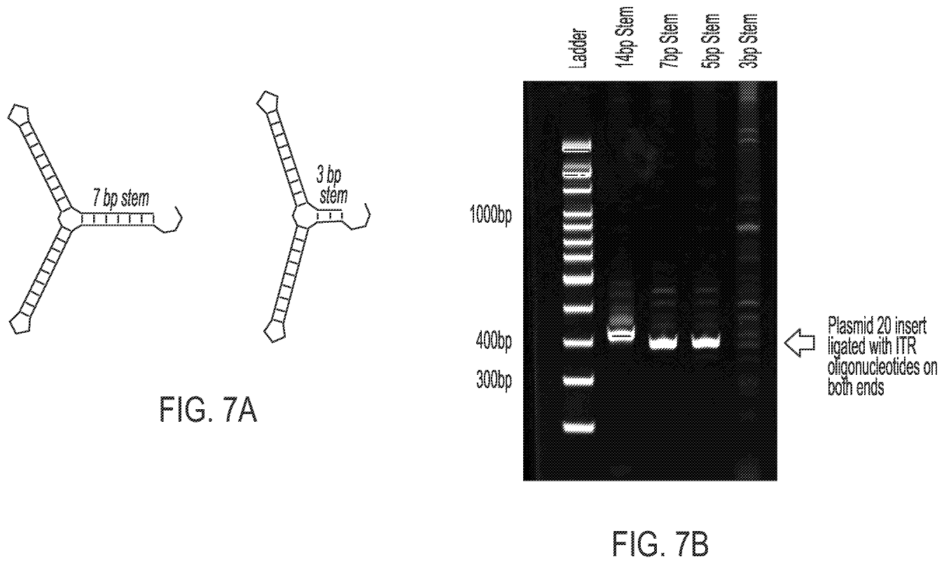

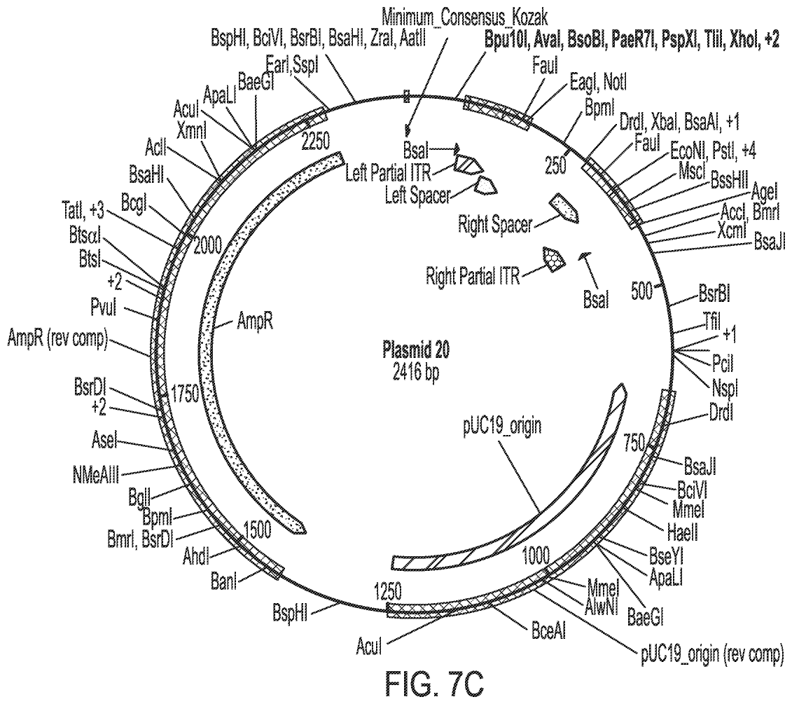

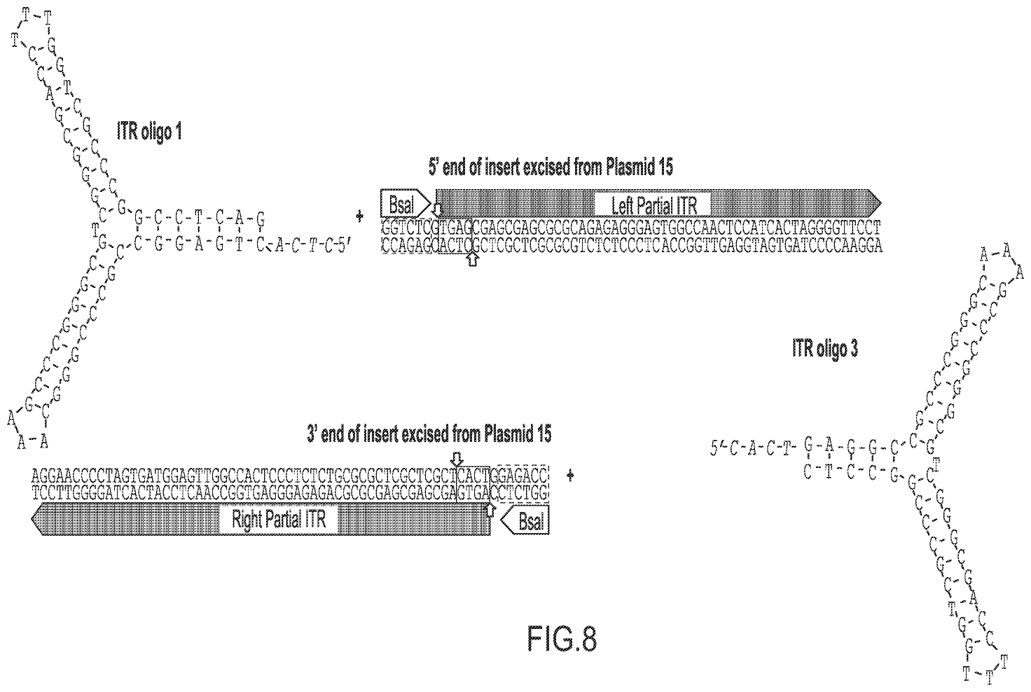

In one embodiment, the nucleotide sequence of the structural element can be modified (e.g., by modifying 1, 2, 3, 4, 5, 6, 7, 8, 9, 10, 11, 12, 13, 14, 15, 16, 17, 18, 19, or 20 or more nucleotides or any range therein) to produce a modified structural element. In one embodiment, the specific modifications to the ITRs are exemplified in SEQ ID NOS: 3, 4, 15-47, 101-116 or 165-187 of WO2019/143885, or shown in FIGS. 7A-7B of International Patent Application Publication No. WO2019/113310, incorporated by reference in its entirety herein (e.g., SEQ ID Nos 97-98, 101-103, 105-108, 111-112, 117-134, 545-54 in WO2019/113310). In some embodiments, an ITR can be modified (e.g., by modifying 1, 2, 3, 4, 5, 6, 7, 8, 9, 10, 11, 12, 13, 14, 15, 16, 17, 18, 19, or 20 or more nucleotides or any range therein). In other embodiments, the ITR can have at least 80%, at least 85%, at least 90%, at least 95%, at least 96%, at least 97%, at least 98%, at least 99%, or more sequence identity with one of the modified ITRs of SEQ ID NOS: 3, 4, 15-47, 101-116 or 165-187, or the RBE-containing section of the A-A′ stem region and C-C′ and B-B′ loops of SEQ ID NO: 3, 4, 15-47, 101-116 or 165-187 of WO2019/143885, or shown in Tables 2-9 (i.e., SEQ ID NO: 110-112, 115-190, 200-468) of International Patent Application Publication No. WO2019/051255.

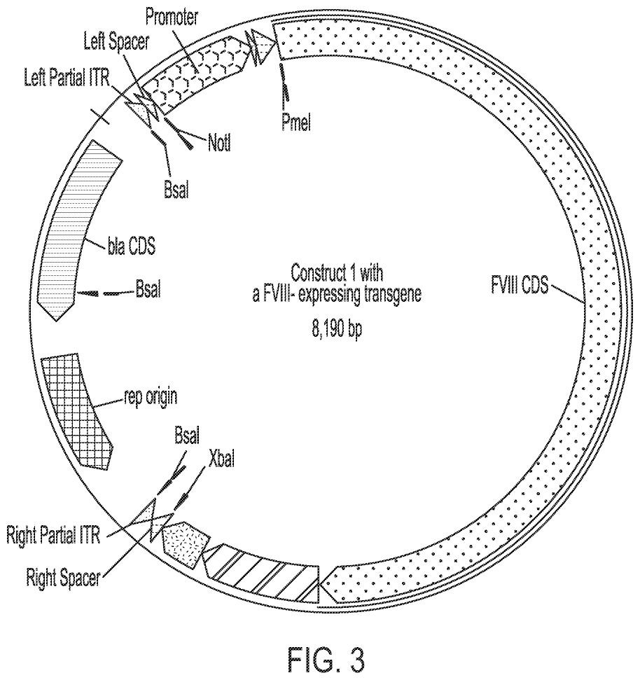

In some embodiments, a modified ITR can for example, comprise removal or deletion of all of a particular arm, e.g., all or part of the A-A′ stem region, or all or part of the B-B′ loop or all or part of the C-C′ loop, or alternatively, the removal of 1, 2, 3, 4, 5, 6, 7, 8, 9 or more base pairs forming the loop so long as the final loop capping the stem (e.g., single arm) is still present (e.g., see ITR-21 in FIG. 7A of WO2019/113310). In some embodiments, a modified ITR can comprise the removal of 1, 2, 3, 4, 5, 6, 7, 8, 9 or more base pairs from the B-B′ arm. In some embodiments, a modified ITR can comprise the removal of 1, 2, 3, 4, 5, 6, 7, 8, 9 or more base pairs from the C-C′ arm (see, e.g., ITR-1 in FIG. 3B, or ITR-45 in FIG. 7A of WO2019/113310). In some embodiments, a modified ITR can comprise the removal of 1, 2, 3, 4, 5, 6, 7, 8, 9 or more base pairs from the C-C′ arm and the removal of 1, 2, 3, 4, 5, 6, 7, 8, 9 or more base pairs from the B-B′ loop. Any combination of removal of base pairs is envisioned, for example, 6 base pairs can be removed in the C-C′ loop and 2 base pairs in the B-B′ loop.

In some embodiments, a modified ITR can have between 1 and 50 (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) nucleotide deletions relative to a full-length wild-type ITR sequence. In some embodiments, a modified ITR can have between 1 and 30 nucleotide deletions relative to a full-length WT ITR sequence. In some embodiments, a modified ITR has between 2 and 20 nucleotide deletions relative to a full-length wild-type ITR sequence.

In some embodiments, a modified ITR does not contain any nucleotide deletions in the RBE-containing portion of the A or A′ stem regions, so as not to interfere with DNA replication (e.g. binding to an RBE by Rep protein, or nicking at a terminal resolution site). In some embodiments, a modified ITR encompassed for use herein has one or more deletions in the B, B′, C, and/or C region as described herein.

In some embodiments, a synthetically produced ceDNA vector comprising a symmetric ITR pair or asymmetric ITR pair comprises a regulatory switch as disclosed herein and at least one modified ITR selected having the nucleotide sequence selected from any of the group consisting of: SEQ ID NO: 3, 4, 15-47, 101-116 or 165-187 of International Patent Application Publication No. WO2019/143885.

In another embodiment, the structure of the structural element can be modified. For example, the structural element a change in the height of the stem and/or the number of nucleotides in the loop. For example, the height of the stem can be about 2, 3, 4, 5, 6, 7, 8, or 9 nucleotides or more or any range therein. In one embodiment, the stem height can be about 5 nucleotides to about 9 nucleotides and functionally interacts with Rep. In another embodiment, the stem height can be about 7 nucleotides and functionally interacts with Rep. In another example, the loop can have 3, 4, 5, 6, 7, 8, 9, or 10 nucleotides or more or any range therein.

In another embodiment, the number of GAGY binding sites or GAGY-related binding sites within the RBE or extended RBE can be increased or decreased. In one example, the RBE or extended RBE, can comprise 1, 2, 3, 4, 5, or 6 or more GAGY binding sites or any range therein. Each GAGY binding site can independently be an exact GAGY sequence or a sequence similar to GAGY as long as the sequence is sufficient to bind a Rep protein.

In another embodiment, the spacing between two elements (such as but not limited to the RBE and a hairpin) can be altered (e.g., increased or decreased) to alter functional interaction with a large Rep protein. For example, the spacing can be about 1, 2, 3, 4, 5, 6, 7, 8, 9, 10, 11, 12, 13, 14, 15, 16, 17, 18, 19, 20, or 21 nucleotides or more or any range therein.

The synthetically produced ceDNA vector described herein can include an ITR structure that is modified with respect to the wild type AAV2 ITR structure disclosed herein, but still retains an operable RBE, trs and RBE′ portion. In some embodiments, such an ITR (wt or modified ITR) is functional. In alternative embodiments, where a ceDNA vector comprises two modified ITRs that are different or asymmetrical to each other, at least one modified ITR is functional and at least one modified ITR is non-functional.

In some embodiments, a synthetically produced ceDNA vector does not have a modified ITR selected from any sequence consisting of, or consisting essentially of: SEQ ID NOs:500-529, as disclosed in International Patent Application Publication No. WO2019/143885. In some embodiments, a ceDNA vector does not have an ITR that is selected from any sequence selected from SEQ ID NOs: 500-529 of WO2019/143885.

In some embodiments, the modified ITR (e.g., the left or right ITR) of the synthetically produced ceDNA vector described herein has modifications within the loop, the truncated loop, or the spacer. Exemplary sequences of ITRs having modifications within the loop, the truncated loop, or the spacer are listed in Table 2 of International Patent Application Publication No. WO2019/051255 (i.e., SEQ ID NOS: 135-190, 200-233 of WO2019/143885); Table 3 of WO2019/051255 (e.g., SEQ ID Nos: 234-263 of WO2019/143885); Table 4 of WO2019/051255 (e.g., SEQ ID NOs: 264-293 of WO2019/143885); Table 5 of WO2019/051255 (e.g., SEQ ID Nos: 294-318 of WO2019/143885); Table 6 of WO2019/051255 (e.g., SEQ ID NO: 319-468 of WO2019/143885); and Tables 7-9 of WO2019/051255 (e.g., SEQ ID Nos: 101-110, 111-112, 115-134 of WO2019/143885) or Table 10A or 10B of WO2019/051255 (e.g., SEQ ID Nos: 9, 100, 469-483, 484-499 of WO2019/143885).

In some embodiments, the modified ITR for use in a synthetically produced ceDNA vector comprising an asymmetric ITR pair, or symmetric mod-ITR pair is selected from any or a combination of those shown in Tables 2, 3, 4, 5, 6, 7, 8, 9 and 10A-10B of WO2019/051255.

Additional exemplary modified ITRs for use in a synthetically produced ceDNA vector comprising an asymmetric ITR pair, or symmetric mod-ITR pair in each of the above classes are provided in Tables 4A and 4B of WO 2019/143885.

In one embodiment, a synthetically produced ceDNA vector comprises, in the 5′ to 3′ direction: a first adeno-associated virus (AAV) inverted terminal repeat (ITR), a nucleotide sequence of interest (for example an expression cassette as described herein) and a second AAV ITR, where the first ITR (5′ ITR) and the second ITR (3′ ITR) are asymmetric with respect to each other—that is, they have a different 3D-spatial configuration from one another. As an exemplary embodiment, the first ITR can be a wild-type ITR and the second ITR can be a mutated or modified ITR, or vice versa, where the first ITR can be a mutated or modified ITR and the second ITR a wild-type ITR. In some embodiment, the first ITR and the second ITR are both mod-ITRs, but have different sequences, or have different modifications, and thus are not the same modified ITRs, and have different 3D spatial configurations. Stated differently, a ceDNA vector with asymmetric ITRs comprises ITRs where any changes in one ITR relative to the WT-ITR are not reflected in the other ITR; or alternatively, where the asymmetric ITRs have a the modified asymmetric ITR pair can have a different sequence and different three-dimensional shape with respect to each other. Exemplary asymmetric ITRs in the ceDNA vector and for use to generate a ceDNA-plasmid are shown in Tables 4A and 4B of WO 2019/143885.

In an alternative embodiment, a synthetically produced ceDNA vector comprises two symmetrical mod-ITRs—that is, both ITRs have the same sequence, but are reverse complements (inverted) of each other. In some embodiments, a symmetrical mod-ITR pair comprises at least one or any combination of a deletion, insertion, or substitution relative to wild type ITR sequence from the same AAV serotype. The additions, deletions, or substitutions in the symmetrical ITR are the same but the reverse complement of each other. For example, an insertion of 3 nucleotides in the C region of the 5′ ITR would be reflected in the insertion of 3 reverse complement nucleotides in the corresponding section in the C′ region of the 3′ ITR.

In alternative embodiments, the modified ITR pair are substantially symmetrical as defined herein—that is, the modified ITR pair can have a different sequence but have corresponding or the same symmetrical three-dimensional shape. For example, one modified ITR can be from one serotype and the other modified ITR be from a different serotype, but they have the same mutation (e.g., nucleotide insertion, deletion or substitution) in the same region. Stated differently, for illustrative purposes only, a 5′ mod-ITR can be from AAV2 and have a deletion in the C region, and the 3′ mod-ITR can be from AAV5 and have the corresponding deletion in the C′ region, and provided the 5′ mod-ITR and the 3′ mod-ITR have the same or symmetrical three-dimensional spatial organization, they are encompassed for use herein as a modified ITR pair.

In some embodiments, a substantially symmetrical mod-ITR pair has the same A-A′ stem region, C-C′ and B-B′ loops in 3D space, e.g., if a modified ITR in a substantially symmetrical mod-ITR pair has a deletion of a C-C′ arm, then the cognate mod-ITR has the corresponding deletion of the C-C′ loop and also has a similar 3D structure of the remaining A and B-B′ loops in the same shape in geometric space of its cognate mod-ITR. By way of example only, substantially symmetrical ITRs can have a symmetrical spatial organization such that their structure is the same shape in geometrical space. This can occur, e.g., when a G-C pair is modified, for example, to a C-G pair or vice versa, or A-T pair is modified to a T-A pair, or vice versa. In some embodiments, such a modified ITR pair are substantially symmetrical as the modified ITR pair has symmetrical stereochemistry.

Table 5 of International Patent Application Publication No. WO2019/143885, provides exemplary symmetric modified ITR pairs (i.e., left modified ITRs and symmetrical right modified ITRs)

In some embodiments, a ceDNA vector comprising an asymmetric ITR pair can comprise an ITR with a modification corresponding to any of the modifications in ITR sequences or ITR partial sequences shown in any one or more of Tables 4A-4B of WO2019/143885, or the sequences shown in FIG. 7A and FIG. 7B of International Patent Application Publication No. WO2019/113310, or disclosed in Tables 2, 3, 4, 5, 6, 7, 8, 9 or 10A-10B of International Patent Application Publication No. WO2019/051255.

### V. Exemplary ceDNA Vectors

As described above, the present disclosure relates to synthetically produced recombinant ceDNA expression vectors and ceDNA vectors that encode a transgene comprising any one of: an asymmetric ITR pair, a symmetrical ITR pair, or substantially symmetrical ITR pair as described above. In certain embodiments, the disclosure relates to synthetically produced recombinant ceDNA vectors having flanking ITRs and a transgene, where the ITRs are asymmetric, symmetrical or substantially symmetrical relative to each other as defined herein, and the ceDNA further comprises a nucleotide sequence of interest (for example an expression cassette comprising the nucleic acid of a transgene) located between the flanking ITRs, wherein said nucleic acid molecule is devoid of viral capsid protein coding sequences.

The synthetically produced ceDNA expression vector may be any ceDNA vector that can be conveniently subjected to recombinant DNA procedures including nucleotide sequence(s) as described herein, provided at least one ITR is altered. The synthetically produced ceDNA vectors of the present disclosure are compatible with the host cell into which the ceDNA vector is to be introduced. In certain embodiments, the synthetically produced ceDNA vectors may be linear. In certain embodiments, the synthetically produced ceDNA vectors may exist as an extrachromosomal entity. In certain embodiments, the synthetically produced ceDNA vectors of the present disclosure may contain an element(s) that permits integration of a donor sequence into the host cell's genome.

Referring now to FIGS. 1A-1B provided herein and furthermore, FIGS. 1A-1G of International Patent Application Publication No. WO2019/143885, schematics of the functional components of two non-limiting examples of ceDNA having asymmetric ITRs or symmetrical or substantially symmetrically ITRs. In some embodiments, the expressible transgene cassette includes, as needed: an enhancer/promoter, one or more homology arms, a donor sequence, a post-transcription regulatory element (e.g., WPRE, e.g., SEQ ID NO: 67 of WO2019/143885)), and a polyadenylation and termination signal (e.g., BGH polyA, e.g., SEQ ID NO: 68 of WO2019/143885).

### Regulatory Elements

The ceDNA vectors as described herein and produced using the synthetic process as described herein can comprise an asymmetric ITR pair or symmetric ITR pair as defined herein, can be further comprise a specific combination of cis-regulatory elements. The cis-regulatory elements include, but are not limited to, a promoter, a riboswitch, an insulator, a mir-regulatable element, a post-transcriptional regulatory element, a tissue- and cell type-specific promoter and an enhancer. In some embodiments, the ITR can act as the promoter for the transgene. In some embodiments, the ceDNA vector comprises additional components to regulate expression of the transgene, for example, regulatory switches as described herein, to regulate the expression of the transgene, or a kill switch, which can kill a cell comprising the ceDNA vector. Regulatory elements, including Regulatory Switches that can be used in the present disclosure are more fully discussed in International Patent Application Publication No. WO2019/051255.

In some embodiments, the second nucleotide sequence includes a regulatory sequence, and a nucleotide sequence encoding a nuclease. In certain embodiments the gene regulatory sequence is operably linked to the nucleotide sequence encoding the nuclease. In certain embodiments, the regulatory sequence is suitable for controlling the expression of the nuclease in a host cell. In certain embodiments, the regulatory sequence includes a suitable promoter sequence, being able to direct transcription of a gene operably linked to the promoter sequence, such as a nucleotide sequence encoding the nuclease(s) of the present disclosure. In certain embodiments, the second nucleotide sequence includes an intron sequence linked to the 5′ terminus of the nucleotide sequence encoding the nuclease. In certain embodiments, an enhancer sequence is provided upstream of the promoter to increase the efficacy of the promoter. In certain embodiments, the regulatory sequence includes an enhancer and a promoter, wherein the second nucleotide sequence includes an intron sequence upstream of the nucleotide sequence encoding a nuclease, wherein the intron includes one or more nuclease cleavage site(s), and wherein the promoter is operably linked to the nucleotide sequence encoding the nuclease.

The ceDNA vectors produced using the synthetic process as described herein can further comprise a specific combination of cis-regulatory elements such as WHP posttranscriptional regulatory element (WPRE) (e.g., SEQ ID NO: 67 of WO2019/143885) and BGH polyA (SEQ ID NO: 68 of WO2019/143885). Suitable expression cassettes for use in expression constructs are not limited by the packaging constraint imposed by the viral capsid.

### Promoters

It will be appreciated by one of ordinary skill in the art that promoters used in the synthetically produced ceDNA vectors of the disclosure should be tailored as appropriate for the specific sequences they are promoting. For example, a guide RNA may not require a promoter at all, since its function is to form a duplex with a specific target sequence on the native DNA to effect a recombination event. In contrast, a nuclease encoded by the ceDNA vector would benefit from a promoter so that it can be efficiently expressed from the vector—and, optionally, in a regulatable fashion.

Expression cassettes of the present disclosure include a promoter, which can influence overall expression levels as well as cell-specificity. For transgene expression, they can include a highly active virus-derived immediate early promoter. Expression cassettes can contain tissue-specific eukaryotic promoters to limit transgene expression to specific cell types and reduce toxic effects and immune responses resulting from unregulated, ectopic expression. In preferred embodiments, an expression cassette can contain a synthetic regulatory element, such as a CAG promoter (SEQ ID NO: 72 of WO2019/143885). The CAG promoter comprises (i) the cytomegalovirus (CMV) early enhancer element, (ii) the promoter, the first exon and the first intron of chicken beta-actin gene, and (iii) the splice acceptor of the rabbit beta-globin gene. Alternatively, an expression cassette can contain an Alpha-1-antitrypsin (AAT) promoter (SEQ ID NO: 73 or SEQ ID NO: 74 of WO2019/143885), a liver specific (LP1) promoter (SEQ ID NO: 75 or SEQ ID NO: 76 of WO2019/143885), or a Human elongation factor-1 alpha (EF1a) promoter (e.g., SEQ ID NO: 77 or SEQ ID NO: 78 of WO2019/143885). In some embodiments, the expression cassette includes one or more constitutive promoters, for example, a retroviral Rous sarcoma virus (RSV) LTR promoter (optionally with the RSV enhancer), or a cytomegalovirus (CMV) immediate early promoter (optionally with the CMV enhancer, e.g., SEQ ID NO: 79 of WO2019/143885). Alternatively, an inducible promoter, a native promoter for a transgene, a tissue-specific promoter, or various promoters known in the art can be used.

Suitable promoters, including those described above, can be derived from viruses and can therefore be referred to as viral promoters, or they can be derived from any organism, including prokaryotic or eukaryotic organisms. Suitable promoters can be used to drive expression by any RNA polymerase (e.g., pol I, pol II, pol III). Exemplary promoters include, but are not limited to the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6, e.g., SEQ ID NO: 80 of WO2019/143885) (Miyagishi et al., Nature Biotechnology 20, 497-500 (2002)), an enhanced U6 promoter (e.g., Xia et al., Nucleic Acids Res. 2003 Sep. 1; 31(17)), a human H1 promoter (H1) (e.g., SEQ ID NO: 81 or SEQ ID NO: 155 of WO2019/143885), a CAG promoter, a human alpha 1-antitrypsin (HAAT) promoter (e.g., SEQ ID NO: 82 of WO2019/143885), and the like. In certain embodiments, these promoters are altered at their downstream intron containing end to include one or more nuclease cleavage sites. In certain embodiments, the DNA containing the nuclease cleavage site(s) is foreign to the promoter DNA.

In one embodiment, the promoter used is the native promoter of the gene encoding the therapeutic protein. The promoters and other regulatory sequences for the respective genes encoding the therapeutic proteins are known and have been characterized. The promoter region used may further include one or more additional regulatory sequences (e.g., native), e.g., enhancers, (e.g. SEQ ID NO: 79 and SEQ ID NO: 83 of WO2019/143885).

Non-limiting examples of suitable promoters or promoter sets for use in accordance with the present disclosure include the CAG promoter of, for example (SEQ ID NO: 72 of WO2019/143885), the HAAT promoter (SEQ ID NO: 82 of WO2019/143885), the human EF1-α promoter (SEQ ID NO: 77 of WO2019/143885) or a fragment of the EF1a promoter (SEQ ID NO: 78 of WO2019/143885), IE2 promoter (e.g., SEQ ID NO: 84 of WO2019/143885) and the rat EF1-α promoter (SEQ ID NO: 85 of WO2019/143885), or 1E1 promoter fragment (SEQ ID NO: 125 of WO2019/143885).

### Polyadenylation Sequences

A sequence encoding a polyadenylation sequence can be included in the synthetically produced ceDNA vector to stabilize an mRNA expressed from the ceDNA vector, and to aid in nuclear export and translation. In one embodiment, the synthetically produced ceDNA vector does not include a polyadenylation sequence. In other embodiments, the vector includes at least 1, at least 2, at least 3, at least 4, at least 5, at least 10, at least 15, at least 20, at least 25, at least 30, at least 40, least 45, at least 50 or more adenine dinucleotides. In some embodiments, the polyadenylation sequence comprises about 43 nucleotides, about 40-50 nucleotides, about 40-55 nucleotides, about 45-50 nucleotides, about 35-50 nucleotides, or any range there between.

The expression cassettes can include a poly-adenylation sequence known in the art or a variation thereof, such as a naturally occurring sequence isolated from bovine BGHpA (e.g., SEQ ID NO: 68 of WO2019/143885) or a virus SV40 pA (e.g., SEQ ID NO: 86 of WO2019/143885), or a synthetic sequence (e.g., SEQ ID NO: 87 of WO2019/143885). Some expression cassettes can also include SV40 late polyA signal upstream enhancer (USE) sequence. In some embodiments, the, USE can be used in combination with SV40 pA or heterologous poly-A signal.

The expression cassettes can also include a post-transcriptional element to increase the expression of a transgene. In some embodiments, Woodchuck Hepatitis Virus (WHP) posttranscriptional regulatory element (WPRE) (e.g., SEQ ID NO: 67 of WO2019/143885) is used to increase the expression of a transgene. Other posttranscriptional processing elements such as the post-transcriptional element from the thymidine kinase gene of herpes simplex virus, or hepatitis B virus (HBV) can be used. Secretory sequences can be linked to the transgenes, e.g., VH-02 and VK-A26 sequences, e.g., SEQ ID NO: 88 and SEQ ID NO: 89 of WO2019/143885.

### Nuclear Localization Sequences

In some embodiments, the vector encoding an RNA guided endonuclease comprises one or more nuclear localization sequences (NLSs), for example, 1, 2, 3, 4, 5, 6, 7, 8, 9, 10, or more NLSs. In some embodiments, the one or more NLSs are located at or near the amino-terminus, at or near the carboxy-terminus, or a combination of these (e.g., one or more NLS at the amino-terminus and/or one or more NLS at the carboxy terminus). When more than one NLS is present, each can be selected independently of the others, such that a single NLS is present in more than one copy and/or in combination with one or more other NLSs present in one or more copies. Non-limiting examples of NLSs are shown in Table 6 of International Patent Application Publication No. WO2019/143885.

Additional Components of ceDNA Vectors

The ceDNA vectors produced using the synthetic process as described herein may contain nucleotides that encode other components for gene expression. For example, to select for specific gene targeting events, a protective shRNA may be embedded in a microRNA and inserted into a recombinant ceDNA vector designed to integrate site-specifically into the highly active locus, such as an albumin locus. Such embodiments may provide a system for in vivo selection and expansion of gene-modified hepatocytes in any genetic background such as described in Nygaard et al., A universal system to select gene-modified hepatocytes in vivo, Gene Therapy, Jun. 8, 2016. The ceDNA vectors of the present disclosure may contain one or more selectable markers that permit selection of transformed, transfected, transduced, or the like cells. A selectable marker is a gene the product of which provides for biocide or viral resistance, resistance to heavy metals, prototrophy to auxotrophs, NeoR, and the like. In certain embodiments, positive selection markers are incorporated into the donor sequences such as NeoR. Negative selections markers may be incorporated downstream the donor sequences, for example a nucleic acid sequence HSV-tk encoding a negative selection marker may be incorporated into a nucleic acid construct downstream the donor sequence.

In embodiments, the ceDNA vector produced using the synthetic process as described herein can be used for gene editing, for example, as disclosed in International Patent Application Publication No. WO2019/113310, and may include one or more of: a 5′ homology arm, a 3′ homology arm, a polyadenylation site upstream and proximate to the 5′ homology arm. Exemplary homology arms are 5′ and 3′ albumin homology arms (SEQ ID NO: 151 and 152 of WO2019/143885) or CCR5 5′- and 3′ homology arms (e.g., SEQ ID NO: 153 and 154 of WO2019/143885).

### Regulatory Switches

A molecular regulatory switch is one which generates a measurable change in state in response to a signal. Such regulatory switches can be usefully combined with the ceDNA vectors produced using the synthetic process as described herein to control the output of expression of the transgene from the ceDNA vector. In some embodiments, the ceDNA vector comprises a regulatory switch that serves to fine tune expression of the transgene. For example, it can serve as a biocontainment function of the ceDNA vector. In some embodiments, the switch is an “ON/OFF” switch that is designed to start or stop (i.e., shut down) expression of the gene of interest in the ceDNA in a controllable and regulatable fashion. In some embodiments, the switch can include a “kill switch” that can instruct the cell comprising the ceDNA vector to undergo cell programmed death once the switch is activated. Exemplary regulatory switches encompassed for use in a ceDNA vector can be used to regulate the expression of a transgene, and are more fully discussed in International Patent Application Publication No. WO2019/113310.

### (i) Binary Regulatory Switches

In some embodiments, the ceDNA vector produced using the synthetic process as described herein comprises a regulatory switch that can serve to controllably modulate expression of the transgene. For example, the expression cassette located between the ITRs of the ceDNA vector may additionally comprise a regulatory region, e.g., a promoter, cis-element, repressor, enhancer etc., that is operatively linked to the gene of interest, where the regulatory region is regulated by one or more cofactors or exogenous agents. By way of example only, regulatory regions can be modulated by small molecule switches or inducible or repressible promoters. Non-limiting examples of inducible promoters are hormone-inducible or metal-inducible promoters. Other exemplary inducible promoters/enhancer elements include, but are not limited to, an RU486-inducible promoter, an ecdysone-inducible promoter, a rapamycin-inducible promoter, and a metallothionein promoter.

### (ii) Small Molecule Regulatory Switches

A variety of art-known small-molecule based regulatory switches are known in the art and can be combined with the synthetically produced ceDNA vectors disclosed herein to form a regulatory-switch controlled ceDNA vector. In some embodiments, the regulatory switch can be selected from any one or a combination of: an orthogonal ligand/nuclear receptor pair, for example retinoid receptor variant/LG335 and GRQCIMFI, along with an artificial promoter controlling expression of the operatively linked transgene, such as that as disclosed in Taylor, et al., BMC Biotechnology 10 (2010): 15; engineered steroid receptors, e.g., modified progesterone receptor with a C-terminal truncation that cannot bind progesterone but binds RU486 (mifepristone) (U.S. Pat. No. 5,364,791); an ecdysone receptor from Drosophila and their ecdysteroid ligands (Saez, et al., PNAS, 97(26)(2000), 14512-14517; or a switch controlled by the antibiotic trimethoprim (TMP), as disclosed in Sando R 3rd; Nat Methods. 2013, 10(11):1085-8. In some embodiments, the regulatory switch to control the transgene or expressed by the ceDNA vector is a pro-drug activation switch, such as that disclosed in U.S. Pat. Nos. 8,771,679, and 6,339,070.

(iii) “Passcode” Regulatory Switches

In some embodiments the regulatory switch can be a “passcode switch” or “passcode circuit”. Passcode switches allow fine tuning of the control of the expression of the transgene from the synthetically produced ceDNA vector when specific conditions occur—that is, a combination of conditions need to be present for transgene expression and/or repression to occur. For example, for expression of a transgene to occur at least conditions A and B must occur. A passcode regulatory switch can be any number of conditions, e.g., at least 2, or at least 3, or at least 4, or at least 5, or at least 6 or at least 7 or more conditions to be present for transgene expression to occur. In some embodiments, at least 2 conditions (e.g., A, B conditions) need to occur, and in some embodiments, at least 3 conditions need to occur (e.g., A, B and C, or A, B and D). By way of an example only, for gene expression from a ceDNA to occur that has a passcode “ABC” regulatory switch, conditions A, B and C must be present. Conditions A, B and C could be as follows; condition A is the presence of a condition or disease, condition B is a hormonal response, and condition C is a response to the transgene expression. For example, if the transgene edits a defective EPO gene, Condition A is the presence of Chronic Kidney Disease (CKD), Condition B occurs if the subject has hypoxic conditions in the kidney, Condition C is that Erythropoietin-producing cells (EPC) recruitment in the kidney is impaired; or alternatively, HIF-2 activation is impaired. Once the oxygen levels increase or the desired level of EPO is reached, the transgene turns off again until 3 conditions occur, turning it back on.

In some embodiments, a passcode regulatory switch or “passcode circuit” encompassed for use in the synthetically produced ceDNA vector comprises hybrid transcription factors (TFs) to expand the range and complexity of environmental signals used to define biocontainment conditions. As opposed to a deadman switch which triggers cell death in the presence of a predetermined condition, the “passcode circuit” allows cell survival or transgene expression in the presence of a particular “passcode”, and can be easily reprogrammed to allow transgene expression and/or cell survival only when the predetermined environmental condition or passcode is present.

Any and all combinations of regulatory switches disclosed herein, e.g., small molecule switches, nucleic acid-based switches, small molecule-nucleic acid hybrid switches, post-transcriptional transgene regulation switches, post-translational regulation, radiation-controlled switches, hypoxia-mediated switches and other regulatory switches known by persons of ordinary skill in the art as disclosed herein can be used in a passcode regulatory switch as disclosed herein. Regulatory switches encompassed for use are also discussed in the review article Kis et al., J R Soc Interface. 12: 20141000 (2015), and summarized in Table 1 of Kis.

### (iv) Nucleic Acid-Based Regulatory Switches to Control Transgene Expression

In some embodiments, the regulatory switch to control the transgene expressed by the synthetically produced ceDNA vector is based on a nucleic-acid based control mechanism. Exemplary nucleic acid control mechanisms are known in the art and are envisioned for use. For example, such mechanisms include riboswitches, such as those disclosed in, e.g., U.S. Patent Application Publication Nos. US2009/0305253, US2008/0269258, US2017/0204477, International Patent Application Publication No. WO2018026762A1, U.S. Pat. No. 9,222,093 and also disclosed in the review by Villa J K et al., Microbiol Spectr. 2018 May; 6(3). Also included are metabolite-responsive transcription biosensors, such as those disclosed in International Patent Application Publication Nos. WO2018/075486 and WO2017/147585. Other art-known mechanisms envisioned for use include silencing of the transgene with an siRNA or RNAi molecule (e.g., miR, shRNA). For example, the ceDNA vector can comprise a regulatory switch that encodes a RNAi molecule that is complementary to the transgene expressed by the ceDNA vector. When such RNAi is expressed even if the transgene is expressed by the ceDNA vector, it will be silenced by the complementary RNAi molecule, and when the RNAi is not expressed when the transgene is expressed by the ceDNA vector the transgene is not silenced by the RNAi.

In some embodiments, the regulatory switch is a tissue-specific self-inactivating regulatory switch, for example as disclosed in U.S. Patent Application Publication No. US2002/0022018, whereby the regulatory switch deliberately switches transgene expression off at a site where transgene expression might otherwise be disadvantageous. In some embodiments, the regulatory switch is a recombinase reversible gene expression system, for example as disclosed in U.S. Patent Application Publication No. US2014/0127162 and U.S. Pat. No. 8,324,436, incorporated by reference in their entireties herein.

### (v) Post-Transcriptional and Post-Translational Regulatory Switches.

In some embodiments, the regulatory switch to control the transgene or gene of interest expressed by the synthetically produced ceDNA vector is a post-transcriptional modification system. For example, such a regulatory switch can be an aptazyme riboswitch that is sensitive to tetracycline or theophylline, as disclosed in U.S. Patent Application Publication No. US2018/0119156, UK Patent Application Publication No. GB201107768, International Patent Application Publication No. WO2001/064956A3, EP Patent No. 2707487 and Beilstein et al., ACS Synth. Biol., 2015, 4 (5), pp 526-534; Zhong et al., Elife. 2016 Nov. 2; 5. Pii: e18858. In some embodiments, it is envisioned that a person of ordinary skill in the art could encode both the transgene and an inhibitory siRNA which contains a ligand sensitive (OFF-switch) aptamer, the net result being a ligand sensitive ON-switch.

### (vi) Other Exemplary Regulatory Switches

Any known regulatory switch can be used in the synthetically produced ceDNA vector to control the gene expression of the transgene expressed by the ceDNA vector, including those triggered by environmental changes. Additional examples include, but are not limited to; the BOC method of Suzuki et al., Scientific Reports 8; 10051 (2018); genetic code expansion and a non-physiologic amino acid; radiation-controlled or ultra-sound controlled on/off switches (see, e.g., Scott S et al., Gene Ther. 2000 July; 7(13):1121-5; U.S. Pat. Nos. 5,612,318; 5,571,797; 5,770,581; 5,817,636; and International Patent Application Publication No. WO1999/025385A1. In some embodiments, the regulatory switch is controlled by an implantable system, e.g., as disclosed in U.S. Pat. No. 7,840,263; US2007/0190028A1 where gene expression is controlled by one or more forms of energy, including electromagnetic energy, that activates promoters operatively linked to the transgene in the ceDNA vector.

In some embodiments, a regulatory switch envisioned for use in the synthetically produced ceDNA vector is a hypoxia-mediated or stress-activated switch, e.g., such as those disclosed in International Patent Application Publication No. WO1999060142A2, U.S. Pat. Nos. 5,834,306; 6,218,179; 6,709,858; U.S. Patent Application Publication No. US2015/0322410; Greco et al., (2004) Targeted Cancer Therapies 9, S368, as well as FROG, TOAD and NRSE elements and conditionally inducible silence elements, including hypoxia response elements (HREs), inflammatory response elements (IREs) and shear-stress activated elements (SSAEs), e.g, as disclosed in U.S. Pat. No. 9,394,526. Such an embodiment is useful for turning on expression of the transgene from the ceDNA vector after ischemia or in ischemic tissues, and/or tumors.

(vii) Kill Switches

Other embodiments of the disclosure relate to a synthetically produced ceDNA vector comprising a kill switch. A kill switch as disclosed herein enables a cell comprising the ceDNA vector to be killed or undergo programmed cell death as a means to permanently remove an introduced ceDNA vector from the subject's system. It will be appreciated by one of ordinary skill in the art that use of kill switches in the synthetically produced ceDNA vectors of the disclosure would be typically coupled with targeting of the ceDNA vector to a limited number of cells that the subject can acceptably lose or to a cell type where apoptosis is desirable (e.g., cancer cells). In all aspects, a “kill switch” as disclosed herein is designed to provide rapid and robust cell killing of the cell comprising the ceDNA vector in the absence of an input survival signal or other specified condition. Stated another way, a kill switch encoded by a ceDNA vector herein can restrict cell survival of a cell comprising a ceDNA vector to an environment defined by specific input signals. Such kill switches serve as a biological biocontainment function should it be desirable to remove the synthetically produced ceDNA vector from a subject or to ensure that it will not express the encoded transgene.

### VI. Related Base Vectors, Constructs, and Kits

A further aspect of this disclosure is directed to the base vectors and double-stranded DNA constructs that have been engineered to facilitate the cell-free synthesis of DNA vectors, such as closed-ended DNA (ceDNA) vectors.

### Base Vectors

The base vectors provided herein are vectors that do not carry any transgene or heterologous nucleic acid. Instead, these base vectors contain a multiple cloning site that is capable of receiving a transgene. Additionally, the base vectors include non-palindromic restriction endonuclease recognition sites and their corresponding cleavage sites that flank the multiple cloning site. The base vectors also further include partial ITRs that flank the multiple cloning site. Thus, in one embodiment, the base vector contains, in the 5′→3′ order: a first non-palindromic restriction endonuclease recognition site and a corresponding first cleavage site, first partial ITR, multiple cloning site, second partial ITR, and a second non-palindromic restriction endonuclease recognition site and a corresponding second cleavage site, as well as all embodiments of the any of the foregoing as disclosed herein, including embodiments of the restriction endonucleases for which the recognition sites are specific. In some embodiments, the base vector further includes an origin of replication and a selectable marker gene. In some embodiments, the base vector further includes one or more spacer regions, such as a spacer between the first partial ITR and the transgene expression cassette, a spacer between the second partial ITR and the transgene expression cassette, and/or a spacer within the multiple cloning site (see e.g., FIG. 2 showing exemplary base vector Plasmid 11).

Exemplary base vectors include Plasmid 17 (SEQ ID NO: 9), Plasmid 20 (SEQ ID NO: 10), Plasmid 18 (SEQ ID NO: 11), Plasmid 15 (SEQ ID NO: 12), Plasmid 1 (SEQ ID NO: 13), Plasmid 11 (SEQ ID NO: 14), Plasmid 8 (SEQ ID NO: 15), Plasmid 7 (SEQ ID NO: 16), and Plasmid 6 (SEQ ID NO: 17), shown below.

Plasmid 17
(SEQ ID NO: 9)
TTCGTCAGATCACCGGGTTGTTCCATATCATCGTGTCCACAAGGGCTTGC
CGGTCAAGTGCCTAAGCTCACTCGAGGGTCTCGAGAGCGAGCGAGCGCGC
AGAGAGGGAGTGGCCAACTCCATCACTAGGGGTTCCTTGTAGTTAATGAT
TAACCCGCCATGCTACTTATCGCGGCCGCAGTTGCATCTTTGTTATACCA
GCGCACGAGTCTGTCGTCATGGCTACAAGACCACGAGAACATGAGCCGGT
GTCTCCAGAGAACTCCATTGTGACTAGGTAGTCTAGAGCATGGCTACGTA
GATAAGTAGCATGGCGGGTTAATCATTAACTACACCTGCAGGAGGAACCC
CTAGTGATGGAGTTGGCCACTCCCTCTCTGCGCGCTCGCTCGCTCTCGAG
ACCACCGGTTTACAGGATAGTCTACTCCCAGTGTCCACATGGTTAGGACC
GAGGGGCTTTGGGTTTAGACACTGGCGACTTCCGCTTCCTCGCTCACTGA
CTCGCTGCGCTCGGTCGTTCGGCTGCGGCGAGCGGTATCAGCTCACTCAA
AGGCGGTAATACGGTTATCCACAGAATCAGGGGATAACGCAGGAAAGAAC
ATGTGAGCAAAAGGCCAGCAAAAGGCCAGGAACCGTAAAAAGGCCGCGTT
GCTGGCGTTTTTCCATAGGCTCCGCCCCCCTGACGAGCATCACAAAAATC
GACGCTCAAGTCAGAGGTGGCGAAACCCGACAGGACTATAAAGATACCAG
GCGTTTCCCCCTGGAAGCTCCCTCGTGCGCTCTCCTGTTCCGACCCTGCC
GCTTACCGGATACCTGTCCGCCTTTCTCCCTTCGGGAAGCGTGGCGCTTT
CTCATAGCTCACGCTGTAGGTATCTCAGTTCGGTGTAGGTCGTTCGCTCC
AAGCTGGGCTGTGTGCACGAACCCCCCGTTCAGCCCGACCGCTGCGCCTT
ATCCGGTAACTATCGTCTTGAGTCCAACCCGGTAAGACACGACTTATCGC
CACTGGCAGCAGCCACTGGTAACAGGATTAGCAGAGCGAGGTATGTAGGC
GGTGCTACAGAGTTCTTGAAGTGGTGGCCTAACTACGGCTACACTAGAAG
AACAGTATTTGGTATCTGCGCTCTGCTGAAGCCAGTTACCTTCGGAAAAA
GAGTTGGTAGCTCTTGATCCGGCAAACAAACCACCGCTGGTAGCGGTGGT
TTTTTTGTTTGCAAGCAGCAGATTACGCGCAGAAAAAAAGGATCTCAAGA
AGATCCTTTGATCTTTTCTACGGGGTCTGACGCTCAGTGGAACGAAAACT
CACGTTAAGGGATTTTGGTCATGAGATTATCAAAAAGGATCTTCACCTAG
ATCCTTTTAAATTAAAAATGAAGTTTTAAATCAATCTAAAGTATATATGA
GTAAACTTGGTCTGACAGTTACCAATGCTTAATCAGTGAGGCACCTATCT
CAGCGATCTGTCTATTTCGTTCATCCATAGTTGCCTGACTCCCCGTCGTG
TAGATAACTACGATACGGGAGGGCTTACCATCTGGCCCCAGTGCTGCAAT
GATACCGCGAGACCCACGCTCACCGGCTCCAGATTTATCAGCAATAAACC
AGCCAGCCGGAAGGGCCGAGCGCAGAAGTGGTCCTGCAACTTTATCCGCC
TCCATCCAGTCTATTAATTGTTGCCGGGAAGCTAGAGTAAGTAGTTCGCC
AGTTAATAGTTTGCGCAACGTTGTTGCCATTGCTACAGGCATCGTGGTGT
CACGCTCGTCGTTTGGTATGGCTTCATTCAGCTCCGGTTCCCAACGATCA
AGGCGAGTTACATGATCCCCCATGTTGTGCAAAAAAGCGGTTAGCTCCTT
CGGTCCTCCGATCGTTGTCAGAAGTAAGTTGGCCGCAGTGTTATCACTCA
TGGTTATGGCAGCACTGCATAATTCTCTTACTGTCATGCCATCCGTAAGA
TGCTTTTCTGTGACTGGTGAGTACTCAACCAAGTCATTCTGAGAATAGTG
TATGCGGCGACCGAGTTGCTCTTGCCCGGCGTCAATACGGGATAATACCG
CGCCACATAGCAGAACTTTAAAAGTGCTCATCATTGGAAAACGTTCTTCG
GGGCGAAAACTCTCAAGGATCTTACCGCTGTTGAGATCCAGTTCGATGTA
ACCCACTCGTGCACCCAACTGATCTTCAGCATCTTTTACTTTCACCAGCG
TTTCTGGGTGAGCAAAAACAGGAAGGCAAAATGCCGCAAAAAAGGGAATA
AGGGCGACACGGAAATGTTGAATACTCATACTCTTCCTTTTTCAATATTA
TTGAAGCATTTATCAGGGTTATTGTCTCATGAGCGGATACATATTTGAAT
GTATTTAGAAAAATAAACAAATAGGGGTTCCGCGCACATTTCCCCGAAAA
GTGCCACCTGACGTCT

According to some embodiments, the base vector is 85% identical to SEQ ID NO: 9. According to some embodiments, the base vector is 90% identical to SEQ ID NO: 9. According to some embodiments, the base vector is 95% identical to SEQ ID NO: 9. According to some embodiments, the base vector is 96% identical to SEQ ID NO: 9. According to some embodiments, the base vector is 97% identical to SEQ ID NO: 9. According to some embodiments, the base vector is 98% identical to SEQ ID NO: 9. According to some embodiments, the base vector is 99% identical to SEQ ID NO: 9. According to some embodiments, the base vector consists of SEQ ID NO: 9.

Plasmid 20
(SEQ ID NO: 10)
TTCGTCAGATCACCGGGTTGTTCCATATCATCGTGTCCACAAGGGCTTGC
CGGTCAAGTGCCTAAGCTCACTCGAGGGTCTCGTGAGCGAGCGAGCGCGC
AGAGAGGGAGTGGCCAACTCCATCACTAGGGGTTCCTTGTAGTTAATGAT
TAACCCGCCATGCTACTTATCGCGGCCGCAGTTGCATCTTTGTTATACCA
GCGCACGAGTCTGTCGTCATGGCTACAAGACCACGAGAACATGAGCCGGT
GTCTCCAGAGAACTCCATTGTGACTAGGTAGTCTAGAGCATGGCTACGTA
GATAAGTAGCATGGCGGGTTAATCATTAACTACACCTGCAGGAGGAACCC
CTAGTGATGGAGTTGGCCACTCCCTCTCTGCGCGCTCGCTCGCTCACGAG
ACCACCGGTTTACAGGATAGTCTACTCCCAGTGTCCACATGGTTAGGACC
GAGGGGCTTTGGGTTTAGACACTGGCGACTTCCGCTTCCTCGCTCACTGA
CTCGCTGCGCTCGGTCGTTCGGCTGCGGCGAGCGGTATCAGCTCACTCAA
AGGCGGTAATACGGTTATCCACAGAATCAGGGGATAACGCAGGAAAGAAC
ATGTGAGCAAAAGGCCAGCAAAAGGCCAGGAACCGTAAAAAGGCCGCGTT
GCTGGCGTTTTTCCATAGGCTCCGCCCCCCTGACGAGCATCACAAAAATC
GACGCTCAAGTCAGAGGTGGCGAAACCCGACAGGACTATAAAGATACCAG
GCGTTTCCCCCTGGAAGCTCCCTCGTGCGCTCTCCTGTTCCGACCCTGCC
GCTTACCGGATACCTGTCCGCCTTTCTCCCTTCGGGAAGCGTGGCGCTTT
CTCATAGCTCACGCTGTAGGTATCTCAGTTCGGTGTAGGTCGTTCGCTCC
AAGCTGGGCTGTGTGCACGAACCCCCCGTTCAGCCCGACCGCTGCGCCTT
ATCCGGTAACTATCGTCTTGAGTCCAACCCGGTAAGACACGACTTATCGC
CACTGGCAGCAGCCACTGGTAACAGGATTAGCAGAGCGAGGTATGTAGGC
GGTGCTACAGAGTTCTTGAAGTGGTGGCCTAACTACGGCTACACTAGAAG
AACAGTATTTGGTATCTGCGCTCTGCTGAAGCCAGTTACCTTCGGAAAAA
GAGTTGGTAGCTCTTGATCCGGCAAACAAACCACCGCTGGTAGCGGTGGT
TTTTTTGTTTGCAAGCAGCAGATTACGCGCAGAAAAAAAGGATCTCAAGA
AGATCCTTTGATCTTTTCTACGGGGTCTGACGCTCAGTGGAACGAAAACT
CACGTTAAGGGATTTTGGTCATGAGATTATCAAAAAGGATCTTCACCTAG
ATCCTTTTAAATTAAAAATGAAGTTTTAAATCAATCTAAAGTATATATGA
GTAAACTTGGTCTGACAGTTACCAATGCTTAATCAGTGAGGCACCTATCT
CAGCGATCTGTCTATTTCGTTCATCCATAGTTGCCTGACTCCCCGTCGTG
TAGATAACTACGATACGGGAGGGCTTACCATCTGGCCCCAGTGCTGCAAT
GATACCGCGAGACCCACGCTCACCGGCTCCAGATTTATCAGCAATAAACC
AGCCAGCCGGAAGGGCCGAGCGCAGAAGTGGTCCTGCAACTTTATCCGCC
TCCATCCAGTCTATTAATTGTTGCCGGGAAGCTAGAGTAAGTAGTTCGCC
AGTTAATAGTTTGCGCAACGTTGTTGCCATTGCTACAGGCATCGTGGTGT
CACGCTCGTCGTTTGGTATGGCTTCATTCAGCTCCGGTTCCCAACGATCA
AGGCGAGTTACATGATCCCCCATGTTGTGCAAAAAAGCGGTTAGCTCCTT
CGGTCCTCCGATCGTTGTCAGAAGTAAGTTGGCCGCAGTGTTATCACTCA
TGGTTATGGCAGCACTGCATAATTCTCTTACTGTCATGCCATCCGTAAGA
TGCTTTTCTGTGACTGGTGAGTACTCAACCAAGTCATTCTGAGAATAGTG
TATGCGGCGACCGAGTTGCTCTTGCCCGGCGTCAATACGGGATAATACCG
CGCCACATAGCAGAACTTTAAAAGTGCTCATCATTGGAAAACGTTCTTCG
GGGCGAAAACTCTCAAGGATCTTACCGCTGTTGAGATCCAGTTCGATGTA
ACCCACTCGTGCACCCAACTGATCTTCAGCATCTTTTACTTTCACCAGCG
TTTCTGGGTGAGCAAAAACAGGAAGGCAAAATGCCGCAAAAAAGGGAATA
AGGGCGACACGGAAATGTTGAATACTCATACTCTTCCTTTTTCAATATTA
TTGAAGCATTTATCAGGGTTATTGTCTCATGAGCGGATACATATTTGAAT
GTATTTAGAAAAATAAACAAATAGGGGTTCCGCGCACATTTCCCCGAAAA
GTGCCACCTGACGTCT

According to some embodiments, the base vector is 85% identical to SEQ ID NO: 10. According to some embodiments, the base vector is 90% identical to SEQ ID NO: 10. According to some embodiments, the base vector is 95% identical to SEQ ID NO: 10. According to some embodiments, the base vector is 96% identical to SEQ ID NO: 10. According to some embodiments, the base vector is 97% identical to SEQ ID NO: 10. According to some embodiments, the base vector is 98% identical to SEQ ID NO: 10. According to some embodiments, the base vector is 99% identical to SEQ ID NO: 10. According to some embodiments, the base vector consists of SEQ ID NO: 10.

Plasmid 18
(SEQ ID NO: 11)
TTCGTCAGATCACCGGGTTGTTCCATATCATCGTGTCCACAAGGGCTTGC
CGGTCAAGTGCCTAAGCTCACTCGAGGGTCTCCAGTGAGCGAGCGAGCGC
GCAGAGAGGGAGTGGCCAACTCCATCACTAGGGGTTCCTTGTAGTTAATG
ATTAACCCGCCATGCTACTTATCGCGGCCGCAGTTGCATCTTTGTTATAC
CAGCGCACGAGTCTGTCGTCATGGCTACAAGACCACGAGAACATGAGCCG
GTGTCTCCAGAGAACTCCATTGTGACTAGGTAGTCTAGAGCATGGCTACG
TAGATAAGTAGCATGGCGGGTTAATCATTAACTACACCTGCAGGAGGAAC
CCCTAGTGATGGAGTTGGCCACTCCCTCTCTGCGCGCTCGCTCGCTCACT
GGAGACCACCGGTTTACAGGATAGTCTACTCCCAGTGTCCACATGGTTAG
GACCGAGGGGCTTTGGGTTTAGACACTGGCGACTTCCGCTTCCTCGCTCA
CTGACTCGCTGCGCTCGGTCGTTCGGCTGCGGCGAGCGGTATCAGCTCAC
TCAAAGGCGGTAATACGGTTATCCACAGAATCAGGGGATAACGCAGGAAA
GAACATGTGAGCAAAAGGCCAGCAAAAGGCCAGGAACCGTAAAAAGGCCG
CGTTGCTGGCGTTTTTCCATAGGCTCCGCCCCCCTGACGAGCATCACAAA
AATCGACGCTCAAGTCAGAGGTGGCGAAACCCGACAGGACTATAAAGATA
CCAGGCGTTTCCCCCTGGAAGCTCCCTCGTGCGCTCTCCTGTTCCGACCC
TGCCGCTTACCGGATACCTGTCCGCCTTTCTCCCTTCGGGAAGCGTGGCG
CTTTCTCATAGCTCACGCTGTAGGTATCTCAGTTCGGTGTAGGTCGTTCG
CTCCAAGCTGGGCTGTGTGCACGAACCCCCCGTTCAGCCCGACCGCTGCG
CCTTATCCGGTAACTATCGTCTTGAGTCCAACCCGGTAAGACACGACTTA
TCGCCACTGGCAGCAGCCACTGGTAACAGGATTAGCAGAGCGAGGTATGT
AGGCGGTGCTACAGAGTTCTTGAAGTGGTGGCCTAACTACGGCTACACTA
GAAGAACAGTATTTGGTATCTGCGCTCTGCTGAAGCCAGTTACCTTCGGA
AAAAGAGTTGGTAGCTCTTGATCCGGCAAACAAACCACCGCTGGTAGCGG
TGGTTTTTTTGTTTGCAAGCAGCAGATTACGCGCAGAAAAAAAGGATCTC
AAGAAGATCCTTTGATCTTTTCTACGGGGTCTGACGCTCAGTGGAACGAA
AACTCACGTTAAGGGATTTTGGTCATGAGATTATCAAAAAGGATCTTCAC
CTAGATCCTTTTAAATTAAAAATGAAGTTTTAAATCAATCTAAAGTATAT
ATGAGTAAACTTGGTCTGACAGTTACCAATGCTTAATCAGTGAGGCACCT
ATCTCAGCGATCTGTCTATTTCGTTCATCCATAGTTGCCTGACTCCCCGT
CGTGTAGATAACTACGATACGGGAGGGCTTACCATCTGGCCCCAGTGCTG
CAATGATACCGCGAGACCCACGCTCACCGGCTCCAGATTTATCAGCAATA
AACCAGCCAGCCGGAAGGGCCGAGCGCAGAAGTGGTCCTGCAACTTTATC
CGCCTCCATCCAGTCTATTAATTGTTGCCGGGAAGCTAGAGTAAGTAGTT
CGCCAGTTAATAGTTTGCGCAACGTTGTTGCCATTGCTACAGGCATCGTG
GTGTCACGCTCGTCGTTTGGTATGGCTTCATTCAGCTCCGGTTCCCAACG
ATCAAGGCGAGTTACATGATCCCCCATGTTGTGCAAAAAAGCGGTTAGCT
CCTTCGGTCCTCCGATCGTTGTCAGAAGTAAGTTGGCCGCAGTGTTATCA
CTCATGGTTATGGCAGCACTGCATAATTCTCTTACTGTCATGCCATCCGT
AAGATGCTTTTCTGTGACTGGTGAGTACTCAACCAAGTCATTCTGAGAAT
AGTGTATGCGGCGACCGAGTTGCTCTTGCCCGGCGTCAATACGGGATAAT
ACCGCGCCACATAGCAGAACTTTAAAAGTGCTCATCATTGGAAAACGTTC
TTCGGGGCGAAAACTCTCAAGGATCTTACCGCTGTTGAGATCCAGTTCGA
TGTAACCCACTCGTGCACCCAACTGATCTTCAGCATCTTTTACTTTCACC
AGCGTTTCTGGGTGAGCAAAAACAGGAAGGCAAAATGCCGCAAAAAAGGG
AATAAGGGCGACACGGAAATGTTGAATACTCATACTCTTCCTTTTTCAAT
ATTATTGAAGCATTTATCAGGGTTATTGTCTCATGAGCGGATACATATTT
GAATGTATTTAGAAAAATAAACAAATAGGGGTTCCGCGCACATTTCCCCG
AAAAGTGCCACCTGACGTCT

According to some embodiments, the base vector is 85% identical to SEQ ID NO: 11. According to some embodiments, the base vector is 90% identical to SEQ ID NO: 11. According to some embodiments, the base vector is 95% identical to SEQ ID NO: 11. According to some embodiments, the base vector is 96% identical to SEQ ID NO: 11. According to some embodiments, the base vector is 97% identical to SEQ ID NO: 11. According to some embodiments, the base vector is 98% identical to SEQ ID NO: 11. According to some embodiments, the base vector is 99% identical to SEQ ID NO: 11. According to some embodiments, the base vector consists of SEQ ID NO: 11.

Plasmid 15
(SEQ ID NO: 12)
GCGCACATTTCCCCGAAAAGTGCCACCTGACGTCTTTCGTCAGATCACCG
GGTTGTTCCATATCATCGTGTCCACAAGGGCTTGCCGGTCAAGTGCCTAA
GCTCACTCGAGGGTCTCGTGAGCGAGCGAGCGCGCAGAGAGGGAGTGGCC
AACTCCATCACTAGGGGTTCCTTGTAGTTAATGATTAACCCGCCATGCTA
CTTATCGCGGCCGCAGTTGCATCTTTGTTATACCAGCGCACGAGTCTGTC
GTCATGGCTACAAGACCACGAGAACATGAGCCGGTGTCTCCAGAGAACTC
CATTGTGACTAGGTAGTCTAGAGCATGGCTACGTAGATAAGTAGCATGGC
GGGTTAATCATTAACTACACCTGCAGGAGGAACCCCTAGTGATGGAGTTG
GCCACTCCCTCTCTGCGCGCTCGCTCGCTCACTGGAGACCACCGGTTTAC
AGGATAGTCTACTCCCAGTGTCCACATGGTTAGGACCGAGGGGCTTTGGG
TTTAGACACTGGCGACTTCCGCTTCCTCGCTCACTGACTCGCTGCGCTCG
GTCGTTCGGCTGCGGCGAGCGGTATCAGCTCACTCAAAGGCGGTAATACG
GTTATCCACAGAATCAGGGGATAACGCAGGAAAGAACATGTGAGCAAAAG
GCCAGCAAAAGGCCAGGAACCGTAAAAAGGCCGCGTTGCTGGCGTTTTTC
CATAGGCTCCGCCCCCCTGACGAGCATCACAAAAATCGACGCTCAAGTCA
GAGGTGGCGAAACCCGACAGGACTATAAAGATACCAGGCGTTTCCCCCTG
GAAGCTCCCTCGTGCGCTCTCCTGTTCCGACCCTGCCGCTTACCGGATAC
CTGTCCGCCTTTCTCCCTTCGGGAAGCGTGGCGCTTTCTCATAGCTCACG
CTGTAGGTATCTCAGTTCGGTGTAGGTCGTTCGCTCCAAGCTGGGCTGTG
TGCACGAACCCCCCGTTCAGCCCGACCGCTGCGCCTTATCCGGTAACTAT
CGTCTTGAGTCCAACCCGGTAAGACACGACTTATCGCCACTGGCAGCAGC
CACTGGTAACAGGATTAGCAGAGCGAGGTATGTAGGCGGTGCTACAGAGT
TCTTGAAGTGGTGGCCTAACTACGGCTACACTAGAAGAACAGTATTTGGT
ATCTGCGCTCTGCTGAAGCCAGTTACCTTCGGAAAAAGAGTTGGTAGCTC
TTGATCCGGCAAACAAACCACCGCTGGTAGCGGTGGTTTTTTTGTTTGCA
AGCAGCAGATTACGCGCAGAAAAAAAGGATCTCAAGAAGATCCTTTGATC
TTTTCTACGGGGTCTGACGCTCAGTGGAACGAAAACTCACGTTAAGGGAT
TTTGGTCATGAGATTATCAAAAAGGATCTTCACCTAGATCCTTTTAAATT
AAAAATGAAGTTTTAAATCAATCTAAAGTATATATGAGTAAACTTGGTCT
GACAGTTACCAATGCTTAATCAGTGAGGCACCTATCTCAGCGATCTGTCT
ATTTCGTTCATCCATAGTTGCCTGACTCCCCGTCGTGTAGATAACTACGA
TACGGGAGGGCTTACCATCTGGCCCCAGTGCTGCAATGATACCGCGAGAC
CCACGCTCACCGGCTCCAGATTTATCAGCAATAAACCAGCCAGCCGGAAG
GGCCGAGCGCAGAAGTGGTCCTGCAACTTTATCCGCCTCCATCCAGTCTA
TTAATTGTTGCCGGGAAGCTAGAGTAAGTAGTTCGCCAGTTAATAGTTTG
CGCAACGTTGTTGCCATTGCTACAGGCATCGTGGTGTCACGCTCGTCGTT
TGGTATGGCTTCATTCAGCTCCGGTTCCCAACGATCAAGGCGAGTTACAT
GATCCCCCATGTTGTGCAAAAAAGCGGTTAGCTCCTTCGGTCCTCCGATC
GTTGTCAGAAGTAAGTTGGCCGCAGTGTTATCACTCATGGTTATGGCAGC
ACTGCATAATTCTCTTACTGTCATGCCATCCGTAAGATGCTTTTCTGTGA
CTGGTGAGTACTCAACCAAGTCATTCTGAGAATAGTGTATGCGGCGACCG
AGTTGCTCTTGCCCGGCGTCAATACGGGATAATACCGCGCCACATAGCAG
AACTTTAAAAGTGCTCATCATTGGAAAACGTTCTTCGGGGCGAAAACTCT
CAAGGATCTTACCGCTGTTGAGATCCAGTTCGATGTAACCCACTCGTGCA
CCCAACTGATCTTCAGCATCTTTTACTTTCACCAGCGTTTCTGGGTGAGC
AAAAACAGGAAGGCAAAATGCCGCAAAAAAGGGAATAAGGGCGACACGGA
AATGTTGAATACTCATACTCTTCCTTTTTCAATATTATTGAAGCATTTAT
CAGGGTTATTGTCTCATGAGCGGATACATATTTGAATGTATTTAGAAAAA
TAAACAAATAGGGGTTCC

According to some embodiments, the base vector is 85% identical to SEQ ID NO: 12. According to some embodiments, the base vector is 90% identical to SEQ ID NO: 12. According to some embodiments, the base vector is 95% identical to SEQ ID NO: 12. According to some embodiments, the base vector is 96% identical to SEQ ID NO: 12. According to some embodiments, the base vector is 97% identical to SEQ ID NO: 12. According to some embodiments, the base vector is 98% identical to SEQ ID NO: 12. According to some embodiments, the base vector is 99% identical to SEQ ID NO: 12. According to some embodiments, the base vector consists of SEQ ID NO: 12.

Plasmid 1
(SEQ ID NO: 13)
TTCGTCAGATCACCGGGTTGTTCCATATCATCGTGTCCACAAGGGCTTGC
CGGTCAAGTGCCTAAGCTCACTCGAGCGTCTCGTGAGCGAGCGAGCGCGC
AGAGAGGGAGTGGCCAACTCCATCACTAGGGGTTCCTTGTAGTTAATGAT
TAACCCGCCATGCTACTTATCGCGGCCGCAGTTGCATCTTTGTTATACCA
GCGCACGAGTCTGTCGTCATGGCTACAAGACCACGAGAACATGAGCCGGT
GTCTCCAGAGAACTCCATTGTGACTAGGTAGTCTAGAGCATGGCTACGTA
GATAAGTAGCATGGCGGGTTAATCATTAACTACACCTGCAGGAGGAACCC
CTAGTGATGGAGTTGGCCACTCCCTCTCTGCGCGCTCGCTCGCTCACGAG
ACGACCGGTTTACAGGATAGTCTACTCCCAGTGTCCACATGGTTAGGACC
GAGGGGCTTTGGGTTTAGACACTGGCGACTTCCGCTTCCTCGCTCACTGA
CTCGCTGCGCTCGGTCGTTCGGCTGCGGCGAGCGGTATCAGCTCACTCAA
AGGCGGTAATACGGTTATCCACAGAATCAGGGGATAACGCAGGAAAGAAC
ATGTGAGCAAAAGGCCAGCAAAAGGCCAGGAACCGTAAAAAGGCCGCGTT
GCTGGCGTTTTTCCATAGGCTCCGCCCCCCTGACGAGCATCACAAAAATC
GACGCTCAAGTCAGAGGTGGCGAAACCCGACAGGACTATAAAGATACCAG
GCGTTTCCCCCTGGAAGCTCCCTCGTGCGCTCTCCTGTTCCGACCCTGCC
GCTTACCGGATACCTGTCCGCCTTTCTCCCTTCGGGAAGCGTGGCGCTTT
CTCATAGCTCACGCTGTAGGTATCTCAGTTCGGTGTAGGTCGTTCGCTCC
AAGCTGGGCTGTGTGCACGAACCCCCCGTTCAGCCCGACCGCTGCGCCTT
ATCCGGTAACTATCGTCTTGAGTCCAACCCGGTAAGACACGACTTATCGC
CACTGGCAGCAGCCACTGGTAACAGGATTAGCAGAGCGAGGTATGTAGGC
GGTGCTACAGAGTTCTTGAAGTGGTGGCCTAACTACGGCTACACTAGAAG
AACAGTATTTGGTATCTGCGCTCTGCTGAAGCCAGTTACCTTCGGAAAAA
GAGTTGGTAGCTCTTGATCCGGCAAACAAACCACCGCTGGTAGCGGTGGT
TTTTTTGTTTGCAAGCAGCAGATTACGCGCAGAAAAAAAGGATCTCAAGA
AGATCCTTTGATCTTTTCTACGGGGTCTGACGCTCAGTGGAACGAAAACT
CACGTTAAGGGATTTTGGTCATGAGATTATCAAAAAGGATCTTCACCTAG
ATCCTTTTAAATTAAAAATGAAGTTTTAAATCAATCTAAAGTATATATGA
GTAAACTTGGTCTGACAGTTACCAATGCTTAATCAGTGAGGCACCTATCT
CAGCGATCTGTCTATTTCGTTCATCCATAGTTGCCTGACTCCCCGTCGTG
TAGATAACTACGATACGGGAGGGCTTACCATCTGGCCCCAGTGCTGCAAT
GATACCGCGAGACCCACGCTCACCGGCTCCAGATTTATCAGCAATAAACC
AGCCAGCCGGAAGGGCCGAGCGCAGAAGTGGTCCTGCAACTTTATCCGCC
TCCATCCAGTCTATTAATTGTTGCCGGGAAGCTAGAGTAAGTAGTTCGCC
AGTTAATAGTTTGCGCAACGTTGTTGCCATTGCTACAGGCATCGTGGTGT
CACGCTCGTCGTTTGGTATGGCTTCATTCAGCTCCGGTTCCCAACGATCA
AGGCGAGTTACATGATCCCCCATGTTGTGCAAAAAAGCGGTTAGCTCCTT
CGGTCCTCCGATCGTTGTCAGAAGTAAGTTGGCCGCAGTGTTATCACTCA
TGGTTATGGCAGCACTGCATAATTCTCTTACTGTCATGCCATCCGTAAGA
TGCTTTTCTGTGACTGGTGAGTACTCAACCAAGTCATTCTGAGAATAGTG
TATGCGGCGACCGAGTTGCTCTTGCCCGGCGTCAATACGGGATAATACCG
CGCCACATAGCAGAACTTTAAAAGTGCTCATCATTGGAAAACGTTCTTCG
GGGCGAAAACTCTCAAGGATCTTACCGCTGTTGAGATCCAGTTCGATGTA
ACCCACTCGTGCACCCAACTGATCTTCAGCATCTTTTACTTTCACCAGCG
TTTCTGGGTGAGCAAAAACAGGAAGGCAAAATGCCGCAAAAAAGGGAATA
AGGGCGACACGGAAATGTTGAATACTCATACTCTTCCTTTTTCAATATTA
TTGAAGCATTTATCAGGGTTATTGTCTCATGAGCGGATACATATTTGAAT
GTATTTAGAAAAATAAACAAATAGGGGTTCCGCGCACATTTCCCCGAAAA
GTGCCACCTGACGTCT

According to some embodiments, the base vector is 85% identical to SEQ ID NO: 13. According to some embodiments, the base vector is 90% identical to SEQ ID NO: 13. According to some embodiments, the base vector is 95% identical to SEQ ID NO: 13. According to some embodiments, the base vector is 96% identical to SEQ ID NO: 13. According to some embodiments, the base vector is 97% identical to SEQ ID NO: 13. According to some embodiments, the base vector is 98% identical to SEQ ID NO: 13. According to some embodiments, the base vector is 99% identical to SEQ ID NO: 13. According to some embodiments, the base vector consists of SEQ ID NO: 13.

Plasmid 11
(SEQ ID NO: 14)
TTCGTCAGATCACCGGGTTGTTCCATATCATCGTGTCCACAAGGGCTTGC
CGGTCAAGTGCCTAAGCTCACTCGAGCGTCTCGTGAGCGAGCGAGCGCGC
AGAGAGGGAGTGGCCAACTCCATCACTAGGGGTTCCTTGTAGTTAATGAT
TAACCCGCCATGCTACTTATCGCGGCCGCAGTTGCATCTTTGTTATACCA
GCGCACGAGTCTGTCGTCATGGCTACAAGACCACGAGAACATGAGCCGGT
GTCTCCAGAGAACTCCATTGTGACTAGGTAGTCTAGAGCATGGCTACGTA
GATAAGTAGCATGGCGGGTTAATCATTAACTACACCTGCAGGAGGAACCC
CTAGTGATGGAGTTGGCCACTCCCTCTCTGCGCGCTCGCTCGCTCACGAG
ACGACCGGTTTACAGGATAGTCTACTCCCAGTGTCCACATGGTTAGGACC
GAGGGGCTTTGGGTTTAGACACTGGCGACTTCCGCTTCCTCGCTCACTGA
CTCGCTGCGCTCGGTCGTTCGGCTGCGGCGAGCGGTATCAGCTCACTCAA
AGGCGGTAATACGGTTATCCACAGAATCAGGGGATAACGCAGGAAAGAAC
ATGTGAGCAAAAGGCCAGCAAAAGGCCAGGAACCGTAAAAAGGCCGCGTT
GCTGGCGTTTTTCCATAGGCTCCGCCCCCCTGACGAGCATCACAAAAATC
GACGCTCAAGTCAGAGGTGGCGAAACCCGACAGGACTATAAAGATACCAG
GCGTTTCCCCCTGGAAGCTCCCTCGTGCGCTCTCCTGTTCCGACCCTGCC
GCTTACCGGATACCTGTCCGCCTTTCTCCCTTCGGGAAGCGTGGCGCTTT
CTCATAGCTCACGCTGTAGGTATCTCAGTTCGGTGTAGGTCGTTCGCTCC
AAGCTGGGCTGTGTGCACGAACCCCCCGTTCAGCCCGACCGCTGCGCCTT
ATCCGGTAACTATCGTCTTGAGTCCAACCCGGTAAGACACGACTTATCGC
CACTGGCAGCAGCCACTGGTAACAGGATTAGCAGAGCGAGGTATGTAGGC
GGTGCTACAGAGTTCTTGAAGTGGTGGCCTAACTACGGCTACACTAGAAG
AACAGTATTTGGTATCTGCGCTCTGCTGAAGCCAGTTACCTTCGGAAAAA
GAGTTGGTAGCTCTTGATCCGGCAAACAAACCACCGCTGGTAGCGGTGGT
TTTTTTGTTTGCAAGCAGCAGATTACGCGCAGAAAAAAAGGATCTCAAGA
AGATCCTTTGATCTTTTCTACGGGGTCTGACGCTCAGTGGAACGAAAACT
CACGTTAAGGGATTTTGGTCATGAGATTATCAAAAAGGATCTTCACCTAG
ATCCTTTTAAATTAAAAATGAAGTTTTAAATCAATCTAAAGTATATATGA
GTAAACTTGGTCTGACAGTTACCAATGCTTAATCAGTGAGGCACCTATCT
CAGCGATCTGTCTATTTCGTTCATCCATAGTTGCCTGACTCCCCGTCGTG
TAGATAACTACGATACGGGAGGGCTTACCATCTGGCCCCAGTGCTGCAAT
GATACCGCGAGACCCACGCTCACCGGCTCCAGATTTATCAGCAATAAACC
AGCCAGCCGGAAGGGCCGAGCGCAGAAGTGGTCCTGCAACTTTATCCGCC
TCCATCCAGTCTATTAATTGTTGCCGGGAAGCTAGAGTAAGTAGTTCGCC
AGTTAATAGTTTGCGCAACGTTGTTGCCATTGCTACAGGCATCGTGGTGT
CACGCTCGTCGTTTGGTATGGCTTCATTCAGCTCCGGTTCCCAACGATCA
AGGCGAGTTACATGATCCCCCATGTTGTGCAAAAAAGCGGTTAGCTCCTT
CGGTCCTCCGATCGTTGTCAGAAGTAAGTTGGCCGCAGTGTTATCACTCA
TGGTTATGGCAGCACTGCATAATTCTCTTACTGTCATGCCATCCGTAAGA
TGCTTTTCTGTGACTGGTGAGTACTCAACCAAGTCATTCTGAGAATAGTG
TATGCGGCGACCGAGTTGCTCTTGCCCGGCGTCAATACGGGATAATACCG
CGCCACATAGCAGAACTTTAAAAGTGCTCATCATTGGAAAACGTTCTTCG
GGGCGAAAACTCTCAAGGATCTTACCGCTGTTGAGATCCAGTTCGATGTA
ACCCACTCGTGCACCCAACTGATCTTCAGCATCTTTTACTTTCACCAGCG
TTTCTGGGTGAGCAAAAACAGGAAGGCAAAATGCCGCAAAAAGGGAATAA
GGGCGACACGGAAATGTTGAATACTCATACTCTTCCTTTTTCAATATTAT
TGAAGCATTTATCAGGGTTATTGTCTCATGAGCGGATACATATTTGAATG
TATTTAGAAAAATAAACAAATAGGGGTTCCGCGCACATTTCCCCGAAAAG
TGCCACCTGACGTCT

According to some embodiments, the base vector is 85% identical to SEQ ID NO: 14. According to some embodiments, the base vector is 90% identical to SEQ ID NO: 14. According to some embodiments, the base vector is 95% identical to SEQ ID NO: 14. According to some embodiments, the base vector is 96% identical to SEQ ID NO: 14. According to some embodiments, the base vector is 97% identical to SEQ ID NO: 14. According to some embodiments, the base vector is 98% identical to SEQ ID NO: 14. According to some embodiments, the base vector is 99% identical to SEQ ID NO: 14. According to some embodiments, the base vector consists of SEQ ID NO: 14.

Plasmid 8
(SEQ ID NO: 15)
TTCGTCAGATCACCGGGTTGTTCCATATCATCGTGTCCACAAGGGCTTGC
CGGTCAAGTGCCTAAGCTCACTCGAGCGTCTCGTGAGCGAGCGAGCGCGC
AGAGAGGGAGTGGCCAACTCCATCACTAGGGGTTCCTTGTAGTTAATGAT
TAACCCGCCATGCTACTTATCGCGGCCGCAGTTGCATCTTTGTTATACCA
GCGCACGAGTCTGTCGTCATGGCTACAAGACCACGAGAACATGAGCCGGT
GTCTCCAGAGAACTCCATTGTGACTAGGTAGTCTAGAGCATGGCTACGTA
GATAAGTAGCATGGCGGGTTAATCATTAACTACACCTGCAGGAGGAACCC
CTAGTGATGGAGTTGGCCACTCCCTCTCTGCGCGCTCGCTCGCTCACGAG
ACGACCGGTTTACAGGATAGTCTACTCCCAGTGTCCACATGGTTAGGACC
GAGGGGCTTTGGGTTTAGACACTGGCGACTTCCGCTTCCTCGCTCACTGA
CTCGCTGCGCTCGGTCGTTCGGCTGCGGCGAGCGGTATCAGCTCACTCAA
AGGCGGTAATACGGTTATCCACAGAATCAGGGGATAACGCAGGAAAGAAC
ATGTGAGCAAAAGGCCAGCAAAAGGCCAGGAACCGTAAAAAGGCCGCGTT
GCTGGCGTTTTTCCATAGGCTCCGCCCCCCTGACGAGCATCACAAAAATC
GACGCTCAAGTCAGAGGTGGCGAAACCCGACAGGACTATAAAGATACCAG
GCGTTTCCCCCTGGAAGCTCCCTCGTGCGCTCTCCTGTTCCGACCCTGCC
GCTTACCGGATACCTGTCCGCCTTTCTCCCTTCGGGAAGCGTGGCGCTTT
CTCATAGCTCACGCTGTAGGTATCTCAGTTCGGTGTAGGTCGTTCGCTCC
AAGCTGGGCTGTGTGCACGAACCCCCCGTTCAGCCCGACCGCTGCGCCTT
ATCCGGTAACTATCGTCTTGAGTCCAACCCGGTAAGACACGACTTATCGC
CACTGGCAGCAGCCACTGGTAACAGGATTAGCAGAGCGAGGTATGTAGGC
GGTGCTACAGAGTTCTTGAAGTGGTGGCCTAACTACGGCTACACTAGAAG
AACAGTATTTGGTATCTGCGCTCTGCTGAAGCCAGTTACCTTCGGAAAAA
GAGTTGGTAGCTCTTGATCCGGCAAACAAACCACCGCTGGTAGCGGTGGT
TTTTTTGTTTGCAAGCAGCAGATTACGCGCAGAAAAAAAGGATCTCAAGA
AGATCCTTTGATCTTTTCTACGGGGTCTGACGCTCAGTGGAACGAAAACT
CACGTTAAGGGATTTTGGTCATGAGATTATCAAAAAGGATCTTCACCTAG
ATCCTTTTAAATTAAAAATGAAGTTTTAAATCAATCTAAAGTATATATGA
GTAAACTTGGTCTGACAGTTACCAATGCTTAATCAGTGAGGCACCTATCT
CAGCGATCTGTCTATTTCGTTCATCCATAGTTGCCTGACTCCCCGTCGTG
TAGATAACTACGATACGGGAGGGCTTACCATCTGGCCCCAGTGCTGCAAT
GATACCGCGAGACCCACGCTCACCGGCTCCAGATTTATCAGCAATAAACC
AGCCAGCCGGAAGGGCCGAGCGCAGAAGTGGTCCTGCAACTTTATCCGCC
TCCATCCAGTCTATTAATTGTTGCCGGGAAGCTAGAGTAAGTAGTTCGCC
AGTTAATAGTTTGCGCAACGTTGTTGCCATTGCTACAGGCATCGTGGTGT
CACGCTCGTCGTTTGGTATGGCTTCATTCAGCTCCGGTTCCCAACGATCA
AGGCGAGTTACATGATCCCCCATGTTGTGCAAAAAAGCGGTTAGCTCCTT
CGGTCCTCCGATCGTTGTCAGAAGTAAGTTGGCCGCAGTGTTATCACTCA
TGGTTATGGCAGCACTGCATAATTCTCTTACTGTCATGCCATCCGTAAGA
TGCTTTTCTGTGACTGGTGAGTACTCAACCAAGTCATTCTGAGAATAGTG
TATGCGGCGACCGAGTTGCTCTTGCCCGGCGTCAATACGGGATAATACCG
CGCCACATAGCAGAACTTTAAAAGTGCTCATCATTGGAAAACGTTCTTCG
GGGCGAAAACTCTCAAGGATCTTACCGCTGTTGAGATCCAGTTCGATGTA
ACCCACTCGTGCACCCAACTGATCTTCAGCATCTTTTACTTTCACCAGCG
TTTCTGGGTGAGCAAAAACAGGAAGGCAAAATGCCGCAAAAAAGGGAATA
AGGGCGACACGGAAATGTTGAATACTCATACTCTTCCTTTTTCAATATTA
TTGAAGCATTTATCAGGGTTATTGTCTCATGAGCGGATACATATTTGAAT
GTATTTAGAAAAATAAACAAATAGGGGTTCCGCGCACATTTCCCCGAAAA
GTGCCACCTGACGTCT

According to some embodiments, the base vector is 85% identical to SEQ ID NO: 15. According to some embodiments, the base vector is 90% identical to SEQ ID NO: 15. According to some embodiments, the base vector is 95% identical to SEQ ID NO: 15. According to some embodiments, the base vector is 96% identical to SEQ ID NO: 15. According to some embodiments, the base vector is 97% identical to SEQ ID NO: 15. According to some embodiments, the base vector is 98% identical to SEQ ID NO: 15. According to some embodiments, the base vector is 99% identical to SEQ ID NO: 15. According to some embodiments, the base vector consists of SEQ ID NO: 15.

Plasmid 7
(SEQ ID NO: 16)
TTCGTCAGATCACCGGGTTGTTCCATATCATCGTGTCCACAAGGGCTTGC
CGGTCAAGTGCCTAAGCTCACTCGAGCGTCTCGTGAGCGAGCGAGCGCGC
AGAGAGGGAGTGGCCAACTCCATCACTAGGGGTTCCTTGTAGTTAATGAT
TAACCCGCCATGCTACTTATCGCGGCCGCAGTTGCATCTTTGTTATACCA
GCGCACGAGTCTGTCGTCATGGCTACAAGACCACGAGAACATGAGCCGGT
GTCTCCAGAGAACTCCATTGTGACTAGGTAGTCTAGAGCATGGCTACGTA
GATAAGTAGCATGGCGGGTTAATCATTAACTACACCTGCAGGAGGAACCC
CTAGTGATGGAGTTGGCCACTCCCTCTCTGCGCGCTCGCTCGCTCACGAG
ACGACCGGTTTACAGGATAGTCTACTCCCAGTGTCCACATGGTTAGGACC
GAGGGGCTTTGGGTTTAGACACTGGCGACTTCCGCTTCCTCGCTCACTGA
CTCGCTGCGCTCGGTCGTTCGGCTGCGGCGAGCGGTATCAGCTCACTCAA
AGGCGGTAATACGGTTATCCACAGAATCAGGGGATAACGCAGGAAAGAAC
ATGTGAGCAAAAGGCCAGCAAAAGGCCAGGAACCGTAAAAAGGCCGCGTT
GCTGGCGTTTTTCCATAGGCTCCGCCCCCCTGACGAGCATCACAAAAATC
GACGCTCAAGTCAGAGGTGGCGAAACCCGACAGGACTATAAAGATACCAG
GCGTTTCCCCCTGGAAGCTCCCTCGTGCGCTCTCCTGTTCCGACCCTGCC
GCTTACCGGATACCTGTCCGCCTTTCTCCCTTCGGGAAGCGTGGCGCTTT
CTCATAGCTCACGCTGTAGGTATCTCAGTTCGGTGTAGGTCGTTCGCTCC
AAGCTGGGCTGTGTGCACGAACCCCCCGTTCAGCCCGACCGCTGCGCCTT
ATCCGGTAACTATCGTCTTGAGTCCAACCCGGTAAGACACGACTTATCGC
CACTGGCAGCAGCCACTGGTAACAGGATTAGCAGAGCGAGGTATGTAGGC
GGTGCTACAGAGTTCTTGAAGTGGTGGCCTAACTACGGCTACACTAGAAG
AACAGTATTTGGTATCTGCGCTCTGCTGAAGCCAGTTACCTTCGGAAAAA
GAGTTGGTAGCTCTTGATCCGGCAAACAAACCACCGCTGGTAGCGGTGGT
TTTTTTGTTTGCAAGCAGCAGATTACGCGCAGAAAAAAAGGATCTCAAGA
AGATCCTTTGATCTTTTCTACGGGGTCTGACGCTCAGTGGAACGAAAACT
CACGTTAAGGGATTTTGGTCATGAGATTATCAAAAAGGATCTTCACCTAG
ATCCTTTTAAATTAAAAATGAAGTTTTAAATCAATCTAAAGTATATATGA
GTAAACTTGGTCTGACAGTTACCAATGCTTAATCAGTGAGGCACCTATCT
CAGCGATCTGTCTATTTCGTTCATCCATAGTTGCCTGACTCCCCGTCGTG
TAGATAACTACGATACGGGAGGGCTTACCATCTGGCCCCAGTGCTGCAAT
GATACCGCGAGACCCACGCTCACCGGCTCCAGATTTATCAGCAATAAACC
AGCCAGCCGGAAGGGCCGAGCGCAGAAGTGGTCCTGCAACTTTATCCGCC
TCCATCCAGTCTATTAATTGTTGCCGGGAAGCTAGAGTAAGTAGTTCGCC
AGTTAATAGTTTGCGCAACGTTGTTGCCATTGCTACAGGCATCGTGGTGT
CACGCTCGTCGTTTGGTATGGCTTCATTCAGCTCCGGTTCCCAACGATCA
AGGCGAGTTACATGATCCCCCATGTTGTGCAAAAAAGCGGTTAGCTCCTT
CGGTCCTCCGATCGTTGTCAGAAGTAAGTTGGCCGCAGTGTTATCACTCA
TGGTTATGGCAGCACTGCATAATTCTCTTACTGTCATGCCATCCGTAAGA
TGCTTTTCTGTGACTGGTGAGTACTCAACCAAGTCATTCTGAGAATAGTG
TATGCGGCGACCGAGTTGCTCTTGCCCGGCGTCAATACGGGATAATACCG
CGCCACATAGCAGAACTTTAAAAGTGCTCATCATTGGAAAACGTTCTTCG
GGGCGAAAACTCTCAAGGATCTTACCGCTGTTGAGATCCAGTTCGATGTA
ACCCACTCGTGCACCCAACTGATCTTCAGCATCTTTTACTTTCACCAGCG
TTTCTGGGTGAGCAAAAACAGGAAGGCAAAATGCCGCAAAAAAGGGAATA
AGGGCGACACGGAAATGTTGAATACTCATACTCTTCCTTTTTCAATATTA
TTGAAGCATTTATCAGGGTTATTGTCTCATGAGCGGATACATATTTGAAT
GTATTTAGAAAAATAAACAAATAGGGGTTCCGCGCACATTTCCCCGAAAA
GTGCCACCTGACGTCT

According to some embodiments, the base vector is 85% identical to SEQ ID NO: 16. According to some embodiments, the base vector is 90% identical to SEQ ID NO: 16. According to some embodiments, the base vector is 95% identical to SEQ ID NO: 16. According to some embodiments, the base vector is 96% identical to SEQ ID NO: 16. According to some embodiments, the base vector is 97% identical to SEQ ID NO: 16. According to some embodiments, the base vector is 98% identical to SEQ ID NO: 16. According to some embodiments, the base vector is 99% identical to SEQ ID NO: 16. According to some embodiments, the base vector consists of SEQ ID NO: 16.

Plasmid 6
(SEQ ID NO: 17)
TTCGTCAGATCACCGGGTTGTTCCATATCATCGTGTCCACAAGGGCTTGC
CGGTCAAGTGCCTAAGCTCACTCGAGCGTCTCGTGAGCGAGCGAGCGCGC
AGAGAGGGAGTGGCCAACTCCATCACTAGGGGTTCCTTGTAGTTAATGAT
TAACCCGCCATGCTACTTATCGCGGCCGCAGTTGCATCTTTGTTATACCA
GCGCACGAGTCTGTCGTCATGGCTACAAGACCACGAGAACATGAGCCGGT
GTCTCCAGAGAACTCCATTGTGACTAGGTAGTCTAGAGCATGGCTACGTA
GATAAGTAGCATGGCGGGTTAATCATTAACTACACCTGCAGGAGGAACCC
CTAGTGATGGAGTTGGCCACTCCCTCTCTGCGCGCTCGCTCGCTCACGAG
ACGACCGGTTTACAGGATAGTCTACTCCCAGTGTCCACATGGTTAGGACC
GAGGGGCTTTGGGTTTAGACACTGGCGACTTCCGCTTCCTCGCTCACTGA
CTCGCTGCGCTCGGTCGTTCGGCTGCGGCGAGCGGTATCAGCTCACTCAA
AGGCGGTAATACGGTTATCCACAGAATCAGGGGATAACGCAGGAAAGAAC
ATGTGAGCAAAAGGCCAGCAAAAGGCCAGGAACCGTAAAAAGGCCGCGTT
GCTGGCGTTTTTCCATAGGCTCCGCCCCCCTGACGAGCATCACAAAAATC
GACGCTCAAGTCAGAGGTGGCGAAACCCGACAGGACTATAAAGATACCAG
GCGTTTCCCCCTGGAAGCTCCCTCGTGCGCTCTCCTGTTCCGACCCTGCC
GCTTACCGGATACCTGTCCGCCTTTCTCCCTTCGGGAAGCGTGGCGCTTT
CTCATAGCTCACGCTGTAGGTATCTCAGTTCGGTGTAGGTCGTTCGCTCC
AAGCTGGGCTGTGTGCACGAACCCCCCGTTCAGCCCGACCGCTGCGCCTT
ATCCGGTAACTATCGTCTTGAGTCCAACCCGGTAAGACACGACTTATCGC
CACTGGCAGCAGCCACTGGTAACAGGATTAGCAGAGCGAGGTATGTAGGC
GGTGCTACAGAGTTCTTGAAGTGGTGGCCTAACTACGGCTACACTAGAAG
AACAGTATTTGGTATCTGCGCTCTGCTGAAGCCAGTTACCTTCGGAAAAA
GAGTTGGTAGCTCTTGATCCGGCAAACAAACCACCGCTGGTAGCGGTGGT
TTTTTTGTTTGCAAGCAGCAGATTACGCGCAGAAAAAAAGGATCTCAAGA
AGATCCTTTGATCTTTTCTACGGGGTCTGACGCTCAGTGGAACGAAAACT
CACGTTAAGGGATTTTGGTCATGAGATTATCAAAAAGGATCTTCACCTAG
ATCCTTTTAAATTAAAAATGAAGTTTTAAATCAATCTAAAGTATATATGA
GTAAACTTGGTCTGACAGTTACCAATGCTTAATCAGTGAGGCACCTATCT
CAGCGATCTGTCTATTTCGTTCATCCATAGTTGCCTGACTCCCCGTCGTG
TAGATAACTACGATACGGGAGGGCTTACCATCTGGCCCCAGTGCTGCAAT
GATACCGCGAGACCCACGCTCACCGGCTCCAGATTTATCAGCAATAAACC
AGCCAGCCGGAAGGGCCGAGCGCAGAAGTGGTCCTGCAACTTTATCCGCC
TCCATCCAGTCTATTAATTGTTGCCGGGAAGCTAGAGTAAGTAGTTCGCC
AGTTAATAGTTTGCGCAACGTTGTTGCCATTGCTACAGGCATCGTGGTGT
CACGCTCGTCGTTTGGTATGGCTTCATTCAGCTCCGGTTCCCAACGATCA
AGGCGAGTTACATGATCCCCCATGTTGTGCAAAAAAGCGGTTAGCTCCTT
CGGTCCTCCGATCGTTGTCAGAAGTAAGTTGGCCGCAGTGTTATCACTCA
TGGTTATGGCAGCACTGCATAATTCTCTTACTGTCATGCCATCCGTAAGA
TGCTTTTCTGTGACTGGTGAGTACTCAACCAAGTCATTCTGAGAATAGTG
TATGCGGCGACCGAGTTGCTCTTGCCCGGCGTCAATACGGGATAATACCG
CGCCACATAGCAGAACTTTAAAAGTGCTCATCATTGGAAAACGTTCTTCG
GGGCGAAAACTCTCAAGGATCTTACCGCTGTTGAGATCCAGTTCGATGTA
ACCCACTCGTGCACCCAACTGATCTTCAGCATCTTTTACTTTCACCAGCG
TTTCTGGGTGAGCAAAAACAGGAAGGCAAAATGCCGCAAAAAAGGGAATA
AGGGCGACACGGAAATGTTGAATACTCATACTCTTCCTTTTTCAATATTA
TTGAAGCATTTATCAGGGTTATTGTCTCATGAGCGGATACATATTTGAAT
GTATTTAGAAAAATAAACAAATAGGGGTTCCGCGCACATTTCCCCGAAAA
GTGCCACCTGACGTCT

According to some embodiments, the base vector is 85% identical to SEQ ID NO: 17. According to some embodiments, the base vector is 90% identical to SEQ ID NO: 17. According to some embodiments, the base vector is 95% identical to SEQ ID NO: 17. According to some embodiments, the base vector is 96% identical to SEQ ID NO: 17. According to some embodiments, the base vector is 97% identical to SEQ ID NO: 17. According to some embodiments, the base vector is 98% identical to SEQ ID NO: 17. According to some embodiments, the base vector is 99% identical to SEQ ID NO: 17. According to some embodiments, the base vector consists of SEQ ID NO: 17.

### Double-Stranded DNA Constructs

As disclosed herein, the cell-free synthesis of DNA vectors involves excising a transgene expression cassette from a double-stranded DNA construct. The double-stranded DNA construct may be prepared, for example, by sub-cloning a transgene expression cassette into a base vector. Thus, in one embodiment, the double-stranded DNA construct contains, in the 5′→3′ order: a first non-palindromic restriction endonuclease recognition site and a corresponding first cleavage site, first partial ITR, transgene expression cassette, second partial ITR, and a second non-palindromic restriction endonuclease recognition site and a corresponding second cleavage site, as well as all embodiments of the any of the foregoing as disclosed herein, including embodiments of the restriction endonucleases for which the recognition sites are specific. In some embodiments, the double-stranded DNA construct further includes an origin of replication and a selectable marker gene. In some embodiments, the base vector further includes one or more spacer regions, such as a spacer between the first partial ITR and the transgene expression cassette and a spacer between the second partial ITR and the transgene expression cassette (see e.g., FIG. 3 showing exemplary double-stranded DNA construct carrying a FVIII-expressing transgene expression cassette, Construct 1).

### Kits

In one embodiment, a kit for preparing a DNA vector, such as a closed-ended DNA (ceDNA) vector includes a base vector as described above, at least one restriction endonuclease capable of cleaving the DNA vector at the multiple cloning site to allow the multiple cloning site to receive a transgene (e.g., BsaI for Plasmid 11; see FIG. 2), at least one restriction endonuclease capable of cleaving at the first and second cleavage sites (e.g., NotI and XbaI for Plasmid 11; see FIG. 2), and a ligase.

In another embodiment, a kit for preparing a DNA vector, such as a closed-ended DNA (ceDNA) vector includes a double-stranded DNA construct as described above, at least one restriction endonuclease capable of cleaving the double-stranded DNA construct at the first and second cleavage sites (e.g., BsaI for Construct 1, see FIG. 3), and a ligase.

Further embodiments of the kits as described above may include one or more ITR oligonucleotides as defined herein.

### VII. Pharmaceutical Compositions

In another aspect, pharmaceutical compositions are provided. The pharmaceutical composition comprises a closed-ended DNA vector, e.g., ceDNA vector produced using the synthetic process as described herein and a pharmaceutically acceptable carrier or diluent.

A closed-ended DNA vector, including a ceDNA vector, produced using the synthetic process as described herein can be incorporated into pharmaceutical compositions suitable for administration to a subject for in vivo delivery to cells, tissues, or organs of the subject. Typically, the pharmaceutical composition comprises a ceDNA vector as disclosed herein and a pharmaceutically acceptable carrier. For example, a closed-ended DNA vector, including a ceDNA vector, produced using the synthetic process as described herein can be incorporated into a pharmaceutical composition suitable for a desired route of therapeutic administration (e.g., parenteral administration). Passive tissue transduction via high pressure intravenous or intra-arterial infusion, as well as intracellular injection, such as intranuclear microinjection or intracytoplasmic injection, are also contemplated. Pharmaceutical compositions for therapeutic purposes can be formulated as a solution, microemulsion, dispersion, liposomes, or other ordered structure suitable to high synthetically produced closed-ended DNA vector, e.g., ceDNA vector concentration. Sterile injectable solutions can be prepared by incorporating the synthetically produced closed-ended DNA vector, e.g., ceDNA vector compound in the required amount in an appropriate buffer with one or a combination of ingredients enumerated above, as required, followed by filtered sterilization including a ceDNA vector can be formulated to deliver a transgene in the nucleic acid to the cells of a recipient, resulting in the therapeutic expression of the transgene or donor sequence therein. The composition can also include a pharmaceutically acceptable carrier.

Pharmaceutically active compositions comprising a closed-ended DNA vector, including a ceDNA vector, produced using the synthetic process as described herein can be formulated to deliver a transgene for various purposes to the cell, e.g., cells of a subject.

Pharmaceutical compositions for therapeutic purposes typically must be sterile and stable under the conditions of manufacture and storage. The composition can be formulated as a solution, microemulsion, dispersion, liposomes, or other ordered structure suitable to high synthetically produced closed-ended DNA vector, e.g. ceDNA vector concentration. Sterile injectable solutions can be prepared by incorporating the synthetically produced closed-ended DNA vector, e.g., ceDNA vector compound in the required amount in an appropriate buffer with one or a combination of ingredients enumerated above, as required, followed by filtered sterilization.

A closed-ended DNA vector, including a ceDNA vector, produced using the synthetic process as described herein as disclosed herein can be incorporated into a pharmaceutical composition suitable for topical, systemic, intra-amniotic, intrathecal, intracranial, intra-arterial, intravenous, intralymphatic, intraperitoneal, subcutaneous, tracheal, intra-tissue (e.g., intramuscular, intracardiac, intrahepatic, intrarenal, intracerebral), intrathecal, intravesical, conjunctival (e.g., extra-orbital, intraorbital, retroorbital, intraretinal, subretinal, choroidal, sub-choroidal, intrastromal, intracameral and intravitreal), intracochlear, and mucosal (e.g., oral, rectal, nasal) administration. Passive tissue transduction via high pressure intravenous or intraarterial infusion, as well as intracellular injection, such as intranuclear microinjection or intracytoplasmic injection, are also contemplated.

In some aspects, the methods provided herein comprise delivering one or more closed-ended DNA vector, including a ceDNA vector, produced using the synthetic process as described herein to a host cell. Also provided herein are cells produced by such methods, and organisms (such as animals, plants, or fungi) comprising or produced from such cells. Methods of delivery of nucleic acids can include lipofection, nucleofection, microinjection, biolistics, liposomes, immunoliposomes, polycation or lipid:nucleic acid conjugates, naked DNA, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Delivery can be to cells (e.g., in vitro or ex vivo administration) or target tissues (e.g., in vivo administration).

Various techniques and methods are known in the art for delivering nucleic acids to cells. For example, a closed-ended DNA vector, including a ceDNA vector, produced using the synthetic process as described herein can be formulated into lipid nanoparticles (LNPs), lipidoids, liposomes, lipid nanoparticles, lipoplexes, or core-shell nanoparticles. Typically, LNPs are composed of nucleic acid (e.g., ceDNA) molecules, one or more ionizable or cationic lipids (or salts thereof), one or more non-ionic or neutral lipids (e.g., a phospholipid), a molecule that prevents aggregation (e.g., PEG or a PEG-lipid conjugate), and optionally a sterol (e.g., cholesterol).

Another method for delivering a closed-ended DNA vector, including a ceDNA vector, produced using the synthetic process as described herein to a cell is by conjugating the nucleic acid with a ligand that is internalized by the cell. For example, the ligand can bind a receptor on the cell surface and internalized via endocytosis. The ligand can be covalently linked to a nucleotide in the nucleic acid. Exemplary conjugates for delivering nucleic acids into a cell are described, example, in International Patent Application Publication Nos. WO2015/006740, WO2014/025805, WO2012/037254, WO2009/082606, WO2009/073809, WO2009/018332, WO2006/112872, WO2004/090108, WO2004/091515 and WO2017/177326.

Nucleic acids and closed-ended DNA vector, including a ceDNA vector, produced using the synthetic process as described herein can also be delivered to a cell by transfection. Useful transfection methods include, but are not limited to, lipid-mediated transfection, cationic polymer-mediated transfection, or calcium phosphate precipitation. Transfection reagents are well known in the art and include, but are not limited to, TurboFect Transfection Reagent (Thermo Fisher Scientific), Pro-Ject Reagent (Thermo Fisher Scientific), TRANSPASS™ P Protein Transfection Reagent (New England Biolabs), CHARIOT™ Protein Delivery Reagent (Active Motif), PROTEOJUICE™ Protein Transfection Reagent (EMD Millipore), 293fectin, LIPOFECTAMINE™ 2000, LIPOFECTAMINE™ 3000 (Thermo Fisher Scientific), LIPOFECTAMINE™ (Thermo Fisher Scientific), LIPOFECTIN™ (Thermo Fisher Scientific), DMRIE-C, CELLFECTIN™ (Thermo Fisher Scientific), OLIGOFECTAMINE™ (Thermo Fisher Scientific), LIPOFECTACE™, FUGENE™ (Roche, Basel, Switzerland), FUGENE™ HD (Roche), TRANSFECTAM™ (Transfectam, Promega, Madison, Wis.), TFX-10™ (Promega), TFX-20™ (Promega), TFX-50™ (Promega), TRANSFECTIN™ (BioRad, Hercules, Calif), SILENTFECT™ (Bio-Rad), Effectene™ (Qiagen, Valencia, Calif.), DC-chol (Avanti Polar Lipids), GENEPORTER™ (Gene Therapy Systems, San Diego, Calif.), DHARMAFECT 1™ (Dharmacon, Lafayette, Colo.), DHARMAFECT 2™ (Dharmacon), DHARMAFECT 3™ (Dharmacon), DHARMAFECT 4™ (Dharmacon), ESCORT™ III (Sigma, St. Louis, Mo.), and ESCORT™ IV (Sigma Chemical Co.). Nucleic acids, such as ceDNA, can also be delivered to a cell via microfluidics methods known to those of skill in the art.

A closed-ended DNA vector, including a ceDNA vector, produced using the synthetic process as described herein can also be administered directly to an organism for transduction of cells in vivo. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Methods for introduction of a closed-ended DNA vector, including a ceDNA vector, produced using the synthetic process as described herein can be delivered into hematopoietic stem cells, for example, by the methods as described, for example, in U.S. Pat. No. 5,928,638.

A closed-ended DNA vector, including a ceDNA vector, produced using the synthetic process as described herein can be added to liposomes for delivery to a cell or target organ in a subject. Liposomes are vesicles that possess at least one lipid bilayer. Liposomes are typical used as carriers for drug/therapeutic delivery in the context of pharmaceutical development. They work by fusing with a cellular membrane and repositioning its lipid structure to deliver a drug or active pharmaceutical ingredient (API). Liposome compositions for such delivery are composed of phospholipids, especially compounds having a phosphatidylcholine group, however these compositions may also include other lipids. Exemplary liposomes and liposome formulations are disclosed in International Patent Application Publication Nos. WO2019/051289 and WO2019/113310, e.g., see the section entitled “Pharmaceutical Formulations”.

Various delivery methods known in the art or modifications thereof can be used to deliver a closed-ended DNA vector, including a ceDNA vector, produced using the synthetic process as described herein in vitro or in vivo. For example, in some embodiments, ceDNA vectors are delivered by making transient penetration in cell membrane by mechanical, electrical, ultrasonic, hydrodynamic, or laser-based energy so that DNA entrance into the targeted cells is facilitated. For example, a ceDNA vector can be delivered by transiently disrupting cell membrane by squeezing the cell through a size-restricted channel or by other means known in the art. In some cases, a ceDNA vector alone is directly injected as naked DNA into skin, thymus, cardiac muscle, skeletal muscle, or liver cells. In some cases, a ceDNA vector is delivered by gene gun. Gold or tungsten spherical particles (1-3 μm diameter) coated with capsid-free AAV vectors can be accelerated to high speed by pressurized gas to penetrate into target tissue cells.

Compositions comprising a closed-ended DNA vector, including a ceDNA vector, produced using the synthetic process as described herein and a pharmaceutically acceptable carrier are specifically contemplated herein. In some embodiments, the ceDNA vector is formulated with a lipid delivery system, for example, liposomes as described herein. In some embodiments, such compositions are administered by any route desired by a skilled practitioner. The compositions may be administered to a subject by different routes including orally, parenterally, sublingually, transdermally, rectally, transmucosally, topically, via inhalation, via buccal administration, intrapleurally, intravenous, intra-arterial, intraperitoneal, subcutaneous, intramuscular, intranasal intrathecal, and intraarticular or combinations thereof. For veterinary use, the composition may be administered as a suitably acceptable formulation in accordance with normal veterinary practice. The veterinarian may readily determine the dosing regimen and route of administration that is most appropriate for a particular animal. The compositions may be administered by traditional syringes, needleless injection devices, “microprojectile bombardment gene guns”, or other physical methods such as electroporation (“EP”), hydrodynamic methods or ultrasound.

In some cases, a closed-ended DNA vector, including a ceDNA vector, produced using the synthetic process as described herein is delivered by hydrodynamic injection, which is a simple and highly efficient method for direct intracellular delivery of any water-soluble compounds and particles into internal organs and skeletal muscle in an entire limb.

In some cases, a closed-ended DNA vector, including a ceDNA vector, produced using the synthetic process as described herein is delivered by ultrasound by making nanoscopic pores in membrane to facilitate intracellular delivery of DNA particles into cells of internal organs or tumors, so the size and concentration of the closed-ended DNA vector have a great role in efficiency of the system. In some cases, closed-ended DNA vectors, including a ceDNA vector, produced using the synthetic process as described herein are delivered by magnetofection by using magnetic fields to concentrate particles containing nucleic acid into the target cells.

In some cases, chemical delivery systems can be used, for example, by using nanomeric complexes, which include compaction of negatively charged nucleic acid by polycationic nanomeric particles, belonging to cationic liposome/micelle or cationic polymers. Cationic lipids used for the delivery method includes, but not limited to monovalent cationic lipids, polyvalent cationic lipids, guanidine containing compounds, cholesterol derivative compounds, cationic polymers, (e.g., poly(ethylenimine), poly-L-lysine, protamine, other cationic polymers), and lipid-polymer hybrid.

### Exosomes

In some embodiments, a closed-ended DNA vector, including a ceDNA vector, produced using the synthetic process as described herein is delivered by being packaged in an exosome. Exosomes are small membrane vesicles of endocytic origin that are released into the extracellular environment following fusion of multivesicular bodies with the plasma membrane. Their surface consists of a lipid bilayer from the donor cell's cell membrane, they contain cytosol from the cell that produced the exosome, and exhibit membrane proteins from the parental cell on the surface. Exosomes are produced by various cell types including epithelial cells, B and T lymphocytes, mast cells (MC) as well as dendritic cells (DC). Some embodiments, exosomes with a diameter between 10 nm and 1 μm, between 20 nm and 500 nm, between 30 nm and 250 nm, between 50 nm and 100 nm are envisioned for use. Exosomes can be isolated for a delivery to target cells using either their donor cells or by introducing specific nucleic acids into them. Various approaches known in the art can be used to produce exosomes containing capsid-free AAV vectors of the present disclosure.

### Microparticle Nanoparticles

In some embodiments, a closed-ended DNA vector, including a ceDNA vector, produced using the synthetic process as described herein is delivered by a lipid nanoparticle. Generally, lipid nanoparticles comprise an ionizable amino lipid (e.g., heptatriaconta-6,9,28,31-tetraen-19-yl 4-(dimethylamino)butanoate, Dlin-MC3-DMA, a phosphatidylcholine (1,2-distearoyl-sn-glycero-3-phosphocholine, DSPC), cholesterol and a coat lipid (polyethylene glycol-dimyristolglycerol, PEG-DMG), for example as disclosed by Tam et al., (2013). Advances in Lipid Nanoparticles for siRNA delivery. Pharmaceuticals 5(3): 498-507.

In some embodiments, a lipid nanoparticle has a mean diameter between about 10 and about 1000 nm. In some embodiments, a lipid nanoparticle has a diameter that is less than 300 nm. In some embodiments, a lipid nanoparticle has a diameter between about 10 and about 300 nm. In some embodiments, a lipid nanoparticle has a diameter that is less than 200 nm. In some embodiments, a lipid nanoparticle has a diameter between about 25 and about 200 nm. In some embodiments, a lipid nanoparticle preparation (e.g., composition comprising a plurality of lipid nanoparticles) has a size distribution in which the mean size (e.g., diameter) is about 70 nm to about 200 nm, and more typically the mean size is about 100 nm or less.

Various lipid nanoparticles known in the art can be used to deliver a closed-ended DNA vector, including a ceDNA vector produced using the synthetic process as described herein. For example, various delivery methods using lipid nanoparticles are described in U.S. Pat. Nos. 9,404,127, 9,006,417 and 9,518,272.

In some embodiments, a closed-ended DNA vector, including a ceDNA vector, produced using the synthetic process as described herein is delivered by a gold nanoparticle. Generally, a nucleic acid can be covalently bound to a gold nanoparticle or non-covalently bound to a gold nanoparticle (e.g., bound by a charge-charge interaction), for example as described by Ding et al., (2014). Gold Nanoparticles for Nucleic Acid Delivery. Mol. Ther. 22(6); 1075-1083. In some embodiments, gold nanoparticle-nucleic acid conjugates are produced using methods described, for example, in U.S. Pat. No. 6,812,334.

### Conjugates

In some embodiments, a closed-ended DNA vector, including a ceDNA vector, produced using the synthetic process as described herein as disclosed herein is conjugated (e.g., covalently bound to an agent that increases cellular uptake. An “agent that increases cellular uptake” is a molecule that facilitates transport of a nucleic acid across a lipid membrane. For example, a nucleic acid can be conjugated to a lipophilic compound (e.g., cholesterol, tocopherol, etc.), a cell penetrating peptide (CPP) (e.g., penetratin, TAT, Syn1B, etc.), and polyamines (e.g., spermine). Further examples of agents that increase cellular uptake are disclosed, for example, in Winkler (2013). Oligonucleotide conjugates for therapeutic applications. Ther. Deliv. 4(7); 791-809.

In some embodiments, a closed-ended DNA vector, including a ceDNA vector, produced using the synthetic process as described herein as disclosed herein is conjugated to a polymer (e.g., a polymeric molecule) or a folate molecule (e.g., folic acid molecule). Generally, delivery of nucleic acids conjugated to polymers is known in the art, for example as described in WO2000/34343 and WO2008/022309. In some embodiments, a ceDNA vector as disclosed herein is conjugated to a poly(amide) polymer, for example as described by U.S. Pat. No. 8,987,377. In some embodiments, a nucleic acid described by the disclosure is conjugated to a folic acid molecule as described in U.S. Pat. No. 8,507,455.

In some embodiments, a closed-ended DNA vector, including a ceDNA vector, produced using the synthetic process as described herein as disclosed herein is conjugated to a carbohydrate, for example as described in U.S. Pat. No. 8,450,467.

### Nanocapsules

Alternatively, nanocapsule formulations of a closed-ended DNA vector, including a ceDNA vector, produced using the synthetic process as described herein as disclosed herein can be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

### Liposomes

A closed-ended DNA vector, including a ceDNA vector, produced using the synthetic process as described herein can be added to liposomes for delivery to a cell or target organ in a subject. Liposomes are vesicles that possess at least one lipid bilayer. Liposomes are typical used as carriers for drug/therapeutic delivery in the context of pharmaceutical development. They work by fusing with a cellular membrane and repositioning its lipid structure to deliver a drug or active pharmaceutical ingredient (API). Liposome compositions for such delivery are composed of phospholipids, especially compounds having a phosphatidylcholine group, however these compositions may also include other lipids.

The formation and use of liposomes is generally known to those of skill in the art. Liposomes have been developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

### Exemplary Liposome and Lipid Nanoparticle (LNP) Compositions

A closed-ended DNA vector, including a ceDNA vector, produced using the synthetic process as described herein can be added to liposomes for delivery to a cell, e.g., a cell in need of expression of the transgene. Liposomes are vesicles that possess at least one lipid bilayer. Liposomes are typical used as carriers for drug/therapeutic delivery in the context of pharmaceutical development. They work by fusing with a cellular membrane and repositioning its lipid structure to deliver a drug or active pharmaceutical ingredient (API). Liposome compositions for such delivery are composed of phospholipids, especially compounds having a phosphatidylcholine group, however these compositions may also include other lipids.

Lipid nanoparticles (LNPs) comprising ceDNA are disclosed in International Patent Application Publication Nos. WO2019/051289 and WO2019/113310, as well as International Patent Application No. PCT/US2021/04043, filed Jul. 16, 2021 and envisioned for use in the methods and compositions as disclosed herein.

In some aspects, the disclosure provides for a liposome formulation that includes one or more compounds with a polyethylene glycol (PEG) functional group (so-called “PEG-ylated compounds”) which can reduce the immunogenicity/antigenicity of, provide hydrophilicity and hydrophobicity to the compound(s) and reduce dosage frequency. Or the liposome formulation simply includes polyethylene glycol (PEG) polymer as an additional component. In such aspects, the molecular weight of the PEG or PEG functional group can be from 62 Da to about 5,000 Da.

In some aspects, the disclosure provides for a liposome formulation that will deliver an API with extended release or controlled release profile over a period of hours to weeks. In some related aspects, the liposome formulation may comprise aqueous chambers that are bound by lipid bilayers. In other related aspects, the liposome formulation encapsulates an API with components that undergo a physical transition at elevated temperature which releases the API over a period of hours to weeks.

In some aspects, the liposome formulation comprises sphingomyelin and one or more lipids disclosed herein. In some aspects, the liposome formulation comprises optisomes.

In some aspects, the disclosure provides for a liposome formulation that includes one or more lipids selected from: N-(carbonyl-methoxypolyethylene glycol 2000)-1,2-distearoyl-sn-glycero-3-phosphoethanolamine sodium salt, (distearoyl-sn-glycero-phosphoethanolamine), MPEG (methoxy polyethylene glycol)-conjugated lipid, HSPC (hydrogenated soy phosphatidylcholine); PEG (polyethylene glycol); DSPE (distearoyl-sn-glycero-phosphoethanolamine); DSPC (distearoylphosphatidylcholine); DOPC (dioleoylphosphatidylcholine); DPPG (dipalmitoylphosphatidylglycerol); EPC (egg phosphatidylcholine); DOPS (dioleoylphosphatidylserine); POPC (palmitoyloleoylphosphatidylcholine); SM (sphingomyelin); MPEG (methoxy polyethylene glycol); DMPC (dimyristoyl phosphatidylcholine); DMPG (dimyristoyl phosphatidylglycerol); DSPG (distearoylphosphatidylglycerol); DEPC (dierucoylphosphatidylcholine); DOPE (dioleoly-sn-glycero-phophoethanolamine). cholesteryl sulphate (CS), dipalmitoylphosphatidylglycerol (DPPG), DOPC (dioleoly-sn-glycero-phosphatidylcholine) or any combination thereof.

In some aspects, the disclosure provides for a liposome formulation comprising phospholipid, cholesterol and a PEG-ylated lipid in a molar ratio of 56:38:5. In some aspects, the liposome formulation's overall lipid content is from 2-16 mg/mL. In some aspects, the disclosure provides for a liposome formulation comprising a lipid containing a phosphatidylcholine functional group, a lipid containing an ethanolamine functional group and a PEG-ylated lipid. In some aspects, the disclosure provides for a liposome formulation comprising a lipid containing a phosphatidylcholine functional group, a lipid containing an ethanolamine functional group and a PEG-ylated lipid in a molar ratio of 3:0.015:2 respectively. In some aspects, the disclosure provides for a liposome formulation comprising a lipid containing a phosphatidylcholine functional group, cholesterol and a PEG-ylated lipid. In some aspects, the disclosure provides for a liposome formulation comprising a lipid containing a phosphatidylcholine functional group and cholesterol. In some aspects, the PEG-ylated lipid is PEG-2000-DSPE. In some aspects, the disclosure provides for a liposome formulation comprising DPPG, soy PC, MPEG-DSPE lipid conjugate and cholesterol.

In some aspects, the disclosure provides for a liposome formulation comprising one or more lipids containing a phosphatidylcholine functional group and one or more lipids containing an ethanolamine functional group. In some aspects, the disclosure provides for a liposome formulation comprising one or more: lipids containing a phosphatidylcholine functional group, lipids containing an ethanolamine functional group, and sterols, e.g. cholesterol. In some aspects, the liposome formulation comprises DOPC/DEPC; and DOPE.

In some aspects, the disclosure provides for a liposome formulation further comprising one or more pharmaceutical excipients, e.g. sucrose and/or glycine.

In some aspects, the disclosure provides for a liposome formulation that is either unilamellar or multilamellar in structure. In some aspects, the disclosure provides for a liposome formulation that comprises multi-vesicular particles and/or foam-based particles. In some aspects, the disclosure provides for a liposome formulation that are larger in relative size to common nanoparticles and about 150 to 250 nm in size. In some aspects, the liposome formulation is a lyophilized powder.

In some aspects, the disclosure provides for a liposome formulation that is made and loaded with ceDNA vectors disclosed or described herein, by adding a weak base to a mixture having the isolated ceDNA outside the liposome. This addition increases the pH outside the liposomes to approximately 7.3 and drives the API into the liposome. In some aspects, the disclosure provides for a liposome formulation having a pH that is acidic on the inside of the liposome. In such cases the inside of the liposome can be at pH 4-6.9, and more preferably pH 6.5. In other aspects, the disclosure provides for a liposome formulation made by using intra-liposomal drug stabilization technology. In such cases, polymeric or non-polymeric highly charged anions and intra-liposomal trapping agents are utilized, e.g. polyphosphate or sucrose octasulfate.

In some aspects, the disclosure provides for a lipid nanoparticle comprising a DNA vector, including a ceDNA vector produced using the synthetic process as described herein and an ionizable lipid. For example, a lipid nanoparticle formulation that is made and loaded with ceDNA obtained by the process as disclosed in International Patent Application Publication No. WO2019/051289. This can be accomplished by high energy mixing of ethanolic lipids with aqueous ceDNA at low pH which protonates the ionizable lipid and provides favorable energetics for ceDNA/lipid association and nucleation of particles. The particles can be further stabilized through aqueous dilution and removal of the organic solvent. The particles can be concentrated to the desired level.

Generally, the lipid particles are prepared at a total lipid to ceDNA (mass or weight) ratio of from about 10:1 to 30:1. In some embodiments, the lipid to ceDNA ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of lipids and ceDNA can be adjusted to provide a desired N/P ratio, for example, N/P ratio of 3, 4, 5, 6, 7, 8, 9, 10 or higher. Generally, the lipid particle formulation's overall lipid content can range from about 5 mg/ml to about 30 mg/mL.

The ionizable lipid is typically employed to condense the nucleic acid cargo, e.g., ceDNA at low pH and to drive membrane association and fusogenicity. Generally, ionizable lipids are lipids comprising at least one amino group that is positively charged or becomes protonated under acidic conditions, for example at pH of 6.5 or lower. Ionizable lipids are also referred to as cationic lipids herein.

Exemplary ionizable lipids are described in International Patent Application Publication Nos. WO2015/095340, WO2015/199952, WO2018/011633, WO2017/049245, WO2015/061467, WO2012/040184, WO2012/000104, WO2015/074085, WO2016/081029, WO2017/004143, WO2017/075531, WO2017/117528, WO2011/022460, WO2013/148541, WO2013/116126, WO2011/153120, WO2012/044638, WO2012/054365, WO2011/090965, WO2013/016058, WO2012/162210, WO2008/042973, WO2010/129709, WO2010/144740, WO2012/099755, WO2013/049328, WO2013/086322, WO2013/086373, WO2011/071860, WO2009/132131, WO2010/048536, WO2010/088537, WO2010/054401, WO2010/054406, WO2010/054405, WO2010/054384, WO2012/016184, WO2009/086558, WO2010/042877, WO2011/000106, WO2011/000107, WO2005/120152, WO2011/141705, WO2013/126803, WO2006/007712, WO2011/038160, WO2005/121348, WO2011/066651, WO2009/127060, WO2011/141704, WO2006/069782, WO2012/031043, WO2013/006825, WO2013/033563, WO2013/089151, WO2017/099823, WO2015/095346, and WO2013/086354, and U.S. Patent Application Publication Nos. US2016/0311759, US2015/0376115, US2016/0151284, US2017/0210697, US2015/0140070, US2013/0178541, US2013/0303587, US2015/0141678, US2015/0239926, US2016/0376224, US2017/0119904, US2012/0149894, US2015/0057373, US2013/0090372, US2013/0274523, US2013/0274504, US2013/0274504, US2009/0023673, US2012/0128760, US2010/0324120, US2014/0200257, US2015/0203446, US2018/0005363, US2014/0308304, US2013/0338210, US2012/0101148, US2012/0027796, US2012/0058144, US2013/0323269, US2011/0117125, US2011/0256175, US2012/0202871, US2011/0076335, US2006/0083780, US2013/0123338, US2015/0064242, US2006/0051405, US2013/0065939, US2006/0008910, US2003/0022649, US2010/0130588, US2013/0116307, US2010/0062967, US2013/0202684, US2014/0141070, US2014/0255472, US2014/0039032, US2018/0028664, US2016/0317458, and US2013/0195920.

In some embodiments, the ionizable lipid is MC3 (6Z,9Z,28Z,31Z)-heptatriaconta-6,9,28,31-tetraen-19-yl-4-(dimethylamino) butanoate (DLin-MC3-DMA or MC3) having the following structure:

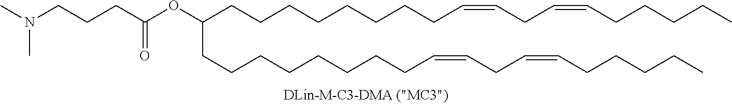 [SMILES: CCCCC/C=C\C/C=C\CCCCCCCCC(CCCCCCCC/C=C\C/C=C\CCCCC)OC(=O)CCCN(C)C]

The lipid DLin-MC3-DMA is described in Jayaraman et al., Angew. Chem. Int. Ed Engl. (2012), 51(34): 8529-8533.

In some embodiments, the ionizable lipid is the lipid ATX-002 as described in International Patent Application Publication No. WO2015/074085.

In some embodiments, the ionizable lipid is (13Z,16Z)-N,N-dimethyl-3-nonyldocosa-13,16-dien-1-amine (Compound 32), as described in International Patent Application Publication No. WO2012/040184.

In some embodiments, the ionizable lipid is Compound 6 or Compound 22 as described in WO2015/199952.

Without limitations, ionizable lipid can comprise 20-90% (mol) of the total lipid present in the lipid nanoparticle. For example, ionizable lipid molar content can be 20-70% (mol), 30-60% (mol) or 40-50% (mol) of the total lipid present in the lipid nanoparticle. In some embodiments, ionizable lipid comprises from about 50 mol % to about 90 mol % of the total lipid present in the lipid nanoparticle.

In some aspects, the lipid nanoparticle can further comprise a non-cationic lipid. Non-ionic lipids include amphipathic lipids, neutral lipids and anionic lipids. Accordingly, the non-cationic lipid can be a neutral uncharged, zwitterionic, or anionic lipid. Non-cationic lipids are typically employed to enhance fusogenicity.

Exemplary non-cationic lipids envisioned for use in the methods and compositions comprising a DNA vector, including a ceDNA vector produced using the synthetic process as described herein are described in International Patent Application Publication Nos. WO2019/051289 and WO2019/113310.

Exemplary non-cationic lipids are described in International Patent Application Publication No. WO2017/099823 and U.S. Patent Application Publication No. US2018/0028664.

The non-cationic lipid can comprise 0-30% (mol) of the total lipid present in the lipid nanoparticle. For example, the non-cationic lipid content is 5-20% (mol) or 10-15% (mol) of the total lipid present in the lipid nanoparticle. In various embodiments, the molar ratio of ionizable lipid to the neutral lipid ranges from about 2:1 to about 8:1.

In some embodiments, the lipid nanoparticles do not comprise any phospholipids. In some aspects, the lipid nanoparticle can further comprise a component, such as a sterol, to provide membrane integrity.

One exemplary sterol that can be used in the lipid nanoparticle is cholesterol and derivatives thereof. Exemplary cholesterol derivatives are described in International Patent Application Publication No. WO2009/127060 and U.S. Patent Application Publication No. US2010/0130588.

The component providing membrane integrity, such as a sterol, can comprise 0-50% (mol) of the total lipid present in the lipid nanoparticle. In some embodiments, such a component is 20-50% (mol) 30-40% (mol) of the total lipid content of the lipid nanoparticle.

In some aspects, the lipid nanoparticle can further comprise a polyethylene glycol (PEG) or a conjugated lipid molecule. Generally, these are used to inhibit aggregation of lipid nanoparticles and/or provide steric stabilization. Exemplary conjugated lipids include, but are not limited to, PEG-lipid conjugates, polyoxazoline (POZ)-lipid conjugates, polyamide-lipid conjugates (such as ATTA-lipid conjugates), cationic-polymer lipid (CPL) conjugates, and mixtures thereof. In some embodiments, the conjugated lipid molecule is a PEG-lipid conjugate, for example, a (methoxy polyethylene glycol)-conjugated lipid. Exemplary PEG-lipid conjugates include, but are not limited to, PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a PEGylated phosphatidylethanoloamine (PEG-PE), PEG succinate diacylglycerol (PEGS-DAG) (such as 4-O-(2′,3′-di(tetradecanoyloxy)propyl-1-O-(w-methoxy(polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, or a mixture thereof. Additional exemplary PEG-lipid conjugates are described, for example, in U.S. Pat. Nos. 5,885,613, 6,287,591 and U.S. Patent Application Publication Nos. US2003/0077829, US2003/0077829, US2005/0175682, US2008/0020058, US2011/0117125, US2010/0130588, US2016/0376224, and US2017/0119904.

In some embodiments, a PEG-lipid is a compound disclosed in U.S. Patent Application Publication No. US2018/0028664.

In some embodiments, a PEG-lipid is disclosed in U.S. Patent Application Publication Nos. US20150376115 or US2016/0376224.

The PEG-DAA conjugate can be, for example, PEG-dilauryloxypropyl, PEG-dimyristyloxypropyl, PEG-dipalmityloxypropyl, or PEG-distearyloxypropyl. The PEG-lipid can be one or more of PEG-DMG, PEG-dilaurylglycerol, PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol (1-[8′-(Cholest-5-en-3[beta]-oxy)carboxamido-3′,6′-dioxaoctanyl]carbamoyl-[omega]-methyl-poly(ethylene glycol), PEG-DMB (3,4-Ditetradecoxylbenzyl-[omega]-methyl-poly(ethylene glycol) ether), and 1,2-dimyristoyl-sn-glycero-3-phosphoethanolamine-N-[methoxy(polyethylene glycol)-2000]. In some examples, the PEG-lipid can be selected from the group consisting of PEG-DMG, 1,2-dimyristoyl-sn-glycero-3-phosphoethanolamine-N-[methoxy(polyethylene glycol)-2000].

Lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (CPL) conjugates can be used in place of or in addition to the PEG-lipid. Exemplary conjugated lipids, i.e., PEG-lipids, (POZ)-lipid conjugates, ATTA-lipid conjugates and cationic polymer-lipids are described in the International Patent Application Publication Noss WO1996/010392, WO1998/051278, WO2002/087541, WO2005/026372, WO2008/147438, WO2009/086558, WO2012/000104, WO2017/117528, WO2017/099823, WO2015/199952, WO2017/004143, WO2015/095346, WO2012/000104, WO2012/000104, and WO2010/006282, U.S. Patent Application Publication Nos. US2003/0077829, US2005/0175682, US2008/0020058, US2011/0117125, US2013/0303587, US2018/0028664, US2015/0376115, US2016/0376224, US2016/0317458, US2013/0303587, US2013/0303587, and US20110123453, and U.S. Pat. Nos. 5,885,613, 6,287,591, 6,320,017, and 6,586,559.

In some embodiments, the one or more additional compound can be a therapeutic agent. The therapeutic agent can be selected from any class suitable for the therapeutic objective. In other words, the therapeutic agent can be selected from any class suitable for the therapeutic objective. In other words, the therapeutic agent can be selected according to the treatment objective and biological action desired. For example, if the ceDNA within the LNP is useful for treating cancer, the additional compound can be an anti-cancer agent (e.g., a chemotherapeutic agent, a targeted cancer therapy (including, but not limited to, a small molecule, an antibody, or an antibody-drug conjugate). In another example, if the LNP containing the ceDNA is useful for treating an infection, the additional compound can be an antimicrobial agent (e.g., an antibiotic or antiviral compound). In yet another example, if the LNP containing the ceDNA is useful for treating an immune disease or disorder, the additional compound can be a compound that modulates an immune response (e.g., an immunosuppressant, immunostimulatory compound, or compound modulating one or more specific immune pathways). In some embodiments, different cocktails of different lipid nanoparticles containing different compounds, such as a ceDNA encoding a different protein or a different compound, such as a therapeutic may be used in the compositions and methods of the disclosure.

In some embodiments, the additional compound is an immune modulating agent. For example, the additional compound is an immunosuppressant. In some embodiments, the additional compound is immune stimulatory agent.

Also provided herein is a pharmaceutical composition comprising the lipid nanoparticle-encapsulated synthetically produced ceDNA vector and a pharmaceutically acceptable carrier or excipient.

In some aspects, the disclosure provides for a lipid nanoparticle formulation further comprising one or more pharmaceutical excipients. In some embodiments, the lipid nanoparticle formulation further comprises sucrose, tris, trehalose and/or glycine.

A closed-ended DNA vector, including a ceDNA vector, produced using the synthetic process as described herein can be complexed with the lipid portion of the particle or encapsulated in the lipid position of the lipid nanoparticle. In some embodiments, a DNA vector, including a ceDNA vector produced using the synthetic process as described herein can be fully encapsulated in the lipid position of the lipid nanoparticle, thereby protecting it from degradation by a nuclease, e.g., in an aqueous solution. In some embodiments, a DNA vector, including a ceDNA vector produced using the synthetic process as described herein in the lipid nanoparticle is not substantially degraded after exposure of the lipid nanoparticle to a nuclease at 37° C. for at least about 20, 30, 45, or 60 minutes. In some embodiments, the ceDNA in the lipid nanoparticle is not substantially degraded after incubation of the particle in serum at 37° C. for at least about 30, 45, or 60 minutes or at least about 2, 3, 4, 5, 6, 7, 8, 9, 10, 12, 14, 16, 18, 20, 22, 24, 26, 28, 30, 32, 34, or 36 hours.

In certain embodiments, the lipid nanoparticles are substantially non-toxic to a subject, e.g., to a mammal such as a human. In some aspects, the lipid nanoparticle formulation is a lyophilized powder.

In some embodiments, lipid nanoparticles are solid core particles that possess at least one lipid bilayer. In other embodiments, the lipid nanoparticles have a non-bilayer structure, i.e., a non-lamellar (i.e., non-bilayer) morphology. Without limitations, the non-bilayer morphology can include, for example, three dimensional tubes, rods, cubic symmetries, etc. For example, the morphology of the lipid nanoparticles (lamellar vs. non-lamellar) can readily be assessed and characterized using, e.g., Cryo-TEM analysis as described in U.S. Patent Application Publication No. US2010/0130588.

In some further embodiments, the lipid nanoparticles having a non-lamellar morphology are electron dense. In some aspects, the disclosure provides for a lipid nanoparticle that is either unilamellar or multilamellar in structure. In some aspects, the disclosure provides for a lipid nanoparticle formulation that comprises multi-vesicular particles and/or foam-based particles.

By controlling the composition and concentration of the lipid components, one can control the rate at which the lipid conjugate exchanges out of the lipid particle and, in turn, the rate at which the lipid nanoparticle becomes fusogenic. In addition, other variables including, e.g., pH, temperature, or ionic strength, can be used to vary and/or control the rate at which the lipid nanoparticle becomes fusogenic. Other methods which can be used to control the rate at which the lipid nanoparticle becomes fusogenic will be apparent to those of ordinary skill in the art based on this disclosure. It will also be apparent that by controlling the composition and concentration of the lipid conjugate, one can control the lipid particle size.

The pKa of formulated cationic lipids can be correlated with the effectiveness of the LNPs for delivery of nucleic acids (see Jayaraman et al., Angewandte Chemie, International Edition (2012), 51(34), 8529-8533; Semple et al., Nature Biotechnology 28, 172-176 (2010). The preferred range of pKa is—5 to ˜7. The pKa of the cationic lipid can be determined in lipid nanoparticles using an assay based on fluorescence of 2-(p-toluidino)-6-napthalene sulfonic acid (TNS).

### VIII. Methods of Delivering Closed-Ended DNA Vectors

In some embodiments, a closed-ended DNA vector, including a ceDNA vector, produced using the synthetic process as described herein can be delivered to a target cell in vitro or in vivo by various suitable methods. A closed-ended DNA vector, including a ceDNA vector, produced using the synthetic process as described herein alone can be applied or injected. A closed-ended DNA vector, including a ceDNA vector, produced using the synthetic process as described herein can be delivered to a cell without the help of a transfection reagent or other physical means. Alternatively, a closed-ended DNA vector, including a ceDNA vector, produced using the synthetic process as described herein can be delivered using any art-known transfection reagent or other art-known physical means that facilitates entry of DNA into a cell, e.g., liposomes, alcohols, polylysine-rich compounds, arginine-rich compounds, calcium phosphate, microvesicles, microinjection, electroporation and the like.

In another embodiment, a closed-ended DNA vector, including a ceDNA vector, produced using the synthetic process as described herein is administered to the CNS (e.g., to the brain or to the eye). The, e.g., ceDNA vector may be introduced into the spinal cord, brainstem (medulla oblongata, pons), midbrain (hypothalamus, thalamus, epithalamus, pituitary gland, substantia nigra, pineal gland), cerebellum, telencephalon (corpus striatum, cerebrum including the occipital, temporal, parietal and frontal lobes, cortex, basal ganglia, hippocampus and portaamygdala), limbic system, neocortex, corpus striatum, cerebrum, and inferior colliculus. The ceDNA vector may also be administered to different regions of the eye such as the retina, cornea and/or optic nerve. The ceDNA vector may be delivered into the cerebrospinal fluid (e.g., by lumbar puncture). The ceDNA vector may further be administered intravascularly to the CNS in situations in which the blood-brain barrier has been perturbed (e.g., brain tumor or cerebral infarct).

In some embodiments, a closed-ended DNA vector, including a ceDNA vector, produced using the synthetic process as described herein can be administered to the desired region(s) of the CNS by any route known in the art, including but not limited to, intrathecal, intra-ocular, intracerebral, intraventricular, intravenous (e.g., in the presence of a sugar such as mannitol), intranasal, intra-aural, intra-ocular (e.g., intra-vitreous, sub-retinal, anterior chamber) and peri-ocular (e.g., sub-Tenon's region) delivery as well as intramuscular delivery with retrograde delivery to motor neurons.

In some embodiments, a closed-ended DNA vector, including a ceDNA vector, produced using the synthetic process as described herein is administered in a liquid formulation by direct injection (e.g., stereotactic injection) to the desired region or compartment in the CNS. In other embodiments, the e.g., synthetically produced ceDNA vector can be provided by topical application to the desired region or by intra-nasal administration of an aerosol formulation. Administration to the eye may be by topical application of liquid droplets. As a further alternative, the e.g., ceDNA vector can be administered as a solid, slow-release formulation (see, e.g., U.S. Pat. No. 7,201,898). In yet additional embodiments, the e.g., synthetically produced ceDNA vector can be used for retrograde transport to treat, ameliorate, and/or prevent diseases and disorders involving motor neurons (e.g., amyotrophic lateral sclerosis (ALS); spinal muscular atrophy (SMA), etc.). For example, the e.g., synthetically produced ceDNA vector can be delivered to muscle tissue from which it can migrate into neurons.

### IX. Additional Uses of the ceDNA Vectors

The compositions and closed-ended DNA vector, including ceDNA vectors, produced using the synthetic process as described herein can be used to express a target gene or transgene for various purposes. In some embodiments, the resulting transgene encodes a protein or functional RNA that is intended to be used for research purposes, e.g., to create a somatic transgenic animal model harboring the transgene, e.g., to study the function of the transgene product. In another example, the transgene encodes a protein or functional RNA that is intended to be used to create an animal model of disease. In some embodiments, the resulting transgene encodes one or more peptides, polypeptides, or proteins, which are useful for the treatment, prevention, or amelioration of disease states or disorders in a mammalian subject. The resulting transgene can be transferred (e.g., expressed in) to a subject in a sufficient amount to treat a disease associated with reduced expression, lack of expression or dysfunction of the gene. In some embodiments the resulting transgene can be expressed in a subject in a sufficient amount to treat a disease associated with increased expression, activity of the gene product, or inappropriate upregulation of a gene that the resulting transgene suppresses or otherwise causes the expression of which to be reduced. In yet other embodiments, the resulting transgene replaces or supplements a defective copy of the native gene. It will be appreciated by one of ordinary skill in the art that the transgene may not be an open reading frame of a gene to be transcribed itself; instead it may be a promoter region or repressor region of a target gene, and the ceDNA vector may modify such region with the outcome of so modulating the expression of a gene of interest.

In some embodiments, the transgene encodes a protein or functional RNA that is intended to be used to create an animal model of disease. In some embodiments, the transgene encodes one or more peptides, polypeptides, or proteins, which are useful for the treatment or prevention of disease states in a mammalian subject. The transgene can be transferred (e.g., expressed in) to a patient in a sufficient amount to treat a disease associated with reduced expression, lack of expression or dysfunction of the gene.

### X. Methods of Use

A synthetically produced closed-ended DNA vector, e.g., ceDNA vector as disclosed herein can also be used in a method for the delivery of a nucleotide sequence of interest (e.g., a transgene) to a target cell (e.g., a host cell). The method may in particular be a method for delivering a transgene to a cell of a subject in need thereof and treating a disease of interest. The disclosure allows for the in vivo expression of a transgene, e.g., a protein, antibody, nucleic acid such as miRNA etc. encoded in the ceDNA vector in a cell in a subject such that therapeutic effect of the expression of the transgene occurs. These results are seen with both in vivo and in vitro modes of closed-ended DNA vector (e.g., ceDNA vector) delivery.

In addition, the disclosure provides a method for the delivery of a transgene in a cell of a subject in need thereof, comprising multiple administrations of the synthetically produced closed-ended DNA vector (e.g. ceDNA vector) of the disclosure comprising said nucleic acid or transgene of interest. Since the ceDNA vector of the disclosure does not induce an immune response like that typically observed against encapsidated viral vectors, such a multiple administration strategy will likely have greater success in a ceDNA-based system.

The synthetically produced closed-ended DNA vector (e.g., ceDNA vector) nucleic acid(s) are administered in sufficient amounts to transfect the cells of a desired tissue and to provide sufficient levels of gene transfer and expression without undue adverse effects. Conventional and pharmaceutically acceptable routes of administration include, but are not limited to, intravenous (e.g., in a liposome formulation), direct delivery to the selected organ (e.g., intraportal delivery to the liver), intramuscular, and other parental routes of administration. Routes of administration may be combined, if desired.

Closed-ended DNA vector (e.g. ceDNA vector) delivery is not limited to delivery gene replacements. For example, the synthetically produced closed-ended DNA vectors (e.g., ceDNA vectors) as described herein may be used with other delivery systems provided to provide a portion of the gene therapy. One non-limiting example of a system that may be combined with the synthetically produced ceDNA vectors in accordance with the present disclosure includes systems which separately deliver one or more co-factors or immune suppressors for effective gene expression of the transgene.

The disclosure also provides for a method of treating a disease in a subject comprising introducing into a target cell in need thereof (in particular a muscle cell or tissue) of the subject a therapeutically effective amount of a synthetically produced closed-ended DNA vector (e.g., ceDNA vector), optionally with a pharmaceutically acceptable carrier. While the, e.g., synthetically produced ceDNA vector can be introduced in the presence of a carrier, such a carrier is not required. The, e.g., synthetically produced ceDNA vector selected comprises a nucleotide sequence of interest useful for treating the disease. In particular, the, e.g., synthetically produced ceDNA vector may comprise a desired exogenous DNA sequence operably linked to control elements capable of directing transcription of the desired polypeptide, protein, or oligonucleotide encoded by the exogenous DNA sequence when introduced into the subject. The e.g., synthetically produced ceDNA vector can be administered via any suitable route as provided above, and elsewhere herein.

The synthetically produced compositions and vectors provided herein can be used to deliver a transgene for various purposes. In some embodiments, the transgene encodes a protein or functional RNA that is intended to be used for research purposes, e.g., to create a somatic transgenic animal model harboring the transgene, e.g., to study the function of the transgene product. In another example, the transgene encodes a protein or functional RNA that is intended to be used to create an animal model of disease. In some embodiments, the transgene encodes one or more peptides, polypeptides, or proteins, which are useful for the treatment or prevention of disease states in a mammalian subject. The transgene can be transferred (e.g., expressed in) to a patient in a sufficient amount to treat a disease associated with reduced expression, lack of expression or dysfunction of the gene.

In principle, the expression cassette can include a nucleic acid or any transgene that encodes a protein or polypeptide that is either reduced or absent due to a mutation or which conveys a therapeutic benefit when overexpressed is considered to be within the scope of the disclosure.

A synthetically produced ceDNA vector is not limited to one species of ceDNA vector. As such, in another aspect, multiple ceDNA vectors comprising different transgenes or the same transgene but operatively linked to different promoters or cis-regulatory elements can be delivered simultaneously or sequentially to the target cell, tissue, organ, or subject. Therefore, this strategy can allow for the gene therapy or gene delivery of multiple genes simultaneously. It is also possible to separate different portions of the transgene into separate ceDNA vectors (e.g., different domains and/or co-factors required for functionality of the transgene) which can be administered simultaneously or at different times, and can be separately regulatable, thereby adding an additional level of control of expression of the transgene. Delivery can also be performed multiple times and, importantly for gene therapy in the clinical setting, in subsequent increasing or decreasing doses, given the lack of an anti-capsid host immune response due to the absence of a viral capsid. It is anticipated that no anti-capsid response will occur as there is no capsid.

The disclosure also provides for a method of treating a disease in a subject comprising introducing into a target cell in need thereof (in particular a muscle cell or tissue) of the subject a therapeutically effective amount of a synthetically produced ceDNA vector as disclosed herein, optionally with a pharmaceutically acceptable carrier. While the ceDNA vector can be introduced in the presence of a carrier, such a carrier is not required. The ceDNA vector implemented comprises a nucleotide sequence of interest useful for treating the disease. In particular, the ceDNA vector may comprise a desired exogenous DNA sequence operably linked to control elements capable of directing transcription of the desired polypeptide, protein, or oligonucleotide encoded by the exogenous DNA sequence when introduced into the subject. The synthetically produced ceDNA vector can be administered via any suitable route as provided above, and elsewhere herein.

### XI. Methods of Treatment

The technology described herein also demonstrates methods for making, as well as methods of using the disclosed synthetically produced ceDNA vectors in a variety of ways, including, for example, ex situ, in vitro and in vivo applications, methodologies, diagnostic procedures, and/or gene therapy regimens.

Provided herein is a method of treating a disease or disorder in a subject comprising introducing into a target cell in need thereof (for example, a muscle cell or tissue, or other affected cell type) of the subject a therapeutically effective amount of a synthetically produced ceDNA vector, optionally with a pharmaceutically acceptable carrier. While the ceDNA vector can be introduced in the presence of a carrier, such a carrier is not required. The synthetically produced ceDNA vector implemented comprises a nucleotide sequence of interest useful for treating the disease. In particular, the synthetically produced ceDNA vector may comprise a desired exogenous DNA sequence operably linked to control elements capable of directing transcription of the desired polypeptide, protein, or oligonucleotide encoded by the exogenous DNA sequence when introduced into the subject. The synthetically produced ceDNA vector can be administered via any suitable route as provided above, and elsewhere herein.

Disclosed herein are ceDNA vector compositions and formulations that include one or more of the synthetically produced ceDNA vectors of the present disclosure together with one or more pharmaceutically-acceptable buffers, diluents, or excipients. Such compositions may be included in one or more diagnostic or therapeutic kits, for diagnosing, preventing, treating or ameliorating one or more symptoms of a disease, injury, disorder, trauma or dysfunction. In one aspect the disease, injury, disorder, trauma or dysfunction is a human disease, injury, disorder, trauma or dysfunction.

Another aspect of the technology described herein provides a method for providing a subject in need thereof with a diagnostically- or therapeutically-effective amount of a synthetically produced ceDNA vector, the method comprising providing to a cell, tissue or organ of a subject in need thereof, an amount of the synthetically produced ceDNA vector as disclosed herein; and for a time effective to enable expression of the transgene from the ceDNA vector thereby providing the subject with a diagnostically- or a therapeutically-effective amount of the protein, peptide, nucleic acid expressed by the ceDNA vector. In a further aspect, the subject is human.

Another aspect of the technology described herein provides a method for diagnosing, preventing, treating, or ameliorating at least one or more symptoms of a disease, a disorder, a dysfunction, an injury, an abnormal condition, or trauma in a subject. In an overall and general sense, the method includes at least the step of administering to a subject in need thereof one or more of the disclosed synthetically produced ceDNA vectors, in an amount and for a time sufficient to diagnose, prevent, treat or ameliorate the one or more symptoms of the disease, disorder, dysfunction, injury, abnormal condition, or trauma in the subject. In a further aspect, the subject is human.

Another aspect is use of the synthetically produced ceDNA vector as a tool for treating or reducing one or more symptoms of a disease or disease states. There are a number of inherited diseases in which defective genes are known, and typically fall into two classes: deficiency states, usually of enzymes, which are generally inherited in a recessive manner, and unbalanced states, which may involve regulatory or structural proteins, and which are typically but not always inherited in a dominant manner. For deficiency state diseases, synthetically produced ceDNA vectors can be used to deliver transgenes to bring a normal gene into affected tissues for replacement therapy, as well, in some embodiments, to create animal models for the disease using antisense mutations. For unbalanced disease states, synthetically produced ceDNA vectors can be used to create a disease state in a model system, which could then be used in efforts to counteract the disease state. Thus the synthetically produced ceDNA vectors and methods disclosed herein permit the treatment of genetic diseases. As used herein, a disease state is treated by partially or wholly remedying the deficiency or imbalance that causes the disease or makes it more severe.

### Host Cells

In some embodiments, the synthetically produced ceDNA vector delivers the transgene into a subject host cell. In some embodiments, the subject host cell is a human host cell, including, for example blood cells, stem cells, hematopoietic cells, CD34⁺ cells, liver cells, cancer cells, vascular cells, muscle cells, pancreatic cells, neural cells, ocular or retinal cells, epithelial or endothelial cells, dendritic cells, fibroblasts, or any other cell of mammalian origin, including, without limitation, hepatic (i.e., liver) cells, lung cells, cardiac cells, pancreatic cells, intestinal cells, diaphragmatic cells, renal (i.e., kidney) cells, neural cells, blood cells, bone marrow cells, or any one or more selected tissues of a subject for which gene therapy is contemplated. In one aspect, the subject host cell is a human host cell.

The present disclosure also relates to recombinant host cells as mentioned above, including synthetically produced ceDNA vectors as described herein. Thus, one can use multiple host cells depending on the purpose as is obvious to the skilled artisan. A construct or synthetically produced ceDNA vector including donor sequence is introduced into a host cell so that the donor sequence is maintained as a chromosomal integrant as described earlier. The term host cell encompasses any progeny of a parent cell that is not identical to the parent cell due to mutations that occur during replication. The choice of a host cell will to a large extent depend upon the donor sequence and its source. The host cell may also be a eukaryote, such as a mammalian, insect, plant, or fungal cell. In one embodiment, the host cell is a human cell (e.g., a primary cell, a stem cell, or an immortalized cell line). In some embodiments, the host cell can be administered the synthetically produced ceDNA vector ex vivo and then delivered to the subject after the gene therapy event. A host cell can be any cell type, e.g., a somatic cell or a stem cell, an induced pluripotent stem cell, or a blood cell, e.g., T-cell or B-cell, or bone marrow cell. In certain embodiments, the host cell is an allogenic cell. For example, T-cell genome engineering is useful for cancer immunotherapies, disease modulation such as HIV therapy (e.g., receptor knock out, such as CXCR4 and CCR5) and immunodeficiency therapies. MHC receptors on B-cells can be targeted for immunotherapy. In some embodiments, gene modified host cells, e.g., bone marrow stem cells, e.g., CD34⁺ cells, or induced pluripotent stem cells can be transplanted back into a patient for expression of a therapeutic protein.

Exemplary Transgenes and Diseases to be Treated with a ceDNA Vector

A closed-ended DNA vector, including a ceDNA vector, produced using the synthetic process as described herein are also useful for correcting a defective gene. As a non-limiting example, DMD gene of Duchene Muscular Dystrophy can be delivered using the synthetically produced ceDNA vectors as disclosed herein.

A synthetically produced ceDNA vector or a composition thereof can be used in the treatment of any hereditary disease. As a non-limiting example, the synthetically produced ceDNA vector or a composition thereof e.g. can be used in the treatment of transthyretin amyloidosis (ATTR), an orphan disease where the mutant protein misfolds and aggregates in nerves, the heart, the gastrointestinal system etc. It is contemplated herein that the disease can be treated by deletion of the mutant disease gene (mutTTR) using the synthetically produced ceDNA vector systems described herein. Such treatments of hereditary diseases can halt disease progression and may enable regression of an established disease or reduction of at least one symptom of the disease by at least 10%.

In another embodiment, a synthetically produced ceDNA vector or a composition thereof can be used in the treatment of ornithine transcarbamylase deficiency (OTC deficiency), hyperammonaemia or other urea cycle disorders, which impair a neonate or infant's ability to detoxify ammonia. As with all diseases of inborn metabolism, it is contemplated herein that even a partial restoration of enzyme activity compared to wild-type controls (e.g., at least 20%, at least 30%, at least 40%, at least 50%, at least 60%, at least 70%, at least 80%, at least 90%, at least 95% or at least 99%) may be sufficient for reduction in at least one symptom OTC and/or an improvement in the quality of life for a subject having OTC deficiency. In one embodiment, a nucleic acid encoding OTC can be inserted behind the albumin endogenous promoter for in vivo protein replacement.

In another embodiment, a synthetically produced ceDNA vector or a composition thereof can be used in the treatment of phenylketonuria (PKU) by delivering a nucleic acid sequence encoding a phenylalanine hydroxylase enzyme to reduce buildup of dietary phenylalanine, which can be toxic to PKU sufferers. As with all diseases of inborn metabolism, it is contemplated herein that even a partial restoration of enzyme activity compared to wild-type controls (e.g., at least 20%, at least 30%, at least 40%, at least 50%, at least 60%, at least 70%, at least 80%, at least 90%, at least 95% or at least 99%) may be sufficient for reduction in at least one symptom of PKU and/or an improvement in the quality of life for a subject having PKU. In one embodiment, a nucleic acid encoding phenylalanine hydroxylase can be inserted behind the albumin endogenous promoter for in vivo protein replacement.

In another embodiment, a synthetically produced ceDNA vector or a composition thereof can be used in the treatment of glycogen storage disease (GSD) by delivering a nucleic acid sequence encoding an enzyme to correct aberrant glycogen synthesis or breakdown in subjects having GSD. Non-limiting examples of enzymes that can be delivered and expressed using the synthetically produced ceDNA vectors and methods as described herein include glycogen synthase, glucose-6-phosphatase, acid-alpha glucosidase, glycogen debranching enzyme, glycogen branching enzyme, muscle glycogen phosphorylase, liver glycogen phosphorylase, muscle phosphofructokinase, phosphorylase kinase, glucose transporter-2 (GLUT-2), aldolase A, beta-enolase, phosphoglucomutase-1 (PGM-1), and glycogenin-1. As with all diseases of inborn metabolism, it is contemplated herein that even a partial restoration of enzyme activity compared to wild-type controls (e.g., at least 20%, at least 30%, at least 40%, at least 50%, at least 60%, at least 70%, at least 80%, at least 90%, at least 95% or at least 99%) may be sufficient for reduction in at least one symptom of GSD and/or an improvement in the quality of life for a subject having GSD. In one embodiment, a nucleic acid encoding an enzyme to correct aberrant glycogen storage can be inserted behind the albumin endogenous promoter for in vivo protein replacement.

The synthetically produced ceDNA vectors described herein are also contemplated for use in the treatment of any of, of Leber congenital amaurosis (LCA), polyglutamine diseases, including polyQ repeats, and alpha-1 antitrypsin deficiency (A1AT). LCA is a rare congenital eye disease resulting in blindness, which can be caused by a mutation in any one of the following genes: GUCY2D, RPE65, SPATA7, AIPL1, LCA5, RPGRIP1, CRX, CRB1, NMNAT1, CEP290, IMPDH1, RD3, RDH12, LRAT, TULP1, KCNJ13, GDF6 and/or PRPH2. It is contemplated herein that the ceDNA vectors and compositions and methods as described herein can be adapted for delivery of one or more of the genes associated with LCA in order to correct an error in the gene(s) responsible for the symptoms of LCA. Polyglutamine diseases include, but are not limited to: dentatorubropallidoluysian atrophy, Huntington's disease, spinal and bulbar muscular atrophy, and spinocerebellar ataxia types 1, 2, 3 (also known as Machado-Joseph disease), 6, 7, and 17. A1AT deficiency is a genetic disorder that causes defective production of alpha-1 antitrypsin, leading to decreased activity of the enzyme in the blood and lungs, which in turn can lead to emphysema or chronic obstructive pulmonary disease in affected subjects. Treatment of a subject with an A1AT deficiency is specifically contemplated herein using the ceDNA vectors or compositions thereof as outlined herein. It is contemplated herein that a ceDNA vector comprising a nucleic acid encoding a desired protein for the treatment of LCA, polyglutamine diseases or A1AT deficiency can be administered to a subject in need of treatment.

In further embodiments, the compositions comprising a synthetically produced ceDNA vector as described herein can be used to deliver a viral sequence, a pathogen sequence, a chromosomal sequence, a translocation junction (e.g., a translocation associated with cancer), a non-coding RNA gene or RNA sequence, a disease associated gene, among others.

Any nucleic acid or target gene of interest may be delivered or expressed by a synthetically produced ceDNA vector as disclosed herein. Target nucleic acids and target genes include, but are not limited to nucleic acids encoding polypeptides, or non-coding nucleic acids (e.g., RNAi, miRs etc.) preferably therapeutic (e.g., for medical, diagnostic, or veterinary uses) or immunogenic (e.g., for vaccines) polypeptides. In certain embodiments, the target nucleic acids or target genes that are targeted by the synthetically produced ceDNA vectors as described herein encode one or more polypeptides, peptides, ribozymes, peptide nucleic acids, siRNAs, RNAis, antisense oligonucleotides, antisense polynucleotides, antibodies, antigen binding fragments, or any combination thereof.

In particular, a gene target or transgene for expression by the synthetically produced ceDNA vector as disclosed herein can encode, for example, but is not limited to, protein(s), polypeptide(s), peptide(s), enzyme(s), antibodies, antigen binding fragments, as well as variants, and/or active fragments thereof, for use in the treatment, prophylaxis, and/or amelioration of one or more symptoms of a disease, dysfunction, injury, and/or disorder. In one aspect, the disease, dysfunction, trauma, injury and/or disorder is a human disease, dysfunction, trauma, injury, and/or disorder.

The expression cassette can also encode polypeptides, sense or antisense oligonucleotides, or RNAs (coding or non-coding; e.g., siRNAs, shRNAs, micro-RNAs, and their antisense counterparts (e.g., antagoMiR)). Expression cassettes can include an exogenous sequence that encodes a reporter protein to be used for experimental or diagnostic purposes, such as β-lactamase, β-galactosidase (LacZ), alkaline phosphatase, thymidine kinase, green fluorescent protein (GFP), chloramphenicol acetyltransferase (CAT), luciferase, and others well known in the art.

Sequences provided in the expression cassette, expression construct of a ceDNA vector described herein can be codon optimized for the host cell. As used herein, the term “codon optimized” or “codon optimization” refers to the process of modifying a nucleic acid sequence for enhanced expression in the cells of the vertebrate of interest, e.g., mouse or human, by replacing at least one, more than one, or a significant number of codons of the native sequence (e.g., a prokaryotic sequence) with codons that are more frequently or most frequently used in the genes of that vertebrate. Various species exhibit particular bias for certain codons of a particular amino acid. Typically, codon optimization does not alter the amino acid sequence of the original translated protein. Optimized codons can be determined using e.g., Aptagen's Gene Forge® codon optimization and custom gene synthesis platform (Aptagen, Inc., 2190 Fox Mill Rd. Suite 300, Herndon, Va. 20171) or another publicly available database.

Many organisms display a bias for use of particular codons to code for insertion of a particular amino acid in a growing peptide chain. Codon preference or codon bias, differences in codon usage between organisms, is afforded by degeneracy of the genetic code, and is well documented among many organisms. Codon bias often correlates with the efficiency of translation of messenger RNA (mRNA), which is in turn believed to be dependent on, inter alia, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

Given the large number of gene sequences available for a wide variety of animal, plant and microbial species, it is possible to calculate the relative frequencies of codon usage (Nakamura, Y., et al., “Codon usage tabulated from the international DNA sequence databases: status for the year 2000” Nucl. Acids Res. 28:292 (2000)).

As noted herein, a synthetically produced ceDNA vector as disclosed herein can encode a protein or peptide, or therapeutic nucleic acid sequence or therapeutic agent, including but not limited to one or more agonists, antagonists, anti-apoptosis factors, inhibitors, receptors, cytokines, cytotoxins, erythropoietic agents, glycoproteins, growth factors, growth factor receptors, hormones, hormone receptors, interferons, interleukins, interleukin receptors, nerve growth factors, neuroactive peptides, neuroactive peptide receptors, proteases, protease inhibitors, protein decarboxylases, protein kinases, protein kinase inhibitors, enzymes, receptor binding proteins, transport proteins or one or more inhibitors thereof, serotonin receptors, or one or more uptake inhibitors thereof, serpins, serpin receptors, tumor suppressors, diagnostic molecules, chemotherapeutic agents, cytotoxins, or any combination thereof.

The synthetically produced ceDNA vectors are also useful for ablating gene expression. For example, in one embodiment a ceDNA vector can be used to express an antisense nucleic acid or functional RNA to induce knockdown of a target gene. As a non-limiting example, expression of CXCR4 and CCR5, HIV receptors, have been successfully ablated in primary human T-cells, See Schumann et al., (2015), PNAS 112(33): 10437-10442. Another gene for targeted inhibition is PD-1, where the synthetically produced ceDNA vector can express an inhibitory nucleic acid or RNAi or functional RNA to inhibit the expression of PD-1. PD-1 expresses an immune checkpoint cell surface receptor on chronically active T cells that happens in malignancy. See Schumann et al., supra.

In some embodiments, a synthetically produced ceDNA vectors is useful for correcting a defective gene by expressing a transgene that targets the diseased gene. Non-limiting examples of diseases or disorders amenable to treatment by a synthetically produced ceDNA vector as disclosed herein, are listed in Tables A-C along with their and their associated genes in U.S. Patent Application Publication No. 2014/0170753.

In alternative embodiments, the synthetically produced ceDNA vectors are used for insertion of an expression cassette for expression of a therapeutic protein or reporter protein in a safe harbor gene, e.g., in an inactive intron. In certain embodiments, a promoter-less cassette is inserted into the safe harbor gene. In such embodiments, a promoter-less cassette can take advantage of the safe harbor gene regulatory elements (promoters, enhancers, and signaling peptides), a non-limiting example of insertion at the safe harbor locus is insertion into to the albumin locus that is described in Blood (2015) 126 (15): 1777-1784. Insertion into Albumin has the benefit of enabling secretion of the transgene into the blood (See e.g., Example 22). In addition, a genomic safe harbor site can be determined using techniques known in the art and described in, for example, Papapetrou, ER & Schambach, A. Molecular Therapy 24(4):678-684 (2016) or Sadelain et al., Nature Reviews Cancer 12:51-58 (2012). It is specifically contemplated herein that safe harbor sites in an adeno associated virus (AAV) genome (e.g., AAVS1 safe harbor site) can be used with the methods and compositions described herein (see e.g., Oceguera-Yanez et al., Methods 101:43-55 (2016) or Tiyaboonchai, A et al., Stem Cell Res 12(3):630-7 (2014). For example, the AAVS1 genomic safe harbor site can be used with the ceDNA vectors and compositions as described herein for the purposes of hematopoietic specific transgene expression and gene silencing in embryonic stem cells (e.g., human embryonic stem cells) or induced pluripotent stem cells (iPS cells). In addition, it is contemplated herein that synthetic or commercially available homology-directed repair donor templates for insertion into an AASV1 safe harbor site on chromosome 19 can be used with the ceDNA vectors or compositions as described herein. For example, homology-directed repair templates, and guide RNA, can be purchased commercially, for example, from System Biosciences, Palo Alto, CA, and cloned into a ceDNA vector.

In some embodiments, the synthetically produced ceDNA vectors are used for expressing a transgene, or knocking out or decreasing expression of a target gene in a T cell, e.g., to engineer the T cell for improved adoptive cell transfer and/or CAR-T therapies (see, e.g., Example 24). In some embodiments, the ceDNA vector as described herein can express transgenes that knock-out genes. Non-limiting examples of therapeutically relevant knock-outs of T cells are described in PNAS (2015) 112(33):10437-10442.

### Additional Diseases for Gene Therapy

In general, the ceDNA vector produced by the synthetic methods as disclosed herein can be used to deliver any transgene in accordance with the description above to treat, prevent, or ameliorate the symptoms associated with any disorder related to gene expression. Illustrative disease states include, but are not-limited to: cystic fibrosis (and other diseases of the lung), hemophilia A, hemophilia B, thalassemia, anemia and other blood disorders, AIDS, Alzheimer's disease, Parkinson's disease, Huntington's disease, amyotrophic lateral sclerosis, epilepsy, and other neurological disorders, cancer, diabetes mellitus, muscular dystrophies (e.g., Duchenne, Becker), Hurler's disease, adenosine deaminase deficiency, metabolic defects, retinal degenerative diseases (and other diseases of the eye), mitochondriopathies (e.g., Leber's hereditary optic neuropathy (LHON), Leigh syndrome, and subacute sclerosing encephalopathy), myopathies (e.g., facioscapulohumeral myopathy (FSHD) and cardiomyopathies), diseases of solid organs (e.g., brain, liver, kidney, heart), and the like. In some embodiments, a ceDNA vector produced by the synthetic production methods as described herein can be advantageously used in the treatment of individuals with metabolic disorders (e.g., ornithine transcarbamylase deficiency).

In some embodiments, a ceDNA vector produced by the synthetic production methods as described herein can be used to treat, ameliorate, and/or prevent a disease or disorder caused by mutation in a gene or gene product. Exemplary diseases or disorders that can be treated with a ceDNA vectors include, but are not limited to, metabolic diseases or disorders (e.g., Fabry disease, Gaucher disease, phenylketonuria (PKU), glycogen storage disease); urea cycle diseases or disorders (e.g., ornithine transcarbamylase (OTC) deficiency); lysosomal storage diseases or disorders (e.g., metachromatic leukodystrophy (MLD), mucopolysaccharidosis Type II (MPSII; Hunter syndrome)); liver diseases or disorders (e.g., progressive familial intrahepatic cholestasis (PFIC); blood diseases or disorders (e.g., hemophilia (A and B), thalassemia, and anemia); cancers and tumors, and genetic diseases or disorders (e.g., cystic fibrosis).

As still a further aspect, a ceDNA vector produced by the synthetic production methods as described herein may be employed to deliver a heterologous nucleotide sequence in situations in which it is desirable to regulate the level of transgene expression (e.g., transgenes encoding hormones or growth factors, as described herein).

Accordingly, in some embodiments, a ceDNA vector produced by the synthetic production methods as described herein can be used to correct an abnormal level and/or function of a gene product (e.g., an absence of, or a defect in, a protein) that results in the disease or disorder. The ceDNA vector can produce a functional protein and/or modify levels of the protein to alleviate or reduce symptoms resulting from, or confer benefit to, a particular disease or disorder caused by the absence or a defect in the protein. For example, treatment of OTC deficiency can be achieved by producing functional OTC enzyme; treatment of hemophilia A and B can be achieved by modifying levels of Factor VIII, Factor IX, and Factor X; treatment of PKU can be achieved by modifying levels of phenylalanine hydroxylase enzyme; treatment of Fabry or Gaucher disease can be achieved by producing functional alpha galactosidase or beta glucocerebrosidase, respectively; treatment of MLD or MPSII can be achieved by producing functional arylsulfatase A or iduronate-2-sulfatase, respectively; treatment of cystic fibrosis can be achieved by producing functional cystic fibrosis transmembrane conductance regulator; treatment of glycogen storage disease can be achieved by restoring functional G6Pase enzyme function; and treatment of PFIC can be achieved by producing functional ATP8B1, ABCB 11, ABCB4, or TJP2 genes.

In alternative embodiments, a ceDNA vector produced by the synthetic production methods as described herein can be used to provide an antisense nucleic acid to a cell in vitro or in vivo. For example, where the transgene is a RNAi molecule, expression of the antisense nucleic acid or RNAi in the target cell diminishes expression of a particular protein by the cell. Accordingly, transgenes which are RNAi molecules or antisense nucleic acids may be administered to decrease expression of a particular protein in a subject in need thereof. Antisense nucleic acids may also be administered to cells in vitro to regulate cell physiology, e.g., to optimize cell or tissue culture systems.

In some embodiments, exemplary transgenes encoded by a ceDNA vector produced by the synthetic production methods as described herein, include, but are not limited to: X, lysosomal enzymes (e.g., hexosaminidase A, associated with Tay-Sachs disease, or iduronate sulfatase, associated, with Hunter Syndrome/MPS II), erythropoietin, angiostatin, endostatin, superoxide dismutase, globin, leptin, catalase, tyrosine hydroxylase, as well as cytokines (e.g., a interferon, 3-interferon, interferon-γ, interleukin-2, interleukin-4, interleukin 12, granulocyte-macrophage colony stimulating factor, lymphotoxin, and the like), peptide growth factors and hormones (e.g., somatotropin, insulin, insulin-like growth factors 1 and 2, platelet derived growth factor (PDGF), epidermal growth factor (EGF), fibroblast growth factor (FGF), nerve growth factor (NGF), neurotrophic factor-3 and 4, brain-derived neurotrophic factor (BDNF), glial derived growth factor (GDNF), transforming growth factor-α and —P, and the like), receptors (e.g., tumor necrosis factor receptor). In some exemplary embodiments, the transgene encodes a monoclonal antibody specific for one or more desired targets. In some exemplary embodiments, more than one transgene is encoded by the ceDNA vector. In some exemplary embodiments, the transgene encodes a fusion protein comprising two different polypeptides of interest. In some embodiments, the transgene encodes an antibody, including a full-length antibody or antibody fragment, as defined herein. In some embodiments, the antibody is an antigen-binding domain or an immunoglobulin variable domain sequence, as that is defined herein. Other illustrative transgene sequences encode suicide gene products (thymidine kinase, cytosine deaminase, diphtheria toxin, cytochrome P450, deoxycytidine kinase, and tumor necrosis factor), proteins conferring resistance to a drug used in cancer therapy, and tumor suppressor gene products.

In a representative embodiment, the transgene expressed by a ceDNA vector produced by the synthetic production methods as described herein can be used for the treatment of muscular dystrophy in a subject in need thereof, the method comprising: administering a treatment-, amelioration- or prevention-effective amount of ceDNA vector described herein, wherein the ceDNA vector comprises a heterologous nucleic acid encoding dystrophin, a mini-dystrophin, a micro-dystrophin, myostatin propeptide, follistatin, activin type II soluble receptor, IGF-1, anti-inflammatory polypeptides such as the Ikappa B dominant mutant, sarcospan, utrophin, a micro-dystrophin, laminin-α2, α-sarcoglycan, β-sarcoglycan, γ-sarcoglycan, δ-sarcoglycan, IGF-1, an antibody or antibody fragment against myostatin or myostatin propeptide, and/or RNAi against myostatin. In particular embodiments, the synthetically produced ceDNA vector can be administered to skeletal, diaphragm and/or cardiac muscle as described elsewhere herein.

In some embodiments, a ceDNA vector produced by the synthetic production methods as described herein can be used to deliver a transgene to skeletal, cardiac or diaphragm muscle, for production of a polypeptide (e.g., an enzyme) or functional RNA (e.g., RNAi, microRNA, antisense RNA) that normally circulates in the blood or for systemic delivery to other tissues to treat, ameliorate, and/or prevent a disorder (e.g., a metabolic disorder, such as diabetes (e.g., insulin), hemophilia (e.g., VIII), a mucopolysaccharide disorder (e.g., Sly syndrome, Hurler Syndrome, Scheie Syndrome, Hurler-Scheie Syndrome, Hunter's Syndrome, Sanfilippo Syndrome A, B, C, D, Morquio Syndrome, Maroteaux-Lamy Syndrome, etc.) or a lysosomal storage disorder (such as Gaucher's disease [glucocerebrosidase], Pompe disease [lysosomal acid.alpha.-glucosidase] or Fabry disease [.alpha.-galactosidase A]) or a glycogen storage disorder (such as Pompe disease [lysosomal acid a glucosidase]). Other suitable proteins for treating, ameliorating, and/or preventing metabolic disorders are described above.

In other embodiments, a ceDNA vector produced by the synthetic production methods as described herein can be used to deliver a transgene in a method of treating, ameliorating, and/or preventing a metabolic disorder in a subject in need thereof. Illustrative metabolic disorders and transgenes encoding polypeptides are described herein. Optionally, the polypeptide is secreted (e.g., a polypeptide that is a secreted polypeptide in its native state or that has been engineered to be secreted, for example, by operable association with a secretory signal sequence as is known in the art).

Another aspect of the disclosure relates to a method of treating, ameliorating, and/or preventing congenital heart failure or PAD in a subject in need thereof, the method comprising administering a ceDNA vector produced by the synthetic production methods as described herein to a mammalian subject, wherein the ceDNA vector comprises a transgene encoding, for example, a sarcoplasmic endoreticulum Ca²⁺-ATPase (SERCA2a), an angiogenic factor, phosphatase inhibitor I (I−1), RNAi against phospholamban; a phospholamban inhibitory or dominant-negative molecule such as phospholamban S16E, a zinc finger protein that regulates the phospholamban gene, β2-adrenergic receptor, .beta.2-adrenergic receptor kinase (BARK), PI3 kinase, calsarcan, a .beta.-adrenergic receptor kinase inhibitor (βARKct), inhibitor 1 of protein phosphatase 1, S100A1, parvalbumin, adenylyl cyclase type 6, a molecule that effects G-protein coupled receptor kinase type 2 knockdown such as a truncated constitutively active βARKct, Pim-1, PGC-1α, SOD-1, SOD-2, EC-SOD, kallikrein, HIF, thymosin-β4, mir-1, mir-133, mir-206 and/or mir-208.

In some embodiments, a ceDNA vector produced by the synthetic production methods as described herein can be administered to the lungs of a subject by any suitable means, optionally by administering an aerosol suspension of respirable particles comprising the ceDNA vectors, which the subject inhales. The respirable particles can be liquid or solid. Aerosols of liquid particles comprising the ceDNA vectors may be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising a ceDNA vector produced by the synthetic production methods as described herein may likewise be produced with any solid particulate medicament aerosol generator, by techniques known in the pharmaceutical art.

In some embodiments, a ceDNA vector produced by the synthetic production methods as described herein can be administered to tissues of the CNS (e.g., brain, eye). In particular embodiments, a ceDNA vector produced by the synthetic production methods as described herein may be administered to treat, ameliorate, or prevent diseases of the CNS, including genetic disorders, neurodegenerative disorders, psychiatric disorders and tumors. Illustrative diseases of the CNS include, but are not limited to Alzheimer's disease, Parkinson's disease, Huntington's disease, Canavan disease, Leigh's disease, Refsum disease, Tourette syndrome, primary lateral sclerosis, amyotrophic lateral sclerosis, progressive muscular atrophy, Pick's disease, muscular dystrophy, multiple sclerosis, myasthenia gravis, Binswanger's disease, trauma due to spinal cord or head injury, Tay Sachs disease, Lesch-Nyan disease, epilepsy, cerebral infarcts, psychiatric disorders including mood disorders (e.g., depression, bipolar affective disorder, persistent affective disorder, secondary mood disorder), schizophrenia, drug dependency (e.g., alcoholism and other substance dependencies), neuroses (e.g., anxiety, obsessional disorder, somatoform disorder, dissociative disorder, grief, post-partum depression), psychosis (e.g., hallucinations and delusions), dementia, paranoia, attention deficit disorder, psychosexual disorders, sleeping disorders, pain disorders, eating or weight disorders (e.g., obesity, cachexia, anorexia nervosa, and bulimia) and cancers and tumors (e.g., pituitary tumors) of the CNS.

Ocular disorders that may be treated, ameliorated, or prevented with a ceDNA vector produced by the synthetic production methods as described herein include ophthalmic disorders involving the retina, posterior tract, and optic nerve (e.g., retinitis pigmentosa, diabetic retinopathy and other retinal degenerative diseases, uveitis, age-related macular degeneration, glaucoma). Many ophthalmic diseases and disorders are associated with one or more of three types of indications: (1) angiogenesis, (2) inflammation, and (3) degeneration. In some embodiments, a ceDNA vector produced by the synthetic production methods as described herein can be employed to deliver anti-angiogenic factors; anti-inflammatory factors; factors that retard cell degeneration, promote cell sparing, or promote cell growth and combinations of the foregoing. Diabetic retinopathy, for example, is characterized by angiogenesis. Diabetic retinopathy can be treated by delivering one or more anti-angiogenic factors either intraocularly (e.g., in the vitreous) or periocularly (e.g., in the sub-Tenon's region). One or more neurotrophic factors may also be co-delivered, either intraocularly (e.g., intravitreally) or periocularly. Additional ocular diseases that may be treated, ameliorated, or prevented with the ceDNA vectors of the disclosure include geographic atrophy, vascular or “wet” macular degeneration, Stargardt disease, Leber Congenital Amaurosis (LCA), Usher syndrome, pseudoxanthoma elasticum (PXE), x-linked retinitis pigmentosa (XLRP), x-linked retinoschisis (XLRS), Choroideremia, Leber hereditary optic neuropathy (LHON), Archomatopsia, cone-rod dystrophy, Fuchs endothelial corneal dystrophy, diabetic macular edema and ocular cancer and tumors.

In some embodiments, inflammatory ocular diseases or disorders (e.g., uveitis) can be treated, ameliorated, or prevented by a ceDNA vector produced by the synthetic production methods as described herein. One or more anti-inflammatory factors can be expressed by intraocular (e.g., vitreous or anterior chamber) administration of a ceDNA vector produced by the synthetic production methods as described herein. In other embodiments, ocular diseases or disorders characterized by retinal degeneration (e.g., retinitis pigmentosa) can be treated, ameliorated, or prevented by the ceDNA vectors of the disclosure. Intraocular (e.g., vitreal administration) of a ceDNA vector produced by the synthetic production methods as described herein encoding one or more neurotrophic factors can be used to treat such retinal degeneration-based diseases. In some embodiments, diseases or disorders that involve both angiogenesis and retinal degeneration (e.g., age-related macular degeneration) can be treated with a ceDNA vector produced by the synthetic production methods as described herein. Age-related macular degeneration can be treated by administering a ceDNA vector produced by the synthetic production methods as described herein encoding one or more neurotrophic factors intraocularly (e.g., vitreous) and/or one or more anti-angiogenic factors intraocularly or periocularly (e.g., in the sub-Tenon's region). Glaucoma is characterized by increased ocular pressure and loss of retinal ganglion cells. Treatments for glaucoma include administration of one or more neuroprotective agents that protect cells from excitotoxic damage using the ceDNA vector as disclosed herein. Accordingly, such agents include N-methyl-D-aspartate (NMDA) antagonists, cytokines, and neurotrophic factors, can be delivered intraocularly, optionally intravitreally using a ceDNA vector produced by the synthetic production methods as described herein.

In other embodiments, a ceDNA vector produced by the synthetic production methods as described herein may be used to treat seizures, e.g., to reduce the onset, incidence or severity of seizures. The efficacy of a therapeutic treatment for seizures can be assessed by behavioral (e.g., shaking, tics of the eye or mouth) and/or electrographic means (most seizures have signature electrographic abnormalities). Thus, a ceDNA vector produced by the synthetic production methods as described herein can also be used to treat epilepsy, which is marked by multiple seizures over time. In one representative embodiment, somatostatin (or an active fragment thereof) is administered to the brain using a ceDNA vector produced by the synthetic production methods as described herein to treat a pituitary tumor. According to this embodiment, a ceDNA vector produced by the synthetic production methods as described herein encoding somatostatin (or an active fragment thereof) is administered by microinfusion into the pituitary. Likewise, such treatment can be used to treat acromegaly (abnormal growth hormone secretion from the pituitary). The nucleic acid (e.g., GenBank Accession No. J00306) and amino acid (e.g., GenBank Accession No. P01166; contains processed active peptides somatostatin-28 and somatostatin-14) sequences of somatostatins as are known in the art. In particular embodiments, the ceDNA vector can encode a transgene that comprises a secretory signal as described in U.S. Pat. No. 7,071,172.

Another aspect of the disclosure relates to the use of a ceDNA vector produced by the synthetic production methods as described herein to produce antisense RNA, RNAi or other functional RNA (e.g., a ribozyme) for systemic delivery to a subject in vivo. Accordingly, in some embodiments, a ceDNA vector produced by the synthetic production methods as described herein can comprise atransgene that encodes an antisense nucleic acid, a ribozyme (e.g., as described in U.S. Pat. No. 5,877,022), RNAs that affect spliceosome-mediated trans-splicing (see, Puttaraju et al., (1999) Nature Biotech. 17:246; U.S. Pat. Nos. 6,013,487 and 6,083,702), interfering RNAs (RNAi) that mediate gene silencing (see, Sharp et al., (2000) Science 287:2431) or other non-translated RNAs, such as “guide” RNAs (Gorman et al., (1998) Proc. Nat. Acad. Sci. USA 95:4929; U.S. Pat. No. 5,869,248 to Yuan et al.), and the like.

In some embodiments, a ceDNA vector produced by the synthetic production methods as described herein can further also comprise a transgene that encodes a reporter polypeptide (e.g., an enzyme such as Green Fluorescent Protein, or alkaline phosphatase). In some embodiments, a transgene that encodes a reporter protein useful for experimental or diagnostic purposes, is selected from any of: β-lactamase, β-galactosidase (LacZ), alkaline phosphatase, thymidine kinase, green fluorescent protein (GFP), chloramphenicol acetyltransferase (CAT), luciferase, and others well known in the art. In some aspects, synthetically produced ceDNA vectors comprising a transgene encoding a reporter polypeptide may be used for diagnostic purposes or as markers of the ceDNA vector's activity in the subject to which they are administered.

In some embodiments, a ceDNA vector produced by the synthetic production methods as described herein can comprise a transgene or a heterologous nucleotide sequence that shares homology with, and recombines with a locus on the host chromosome. This approach may be utilized to correct a genetic defect in the host cell.

In some embodiments, a ceDNA vector produced by the synthetic production methods as described herein can comprise a transgene that can be used to express an immunogenic polypeptide in a subject, e.g., for vaccination. The transgene may encode any immunogen of interest known in the art including, but not limited to, immunogens from human immunodeficiency virus, influenza virus, gag proteins, tumor antigens, cancer antigens, bacterial antigens, viral antigens, and the like.

Testing for Successful Gene Expression Using a ceDNA Vector

Assays well known in the art can be used to test the efficiency of gene delivery by a synthetically produced ceDNA vector and can be performed in both in vitro and in vivo models. Knock-in or knock-out of a desired transgene by a synthetically produced ceDNA can be assessed by one skilled in the art by measuring mRNA and protein levels of the desired transgene (e.g., reverse transcription PCR, western blot analysis, and enzyme-linked immunosorbent assay (ELISA)). Nucleic acid alterations by synthetically produced ceDNA (e.g., point mutations, or deletion of DNA regions) can be assessed by deep sequencing of genomic target DNA. In one embodiment, synthetically produced ceDNA comprises a reporter protein that can be used to assess the expression of the desired transgene, for example by examining the expression of the reporter protein by fluorescence microscopy or a luminescence plate reader. For in vivo applications, protein function assays can be used to test the functionality of a given gene and/or gene product to determine if gene expression has successfully occurred. For example, it is envisioned that a point mutation in the cystic fibrosis transmembrane conductance regulator gene (CFTR) inhibits the capacity of CFTR to move anions (e.g., Cl⁻) through the anion channel, can be corrected by delivering a functional (i.e., non-mutated) CFTR gene to the subject with a ceDNA vector. Following administration of a ceDNA vector, one skilled in the art can assess the capacity for anions to move through the anion channel to determine if the CFTR gene has been delivered and expressed. One skilled will be able to determine the best test for measuring functionality of a protein in vitro or in vivo.

It is contemplated herein that the effects of gene expression of the transgene from the ceDNA vector in a cell or subject can last for at least 1 month, at least 2 months, at least 3 months, at least four months, at least 5 months, at least six months, at least 10 months, at least 12 months, at least 18 months, at least 2 years, at least 5 years, at least 10 years, at least 20 years, or can be permanent.

In some embodiments, a transgene in the expression cassette, expression construct, or ceDNA vector described herein can be codon optimized for the host cell. As used herein, the term “codon optimized” or “codon optimization” refers to the process of modifying a nucleic acid sequence for enhanced expression in the cells of the vertebrate of interest, e.g., mouse or human (e.g., humanized), by replacing at least one, more than one, or a significant number of codons of the native sequence (e.g., a prokaryotic sequence) with codons that are more frequently or most frequently used in the genes of that vertebrate. Various species exhibit particular bias for certain codons of a particular amino acid. Typically, codon optimization does not alter the amino acid sequence of the original translated protein. Optimized codons can be determined using e.g., Aptagen's Gene Forge® codon optimization and custom gene synthesis platform (Aptagen, Inc.) or another publicly available database.

### XII. Administration

In particular embodiments, more than one administration (e.g., two, three, four or more administrations) may be employed to achieve the desired level of gene expression over a period of various intervals, e.g., daily, weekly, monthly, yearly, etc.

Exemplary modes of administration of a closed-ended DNA vector, including a ceDNA vector, produced using the synthetic process as described herein includes oral, rectal, transmucosal, intranasal, inhalation (e.g., via an aerosol), buccal (e.g., sublingual), vaginal, intrathecal, intraocular, transdermal, intraendothelial, in utero (or in ovo), parenteral (e.g., intravenous, subcutaneous, intradermal, intracranial, intramuscular [including administration to skeletal, diaphragm and/or cardiac muscle], intrapleural, intracerebral, and intraarticular), topical (e.g., to both skin and mucosal surfaces, including airway surfaces, and transdermal administration), intralymphatic, and the like, as well as direct tissue or organ injection (e.g., to liver, eye, skeletal muscle, cardiac muscle, diaphragm muscle or brain).

Administration of a closed-ended DNA vector, including a ceDNA vector, produced using the synthetic process as described herein can be to any site in a subject, including, without limitation, a site selected from the group consisting of the brain, a skeletal muscle, a smooth muscle, the heart, the diaphragm, the airway epithelium, the liver, the kidney, the spleen, the pancreas, the skin, and the eye. Administration of the synthetically produced ceDNA vector can also be to a tumor (e.g., in or near a tumor or a lymph node). The most suitable route in any given case will depend on the nature and severity of the condition being treated, ameliorated, and/or prevented and on the nature of the particular ceDNA vector that is being used. Additionally, a ceDNA vector produced using the synthetic process as described herein permits one to administer more than one transgene in a single vector, or multiple ceDNA vectors (e.g. a ceDNA cocktail).

Administration of a closed-ended DNA vector, including a ceDNA vector, produced using the synthetic process as described herein to skeletal muscle according to the present disclosure includes but is not limited to administration to skeletal muscle in the limbs (e.g., upper arm, lower arm, upper leg, and/or lower leg), back, neck, head (e.g., tongue), thorax, abdomen, pelvis/perineum, and/or digits. The synthetically produced ceDNA vector can be delivered to skeletal muscle by intravenous administration, intra-arterial administration, intraperitoneal administration, limb perfusion, (optionally, isolated limb perfusion of a leg and/or arm; see, e.g. Arruda et al., (2005) Blood 105: 3458-3464), and/or direct intramuscular injection. In particular embodiments, the ceDNA vector as disclosed herein is administered to a limb (arm and/or leg) of a subject (e.g., a subject with muscular dystrophy such as DMD) by limb perfusion, optionally isolated limb perfusion (e.g., by intravenous or intra-articular administration. In certain embodiments, a DNA vector, including a ceDNA vector produced using the synthetic process as described herein can be administered without employing “hydrodynamic” techniques.

Administration of a closed-ended DNA vector, including a ceDNA vector, produced using the synthetic process as described herein to cardiac muscle includes administration to the left atrium, right atrium, left ventricle, right ventricle and/or septum. The synthetically produced ceDNA vector as described herein can be delivered to cardiac muscle by intravenous administration, intra-arterial administration such as intra-aortic administration, direct cardiac injection (e.g., into left atrium, right atrium, left ventricle, right ventricle), and/or coronary artery perfusion. Administration to diaphragm muscle can be by any suitable method including intravenous administration, intra-arterial administration, and/or intra-peritoneal administration. Administration to smooth muscle can be by any suitable method including intravenous administration, intra-arterial administration, and/or intra-peritoneal administration. In one embodiment, administration can be to endothelial cells present in, near, and/or on smooth muscle.

In some embodiments, a DNA vector, including a ceDNA vector produced using the synthetic process as described herein is administered to skeletal muscle, diaphragm muscle and/or cardiac muscle (e.g., to treat, ameliorate and/or prevent muscular dystrophy or heart disease (e.g., PAD or congestive heart failure).

### Ex Vivo Treatment

In some embodiments, cells are removed from a subject, a closed-ended DNA vector, including a ceDNA vector, produced using the synthetic process as described herein is introduced therein, and the cells are then replaced back into the subject. Methods of removing cells from subject for treatment ex vivo, followed by introduction back into the subject are known in the art (see, e.g., U.S. Pat. No. 5,399,346). Alternatively, a closed-ended DNA vector, including a ceDNA vector, produced using the synthetic process as described herein is introduced into cells from another subject, into cultured cells, or into cells from any other suitable source, and the cells are administered to a subject in need thereof.

Cells transduced with a closed-ended DNA vector, including a ceDNA vector, produced using the synthetic process as described herein are preferably administered to the subject in a “therapeutically-effective amount” in combination with a pharmaceutical carrier. Those of ordinary skill in the art will appreciate that the therapeutic effects need not be complete or curative, as long as some benefit is provided to the subject.

In some embodiments, a closed-ended DNA vector, including a ceDNA vector, produced using the synthetic process as described herein can encode a transgene (sometimes called a heterologous nucleotide sequence) that is any polypeptide that is desirably produced in a cell in vitro, ex vivo, or in vivo. For example, in contrast to the use of the ceDNA vectors in a method of treatment as previously discussed herein, in some embodiments a closed-ended DNA vector, including a ceDNA vector, produced using the synthetic process as described herein may be introduced into cultured cells and the expressed gene product isolated therefrom, e.g., for the production of antigens or vaccines.

A closed-ended DNA vector, including a ceDNA vector, produced using the synthetic process as described herein can be used in both veterinary and medical applications. Suitable subjects for ex vivo gene delivery methods as described above include both avians (e.g., chickens, ducks, geese, quail, turkeys and pheasants) and mammals (e.g., humans, bovines, ovines, caprines, equines, felines, canines, and lagomorphs), with mammals being preferred. Human subjects are most preferred. Human subjects include neonates, infants, juveniles, and adults.

One aspect of the technology described herein relates to a method of delivering a transgene to a cell. Typically, for in vitro methods, a closed-ended DNA vector, including a ceDNA vector, produced using the synthetic process as described herein may be introduced into the cell using the methods as disclosed herein, as well as other methods known in the art. A closed-ended DNA vector, including a ceDNA vector, produced using the synthetic process as described herein disclosed herein are preferably administered to the cell in a biologically-effective amount. If a closed-ended DNA vector, including a ceDNA vector, produced using the synthetic process as described herein is administered to a cell in vivo (e.g., to a subject), a biologically-effective amount of the ceDNA vector is an amount that is sufficient to result in transduction and expression of the transgene in a target cell.

### Dose Ranges

In vivo and/or in vitro assays can optionally be employed to help identify optimal dosage ranges for use of the synthetically produced ceDNA vector. The precise dose to be employed in the formulation will also depend on the route of administration, and the seriousness of the condition, and should be decided according to the judgment of the person of ordinary skill in the art and each subject's circumstances. Effective doses can be extrapolated from dose-response curves derived from in vitro or animal model test systems.

A closed-ended DNA vector, including a ceDNA vector, produced using the synthetic process as described herein is administered in sufficient amounts to transfect the cells of a desired tissue and to provide sufficient levels of gene transfer and expression without undue adverse effects. Conventional and pharmaceutically acceptable routes of administration include, but are not limited to, those described above in the “Administration” section, such as direct delivery to the selected organ (e.g., intraportal delivery to the liver), oral, inhalation (including intranasal and intratracheal delivery), intraocular, intravenous, intramuscular, subcutaneous, intradermal, intratumoral, and other parental routes of administration. Routes of administration can be combined, if desired.

The dose of the amount of a synthetically produced ceDNA vector required to achieve a particular “therapeutic effect,” will vary based on several factors including, but not limited to: the route of nucleic acid administration, the level of gene or RNA expression required to achieve a therapeutic effect, the specific disease or disorder being treated, and the stability of the gene(s), RNA product(s), or resulting expressed protein(s). One of skill in the art can readily determine a synthetically produced ceDNA vector dose range to treat a patient having a particular disease or disorder based on the aforementioned factors, as well as other factors that are well known in the art.

Dosage regime can be adjusted to provide the optimum therapeutic response. For example, the oligonucleotide can be repeatedly administered, e.g., several doses can be administered daily or the dose can be proportionally reduced as indicated by the exigencies of the therapeutic situation. One of ordinary skill in the art will readily be able to determine appropriate doses and schedules of administration of the subject oligonucleotides, whether the oligonucleotides are to be administered to cells or to subjects.

A “therapeutically effective amount” or “therapeutically effective dose” will fall in a relatively broad range that can be determined through clinical trials and will depend on the particular application (neural cells will require very small amounts, while systemic injection would require large amounts). For example, for direct in vivo injection into skeletal or cardiac muscle of a human subject, a therapeutically effective dose will be on the order of from about 1 μg to 100 g of the ceDNA vector. If exosomes or microparticles are used to deliver a DNA vector, including a ceDNA vector produced using the synthetic process as described herein, then a therapeutically effective dose can be determined experimentally, but is expected to deliver from 1 μg to about 100 g of vector. Moreover, a therapeutically effective dose is an amount ceDNA vector that expresses a sufficient amount of the transgene to have an effect on the subject that results in a reduction in one or more symptoms of the disease, but does not result in significant off-target or significant adverse side effects.

Formulation of pharmaceutically-acceptable excipients and carrier solutions is well-known to those of skill in the art, as is the development of suitable dosing and treatment regimens for using the particular compositions described herein in a variety of treatment regimens.

For in vitro transfection, an effective amount of a closed-ended DNA vector, including a ceDNA vector, produced using the synthetic process as described herein to be delivered to cells (1×10⁶ cells) will be on the order of 0.1 to 100 μg ceDNA vector, preferably 1 to 20 μg, and more preferably 1 to 15 μg or 8 to 10 μg. Larger ceDNA vectors will require higher doses. If exosomes or microparticles are used, an effective in vitro dose can be determined experimentally but would be intended to deliver generally the same amount of the ceDNA vector.

Treatment can involve administration of a single dose or multiple doses. In some embodiments, more than one dose can be administered to a subject; in fact multiple doses can be administered as needed, because the synthetically produced ceDNA vector elicits does not elicit an anti-capsid host immune response due to the absence of a viral capsid, and its formulation does not contain unwanted cellular contaminants due to its synthetic production. As such, one of skill in the art can readily determine an appropriate number of doses. The number of doses administered can, for example, be on the order of 1-100, preferably 2-20 doses.

Without wishing to be bound by any particular theory, the lack of typical anti-viral immune response elicited by administration of a synthetically produced ceDNA vector as described by the disclosure (i.e., the absence of capsid components) allows the synthetically produced ceDNA vector to be administered to a host on multiple occasions. In some embodiments, the number of occasions in which a heterologous nucleic acid is delivered to a subject is in a range of 2 to 10 times (e.g., 2, 3, 4, 5, 6, 7, 8, 9, or 10 times). In some embodiments, a synthetically produced ceDNA vector is delivered to a subject more than 10 times.

In some embodiments, a dose of a synthetically produced ceDNA vector is administered to a subject no more than once per calendar day (e.g., a 24-hour period). In some embodiments, a dose of a synthetically produced ceDNA vector is administered to a subject no more than once per 2, 3, 4, 5, 6, or 7 calendar days. In some embodiments, a dose of a synthetically produced ceDNA vector is administered to a subject no more than once per calendar week (e.g., 7 calendar days). In some embodiments, a dose of a synthetically produced ceDNA vector is administered to a subject no more than bi-weekly (e.g., once in a two calendar week period). In some embodiments, a dose of a synthetically produced ceDNA vector is administered to a subject no more than once per calendar month (e.g., once in 30 calendar days). In some embodiments, a dose of a synthetically produced ceDNA vector is administered to a subject no more than once per six calendar months. In some embodiments, a dose of a synthetically produced ceDNA vector is administered to a subject no more than once per calendar year (e.g., 365 days or 366 days in a leap year).

### Unit Dosage Forms

In some embodiments, the pharmaceutical compositions can conveniently be presented in unit dosage form. A unit dosage form will typically be adapted to one or more specific routes of administration of the pharmaceutical composition. In some embodiments, the unit dosage form is adapted for administration by inhalation. In some embodiments, the unit dosage form is adapted for administration by a vaporizer. In some embodiments, the unit dosage form is adapted for administration by a nebulizer. In some embodiments, the unit dosage form is adapted for administration by an aerosolizer. In some embodiments, the unit dosage form is adapted for oral administration, for buccal administration, or for sublingual administration. In some embodiments, the unit dosage form is adapted for intravenous, intramuscular, or subcutaneous administration. In some embodiments, the unit dosage form is adapted for intrathecal or intracerebroventricular administration. In some embodiments, the pharmaceutical composition is formulated for topical administration. The amount of active ingredient which can be combined with a carrier material to produce a single dosage form will generally be that amount of the compound which produces a therapeutic effect.

### XIII. Various Applications

The compositions and closed-ended DNA vector, including ceDNA vectors, produced using the synthetic process as described herein can be used to deliver a transgene for various purposes as described above. In some embodiments, a transgene can encode a protein or be a functional RNA, and in some embodiments, can be a protein or functional RNA that is modified for research purposes, e.g., to create a somatic transgenic animal model harboring one or more mutations or a corrected gene sequence, e.g., to study the function of the target gene. In another example, the transgene encodes a protein or functional RNA to create an animal model of disease.

In some embodiments, the transgene encodes one or more peptides, polypeptides, or proteins, which are useful for the treatment, amelioration, or prevention of disease states in a mammalian subject. The transgene expressed by the synthetically produced ceDNA vector is administered to a patient in a sufficient amount to treat a disease associated with an abnormal gene sequence, which can result in any one or more of the following: reduced expression, lack of expression or dysfunction of the target gene.

In some embodiments, a DNA vector, including a ceDNA vector, produced using the synthetic process as described herein are envisioned for use in diagnostic and screening methods, whereby a transgene is transiently or stably expressed in a cell culture system, or alternatively, a transgenic animal model.

Another aspect of the technology described herein provides a method of transducing a population of mammalian cells. In an overall and general sense, the method includes at least the step of introducing into one or more cells of the population, a composition that comprises an effective amount of one or more of the synthetically produced ceDNA disclosed herein.

Additionally, the present disclosure provides compositions, as well as therapeutic and/or diagnostic kits that include one or more of the disclosed closed-ended DNA vector, including a ceDNA vector composition, produced using the synthetic process as described herein, formulated with one or more additional ingredients, or prepared with one or more instructions for their use.

A cell to be administered a closed-ended DNA vector, including a ceDNA vector, produced using the synthetic process as described herein may be of any type, including but not limited to neural cells (including cells of the peripheral and central nervous systems, in particular, brain cells), lung cells, retinal cells, epithelial cells (e.g., gut and respiratory epithelial cells), muscle cells, dendritic cells, pancreatic cells (including islet cells), hepatic cells, myocardial cells, bone cells (e.g., bone marrow stem cells), hematopoietic stem cells, spleen cells, keratinocytes, fibroblasts, endothelial cells, prostate cells, germ cells, and the like. Alternatively, the cell may be any progenitor cell. As a further alternative, the cell can be a stem cell (e.g., neural stem cell, liver stem cell). As still a further alternative, the cell may be a cancer or tumor cell. Moreover, the cells can be from any species of origin, as indicated above.

### EXAMPLES

The following examples are provided by way of illustration not limitation. It will be appreciated by one of ordinary skill in the art that a DNA vector, including a ceDNA vector produced using the synthetic process as described herein can be constructed from any of the symmetric or asymmetric ITR configurations, comprising any of wild-type or modified ITRs as described herein, and that the following exemplary methods can be used to construct and assess the activity of such ceDNA vectors. While the methods are exemplified with certain ceDNA vectors, they are applicable to any DNA vector, including any ceDNA vector, in keeping with the description.

### Example 1: Insect Cell-Based Production of ceDNA

For comparative purposes, Example 1 describes the production of ceDNA vectors using an insect cell-based method and a polynucleotide construct template, and is also described in Example 1 of International Patent Application Publication No. WO2019/051255. For example, a polynucleotide construct template used for generating the ceDNA vectors of the present disclosure according to Example 1 can be a ceDNA-plasmid, a ceDNA-Bacmid, and/or a ceDNA-baculovirus. Without being limited to theory, in a permissive host cell, in the presence of e.g., Rep, the polynucleotide construct template having two symmetric ITRs and an expression construct, where at least one of the ITRs is modified relative to a wild-type ITR sequence, replicates to produce ceDNA vectors. ceDNA vector production undergoes two steps: first, excision (“rescue”) of template from the template backbone (e.g. ceDNA-plasmid, ceDNA-bacmid, ceDNA-baculovirus genome etc.) via Rep proteins, and second, Rep mediated replication of the excised ceDNA vector.

An exemplary method to produce ceDNA vectors in a method using insect cell is from a ceDNA-plasmid as described herein. Referring to FIG. 1A and FIG. 1B, the polynucleotide construct template of each of the ceDNA-plasmids includes both a left ITR and a right ITR (both wild-type in FIG. 1A to produce a ceDNA with symmetric substantially symmetric ITRs; asymmetric ITRs in FIG. 1B) with the following between the ITR sequences: (i) an enhancer/promoter; (ii) a cloning site for a transgene; (iii) a posttranscriptional response element (e.g. the woodchuck hepatitis virus posttranscriptional regulatory element (WPRE)); and (iv) a poly-adenylation signal (e.g. from bovine growth hormone gene (BGHpA). Unique restriction endonuclease recognition sites (R1-R6) (shown in FIG. 1A and FIG. 1B) were also introduced between each component to facilitate the introduction of new genetic components into the specific sites in the construct. R3 (PmeI) GTTTAAAC (SEQ ID NO: 123) and R4 (Pacd) TTAATTAA (SEQ ID NO: 124) enzyme sites are engineered into the cloning site to introduce an open reading frame of a transgene. These sequences were cloned into a pFastBac HT B plasmid obtained from ThermoFisher Scientific.

Production of ceDNA-bacmids

DH10Bac competent cells (MAX EFFICIENCY® DH10Bac™ Competent Cells, Thermo Fisher) were transformed with either test or control plasmids following a protocol according to the manufacturer's instructions. Recombination between the plasmid and a baculovirus shuttle vector in the DH10Bac cells were induced to generate recombinant ceDNA-bacmids. The recombinant bacmids were selected by screening a positive selection based on blue-white screening in E. coli (Φ80dlacZΔM15 marker provides α-complementation of the β-galactosidase gene from the bacmid vector) on a bacterial agar plate containing X-gal and IPTG with antibiotics to select for transformants and maintenance of the bacmid and transposase plasmids. White colonies caused by transposition that disrupts the β-galactoside indicator gene were picked and cultured in 10 ml of media.

The recombinant ceDNA-bacmids were isolated from the E. coli and transfected into Sf9 or Sf21 insect cells using FugeneHD to produce infectious baculovirus. The adherent Sf9 or Sf21 insect cells were cultured in 50 ml of media in T25 flasks at 25° C. Four days later, culture medium (containing the P0 virus) was removed from the cells, filtered through a 0.45 μm filter, separating the infectious baculovirus particles from cells or cell debris.

Optionally, the first generation of the baculovirus (P0) was amplified by infecting naïve Sf9 or Sf21 insect cells in 50 to 500 ml of media. Cells were maintained in suspension cultures in an orbital shaker incubator at 130 rpm at 25° C., monitoring cell diameter and viability, until cells reach a diameter of 18-19 nm (from a naïve diameter of 14-15 nm), and a density of ˜4.0E+6 cells/mL. Between 3 and 8 days post-infection, the P1 baculovirus particles in the medium were collected following centrifugation to remove cells and debris then filtration through a 0.45 μm filter.

The ceDNA-baculovirus comprising the test constructs were collected and the infectious activity, or titer, of the baculovirus was determined. Specifically, four×20 ml Sf9 cell cultures at 2.5E+6 cells/ml were treated with P1 baculovirus at the following dilutions: 1/1000, 1/10,000, 1/50,000, 1/100,000, and incubated at 25-27° C. Infectivity was determined by the rate of cell diameter increase and cell cycle arrest, and change in cell viability every day for 4 to 5 days.

A “Rep-plasmid” was produced in a pFASTBAC™-Dual expression vector (ThermoFisher) comprising both the Rep78 (SEQ ID NO: 131 or 133 of International Patent Application Publication No. WO2019/143385) or Rep68 (SEQ ID NO: 130 of WO2019/143385) and Rep52 (SEQ ID NO: 132 of WO2019/143385) or Rep40 (SEQ ID NO: 129 of WO2019/143385). The Rep-plasmid was transformed into the DH10Bac competent cells (MAX EFFICIENCY® DH10Bac™ Competent Cells (Thermo Fisher) following a protocol provided by the manufacturer. Recombination between the Rep-plasmid and a baculovirus shuttle vector in the DH10Bac cells were induced to generate recombinant bacmids (“Rep-bacmids”). The recombinant bacmids were selected by a positive selection that included-blue-white screening in E. coli (Φ80dlacZΔM15 marker provides α-complementation of the β-galactosidase gene from the bacmid vector) on a bacterial agar plate containing X-gal and IPTG. Isolated white colonies were picked and inoculated in 10 ml of selection media (kanamycin, gentamicin, tetracycline in LB broth). The recombinant bacmids (Rep-bacmids) were isolated from the E. coli and the Rep-bacmids were transfected into Sf9 or Sf21 insect cells to produce infectious baculovirus.

The Sf9 or Sf21 insect cells were cultured in 50 ml of media for 4 days, and infectious recombinant baculovirus (“Rep-baculovirus”) were isolated from the culture. Optionally, the first generation Rep-baculovirus (P0) were amplified by infecting naïve Sf9 or Sf21 insect cells and cultured in 50 to 500 ml of media. Between 3 and 8 days post-infection, the P1 baculovirus particles in the medium were collected either by separating cells by centrifugation or filtration or another fractionation process. The Rep-baculovirus were collected and the infectious activity of the baculovirus was determined. Specifically, four×20 mL Sf9 cell cultures at 2.5×10⁶ cells/mL were treated with P1 baculovirus at the following dilutions, 1/1000, 1/10,000, 1/50,000, 1/100,000, and incubated. Infectivity was determined by the rate of cell diameter increase and cell cycle arrest, and change in cell viability every day for 4 to 5 days.

ceDNA Vector Generation and Characterization

Sf9 insect cell culture media containing either (1) a sample-containing a ceDNA-bacmid or a ceDNA-baculovirus, and (2) Rep-baculovirus described above were then added to a fresh culture of Sf9 cells (2.5E+6 cells/ml, 20 ml) at a ratio of 1:1000 and 1:10,000, respectively. The cells were then cultured at 130 rpm at 25° C. 4-5 days after the co-infection, cell diameter and viability are detected. When cell diameters reached 18-20 nm with a viability of ˜70-80%, the cell cultures were centrifuged, the medium was removed, and the cell pellets were collected. The cell pellets are first resuspended in an adequate volume of aqueous medium, either water or buffer. The ceDNA vector was isolated and purified from the cells using Qiagen MIDI PLUS™ purification protocol (Qiagen, 0.2 mg of cell pellet mass processed per column).

Yields of ceDNA vectors produced and purified from the Sf9 insect cells were initially determined based on UV absorbance at 260 nm. The purified ceDNA vectors can be assessed for proper closed-ended configuration using the electrophoretic methodology described in Example 7.

### Example 2: Preparation of Base Vectors as Template for Double-Stranded DNA Construct

Using a forward primer having the sequence of 5′-TTCCGCTTCCTCGCTCACTG-3′ (SEQ ID NO: 42) and a reverse primer having the sequence of 5′-AGACGTCAGGTGGCACTTTTC-3′ (SEQ ID NO: 43), the backbone of a pUC19 plasmid (New England Biolabs) containing the ampicillin resistance gene and the origin of replication components is amplified. The PCR amplification product is treated with Fast Digest DpnI to eliminate the template and then purified using the Zymo Research DNA Clean and Concentration Kit. Separately, two synthesized gBlock™ DNA fragments are provided (Integrated DNA Technologies). These gBlock™ fragments contained several key components such as the homologous regions with the pUC19 backbone (SEQ ID NO: 44 and SEQ ID NO: 45), the partial ITR sequences (A-A′ and D-D′ stem regions, see e.g., Table 5), 2 Type IIS restriction endonuclease recognition sites, left and right spacer regions (see e.g., Table 2), and a placeholder region having a random nucleotide sequence that serves to separate the restriction endonuclease recognition sites that the transgene expression cassette is cloned into. For example, exemplary base vector Plasmid 11 as shown in FIG. 2 has a placeholder Sp200_CG500 which is a 200 bp spacer with random nucleotide sequences that serve to separate NotI and XbaI recognition sites. The placeholder Sp200_CG500 is substituted with a Factor VIII-expressing transgene expression cassette to produce Construct 1, as described in Example 3.

TABLE 4
Exemplary Homologous Regions with pUC 19 Backbone

| Homologous | |
|---|---|
| Region | 5′ → 3′ Nucleotide Sequence |
| Left | GCGCACATTTCCCCGAAAAGTGCCACCTGACGTCT |
| | (SEQ ID NO: 44) |
| Right | TTCCGCTTCCTCGCTCACTGACTCGCTGCGCTCGG |
| | (SEQ ID NO: 45) |

TABLE 5
Exemplary AAV2 Left and Right Partial ITR Sequences

| Partial ITR | 5′ → 3′ Nucleotide Sequence |
|---|---|
| AAV2 Left | TGAGCGAGCGAGCGCGCAGAGAGGGAGTGGCCAACTCCA |
| | TCACTAGGGGTTCCT (SEQ ID NO: 46) |
| AAV2 Right | AGGAACCCCTAGTGATGGAGTTGGCCACTCCCTCTCTGC |
| | GCGCTCGCTCGCTCA (SEQ ID NO: 47) |

Through homology cloning, the pUC19 backbone that has been amplified and the 2 gBlock™ fragments are ligated and the ligation mixture is used to transform in E. coli and plated on selection medium. Several colonies are selected from the plate and the plasmid DNA purified from the colonies is sequenced for verification. The base vector serves as the template for the preparation of the double-stranded construct, which is described in Example 3.

### Example 3: Preparation of Double-Stranded DNA Construct as Template for Cell-Free Synthesis of ceDNA

### General Overview

The double-stranded DNA construct from which the insert containing the transgene expression cassette is excised from a base vector having a rep origin to enable replication, a multiple cloning site containing recognition sites for at least one Type IIS restriction endonuclease, and at least one gene that enables selection, such as an antibiotic resistance gene. FIG. 2 shows the map of an exemplary base vector, Plasmid 11, that has a placeholder region Sp200_CG500 which is a 200 bp spacer with random nucleotide sequences that serve to separate NotI and XbaI recognition sites, left and right spacers, left and right partial ITRs that facilitate the cell-free synthesis of ceDNA, rep origin, and bla coding sequence that confers ampicillin resistance to the vector. Plasmid 11 serves as a base vector where the spacer Sp200_CG500 is excised and a transgene expression cassette of interest is sub-cloned into the NotI and XbaI sites, to produce Construct 1, whose map is shown in FIG. 3, which has the same genetic elements as its base vector Plasmid 11 with the exception of the placeholder Sp200_CG500 being substituted with a Factor VIII-expressing transgene that can be provided via DNA synthesis, by PCR chain assembly, or by excising such a molecule from a plasmid or other vector. See, e.g., FIG. 11B of International Patent Application Publication No. WO2019/143885.

To prepare a double-stranded DNA construct, abase vector is digested with XbaI and NotI to release the spacer (filler or placeholder) region. The digest is then run on an agarose gel where two bands representing two fragments should be present: the placeholder region and the base vector backbone. The base vector backbone is gel extracted.

A transgene expression cassette of interest is PCR-amplified from a template vector carrying the cassette, for example, using a single primer having the sequence 5′-TGATTAACCCGCCATGCTACTTAT-3′(SEQ ID NO: 48) with Q5 High Fidelity polymerase. The PCR reaction is then treated with fast digest DpnI to eliminate the plasmid template. After that, the PCR reaction is purified using the Zymo Research DNA Clean and Concentration Kit.

Through homology cloning, the base vector backbone and PCR-amplified transgene expression cassette are ligated and the ligation mixture is used to transform in E. coli and plated on selection medium. Several colonies are selected from the plate and the plasmid DNA purified from the colonies is sequenced for verification. The double-stranded construct serves as the template for the cell-free synthesis of ceDNA, which is described in Example 4.

### Example 4: Cell-Free Synthesis of ceDNA

### General Overview

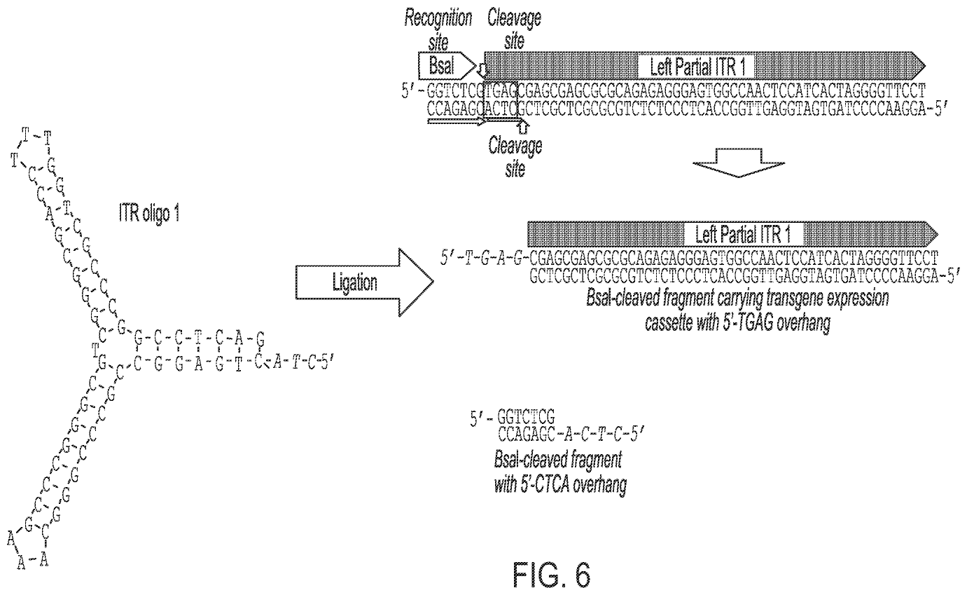

One exemplary cell-free synthetic method of producing a ceDNA vector having symmetrical ITRs with one type of ITR oligonucleotide is illustrated in FIG. 4. Briefly, the transgene expression cassette (in diagonal stripes) is excised from a double-stranded DNA construct using at least one Type IIS restriction endonuclease, e.g., BsaI. This is then followed by ligation (e.g., with a ligase such as T4 ligase or an AAV Rep protein) of the excised insert (containing the transgene expression cassette) into the ITR oligonucleotides (indicated as ITR oligos in FIG. 4), which is a single-stranded oligonucleotide that self-anneals to form an ITR-like three-dimensional configuration. Digestion with the Type IIS restriction endonuclease(s) such as BsaI creates cohesive overhangs at both 5′ and 3′ ends of the excised insert that are compatible with the overhangs of the ITR oligonucleotide. The uniqueness of how Type IIS restriction endonucleases recognize nucleotide sequences and cleave DNA is illustrated in FIG. 5 and FIG. 6 and explained in the detailed description herein. The design of the ITR oligonucleotide and insert overhang sequences drives the high specificity of the ligation process such that the ITR oligonucleotide overhangs and the insert overhangs are compatible with each other. In that way, the ITR oligonucleotides cannot self-ligate to form an ITR oligonucleotide dimer. The fragments from the plasmid backbone after BsaI digestion cannot ligate to the ITR oligonucleotides. Copies of the transgene expression cassette where the BsaI digestion is incomplete are unable to ligate into the ITR oligonucleotides and will be re-cleaved by BsaI. In situations where the excised insert and the plasmid fragments re-ligate into the original construct, the BsaI recognition sites are re-generated and therefore allow the construct to be cleaved again by BsaI. Conversely, the desired ceDNA product having the insert ligated to the ITR oligonucleotide at both ends of the insert are not susceptible to cleavage by BsaI because the BsaI recognition site is not present in the ceDNA. Instead, the desired ceDNA product contains unique junction sequences where the ligations occur (represented by the white circles in FIG. 4) that are present only in the ceDNA product but not in the DNA construct starting material nor the ITR oligonucleotides. Due to the unique activity of Type IIS restriction endonucleases, the generation of unique overhangs on the insert ends and the design of unique overhangs on the ITR oligonucleotides that drive the high specificity of the sub-cloning process, the digestion and ligation can take place in a single reaction vessel without a need to purify the digestion products prior to ligation. After the ligation is complete, the sample is selectively enriched via DNA exonuclease treatment, e.g., T5 exonuclease treatment, that selectively targets and degrades open-ended DNA fragments and intermediates, but not ceDNA which is closed-ended.

Detailed Procedure for Cell-Free Synthesis of ceDNA

Lyophilized ITR oligonucleotides are re-suspended in 1×TE buffer at 100 μM concentration to create a stock, which are diluted 1:10 in Duplex Buffer (30 mM HEPES, pH 7.5; 100 mM potassium acetate) to give a final concentration of 10 μM for use. To induce self-annealing, the diluted ITR oligonucleotides are incubated at 95° C. for 10 min, which are the removed and immediately placed in an iced water bath to rapidly cool.

Next, an enzymatic combined digestion and ligation reaction mixture is set up with a double-stranded construct (e.g., Construct 1 carrying a FVIII-expressing transgene), one or more Type IIS restriction endonuclease (e.g., BsaI), and T4 ligase. Table 6 below provides the recipes for setting up a 800-μL reaction and a 32-mL reaction, where a single ITR oligonucleotide is used.

TABLE 6
800-μL and 32-mL endonuclease digestion
and ligation reaction mixtures

| Reagent | Volume (μL) | Volume (mL) |
|---|---|---|
| Nuclease-Free Water | 20 | 0.8 |
| Double-Stranded Construct | 400 | 16.0 |
| (2 mg/mL) | | |
| 10X T4 DNA Ligase Buffer | 80 | 3.2 |
| BsaI (20 U/μL) | 40 | 1.6 |
| T4 DNA Ligase (2,000 U/μL) | 20 | 0.8 |
| Annealed oligonucleotide (10 μM) | 240 | 9.6 |

The digestion/ligation reaction mixture is incubated at 37° C. for 2.5 h, followed by a heat inactivation of the T4 ligase at 65-75° C. for 30 min. After that, the T5 exonuclease reaction that removes residual open-ended DNA fragments from the construct backbone and unligated ITR oligonucleotides and inserts at 37° C. for 1-1.5 h is set up as follows:

TABLE 7
1000-μL and 40-mL exonuclease digestion reaction mixtures

| Reagent | Volume (μL) | Volume (mL) |
|---|---|---|
| Digestion/Ligation Solution | 800 | 32.0 |
| Nuclease-Free Water | 60 | 2.4 |
| NEBuffer 4 | 100 | 4.0 |
| T5 Exonuclease (10 U/μL) | 40 | 1.6 |

After T5 exonuclease digestion, the reaction mixture is purified using the ZymoPURE Gigaprep Kit according to the manufacturer's instructions. Alternatively or additionally, the reaction mixture is mixed with a diluent buffer (50 mM sodium phosphate, 50 mM EDTA, pH 7.0) and loaded onto a packed DMAE resin column for purification.

### Example 5: Design and Preparation of Self-Annealing ITR Oligonucleotides

As described in Example 3 and as illustrated in FIGS. 4-6, an exemplary cell-free synthetic method of producing a ceDNA vector involves ligating the excised double-stranded insert that contains the transgene expression cassette to 5′ and 3′ ITR oligonucleotides. ITR oligonucleotides can be provided by any method of DNA synthesis (e.g., in vitro DNA synthesis methodologies) and are provided as linear molecules with a free 5′ end and free 3′ end. The ITR oligonucleotides then self-anneal to form secondary base-pairing structures (e.g., stem-loops or hairpins), but the primary structure is a linear single-strand molecule. Furthermore, when the ITR oligonucleotide self-anneals to form the secondary three-dimensional structure, a cohesive overhang is formed at either the 5′ or 3′ end of the oligonucleotide. Table 8 shows exemplary ITR oligonucleotides, which can be ligated to the 5′ and 3′ ends of a double stranded DNA construct as illustrated in FIG. 4.

TABLE 8
Exemplary ITR Oligonucleotides for Cell-Free Synthetic Production of ceDNA

| | | Overhang | | |
|---|---|---|---|---|
| | 5′ → 3′ | formed | | |
| | sequence of | when self- | Oligo- | |
| Synthetic ITR | ITR oligo- | annealed | nucleotide | |
| oligonucleotide | nucleotide | (5′ → 3′) | type | Description |
| ITR oligo 1 | CTCACTGAGGCCGCC | CTCA | 5′- | AAV2 wt-ITR |
| (SEQ ID NO: 1) | CGGGCAAAGCCCGG | | phosphor- | sequence; generates |
| | GCGTCGGGCGACCTT | | ylated | 7-bp stem with 5′- |
| | TGGTCGCCCGGCCTC | | | CTCA overhang. |
| | AG | | | |
| ITR oligo 2 | CTCTCTGAGGCCGCC | CTCT | 5′- | AAV2 wt-ITR |
| (SEQ ID NO: 2) | CGGGCAAAGCCCGG | | phosphor- | sequence; generates |
| | GCGTCGGGCGACCTT | | ylated | 7-bp stem with 5′- |
| | TGGTCGCCCGGCCTC | | | CTCT overhang. |
| | AG | | | |
| ITR oligo 3 | CACTGAGGCCGGGCG | CACT | 5′- | Mutant ITR sequence |
| (SEQ ID NO: 3) | ACCAAAGGTCGCCCG | | phosphor- | with 9-bp deletion; |
| | ACGCCCGGGCGGCCT | | ylated | generates 5-bp stem |
| | C | | | with 5′-CACT |
| | | | | overhang. |
| ITR oligo 4 | CACTGAGGCCGCCCG | CACT | 5′ - | AAV2 wt-ITR |
| (SEQ ID NO: 4) | GGCAAAGCCCGGGC | | phosphor- | sequence; generates |
| | GTCGGGCGACCTTTG | | ylated | 5-bp stem with 5′- |
| | GTCGCCCGGCCTC | | | CACT overhang. |
| ITR oligo 5 | CTCAGGCCGCCCGGG | CTCA | 5′ - | AAV2 wt-ITR |
| (SEQ ID NO: 5) | CAAAGCCCGGGCGTC | | phosphor- | sequence; generates |
| | GGGCGACCTTTGGTC | | ylated | 3-bp stem with 5′- |
| | GCCCGGCC | | | CTCA overhang. |
| ITR oligo 6 | CTCACTGAGGCCGCC | CTC | 15′- | AAV2 wt-ITR |
| (SEQ ID NO: 6) | CGGGCAAAGCCCGG | | phosphor- | sequence; generates |
| | GCGTCGGGCGACCTT | | ylated | 8-bp stem with 5′- |
| | TGGTCGCCCGGCCTC | | | CTC overhang. |
| | AGT | | | |
| ITR oligo 7 | GCTCACTGAGGCCGC | GCT | 5′- | AAV2 wt-ITR |
| (SEQ ID NO: 7) | CCGGGCAAAGCCCG | | phosphor- | sequence; generates |
| | GGCGTCGGGCGACCT | | ylated | 9-bp stem with 5′- |
| | TTGGTCGCCCGGCCT | | | GCT overhang. |
| | CAGTG | | | |
| ITR oligo 8 | CTCAGAGTGAGGTGA | CTCA | 5′- | AAV2 wt-ITR |
| (SEQ ID NO: 8) | GGCCGCCCGGGCAA | | phosphor- | sequence; generates |
| | AGCCCGGGCGTCGGG | | ylated | 14-bp stem with 5′- |
| | CGACCTTTGGTCGCC | | | CTCA overhang. |
| | CGGCCTCACCTCACT | | | |
| | C | | | |

As disclosed herein, the ceDNA vectors prepared using the cell-free synthetic methods disclosed herein can comprise wild-type ITRs (WT-ITRs, see e.g., FIGS. 6A-6B of International Patent Application Publication No. WO2019/143885) or modified ITRs (both symmetrical and asymmetrical, see e.g., FIGS. 7A-7B of WO2019/143385). Modified ITRs can include deletion, insertion, or substitution of one or more nucleotides from the wild-type ITR in the sequences forming B and B′ arm and/or C and C′ arm (see e.g., Table 3 of WO2019/143885). In addition to the ITR oligonucleotides listed in Table 8 above, ITR oligonucleotides fully or partially incorporating any of the wild-type or modified ITR nucleotide sequences listed in Tables 2, 4A, 4B, and 5 of WO2019/143885 in combination with the appropriate overhang sequence can be designed and synthesized. For ITR oligonucleotides that partially incorporate an ITR nucleotide sequence, full ITR sequences are generated in the ceDNA product after ligation of the ITR oligonucleotides to the insert when the partial ITR sequence on the ITR oligonucleotide combines with a corresponding partial ITR sequence found at the 5′ and 3′ ends of the insert.

As part of the synthesis process, one or more restriction endonuclease sites can be introduced into the stem portion of the ITR. FIGS. 6A-7E of WO2019/143885 provide exemplary ITR oligonucleotide sequences and structures, including embodiments where restriction endonuclease sites are incorporated.

To induce the self-annealing of the ITR oligonucleotides to form an ITR-like three-dimensional configuration, the procedure described in Example 2 is used. Alternatively, the oligonucleotides are mixed in equal molar amounts in a suitable buffer: e.g. 100 mM potassium acetate; 30 mM HEPES, pH 7.5) and heated to 94° C. for 2 minutes and gradually cooled. For oligos without significant secondary structure, the cooling step can be as simple as transferring samples from the heat block or water bath to room-temperature. For oligos predicted to have a lot of secondary structure, a more gradual cooling/annealing step is beneficial. This is done by placing the oligo solution in a water bath or heat block and unplugging/turning off the machine. The annealed oligonucleotides can be diluted in a nuclease-free buffer and stored in their double-stranded annealed form at 4° C.

### Stem Length of ITR Oligonucleotides

In addition to the design and formation of cohesive overhangs that drive the specificity of the ligation reaction in the cell-free ceDNA production methods described herein, the inventors have found that the length of the stem region of the folded ITR oligonucleotides (i.e., ITR oligonucleotides having the three-dimensional stem-loop structure) plays an important role towards the folding of the oligonucleotide and ceDNA production. FIG. 7A are schematic diagrams showing a folded ITR oligonucleotide having a 7-bp stem and another folded ITR oligonucleotide having a 3-bp stem. FIG. 7B is an agarose gel electrophoresis image showing the ligation product of 4 different reactions whereby a ˜320 bp insert excised from Plasmid 20 via BsaI digestion was ligated with ITR oligonucleotides having 14-bp, 7-bp, 5-bp, and 3-bp stems, respectively. The ˜320 bp insert excised from Plasmid 20 includes, as shown in the plasmid map of FIG. 7C, the left partial ITR, left spacer, right spacer, and right partial ITR flanked by two BsaI recognition sites. The sizes of the ligation products from the 4 different reactions were expected to be: 480 bp (14-bp stem), 380 bp (7-bp stem), 380 bp (5-bp stem), and 375 bp (3-bp stem). As shown in FIG. 7B, the reactions using ITR oligonucleotides having 14-bp, 7-bp, and 5-bp stems yielded ligation products having the expected sizes as set forth above, but the reaction using ITR oligonucleotides having 3-bp stems did not yield any ligation product.

### Example 6: Proof of Concept of Cell-Free Synthesis of ceDNA Including ceDNA Having Asymmetric ITRs

The cell-free synthetic methods of ceDNA described herein are compatible with different Type IIS restriction endonucleases and different ITR oligonucleotide overhangs. Specifically, the base vectors and ITR oligonucleotides can be designed and modified in such a manner in order support ceDNA synthesis with different Type IIS restriction endonucleases (i.e., by incorporating the appropriate recognition sites), with different insert overhangs, with different ITR oligonucleotide overhangs, and with different ITR oligonucleotides. Table 9 below provides a list of exemplary base vectors, their corresponding Type IIS restriction endonucleases, ITR oligonucleotides, and ITR oligonucleotide overhangs.

TABLE 9
Exemplary Base Vectors and Corresponding Type IIS Restriction
Endonucleases, ITR Oligonucleotides, and
ITR Oligonucleotide Overhangs

| | Type IIS | | Overhang formed |
|---|---|---|---|
| | restriction | ITR | when self-annealed |
| Base vector | endonuclease | oligonucleotide | (5′ → 3′) |
| Plasmid 17 | BsaI | ITR oligo 2 | CTCT |
| (SEQ ID NO: 9) | | | |
| Plasmid 20 | BsaI | ITR oligo 1 | CTCA |
| (SEQ ID NO: 10) | | | |
| Plasmid 18 | BsaI | ITR oligo 3 | CACT |
| (SEQ ID NO: 11) | | | |
| Plasmid 15 | BsaI | ITR oligo 1 | CTCA |
| (SEQ ID NO: 12) | | ITR oligo 3 | CACT |
| Plasmid 1 | Esp3I | ITR oligo 1 | CTCA |
| (SEQ ID NO: 13) | | | |
| Plasmid 11 | BbsI | ITR oligo 1 | CTCA |
| (SEQ ID NO: 14) | | | |
| Plasmid 8 | SapI | ITR oligo 6 | CTC |
| (SEQ ID NO: 15) | | | |
| Plasmid 7 | SapI | ITR oligo 7 | GCT |
| (SEQ ID NO: 16) | | | |
| Plasmid 6 | SapI | ITR oligo 6 | CTC |
| (SEQ ID NO: 17) | | ITR oligo 7 | GCT |

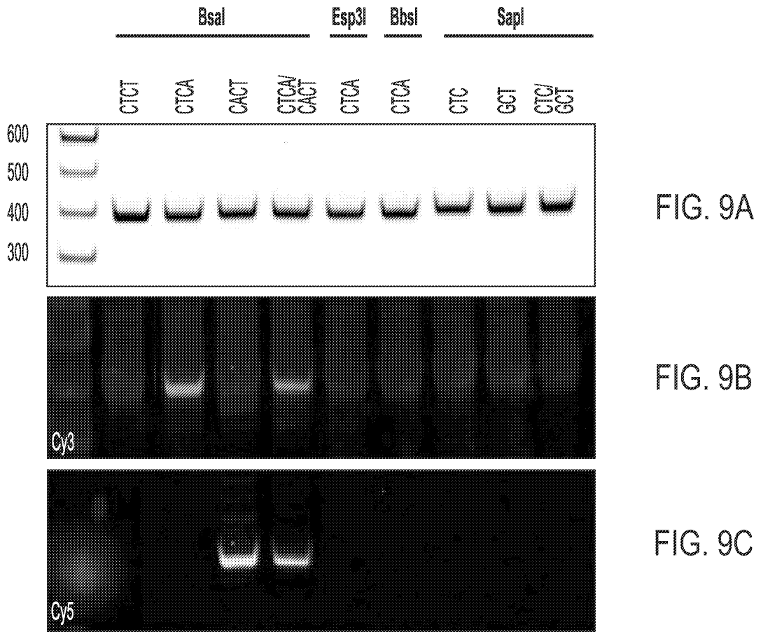

Proof-of-concept experiments were conducted whereby the base vectors listed in Table 9 were excised with their respective Type IIS restriction endonuclease to obtain an insert (carrying no transgene expression cassette) having overhangs at the 5′ and 3′ ends that are compatible with the overhangs of their respective ITR oligonucleotide(s) as set forth in Table 9, and are subsequently ligated with these ITR oligonucleotide(s). The agarose gel image in FIG. 9A indicates that the Type IIS restriction endonuclease digestion and subsequent ligation with ITR oligonucleotides were both successful for all of the reactions set up as outlined in Table 9 as the DNA bands shown in the gel had the expected size of ˜400 bp.

Notably, these proof-of-concept studies also proved the viability of cell-free synthesis of ceDNA having asymmetric ITRs. For example, base vector Plasmid 15 was designed to be digested with BsaI to produce orthogonal overhangs at the 5′ and 3′ ends of the insert, one of which would be compatible with the 5′-CTCA-3′ overhang presented by ITR oligo 1 while the other would be compatible with the 5′-CACT-3′ overhang presented by ITR oligo 3. In other words, the design of Plasmid 15, ITR oligo 1, and ITR oligo 3 control the directionality and specificity of the ligation reaction. This allows ceDNA having asymmetric ITRs to be prepared. Indeed, as noted in Table 9, ITR oligo 3 contains a mutant ITR sequence with a 9-bp deletion. Labelling ITR oligo 1 with Cy3 and ITR oligo 3 with Cy5 allowed confirmation of the ligation specificity at both the 5′ and 3′ ends of the insert (see FIGS. 9B and 9C). Asymmetric ITRs and ceDNA having asymmetric ITRs are well-defined herein and also in International Patent Application Publication Nos. WO2019/143885 and WO2017/152149. With the appropriate design and modification of the base vectors and ITR oligonucleotides, ceDNA having asymmetric ITRs and whereby at least one of the ITRs has modifications located in the A-A′ stem region, the B-B′ loop, the C-C′ loop, and/or D-D′ stem region can be prepared using the cell-free synthetic methods provided herein. Using the AAV serotype 2 ITR as an example only, FIG. 10 shows the locations of A-A′ and D-D′ stem regions as well as the B-B′ and C-C′ loops in an ITR having a stem-loop structure.

### Example 7: Purifying and Confirming ceDNA Production, Closed-Endedness, and Purity by Electrophoresis

Any of the ceDNA vector products prepared using the cell-free synthetic methods described herein can be purified, e.g., to remove impurities, unused components, or byproducts using methods commonly known by a skilled artisan; and/or can be analyzed to confirm that DNA vector produced, (in this instance, a ceDNA vector) is the desired molecule. An exemplary method for purification of the DNA vector, e.g., ceDNA is using Qiagen Midi Plus purification protocol (Qiagen) and/or by gel purification.

### Non-Denaturing Agarose Gel Electrophoresis

ceDNA vector production can be assessed, for example, by agarose gel electrophoresis under native or denaturing conditions. Non-denaturing gel analysis confirms the success of the restriction endonuclease digestion/ligation reaction and the exonuclease digestion reaction of the cell-free ceDNA production described herein. FIG. 11 is an agarose gel image showing 3 samples in Lanes 1, 2, and 3 involving cell-free synthesis of FVIII-ceDNA using Construct 1 as the double-stranded DNA construct supplying a Factor VIII-expressing transgene expression cassette. The sample in Lane 1 showed a single thick band above the 6 kb band of the ladder that was indicative of the 8190-bp undigested Plasmid 11 (map shown in FIG. 6). The sample in Lane 2 is the BsaI digestion and ligation reaction showing three bands as expected: The intense band aligned with the 6-kb band of the ladder and corresponded with the 6083-bp insert containing the transgene expression cassette and ITRs; the two other bands corresponded to the 1161-bp and 946-bp fragments from the plasmid backbone. The sample in Lane 3 showed that after the exonuclease digestion, only the ceDNA ligation product (˜6.3 kb) remained as expected because, contrary to the open-ended plasmid backbone fragments, the closed-ended ligation product was not susceptible to the exonuclease digestion.

### Denaturing Agarose Gel Electrophoresis

Denaturing gel analysis can also confirm the success of the restriction endonuclease digestion/ligation reaction and the exonuclease digestion reaction of the cell-free ceDNA production described herein. Using the cell-free synthesis of FVIII-ceDNA from Construct 1 as an illustrative example, following electrophoresis on a denaturing gel (e.g., containing urea), a linear and open-ended double-stranded DNA would have a predicted size of ˜6.3 kb, which was confirmed by and corresponded to Band C shown in Lane 2 containing the uncut linear PCR control sample (see FIG. 12).

The denaturing conditions of the agarose gel electrophoresis separate the two complementary DNA strands in a DNA molecule. Therefore, the desired FVIII-ceDNA ligation product, which was a closed-ended double-stranded DNA, was predicted to resolve at ˜12.6 kb as the two DNA strands which were linked had been unfolded and would be twice the length of the single strands (˜6.3 kb). Moreover, the FVIII-ceDNA ligation product may contain one or more nicks or gaps. The basis for the presence of nicks and gaps in ceDNA is described in detail in International Patent Application Publication No. WO2021/011840. Additionally, in the context of cell-free and enzyme-based synthesis of ceDNA that is contemplated in this disclosure (i.e., use of restriction endonucleases for digestion and a ligase for ligation), the presence of nicks and gaps in ceDNA could be attributed to the following reasons: (i) The ligase was not able to fully ligate the ITR oligonucleotides and the insert; (ii) restriction endonuclease slippage; and (iii) The nicks and gaps were contained in the starting materials such as the double-stranded construct carrying the transgene expression cassette (due to mechanical stress during plasmid handling such as multiple freeze-thaw cycles vortexing the construct).

Accordingly, denatured unnicked FVIII-ceDNA that was super-coiled was expected to run at a higher position on the agarose gel as compared to the nicked FVIII-ceDNA that was in a nicked form. Corroboratively, as shown in Lane 1 of FIG. 12, the denaturing gel analysis indicated that a majority of the DNA resolved at a size that was consistent with the closed-ended form at ˜15,000 kb (Band A); whereas a small amount of a single-nicked form of FVIII-ceDNA resolved at just below the Band A as Band B.

Furthermore, FVIII-ceDNA which contained a known BglII restriction site was digested with the enzyme and analyzed on the denaturing gel. A linear and open-ended ˜6.3-kb double-stranded DNA was expected to separate into two single-stranded DNA molecules that were sized at ˜3.8 kb and another two single-stranded DNA molecules that are sized at ˜2.5 kb. In contrast, the desired FVIII-ceDNA ligation product, which was a closed-ended ˜12.6-kb double-stranded DNA, was predicted to resolve as two bands: a ˜7.6-kb band (i.e., 2 joined ˜3.8-kb single-stranded DNA) and a ˜5-kb band (i.e., 2 joined ˜2.5-kb single-stranded DNA). Lane 3 in FIG. 12 shows the BglII-digested FVIII-ceDNA sample. Specifically, Lane 3 showed strong bands D and E that respectively corresponded with the ˜7.6-kb and ˜5-kb bands resulting from the BgIII-digested closed-ended double-stranded DNA, thereby indicating once again that a majority of the DNA present in the sample was the closed-ended form. Faint bands could also be seen in Lane 3, which corresponded with the ˜3.8-kb and ˜2.5-kb BglII-digested fragments from the open-ended form, thereby indicating a trace amount of open-ended DNA being present in the purified drug substance sample.

### Example 8: Chromatography Purification of ceDNA

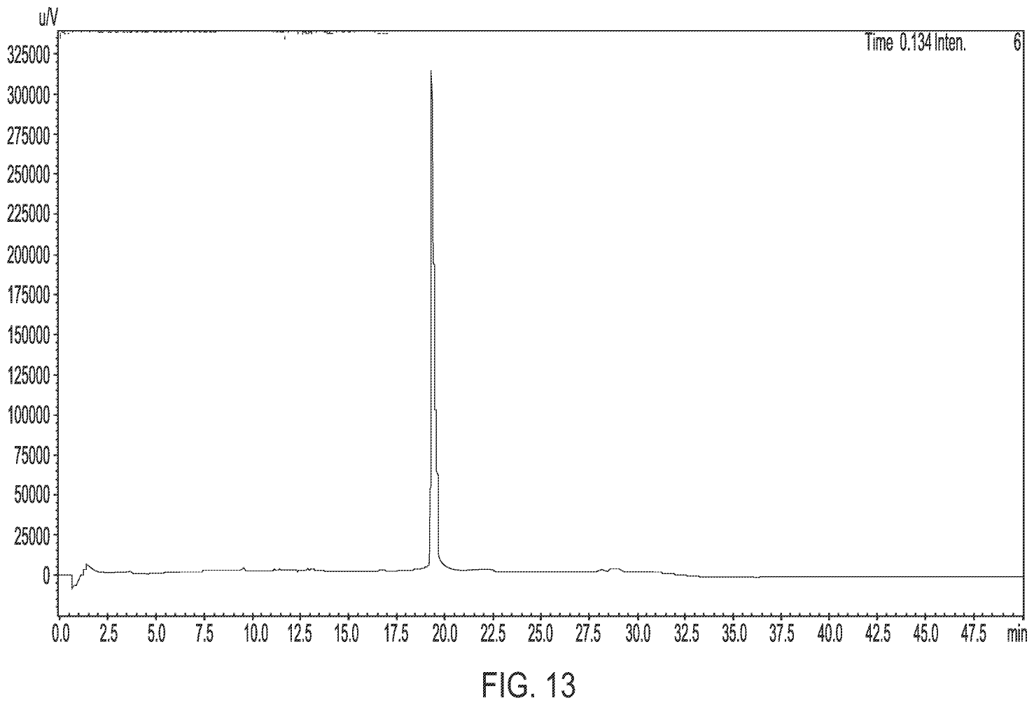

Next, ion-exchange chromatography (IEX) experiments were performed to further purify and to assess the purity of ceDNA generated using the cell-free synthesis methods described herein. Briefly, a weak anion-exchange chromatography method was utilized to separate the size variant species of the FVIII-ceDNA drug substance sample based on their net negative charge, which would be directly proportional to the length of their phosphate backbone, allowed for a sized-based separate means of the analyte net charge. A monolith resin with tertiary amine functional groups was used as the stationary phase and a gradient of increasing potassium chloride salt concentration was used to selectively elute the analytes and detect by an online UV detector at 260 nm. The FVIII-ceDNA drug substance sample eluted as a single peak, as shown in the chromatogram of FIG. 13, thereby indicating that the drug substance existed as a single molecular species, i.e., >99% monomer. This suggests that the cell-free synthesis methods described herein are superior to conventional cell-based (e.g., insect cells such as Sf9) production methods in terms of purity. Cell-based production of gene therapy vectors, including AAV vectors, have been known to face challenges that include contamination with multiple categories of sub-monomeric or sub-genomic particles with either snapback genomes or vector genomes with deletions in the mid-regions (see Zhang et al., (2020) bioRxiv 2020.08.01.230755).

### Example 9: Confirming ceDNA Production by DNA Sequencing Analysis

Any of the ceDNA vector products prepared using the cell-free synthetic methods described herein can also be confirmed by DNA sequencing analysis. Specifically, the ceDNA vector product was predicted to contain a sequence at each of the ligation junctions at both ends of the vector. These junction sequences are not present in either the double-stranded DNA construct or the ITR oligonucleotides, but are unique to the ceDNA product. FIG. 14 shows the DNA sequence analysis of FVIII-ceDNA, which is prepared by excising an insert carrying a Factor VIII-expressing transgene expression cassette from Construct 1 and ligating the insert with ITR oligo 1 at both ends of the insert.

Attention was drawn to the sequence regions encompassing the 5′ and 3′ (or left and right) ligation junctions, i.e., (i) Left Region: nucleotide 78 to nucleotide 151 of FVIII-ceDNA that is at the 5′ end of FVIII-ceDNA; and (ii) Right Region: nucleotide 6107 to nucleotide 619 of FVIII-ceDNA that is at the 3′ end of FVIII-ceDNA. The FVIII-ceDNA nucleotide sequence in the Left Region was compared to Construct 1 DNA Sequencing Data (Left)—Sample 1 and the known sequence of ITR oligo 1 (SEQ ID NO: 1) to obtain a unique junction sequence of 5′-TGAGCGAGCGAGCGCG-3′ (Left Junction 1 in FIG. 14). Additionally, the FVIII-ceDNA nucleotide sequence in the Left Region was compared to Construct 1 DNA Sequencing Data (Left)—Sample 2 (derived from a different sequencing sample) and the known sequence of ITR oligo 1 (SEQ ID NO: 1) to obtain a shorter unique junction sequence of 5′-TGAGCGAGCGAG-3′ (Left Junction 2 in FIG. 14, SEQ ID NO: 41). In a similar manner, the FVIII-ceDNA nucleotide sequence in the Right Region was compared to Construct 1 DNA Sequencing Data (Right) and the known sequence of ITR oligo 1 (SEQ ID NO: 1) to obtain a unique junction sequence of 5′-CGCTC-3′ (Right Junction in FIG. 14).

### Example 10: Scalability of Cell-Free Synthesis of ceDNA

Advantageously, the cell-free synthetic methods provided herein are readily scalable from, as demonstrated in Example 4, ˜1-mL reactions (e.g., in single Eppendorf tubes or 96-well plates) to ˜50 mL reactions. The inventors have demonstrated that these cell-free synthetic methods are further and directly scalable to high-throughput, automated bioreactor systems for large-scale manufacturing of ceDNA. The agarose gel image in FIG. 15 shows the ceDNA products prepared in a 1-mL eppendorf tube (exonuclease digestion reaction volume=1 mL) and in a Ambr® bioreactor system (exonuclease digestion reaction volume=137.5 mL), each using either 2 mg/mL or 0.4 mg/mL of Construct 1 as the double-stranded construct base material. Comparable yield and purity levels were obtained in the reactions at both scales.

### Example 11: Protein Expression from Synthetically Prepared ceDNA in Mice

In vivo protein expression of a Factor VIII (FVIII)-expressing transgene from the synthetically produced ceDNA vectors described above was assessed in vivo in mice in comparison with corresponding ceDNA vectors prepared using the traditional Sf9 cell-based process, via hydrodynamic tail vein injection and also via intravenous administration of lipid nanoparticle formulations containing the ceDNA vectors.

### Hydrodynamic Tail Vein Injection of FVIII-ceDNA Vectors

A well-known method of introducing nucleic acid to the liver in rodents is by hydrodynamic tail vein injection. In this system, the pressurized injection in a large volume of non-encapsulated nucleic acid results in a transient increase in cell permeability and delivery directly into tissues and cells. This provides an experimental mechanism to bypass many of the host immune systems, such as macrophage delivery, providing the opportunity to observe delivery and expression in the absence of such activity.

The hydrodynamic tail vein injection of ceDNA study was designed as follows in Table 10. The objective of this study was to evaluate FVIII expression after hydrodynamic delivery of ceDNA produced by the synthetic and cell-based processes.

TABLE 10
Hydrodynamic tail vein injection study design

| | Animals | | | | Dosing | Terminal |
|---|---|---|---|---|---|---|
| | per | | Dose Level | Dose | Regimen | Time |
| Group | Group | Treatment | (μg/animal) | Volume | (ROA) | Point |
| A | 2 | PBS | NA | 90- | Once on | Day 1 |
| B | 4 | Synthetic | 0.002 | 100 | Day 0 | |
| C | 4 | FVIII- | 0.001 | ml/kg | by IV | |
| D | 4 | ceDNA | 0.05 | (set | Hydro- | |
| E | 4 | | 0.25 | volume | dynamic | |
| F | 4 | Cell-Based | 0.002 | based | | |
| G | 4 | FVIII- | 0.001 | on the | | |
| H | 4 | ceDNA | 0.05 | smallest | | |
| I | 4 | (Lot 1) | 0.25 | animal | | |
| J | 4 | Cell-Based | 0.002 | in each | | |
| K | 4 | FVIII- | 0.001 | dose | | |
| L | 4 | ceDNA | 0.05 | group- | | |
| M | 4 | (Lot 2) | 0.25 | rounded | | |
| | | | | to the | | |
| | | | | nearest | | |
| | | | | 0.1 mL) | | |

IV = intravenous;
ROA = route of administration

50 approximately 4-week-old male CD-1 mice were given a single hydrodynamic tail vein intravenous administration of 0.002 μg/animal, or 0.01 μg/animal, 0.05 μg/animal, 0.25 μg/animal in a 90-100 mL/kg volume of (i) Synthetic FVIII-ceDNA which is ceDNA carrying a FVIII-expressing transgene cassette that was prepared using the cell-free synthetic methods described herein; (ii) a first lot of the FVIII-ceDNA with the same a FVIII-expressing transgene cassette that was prepared using the traditional cell-based method using Sf9 insect cells (Lot 1 contains >49% of the monomeric species of ceDNA); and (iii) a second lot of the same FVIII-ceDNA prepared using Sf9 cells (Lot 2 contains ?72% of the monomeric species of ceDNA). Mice were assessed at 60-120 min on Day 0 post-injection, at the end of Day 0, and on Day 1 prior to necropsy. Body weights of mice were also recorded on Days 0 and 1. Mice were then euthanized 24 hours post-injection (±5%) and the whole blood was used to provide the plasma for evaluating plasma concentration (IU/mL) of FVIII.

FIG. 16A is graph showing the plasma concentration of FVIII when the mice were administered with increasing dose levels of FVIII-ceDNA (synthetic or cell-based). All ceDNA samples, whether synthetically prepared or prepared in Sf9 cell culture, exhibited dose responsiveness whereby the measured FVIII plasma concentration steadily increased as the administered dose levels increased from 0.002 μg/animal to 0.25 μg/animal. In this hydrodynamic tail vein injection study, it can be seen that the FVIII expression level of the synthetically prepared ceDNA was equivalent to the expression levels of the Sf9-produced ceDNA. The expression level of Lot 2 Sf9-produced ceDNA were slightly higher than Lot 1, which was not unexpected because Lot 2 contained a higher concentration of the monomeric species of ceDNA than Lot 1, and was therefore of higher purity.

### Intravenous Injection of FVIII-ceDNA LNP Formulations

The objective of this study was to determine FVIII expression and activity after intravenous delivery of ceDNA formulated as LNP compositions. The study design was as follows:

TABLE 11
Intravenous administration of ceDNA LNP formulations study design

| | Animals | Treatment | Dose | | Dosing | Terminal |
|---|---|---|---|---|---|---|
| | per | (Drug | Level (μg/ | Dose | Regimen | Time |
| Group | Group | Substance) | animal) | Volume | (ROA) | Point |
| A | 3 | PBS | NA | 5 | Once on | Day 42 |
| B | 7 | Synthetic | 2.0 | mL/kg | Day 0 | |
| | | FVIII- | | | by IV | |
| | | ceDNA | | | | |
| C | 7 | Cell-Based | 2.0 | | | |
| | | FVIII- | | | | |
| | | ceDNA | | | | |

IV = intravenous;
ROA = route of administration

17 approximately 5-week-old male Rag2 mice (which are knockout mice producing no mature T cells or B cells) were given a single intravenous administration of 2.0 μg/animal in a 5 mL/kg volume of LNP compositions carrying: (i) Synthetic FVIII-ceDNA which is ceDNA carrying a FVIII-expressing transgene cassette that was prepared using the cell-free synthetic methods described herein; and (ii) the FVIII-ceDNA with the same a FVIII-expressing transgene cassette that was prepared using the traditional cell-based method using Sf9 insect cells. The LNP compositions were prepared using the methods described in International Patent Application No. PCT/US2021/042033, filed Jul. 16, 2021. The LNP composition of the ionizable lipid, cholesterol, pegylated lipid, etc. and concentrations thereof were kept the same between the formulation encapsulating the synthetic FVIII-ceDNA and the formulation encapsulating the Sf9-produced.

FIG. 16B is graph showing the plasma concentration of FVIII when the mice were administered with 2.0 μg/animal of FVIII-ceDNA formulated as LNP compositions (synthetic or cell-based). The FVIII expression levels in mice treated with synthetic FVIII-ceDNA were about twice higher than the expression levels in mice treated with Sf9-produced FVIII ceDNA and these high expression levels were maintained throughout the study period that lasted for 42 days.

### Example 12: DNA Template Amplification at a High Scale

As an alternative to DNA template scaling using E. coli fermentation for plasmid production, a new approach was developed for more rapid and cost-effective plasmid or DNA production using cell-free DNA amplification methods. This cell-free method involves rolling circle and multiple strand displacement (MSD) amplification of DNA plasmid template by >1000 fold and subsequent conversion of the resultant products into ceDNA molecules using type II endonuclease, ligase, ITR oligos, and exonuclease enzymes.

Phi29 polymerase derived from Bacillus subtilis phage is a highly processive enzyme which operates isothermally due to its native strand displacement capabilities. Phi29 polymerase exhibits as a very low error rate of 10⁻⁶-10⁻⁷ due to its 3′ to 5′ exonuclease proofreading activity. Due to its high processivity and the isothermal properties, Phi29 provides great advantages for the scalable production of DNA, particularly when compared to PCR methods which require high temperature thermocycling. Additionally, mutant versions of Phi29 have also provided improved thermostability and enhanced processivity which allow for production intensification and process durability. For these reasons, the phi29 polymerase was employed in the present method involving rolling circle and multiple displacement amplification of template DNA plasmid containing a ceDNA insert (e.g., expression cassette).

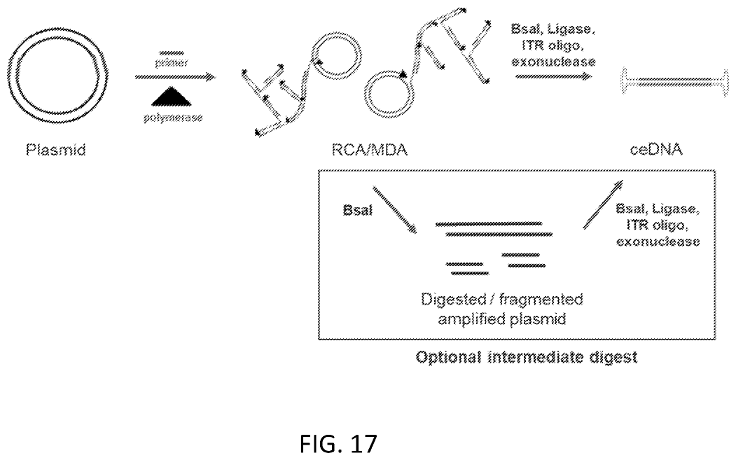

Method development included characterizing the amplification reaction to drive production of high-quality amplified plasmid DNA that was at least equivalent, or superior, to E. coli produced plasmid, and ensuring ease in subsequent converting reaction to ceDNA. To characterize the amplification part of the reaction, an intermediate BsaI digest of the amplified plasmid was applied for evaluating plasmid quality based on DNA banding profiles. The digested material was then capable of being converted into ceDNA by treatment with ligase, ITR oligos, and exonuclease. As a more simple alternative, the amplified plasmid was directly treated with BsaI, ligase, ITR oligos, and exonuclease to generate ceDNA (FIG. 17).

Briefly, amplification was performed using an engineered, thermostable EquiPhi29™ enzyme which typically functions at temperatures of 42-45° C. for rapid amplification kinetics. Forward and reverse primers were designed to target a conserved region in the plasmid backbone near the promoter site of the ampicillin resistance gene. Using a range of different primer concentrations during the annealing step and the amplification reaction yielded high product purity and multiple orders of magnitude increases in DNA amplification.

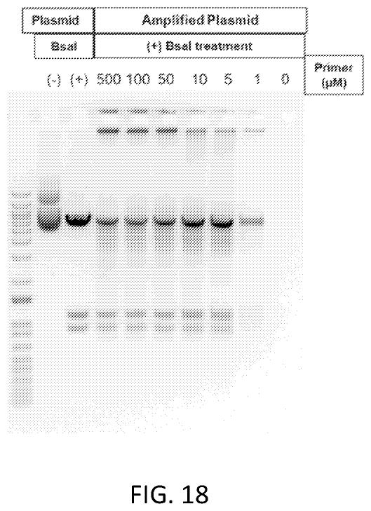

To assess product quality of the amplified plasmid, the crude multiple displacement amplified product was treated with BsaI and compared directly with the E. coli produced plasmid template using agarose gel DNA banding analysis. It was found that the primer concentration had strong effects on digestibility (FIG. 18). For example, for primer concentrations greater than 50 uM, a residual high molecular weight product was consistently observed in the well which was suspected to be DNA that was either inaccessible to enzyme digestion or a large single stranded byproduct (FIG. 18). It was found that a low primer annealing concentration, e.g., between 10-50 uM resulted high amplification levels while also ensuring product quality.

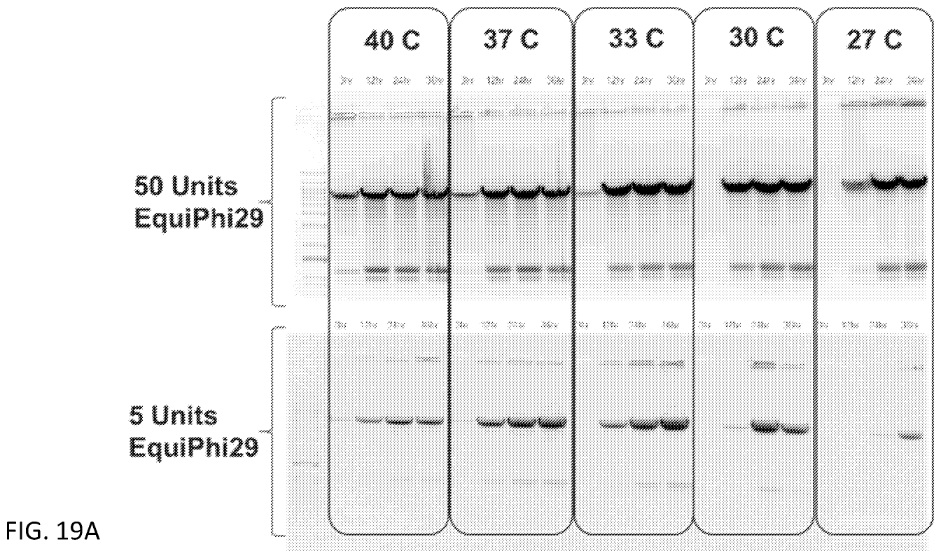

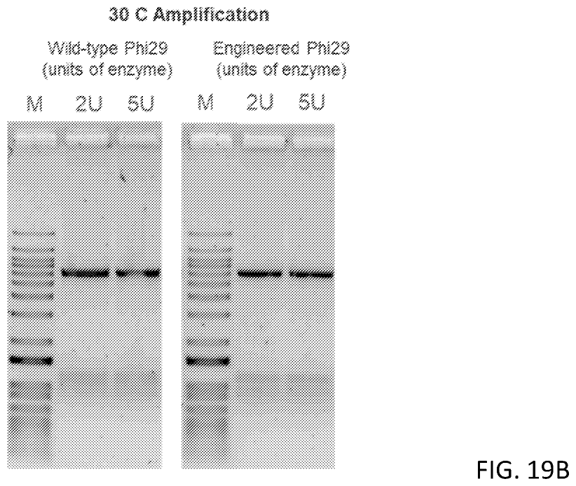

To characterize and enhance the plasmid amplification reaction further, the EquiPhi29™ enzyme quantity and amplification reaction temperature were also evaluated. As shown in FIGS. 19A and 19B, amplification reaction kinetics were heavily influenced by reaction temperature and polymerase amounts. Higher reaction temperatures drove faster kinetics and substantial product generation within 3 hours. However, at the lower temperatures, significantly greater product yields were achieved with increasingly longer reaction times (FIG. 19A). Additionally, at a reduced concentration of polymerase, the kinetics of product generation were slowed. However, it was noted that lowering the reaction temperature and reducing phi29 amounts, also resulted in enhanced product quality as shown in FIG. 19A, particularly with reduced band smearing, indicative of byproduct production, and high intensity of expected plasmid DNA banding. An analysis of amplified plasmid DNA banding demonstrated a very similar product quality profile for wild-type and engineered Phi29 polymerase enzyme (EquiPhi29™), demonstrating direct applicability, but at a yield roughly 10-fold less than that achieved with the engineered Phi29 (FIG. 19B).

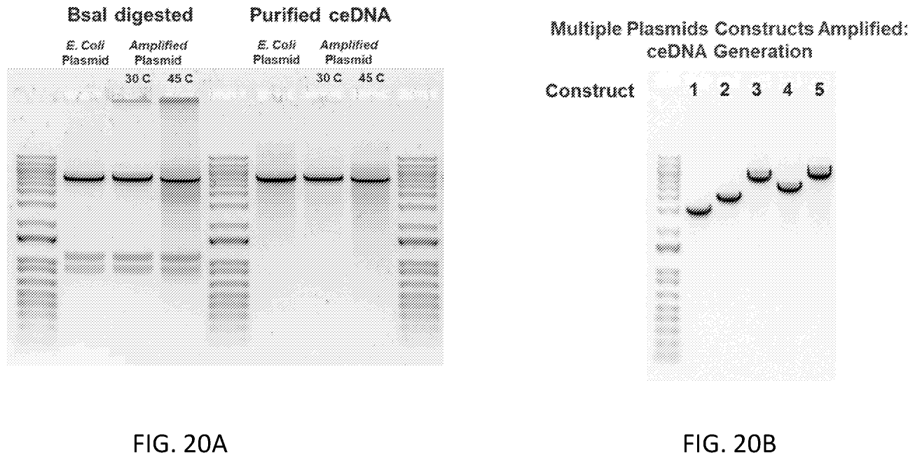

The amplified plasmid was then further analyzed for enzymatic conversion into ceDNA product. The evaluation included using an E. coli derived plasmid and MDA plasmid products generated using the same E. coli derived plasmid template based on the 30° C. and 45° C. amplification reactions. As shown in FIG. 20A, the BsaI digested plasmid profile showed distinct plasmid banding for all samples, while the amplified reaction products contained a high molecular weight species in the wells. The 45° C. process also contained a high level of byproducts resulted in additional band smearing. When the plasmid samples were treated with BsaI, ligase, ITR oligos, and exonuclease, all produced similar quality of ceDNA, demonstrating the robustness of the enzymatic ceDNA assembly process. Furthermore, to evaluate the applicability of the enzymatic plasmid amplification and ceDNA assembly method, 5 different plasmid constructs were amplified and enzymatically converted into ceDNA as described herein. The ceDNA construct sizes (ranging from 2.9kb-6.2kb) and DNA banding quality profiles were confirmed in agarose gel as shown in FIG. 20B.

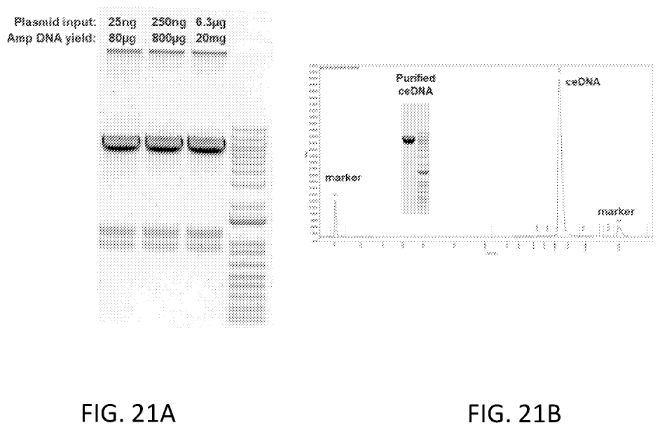

To evaluate scalability of the enzymatic amplification and enzymatic assembly method, the reaction mixture was prepared at different volumes (100 μl, 1 mL, and 25 mL reaction volumes) and evaluated for yield and consistency of amplified plasmid quality. As shown in FIG. 21A, all amplified plasmid materials demonstrated high product quality and increased amplification levels (e.g., approximately 0.8 μg/μL crude yield for all volumes, or about a 3000-fold amplification of input template DNA) for all tested reaction volume scales. To further demonstrate scalability of the entire ceDNA generation method, the amplicon materials were enzymatically converted into ceDNA using the method described herein. The resultant ceDNA was purified using anion exchange columns and the purity was determined using an Agilent Fragment Analyzer. It was shown that the present amplification and subsequent conversion method resulted in successful production of remarkably pure, synthetically made, closed-ended DNA (FIG. 21B, showing large ceDNA peak and lack of other DNA byproducts).

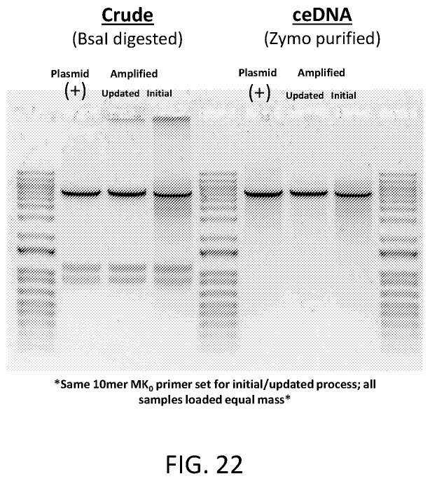

Finally, the enzymatic amplification and enzymatic assembly method was compared to the conventional reaction conditions. FIG. 22 depicts agarose gel analysis comparing plasmid amplification using different amounts of polymerase enzyme, reaction temperatures, and reaction time lengths. As shown, the process described herein (“updated process”: 0.25 ng/μl plasmid template, 25 μM annealing primer, 30° C., 0.05 U/μl EquiPhi29™, 4 mM dNTPs, 18-26 hours) allowed for the use of a 10-fold lower concentration of enzyme (reducing costs and potential supply constraints), while resulting in a substantially enhanced product quality, showing reduced smearing and reduced high-molecular weight species (non-accessible to enzymatic manipulation), as compared to conventional reaction conditions (“initial process”: 0.25 ng/μl plasmid template, 10 μM annealing primer, 45° C., 0.5 U/μl EquiPhi29™, 5 mM dNTPs, 3 hours).

The results disclosed herein demonstrated a scalable, robust, cell-free, enzymatic method to generate large quantities of completely synthetic closed-ended DNA molecules.

### REFERENCES

All publications and references, including but not limited to patents, published and unpublished patent applications, cited in this specification and Examples herein are each incorporated by reference in its entirety, as if each individual publication or reference were specifically and individually indicated to be incorporated by reference herein as being fully set forth. Any patent application to which this application claims priority is also incorporated by reference herein in the manner described above for publications and references.

What is claimed is:

1. A method of producing a closed-ended DNA (ceDNA) vector, the method comprising:
(a) contacting a double-stranded DNA construct having a sense strand and an antisense strand with at least a first restriction endonuclease and at least a second restriction endonuclease, wherein:
the construct comprises:
a transgene expression cassette,
a first non-palindromic restriction endonuclease recognition site and a corresponding first cleavage site upstream of the transgene expression cassette, and
a second non-palindromic restriction endonuclease recognition site and a corresponding second cleavage site downstream of the transgene expression cassette; and
wherein the first restriction endonuclease is capable of cleaving the double-stranded DNA construct at the first cleavage site, and wherein the second restriction endonuclease is capable of cleaving the double-stranded DNA construct at the second cleavage site, and wherein contacting the double-stranded DNA construct with the first restriction endonuclease and the second restriction endonuclease releases an insert having a first end comprising a first single-stranded overhang and a second end comprising a second single-stranded overhang;
(b) ligating the first end to a first oligonucleotide comprising one or more hairpin structures; and
(c) ligating the second end to a second oligonucleotide comprising one or more hairpin structures;
thereby producing a ceDNA vector.

2. The method of claim 1, wherein the first oligonucleotide comprises an inverted terminal repeat (ITR).

3. The method of claim 1 or claim 2, wherein the second oligonucleotide comprises an ITR.

4. The method of any one of claims 1-3, wherein the first oligonucleotide and the second oligonucleotide are different.

5. The method of any one of claims 1-3, wherein the first oligonucleotide and the second oligonucleotide are the same.

6. The method of any one of claims 1-5 wherein the first restriction endonuclease and the second restriction endonuclease are different restriction endonucleases.

7. The method of any one of claims 1-3 or 5, wherein the first restriction endonuclease and the second restriction endonuclease are the same restriction endonuclease.

8. The method of any one of claims 1-7, wherein each of the oligonucleotides independently includes 1, 2, 3, 4, or more stem-loop regions.

9. The method of any one of claims 1-8, wherein each of the oligonucleotides independently includes 2 or 3 stem-loop regions.

10. The method of any one of claims 1-9, wherein the first non-palindromic restriction endonuclease recognition site and the corresponding first cleavage site are separate and distinct sites from each other, and wherein both sites are located upstream of the transgene expression cassette.

11. The method of any one of claims 1-10, wherein the first cleavage site is about 1 to about 22 nucleotides away from the first non-palindromic restriction endonuclease recognition site in at least one of the sense strand and the antisense strand of the construct.

12. The method of claim 11, wherein the first cleavage site is about 1 to about 8 nucleotides away from the first non-palindromic restriction endonuclease recognition site in at least one of the sense strand and the antisense strand of the construct.

13. The method of any one of claims 1-12, wherein the second non-palindromic restriction endonuclease recognition site and the corresponding second cleavage site are separate and distinct sites from each other, and wherein both sites are located downstream of the expression cassette.

14. The method of any one of claims 1-13, wherein the second cleavage site is about 1 to about 22 nucleotides away of the second non-palindromic restriction endonuclease recognition site in at least one of the sense strand and the antisense strand of the construct.

15. The method of claim 14, wherein the second cleavage site is about 1 to about 8 nucleotides away from the second non-palindromic restriction endonuclease recognition site in at least one of the sense and the antisense strand of the construct.

16. The method of any one of claims 1-15, wherein the first non-palindromic restriction endonuclease recognition site and the second non-palindromic restriction endonuclease recognition site are each a double-stranded polynucleotide having different 5′ to 3′ nucleotide sequences in each of the sense strand and the antisense strand.

17. The method of any one of claims 1-16, wherein one or both of the single-stranded overhangs at the ends of the insert are 5′ overhangs.

18. The method of any one of claims 1-17, wherein one or both of the single-stranded overhangs at the ends of the insert are 3′ overhangs.

19. The method of any one of claims 1-18, wherein the first oligonucleotide comprising one or more hairpin structures and the second oligonucleotide comprising one or more hairpin structures are each a single-stranded oligonucleotide that self-anneals to form a three-dimensional configuration.

20. The method of claim 19, wherein the three-dimensional configuration is a T- or Y-shaped stem-loop structure.

21. The method of any one of claims 1-20, wherein the first oligonucleotide comprising one or more hairpin structures and the second oligonucleotide comprising one or more hairpin structures each self-anneal to further form a single-stranded overhang at either the 5′ end or the 3′ end of each oligonucleotide.

22. The method of claim 21, wherein the first oligonucleotide and the second oligonucleotide each self-anneal to further form a single-stranded overhang at the 5′ end of each oligonucleotide.

23. The method of claim 22, wherein the first oligonucleotide and the second oligonucleotide each self-anneal to further form a single-stranded overhang at the 3′ end of each oligonucleotide.

24. The method of any one of claims 1-23, wherein the 5′ end of each oligonucleotide is phosphorylated.

25. The method of any one of claims 21-24, wherein the single-stranded overhang of the first oligonucleotide and the single-stranded overhang of the second oligonucleotide are each about 1 to about 12 nucleotides in length.

26. The method of any one of claims 21-25, wherein the single-stranded overhang of the first oligonucleotide and the single-stranded overhang of the second oligonucleotide are each about 1 to about 8 nucleotides in length.

27. The method of any one of claims 21-26, wherein the single-stranded overhang of the first oligonucleotide and the single-stranded overhang of the second oligonucleotide are each about 2 to about 6 nucleotides in length.

28. The method of any one of claims 21-27, wherein the single-stranded overhang of the first oligonucleotide and the single-stranded overhang of the second oligonucleotide are each about 3, about 4, about 5, or about 6 nucleotides in length.

29. The method of any one of claims 21-28, wherein the single-stranded overhang of the first oligonucleotide and the single-stranded overhang of the second oligonucleotide are each 3 or 4 nucleotides in length.

30. The method of any one of claims 25-29, wherein the 5′ to 3′ nucleotide sequences of the single-stranded overhang of the first oligonucleotide and the single-stranded overhang of the second oligonucleotide are non-complementary to each other.

31. The method of any one of claims 25-30, wherein the 5′ to 3′ nucleotide sequences of the single-stranded overhang of the first oligonucleotide and the single-stranded overhang of the second oligonucleotide are the same.

32. The method of claim 31, wherein the first oligonucleotide and the second oligonucleotide have the same nucleotide sequence.

33. The method of any one of claims 25-32, wherein the single-stranded overhangs at each end of the insert comprise the same 5′ to 3′ nucleotide sequence.

34. The method of any one of claims 25-33, wherein the single-stranded overhang of the first oligonucleotide and the single-stranded overhang of the second oligonucleotide are each complementary to both of the single-stranded overhangs at the ends of the insert.

35. The method of any one of claims 25-34, wherein the 5′ to 3′ nucleotide sequences of the single-stranded overhang of the first oligonucleotide and the single-stranded overhang of the second oligonucleotide are different.

36. The method of any one of claims 1-35, wherein the first oligonucleotide and the second oligonucleotide comprise different nucleotide sequences.

37. The method of any one of claims 1-36, wherein the 5′ to 3′ nucleotide sequences of the single-stranded overhangs at each end of the insert are different.

38. The method of any one of claims 1-37, wherein the single-stranded overhang of the first oligonucleotide and the single-stranded overhang of the second oligonucleotide are each complementary to only one of the single-stranded overhangs at the ends of the insert.

39. The method of any one of claims 21-38, wherein the single-stranded overhang of the first oligonucleotide and/or the single-stranded overhang of the second oligonucleotide comprise a 5′ to 3′ nucleotide sequence selected from the group consisting of CTCT, CTCA, CACT, CTC, and GCT.

40. The method of any one of claims 1-39, wherein one or both of the first oligonucleotide and the second oligonucleotide is synthetic.

41. The method of any one of claims 1-40, wherein the first oligonucleotide and the second oligonucleotide are each about 40 nucleotides to about 75 nucleotides in length.

42. The method of any one of claims 1-41, wherein the first oligonucleotide and the second oligonucleotide are each about 45 nucleotides to about 65 nucleotides in length.

43. The method of any one of claims 1-42, wherein the first oligonucleotide and the second oligonucleotide each independently comprise a nucleotide sequence selected from the group consisting of SEQ ID NO: 1; SEQ ID NO: 2; SEQ ID NO: 3; SEQ ID NO: 4; SEQ ID NO: 5; SEQ ID NO: 6; SEQ ID NO: 7; and SEQ ID NO: 8.

44. The method of any one of claims 1-43, wherein each hairpin structure and/or each T- or Y-shaped stem-loop structure of the first oligonucleotide and each hairpin structure and/or each T- or Y-shaped stem-loop structure of the second oligonucleotide comprises a stem region that is at least about 4 base pairs in length.

45. The method of any one of claims 1-44, wherein each hairpin structure and/or each T- or Y-shaped stem-loop structure of the first oligonucleotide and each hairpin structure and/or each T- or Y-shaped stem-loop structure of the second oligonucleotide comprises a stem region that is about 4 base pairs to about 20 base pairs in length.

46. The method of any one of claims 1-45, wherein each hairpin structure and/or each T- or Y-shaped stem-loop structure of the first oligonucleotide and each hairpin structure and/or each T- or Y-shaped stem-loop structure of the second oligonucleotide comprises a stem region that is about 4 base pairs to about 15 base pairs in length.

47. The method of any one of claims 1-46, wherein each hairpin structure and/or each T- or Y-shaped stem-loop structure of the first oligonucleotide and each hairpin structure and/or each T- or Y-shaped stem-loop structure of the second oligonucleotide comprises a stem region that is about 4 base pairs to about 6 base pairs in length.

48. The method of any one of claims 1-47, wherein each hairpin structure and/or each T- or Y-shaped stem-loop structure of the first oligonucleotide and each hairpin structure and/or each T- or Y-shaped stem-loop structure of the second oligonucleotide comprises a stem region that is about 6 base pairs to about 8 base pairs in length.

49. The method of any one of claims 44-48, wherein the stem region length does not include any single-stranded overhang.

50. The method of any one of claims 1-49, wherein at least one of the restriction endonucleases is a Type IIS restriction endonuclease.

51. The method of any one of claims 1-50, wherein the first restriction endonuclease and the second restriction endonuclease are the same restriction endonuclease.

52. The method of any one of claims 1-50, wherein the first restriction endonuclease and the second restriction endonuclease are different restriction endonucleases.

53. The method of any one of claims 1-52, wherein each of the first and second restriction endonucleases is a Type IIS restriction endonuclease.

54. The method of any one of claims 50-53, wherein the Type IIS restriction endonuclease is selected from group consisting of AcuI, AlwI, Alw26I, BasI, BbsI, BbvI, BceAI, BcgI, BCiVI, BcoDI, BruAI, BmrI, BpiI, BpuEI, BsaI, BsaXI, BseGI, BseRI, BsgI, BsmAI, BsmBI, BsmFI, BsmI, BspCNI, BspMI, BspQI, BsrDI, BsrI, BtgZI, BtsCI, BtsI, MutI, CspCI, Earl, EciI, Eco31I, Esp3I, FauI, FokI, HgaI, HphI, HpyAV, LguI, MboII, MlyI, MmeI, MnlI, Mva1269I, NmeAIII, PaqCI, PleI, SapI, SfaNI, and an isoschizomer thereof.

55. The method of any one of claims 1-52, wherein the each of the first and second restriction endonucleases is a Type IIS restriction endonuclease independently selected from the group consisting of AcuI, AlwI, Alw26I, BasI, BbsI, BbvI, BceAI, BcgI, BCVI, BcoDI, BruAI, BmrI, BpiI, BpuEI, BsaI, BsaXI, BseGI, BseRI, BsgI, BsmAI, BsmBI, BsmFI, BsmI, BspCNI, BspMI, BspQI, BsrDI, BsrI, BtgZI, BtsCI, BtsI, MutI, CspCI, Earl, EciI, Eco31I, Esp3I, FauI, FokI, HgaI, HphI, HpyAV, LguI, MboII, MlyI, MmeI, MnlI, Mva1269I, NmeAIII, PaqCI, PleI, SapI, SfaNI, and an isoschizomer thereof.

56. The method of any one of claims 50-55, wherein the at least one Type IIS restriction endonuclease is selected from group consisting of BbsI, BsaI, Esp3I, and SapI, and an isoschizomer thereof.

57. The method of any one of claims 50-56, wherein the at least one Type IIS restriction endonuclease is BsaI or an isoschizomer thereof.

58. The method of any one of claims 50-56, wherein the at least one Type IIS restriction endonuclease is Esp3I or an isoschizomer thereof.

59. The method of any one of claims 1-58, wherein after the ligating, the first non-palindromic restriction endonuclease recognition site and the second non-palindromic restriction endonuclease recognition site are not regenerated in the resulting ceDNA vector.

60. The method of any one of claims 1-59, wherein the double-stranded DNA construct further comprises a least a first partial ITR and a second partial ITR each flanking the transgene expression cassette.

61. The method of claim 60, wherein the first partial ITR is upstream of the transgene expression cassette and downstream of the first non-palindromic restriction endonuclease recognition site and the corresponding first cleavage site.

62. The method of any one of claims 60-61, wherein the second partial ITR is downstream of the transgene expression cassette and upstream of the second non-palindromic restriction endonuclease recognition site and the corresponding second cleavage site.

63. The method of any one of claims 60-62, wherein the first cleavage site is adjacent to the first partial ITR and the second cleavage site is adjacent to the second partial ITR.

64. The method of any one of claims 60-63, wherein the double-stranded DNA construct further comprises a first spacer between the first partial ITR and the transgene expression cassette.

65. The method of any one of claims 60-64, wherein the double-stranded DNA construct further comprises a second spacer between the second partial ITR and the transgene expression cassette.

66. The method of any one of claims 1-65, wherein the double-stranded DNA construct is selected from the group consisting of a bacmid, a plasmid, a minicircle, and a linear double-stranded DNA molecule.

67. The method of any one of claims 1-66, wherein the resulting ceDNA vector comprises the transgene expression cassette and at least a first ITR and a second ITR each flanking the transgene expression cassette.

68. The method of claim 67, wherein the first ITR is upstream of the transgene expression cassette.

69. The method of any one of claims 67-68, wherein the second ITR is downstream of the transgene expression cassette.

70. The method of any one of claims 67-69, wherein the first ITR comprises nucleotide sequences from the first oligonucleotide and the first partial ITR.

71. The method of any one of claims 67-70, wherein the second ITR comprises nucleotide sequences from the second oligonucleotide and the second partial ITR.

72. The method of any one of claims 67-71, wherein the first ITR is devoid of the first non-palindromic restriction endonuclease recognition site.

73. The method of any one of claims 67-72, wherein the second ITR is devoid of the second non-palindromic restriction endonuclease recognition site.

74. The method of any one of claims 67-73, wherein the first ITR and the second ITR each comprise a hairpin structure and/or a T- or Y-shaped stem-loop structure.

75. The method of claim 74, wherein the first ITR and the second ITR each comprise a T- or Y-shaped stem-loop structure.

76. The method of any one of claims 20-75, wherein the T- or Y-shaped stem-loop structure comprises a stem comprising A-A′ and D-D′ stem regions and two B-B′ and C-C′ loops.

77. The method of any one of claims 67-76, wherein one or both of the first ITR and the second ITR is an adeno-associated virus (AAV) ITR or an AAV-derived ITR.

78. The method of any one of claims 67-77, wherein one or both of the first ITR and the second ITR is a wild-type ITR.

79. The method of claim 78, wherein both the first ITR and the second ITR are wild-type ITRs.

80. The method of any one of claims 67-79, wherein one or both of the first ITR and the second ITR is a modified ITR.

81. The method of any one of claims 67-80, wherein the first ITR and the second ITR are symmetrical or substantially symmetrical to each other.

82. The method of any one of claims 67-81, wherein the first ITR and the second ITR are asymmetrical ITRs.

83. The method of any one of claims 67-82, wherein the one or both of the first ITR and the second ITR comprises one or more modifications selected from the group consisting of an addition, a deletion, a truncation, and a point mutation.

84. The method of claim 83, wherein the one or more modifications are located in the A-A′ stem region, the B-B′ loop, the C-C′ loop, and/or D-D′ stem region of one or both of the first ITR and the second ITR.

85. The method of any one of claims 83-84, wherein the one or more modifications are located in the B-B′ loop and/or the C-C′ loop of one or both of the first ITR and the second ITR.

86. The method of claim 85, wherein the B-B′ loop and the C-C′ loop of one of the first ITR and the second ITR are truncated.

87. The method of any one of claims 67-86, wherein the transgene expression cassette further comprises a first spacer between the first ITR and the transgene expression cassette.

88. The method of any one of claims 67-86, wherein the transgene expression cassette further comprises a first spacer between the second ITR and the transgene expression cassette.

89. The method of any one of claims 67-88, wherein the transgene expression cassette further comprises a first spacer between the first ITR and the transgene expression cassette, and a second spacer between the second ITR and the transgene expression cassette.

90. The method of any one of claims 1-89, wherein the transgene expression cassette comprises a transgene.

91. The method of claim 90, wherein the transgene encodes a therapeutic protein.

92. The method of claim 91, wherein the therapeutic protein is selected from the group consisting of an enzyme, a coagulation factor or co-factor, an antibody or an antigen-binding fragment thereof, an antigen, a gene-editing protein, and a cytotoxic protein.

93. The method of any one of claims 1-92, wherein the transgene expression cassette further comprises a genetic element selected from the group consisting of a promoter, an enhancer, an intron, a posttranscriptional regulatory element, and a polyadenylation signal.

94. The method of claim 93, wherein the posttranscriptional regulatory element comprises a WHP posttranscriptional regulatory element (WPRE).

95. The method of any one of claims 1-94, wherein the ligating is effected by a ligase or an AAV Rep protein.

96. The method of claim 95, wherein the ligase is T4 ligase.

97. The method of any one of claims 1-96, wherein the method further comprises isolating or purifying the resulting ceDNA vector.

98. The method of any one of claims 1-97, wherein the method further comprises isolating or purifying the insert prior to the ligating.

99. The method of any one of claims 1-97, wherein the method does not comprise isolating or purifying the insert prior to the ligating.

100. The method of any one of claims 1-99, wherein steps (a), (b), and (c) are performed in a single reaction vessel.

101. The method of any one of claims 1-100, wherein the resulting ceDNA vector comprises at least about 85%, at least about 90%, at least about 95%, at least about 96%, at least about 97%, at least about 98%, or at least about 99% of a monomeric species of the ceDNA vector.

102. A closed-ended DNA (ceDNA) vector produced by the method of any one of claims 1-101.

103. A pharmaceutical composition comprising the closed-ended DNA (ceDNA) vector produced by the method of any one of claims 1-101 and at least one pharmaceutically acceptable excipient.

104. A lipid nanoparticle composition comprising the closed-ended DNA (ceDNA) vector produced by the method of any one of claims 1-101.

105. An isolated host cell comprising the closed-ended DNA (ceDNA) vector produced by the method of any one of claims 1-101.

106. A transgenic animal comprising the closed-ended DNA (ceDNA) vector produced by the method of any one of claims 1-101.

107. A method of treating a disorder, disease, or condition in a subject, the method comprising administering to the subject a therapeutically effective amount of the closed-ended DNA (ceDNA) vector produced by the method of any one of claims 1-101, or the pharmaceutical composition of claim 103, or the lipid nanoparticle composition of claim 104.

108. The method of claim 107, wherein the disorder, disease, or condition is a genetic disorder, disease, or condition.

109. A method of delivering a therapeutic protein to a subject, the method comprising administering to the subject a therapeutically effective amount of the closed-ended DNA (ceDNA) vector produced by the method of any one of claims 1-101, or the pharmaceutical composition of claim 103, or the lipid nanoparticle composition of claim 104.

110. The method of claim 109, wherein the therapeutic protein is selected from the group consisting of an enzyme, a coagulation factor or co-factor, an antibody or an antigen-binding fragment thereof, an antigen, a gene-editing protein, and a cytotoxic protein.

111. An inverted terminal repeat (ITR) nucleotide sequence selected from the group consisting of:

| SEQ | Left | |
|---|---|---|
| ID | or | |
| NO: | Right | Sequence (5′ to 3′) |
| 18 | Left | CTCACTGAGGCCGCCCGGGCAAAGCCCGGGCGT |
| | | CGGGCGACCTTTGGTCGCCCGGCCTCAG |
| 19 | Left | CTCACTGAGGCCGCCCGGGCAAAGCCCGGGCGT |
| | | CGGGCGACCTTTGGTCGCCCGGCCTCAGTGAGC |
| | | GAGCGAGCGCGCAGAGAGGGAGTGGCCAACTC |
| | | CATCACTAGGGGTTCCT |
| 20 | Left | CTCTCTGAGGCCGCCCGGGCAAAGCCCGGGCGT |
| | | CGGGCGACCTTTGGTCGCCCGGCCTCAG |
| 21 | Left | CTCTCTGAGGCCGCCCGGGCAAAGCCCGGGCGT |
| | | CGGGCGACCTTTGGTCGCCCGGCCTCAGTGAGC |
| | | GAGCGAGCGCGCAGAGAGGGAGTGGCCAACTC |
| | | CATCACTAGGGGTTCCT |
| 22 | Left | CACTGAGGCCGCCCGGGCAAAGCCCGGGCGTC |
| | | GGGCGACCTTTGGTCGCCCGGCCTCAG |
| 23 | Left | CACTGAGGCCGCCCGGGCAAAGCCCGGGCGTC |
| | | GGGCGACCTTTGGTCGCCCGGCCTCAGTGAGC |
| | | GAGCGAGCGCGCAGAGAGGGAGTGGCCAACT |
| | | CCATCACTAGGGGTTCCT |
| 24 | Left | CTCACTGAGGCCGCCCGGGCAAAGCCCGGGCG |
| | | TCGGGCGACCTTTGGTCGCCCGGCCTCAG |
| 25 | Left | CTCACTGAGGCCGCCCGGGCAAAGCCCGGGCG |
| | | TCGGGCGACCTTTGGTCGCCCGGCCTCAGTGAG |
| | | CGAGCGAGCGCGCAGAGAGGGAGTGGCCAACT |
| | | CCATCACTAGGGGTTCCT |
| 26 | Right | CTGAGGCCGGGCGACCAAAGGTCGCCCGACGCC |
| | | CGGGCTTTGCCCGGGCGGCCTCAGTGAG |
| 27 | Right | AGGAACCCCTAGTGATGGAGTTGGCCACTCCCTC |
| | | TCTGCGCGCTCGCTCGCTCACTGAGGCCGGGCGA |
| | | CCAAAGGTCGCCCGACGCCCGGGCTTTGCCCGGG |
| | | CGGCCTCAGTGAG |
| 28 | Right | CTGAGGCCGGGCGACCAAAGGTCGCCCGACGCCC |
| | | GGGCGGCCTC |
| 29 | Right | AGGAACCCCTAGTGATGGAGTTGGCCACTCCCTCT |
| | | CTGCGCGCTCGCTCGCTCACTGAGGCCGGGCGACC |
| | | AAAGGTCGCCCGACGCCCGGGCGGCCTC |
| 30 | Right | CTGAGGCCGGGCGACCAAAGGTCGCCCGACGCCC |
| | | GGGCTTTGCCCGGGCGGCCTCAGTGAG |
| 31 | Right | AGGAACCCCTAGTGATGGAGTTGGCCACTCCCTCTC |
| | | TGCGCGCTCGCTCGCTCACTGAGGCCGGGCGACCAA |
| | | AGGTCGCCCGACGCCCGGGCTTTGCCCGGGCGGCCT |
| | | CAGTGAG |

112. The ITR nucleotide sequence of claim 111, wherein the nucleotide sequence further includes a spacer selected from the group consisting of:

| SEQ | Left | |
|---|---|---|
| ID | or | |
| NO: | Right | Sequence (5′ to 3′) |
| 32 | Left | TGTAGTTAATGATTAACCCGCCATGCTACTTAT |
| | | CGCGGCCGC |
| 33 | Left | TGTAGTTAATGATTAACCCGCCATGCTACTTAT |
| | | CTACGTAGCCATGCATATG |
| 34 | Left | TGTAGTTAATGATTAACCCGCCATGCTACTTATC |
| 35 | Left | TGTAGTTAATGATTAACCCACCATGCTACTTATG |
| | | GCCTGCAGG |
| 36 | Left | TGTAGTTAATGATTAACCCACCATGCTACTTATC |
| | | ACCAATTG |
| 37 | Right | TCTAGAGCATGGCTACGTAGATAAGTAGCATGG |
| | | CGGGTTAATCATTAACTACACCTGCAGG |
| 38 | Right | GCTAGCCCACAATCTGCCTCCCAGTAGTACATG |
| | | ACATTAGTTTATTAATAGCCT |
| 39 | Left | TCCACAATCTGCCTCCCAGTAGTACATGACATT |
| | | AGTTTATTAATAGCCT |
| 40 | Right | CTTAAGCATGGCTACGTAGATAAGTAGCATGGC |
| | | GGGTTAATCATTAACTACACCTGCAGG |

113. A closed-ended DNA (ceDNA) vector comprising a transgene expression cassette and at least a first inverted terminal repeat (ITR) and a second ITR flanking the transgene expression cassette; wherein the first ITR and the second ITR each comprise a nucleotide sequence selected from the group consisting of the ITR nucleotide sequences of any one of claims 111-112.

114. The ceDNA vector of claim 113, wherein the vector comprises double-stranded DNA.

115. A pharmaceutical composition comprising the ceDNA vector of any one of claims 113-114 and at least one pharmaceutically acceptable excipient.

116. A lipid nanoparticle composition comprising the ceDNA vector of any one of claims 113-114.

117. An isolated host cell comprising the ceDNA vector of any one of claims 113-114.

118. A transgenic animal comprising the ceDNA vector of any one of claims 113-114.

119. A method of treating a disorder, disease, or condition in a subject, the method comprising administering to the subject a therapeutically effective amount of the ceDNA vector of any one of claims 113-114, or the pharmaceutical composition of claim 115, or the lipid nanoparticle composition of claim 116.

120. The method of claim 119, wherein the disorder, disease, or condition is a genetic disorder, disease, or condition.

121. A method of delivering a therapeutic protein to a subject, the method comprising administering to the subject a therapeutically effective amount of the ceDNA vector of any one of claims 113-114, or the pharmaceutical composition of claim 115, or the lipid nanoparticle composition of claim 116.

122. The method of claim 121, wherein the therapeutic protein is selected from the group consisting of an enzyme, an antibody or an antigen-binding fragment thereof, an antigen, a gene-editing protein, and a cytotoxic protein.

123. A DNA vector for use in synthetic production of a closed-ended DNA vector (ceDNA), comprising:
a multiple cloning site capable of receiving a transgene;
a first non-palindromic restriction endonuclease recognition site and a corresponding first cleavage site upstream of the multiple cloning site;
a second non-palindromic restriction endonuclease recognition site and a corresponding second cleavage site downstream of the multiple cloning site; and
a first partial ITR and a second partial ITR each flanking the multiple cloning site.

124. The DNA vector of claim 123, wherein the first partial ITR is upstream of the multiple cloning site and downstream of the first non-palindromic restriction endonuclease recognition site and the corresponding first cleavage site.

125. The DNA vector of any one of claims 123-124, wherein the second partial ITR is downstream of the multiple cloning site and upstream of the second non-palindromic restriction endonuclease recognition site and the corresponding second cleavage site.

126. The DNA vector of any one of claims 123-125, further comprising one or more spacers.

127. The DNA vector of any one of claims 123-126, further comprising an origin of replication and a selectable marker gene.

128. The DNA vector of any one of claims 123-127, wherein the multiple cloning site is capable of receiving a transgene and one or more additional genetic elements selected from the group consisting of a promoter, an enhancer, an intron, a posttranscriptional regulatory element and a polyadenylation signal.

129. The DNA vector of any one of claims 123-128, wherein the first non-palindromic restriction endonuclease recognition site is specific for a first restriction endonuclease, and the second non-palindromic restriction endonuclease recognition site is specific for at least a second restriction endonuclease.

130. The DNA vector of claim 129, wherein the first restriction endonuclease and the second restriction endonuclease are the same restriction endonuclease.

131. The DNA vector of claim 129, wherein the first restriction endonuclease and the second restriction endonuclease are different restriction endonucleases.

132. The DNA vector of any one of claims 129-131, wherein at least one of the restriction endonucleases is a Type IIS restriction endonuclease.

133. The DNA vector of any one of claims 129-132, wherein each of the first and second restriction endonucleases is a Type IIS restriction endonuclease.

134. The DNA vector of any one of claims 132-133, wherein the Type IIS restriction endonuclease is selected from group consisting of AcuI, AlwI, Alw26I, BasI, BbsI, BbvI, BceAI, BcgI, BCiVI, BcoDI, BruAI, BmrI, BpiI, BpuEI, BsaI, BsaXI, BseGI, BseRI, BsgI, BsmAI, BsmBI, BsmFI, BsmI, BspCNI, BspMI, BspQI, BsrDI, BsrI, BtgZI, BtsCI, BtsI, MutI, CspCI, Earl, EciI, Eco31I, Esp3I, FauI, FokI, HgaI, HphI, HpyAV, LguI, MboII, MlyI, MmeI, MnlI, Mva1269I, NmeAIII, PaqCI, PleI, SapI, SfaNI, and an isoschizomer thereof.

135. The DNA vector of any one of claims 133-134, wherein each Type IIS restriction endonuclease is independently selected from group consisting of AcuI, AlwI, Alw26I, BasI, BbsI, BbvI, BceAI, BcgI, BCiVI, BcoDI, BruAI, BmrI, BpiI, BpuEI, BsaI, BsaXI, BseGI, BseRI, BsgI, BsmAI, BsmBI, BsmFI, BsmI, BspCNI, BspMI, BspQI, BsrDI, BsrI, BtgZI, BtsCI, BtsI, MutI, CspCI, Earl, EciI, Eco31I, Esp3I, FauI, FokI, HgaI, HphI, HpyAV, LguI, MboII, MlyI, MmeI, MnlI, Mva1269I, NmeAIII, PaqCI, PleI, SapI, SfaNI, and an isoschizomer thereof.

136. The DNA vector of any one of claims 131-135, wherein the Type IIS restriction endonuclease is selected from group consisting of BbsI, BsaI, Esp3I, and SapI, and an isoschizomer thereof.

137. The DNA vector of any one of claims 123-136, wherein the DNA vector comprises a nucleotide sequence selected from the group consisting of SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; and SEQ ID NO: 17.

138. A kit for preparing a closed-ended DNA (ceDNA) vector comprising a transgene, the kit comprising:
the DNA vector of any one of claims 123-137;
at least one restriction endonuclease capable of cleaving the DNA vector at the multiple cloning site to allow the multiple cloning site to receive a transgene;
at least one restriction endonuclease capable of cleaving at the first and second cleavage sites;
a ligase; and
instructions for use.

139. The kit of claim 138, further comprising at least one oligonucleotide comprising one or more hairpin structures.

140. A double-stranded circular DNA construct engineered to facilitate preparation of a closed-ended DNA (ceDNA) vector comprising a transgene expression cassette, the double-stranded circular DNA construct comprising:
a transgene expression cassette;
a first non-palindromic restriction endonuclease recognition site and a corresponding first cleavage site upstream of the transgene expression cassette;
a second non-palindromic restriction endonuclease recognition site and a corresponding second cleavage site downstream of the transgene expression cassette; and
a first partial ITR and a second partial ITR each flanking the transgene expression cassette.

141. The double-stranded circular DNA construct of claim 140, wherein the first partial ITR is upstream of the transgene expression cassette and downstream of the first non-palindromic restriction endonuclease recognition site and the corresponding first cleavage site.

142. The double-stranded circular DNA construct of anyone of claims 140-141, wherein the second partial ITR is downstream of the transgene expression cassette and upstream of the second non-palindromic restriction endonuclease recognition site and the corresponding second cleavage site.

143. The double-stranded circular DNA construct of any one of claims 140-142, wherein the first non-palindromic restriction endonuclease recognition site is specific for a first restriction endonuclease and the second non-palindromic restriction endonuclease recognition site are specific for at least a second restriction endonuclease.

144. The double-stranded circular DNA construct of claim 143, wherein the first restriction endonuclease and the second restriction endonuclease are the same restriction endonuclease.

145. The double-stranded circular DNA construct of claim 143, wherein the first restriction endonuclease and the second restriction endonuclease are different restriction endonucleases.

146. The double-stranded circular DNA construct of any one of claims 140-145, wherein at least one of the restriction endonucleases is a Type IIS restriction endonuclease.

147. The double-stranded circular DNA construct of any one of claims 143-146, wherein each of the first and second restriction endonucleases is a Type IIS restriction endonuclease.

148. The double-stranded circular DNA construct of any one of claims 146-147, wherein the Type IIS restriction endonuclease is selected from group consisting of AcuI, AlwI, Alw26I, BasI, BbsI, BbvI, BceAI, BcgI, BCiVI, BcoDI, BruAI, BmrI, BpiI, BpuEI, BsaI, BsaXI, BseGI, BseRI, BsgI, BsmAI, BsmBI, BsmFI, BsmI, BspCNI, BspMI, BspQI, BsrDI, BsrI, BtgZI, BtsCI, BtsI, MutI, CspCI, Earl, EciI, Eco31I, Esp3I, FauI, FokI, HgaI, HphI, HpyAV, LguI, MboII, MlyI, MmeI, MnlI, Mva1269I, NmeAIII, PaqCI, PleI, SapI, SfaNI, and an isoschizomer thereof.

149. The double-stranded circular DNA construct of any one of claims 146-148, wherein each Type IIS restriction endonuclease is independently selected from group consisting of AcuI, AlwI, Alw26I, BasI, BbsI, BbvI, BceAI, BcgI, BCVI, BcoDI, BruAI, BmrI, BpiI, BpuEI, BsaI, BsaXI, BseGI, BseRI, BsgI, BsmAI, BsmBI, BsmFI, BsmI, BspCNI, BspMI, BspQI, BsrDI, BsrI, BtgZI, BtsCI, BtsI, MutI, CspCI, Earl, EciI, Eco31I, Esp3I, FauI, FokI, HgaI, HphI, HpyAV, LguI, MboII, MlyI, MmeI, MnlI, Mva1269I, NmeAIII, PaqCI, PleI, SapI, SfaNI, and an isoschizomer thereof.

150. The double-stranded circular DNA construct of any one of claims claim 146-148, wherein the at least one Type IIS restriction endonuclease is selected from group consisting of BbsI, BsaI, Esp3I, and SapI, and an isoschizomer thereof.

151. A kit for preparing a closed-ended DNA (ceDNA) vector comprising a transgene expression cassette, the kit comprising:
the double-stranded DNA construct of any one of claims 140-150;
at least one restriction endonuclease capable of cleaving the double-stranded DNA construct at the first and second cleavage sites;
a ligase; and
instructions for use.

152. The kit of claim 151, further comprising at least one oligonucleotide comprising one or more hairpin structures.

153. A method of producing a double-stranded DNA construct from a plasmid template via rolling-circle amplification, comprising the steps of:
(a) contacting the plasmid template with a thermostable polymerase having strand-displacement activity, wherein the ratio of plasmid template concentration (in ng/μl) to polymerase concentration (in U/μl) is greater than about 1;
(b) contacting the plasmid template with an oligonucleotide primer and dNTPs;
(c) incubating the plasmid template, the polymerase, the oligonucleotide primer, and the dNTPs at a temperature of about 40° C. or less, for a time period of at least about 5 hours;
thereby producing a double-stranded DNA construct.

154. The method of claim 153, wherein the ratio of plasmid template concentration (in ng/μl) to polymerase concentration (in U/μl) is greater than about 1, about 2, about 3, about 4, about 5, about 6, about 7, about 8, about 9, about 10, about 11, about 12, about 13, about 14, about 15, about 16, about 17, about 18, about 19, or about 20.

155. The method of any one of claims 153-154, wherein the plasmid template concentration is about 0.01 ng/μl, about 0.05 ng/μl, about 0.1 ng/μl, about 0.15 ng/μl, about 0.2 ng/μl, about 0.21 ng/μl, about 0.22 ng/μl, about 0.23 ng/μl, about 0.24 ng/μl, about 0.2 ng/μl 5, about 0.26 ng/μl, about 0.27 ng/μl, about 0.28 ng/μl, about 0.29 ng/μl, about 0.3 ng/μl, about 0.35 ng/μl, about 0.4 ng/μl, about 0.45 ng/μl, about 0.5 ng/μl, about 0.6 ng/μl, about 0.7 ng/μl, about 0.8 ng/μl, about 0.9 ng/μl, or about 1.0 ng/μl.

156. The method of any one of claims 153-155, wherein the polymerase concentration is about 0.01 U/μl, about 0.02 U/μl, about 0.03 U/μl, about 0.04 U/μl, about 0.05 U/μl, about 0.06 U/μl, about 0.07 U/μl, about 0.08 U/μl, about 0.09 U/μl, about 0.1 U/μl, about 0.15 U/μl, about 0.2 U/μl, about 0.25 U/μl, about 0.3 U/μl, about 0.35 U/μl, about 0.4 U/μl, or about 0.45 U/μl.

157. The method of any one of claims 153-156, wherein the temperature in step (c) is less than about 40° C., about 39° C., about 38° C., about 37° C., about 36° C., about 35° C., about 34° C., about 33° C., about 32° C., about 31° C., about 30° C., about 29° C., about 28° C., about 27° C., about 26° C., about 25° C., about 24° C., about 23° C., about 22° C., or about 21° C.

158. The method of any one of claims 153-157, wherein the time period is at least about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 21 hours, about 33 hours, about 34 hours, about 35 hours, about 36 hours, about 37 hours, about 38 hours, about 39 hours, or about 40 hours.

159. The method of any one of claims 153-158, is wherein the time period is less than about 6 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 21 hours, about 33 hours, about 34 hours, about 35 hours, about 36 hours, about 37 hours, about 38 hours, about 39 hours, or about 40 hours.

160. The method of any one of claims 153-159, wherein the plasmid template concentration is about 0.25 ng/μl, the temperature is about 30° C., the polymerase concentration is about 0.05 U/μl, and the time period is about 18-26 hours.

161. The method of any one of claims 153-160, wherein the oligonucleotide primer concentration is less than about 50 μM.

162. The method of any one of claims 153-161, wherein the oligonucleotide primer concentration is at least about 10 μM.

163. The method of any one of claims 153-162, wherein the oligonucleotide primer concentration is at least about 10 μM and less than about 50 μM.

164. The method of any one of claims 153-163, wherein the thermostable polymerase is Phi29 DNA polymerase or a derivative or variant thereof.

165. The method of claim 164, wherein the thermostable polymerase is EQUIPHI29™.

166. The method of any one of claims 153-165, wherein the method is performed in a total reaction volume of at least about 100 μl.

167. The method of any one of claims 153-166, wherein the method is performed in a total reaction volume of at least about 100 μl, about 200 μl, about 300 μl, about 400 μl, about 500 μl, about 600 μl, about 700 μl, about 800 μl, about 900 μl, about 1 ml, about 2 ml, about 3 ml, about 4 ml, about 5 ml, about 6 ml, about 7 ml, about 8 ml, about 9 ml, about 10 ml, about 15 ml, about 20 ml, about 25 ml, about 30 ml, about 35 ml, about 40 ml, about 45 ml, about 50 ml, about 55 ml, about 60 ml, about 65 ml, about 70 ml, about 75 ml, about 80 ml, about 85 ml, about 90 ml, about 95 ml, about 100 ml, about 200 ml, about 300 ml, about 400 ml, about 500 ml, about 600 ml, about 700 ml, about 800 ml, about 900 ml, about 1 L, about 2 L, about 3 L, 4 L, about 5 L, about 6 L, about 7 L, about 8 L, about 9 L, about 10 L, about 20 L, about 30 L, about 40 L, about 50 L, about 60 L, about 70 L, about 80 L, about 90 L, about 100 L, about 200 L, about 300 L, about 400 L, about 500 L, about 600 L, about 700 L, about 800 L, about 900 L, or about 1000 L.

168. The method of any one of claims 153-167, wherein the method is performed in a reaction vessel that has a capacity of at least twice the total reaction volume.

169. The method of any one of claims 153-168, wherein the oligonucleotide primer hybridizes to a backbone sequence in the plasmid template.

170. The method of any one of claims 153-169, wherein the oligonucleotide primer is a universal primer.

171. The method of any one of claims 153-170, wherein the dNTP concentration is about 4 mM.

172. A double-stranded DNA construct produced by the method of any one of claims 153-171.

173. A method of producing a closed-ended DNA (ceDNA) vector, the method comprising:
(a) producing a double-stranded DNA construct using the method of any one of claims 153-171;
(b) performing the method of any one of claims 1-101 to produce a ceDNA vector from the double-stranded DNA construct.

174. A ceDNA vector produced by the method of claim 173.

175. A pharmaceutical composition comprising the ceDNA vector produced by the method of claim 173 and at least one pharmaceutically acceptable excipient.

176. A lipid nanoparticle composition comprising ceDNA vector produced by the method of 173.

177. A method of preparing a closed-ended DNA (ceDNA) vector, the method comprising:
(a) contacting a double-stranded DNA construct with at least one restriction endonuclease, wherein:
the construct comprises:
a transgene expression cassette;
a first non-palindromic restriction endonuclease recognition site and a corresponding first cleavage site upstream of the transgene expression cassette, and
a second non-palindromic restriction endonuclease recognition site and a corresponding second cleavage site downstream of the transgene expression cassette; and
the at least one restriction endonuclease is capable of cleaving the construct at the first and second cleavage sites to release an insert having single-stranded overhangs at the 5′ and 3′ ends of the insert; and
(b) ligating the 5′ and 3′ ends of the insert to a first inverted terminal repeat (ITR) oligonucleotide and a second ITR oligonucleotide to form the ceDNA vector.


SEQUENCE LISTING (67 sequences)

SEQ ID NO: 1 - DNA, 61 bp, synthetic construct
  misc_feature 1..61: note = Synthetic
  source 1..61: mol_type = other DNA
ctcactgaggccgcccgggcaaagcccgggcgtcgggcgacctttggtcgcccggcctcag

SEQ ID NO: 2 - DNA, 61 bp, synthetic construct
  misc_feature 1..61: note = Synthetic
  source 1..61: mol_type = other DNA
ctctctgaggccgcccgggcaaagcccgggcgtcgggcgacctttggtcgcccggcctcag

SEQ ID NO: 3 - DNA, 46 bp, synthetic construct
  misc_feature 1..46: note = Synthetic
  source 1..46: mol_type = other DNA
cactgaggccgggcgaccaaaggtcgcccgacgcccgggcggcctc

SEQ ID NO: 4 - DNA, 57 bp, synthetic construct
  misc_feature 1..57: note = Synthetic
  source 1..57: mol_type = other DNA
cactgaggccgcccgggcaaagcccgggcgtcgggcgacctttggtcgcccggcctc

SEQ ID NO: 5 - DNA, 53 bp, synthetic construct
  misc_feature 1..53: note = Synthetic
  source 1..53: mol_type = other DNA
ctcaggccgcccgggcaaagcccgggcgtcgggcgacctttggtcgcccggcc

SEQ ID NO: 6 - DNA, 62 bp, synthetic construct
  misc_feature 1..62: note = Synthetic
  source 1..62: mol_type = other DNA
ctcactgaggccgcccgggcaaagcccgggcgtcgggcgacctttggtcgcccggcctcagt

SEQ ID NO: 7 - DNA, 64 bp, synthetic construct
  misc_feature 1..64: note = Synthetic
  source 1..64: mol_type = other DNA
gctcactgaggccgcccgggcaaagcccgggcgtcgggcgacctttggtcgcccggcctcagtg

SEQ ID NO: 8 - DNA, 75 bp, synthetic construct
  misc_feature 1..75: note = Synthetic
  source 1..75: mol_type = other DNA
ctcagagtgaggtgaggccgcccgggcaaagcccgggcgtcgggcgacctttggtcgcccggcctcacctcactc

SEQ ID NO: 9 - DNA, 2416 bp, synthetic construct
  misc_feature 1..2416: note = Synthetic
  source 1..2416: mol_type = other DNA
ttcgtcagatcaccgggttgttccatatcatcgtgtccacaagggcttgccggtcaagtgcctaagctcactcgagggtctcgagagcgagcgagcgcgcagagagggagtggccaactccatcactaggggttccttgtagttaatgattaacccgccatgctacttatcgcggccgcagttgcatctttgttataccagcgcacgagtctgtcgtcatggctacaagaccacgagaacatgagccggtgtctccagagaactccattgtgactaggtagtctagagcatggctacgtagataagtagcatggcgggttaatcattaactacacctgcaggaggaacccctagtgatggagttggccactccctctctgcgcgctcgctcgctctcgagaccaccggtttacaggatagtctactcccagtgtccacatggttaggaccgaggggctttgggtttagacactggcgacttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtct

SEQ ID NO: 10 - DNA, 2416 bp, synthetic construct
  misc_feature 1..2416: note = Synthetic
  source 1..2416: mol_type = other DNA
ttcgtcagatcaccgggttgttccatatcatcgtgtccacaagggcttgccggtcaagtgcctaagctcactcgagggtctcgtgagcgagcgagcgcgcagagagggagtggccaactccatcactaggggttccttgtagttaatgattaacccgccatgctacttatcgcggccgcagttgcatctttgttataccagcgcacgagtctgtcgtcatggctacaagaccacgagaacatgagccggtgtctccagagaactccattgtgactaggtagtctagagcatggctacgtagataagtagcatggcgggttaatcattaactacacctgcaggaggaacccctagtgatggagttggccactccctctctgcgcgctcgctcgctcacgagaccaccggtttacaggatagtctactcccagtgtccacatggttaggaccgaggggctttgggtttagacactggcgacttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtct

SEQ ID NO: 11 - DNA, 2420 bp, synthetic construct
  misc_feature 1..2420: note = Synthetic
  source 1..2420: mol_type = other DNA
ttcgtcagatcaccgggttgttccatatcatcgtgtccacaagggcttgccggtcaagtgcctaagctcactcgagggtctccagtgagcgagcgagcgcgcagagagggagtggccaactccatcactaggggttccttgtagttaatgattaacccgccatgctacttatcgcggccgcagttgcatctttgttataccagcgcacgagtctgtcgtcatggctacaagaccacgagaacatgagccggtgtctccagagaactccattgtgactaggtagtctagagcatggctacgtagataagtagcatggcgggttaatcattaactacacctgcaggaggaacccctagtgatggagttggccactccctctctgcgcgctcgctcgctcactggagaccaccggtttacaggatagtctactcccagtgtccacatggttaggaccgaggggctttgggtttagacactggcgacttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtct

SEQ ID NO: 12 - DNA, 2418 bp, synthetic construct
  misc_feature 1..2418: note = Synthetic
  source 1..2418: mol_type = other DNA
gcgcacatttccccgaaaagtgccacctgacgtctttcgtcagatcaccgggttgttccatatcatcgtgtccacaagggcttgccggtcaagtgcctaagctcactcgagggtctcgtgagcgagcgagcgcgcagagagggagtggccaactccatcactaggggttccttgtagttaatgattaacccgccatgctacttatcgcggccgcagttgcatctttgttataccagcgcacgagtctgtcgtcatggctacaagaccacgagaacatgagccggtgtctccagagaactccattgtgactaggtagtctagagcatggctacgtagataagtagcatggcgggttaatcattaactacacctgcaggaggaacccctagtgatggagttggccactccctctctgcgcgctcgctcgctcactggagaccaccggtttacaggatagtctactcccagtgtccacatggttaggaccgaggggctttgggtttagacactggcgacttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttcc

SEQ ID NO: 13 - DNA, 2416 bp, synthetic construct
  misc_feature 1..2416: note = Synthetic
  source 1..2416: mol_type = other DNA
ttcgtcagatcaccgggttgttccatatcatcgtgtccacaagggcttgccggtcaagtgcctaagctcactcgagcgtctcgtgagcgagcgagcgcgcagagagggagtggccaactccatcactaggggttccttgtagttaatgattaacccgccatgctacttatcgcggccgcagttgcatctttgttataccagcgcacgagtctgtcgtcatggctacaagaccacgagaacatgagccggtgtctccagagaactccattgtgactaggtagtctagagcatggctacgtagataagtagcatggcgggttaatcattaactacacctgcaggaggaacccctagtgatggagttggccactccctctctgcgcgctcgctcgctcacgagacgaccggtttacaggatagtctactcccagtgtccacatggttaggaccgaggggctttgggtttagacactggcgacttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtct

SEQ ID NO: 14 - DNA, 2416 bp, synthetic construct; identical to SEQ ID NO: 13
  misc_feature 1..2416: note = Synthetic
  source 1..2416: mol_type = other DNA

SEQ ID NO: 15 - DNA, 2416 bp, synthetic construct; identical to SEQ ID NO: 13
  misc_feature 1..2416: note = Synthetic
  source 1..2416: mol_type = other DNA

SEQ ID NO: 16 - DNA, 2416 bp, synthetic construct; identical to SEQ ID NO: 13
  misc_feature 1..2416: note = Synthetic
  source 1..2416: mol_type = other DNA

SEQ ID NO: 17 - DNA, 2416 bp, synthetic construct; identical to SEQ ID NO: 13
  misc_feature 1..2416: note = Synthetic
  source 1..2416: mol_type = other DNA

SEQ ID NO: 18 - DNA, 61 bp, synthetic construct; identical to SEQ ID NO: 1
  misc_feature 1..61: note = Synthetic
  source 1..61: mol_type = other DNA

SEQ ID NO: 19 - DNA, 115 bp, synthetic construct
  misc_feature 1..115: note = Synthetic
  source 1..115: mol_type = other DNA
ctcactgaggccgcccgggcaaagcccgggcgtcgggcgacctttggtcgcccggcctcagtgagcgagcgagcgcgcagagagggagtggccaactccatcactaggggttcct

SEQ ID NO: 20 - DNA, 61 bp, synthetic construct; identical to SEQ ID NO: 2
  misc_feature 1..61: note = Synthetic
  source 1..61: mol_type = other DNA

SEQ ID NO: 21 - DNA, 115 bp, synthetic construct
  misc_feature 1..115: note = Synthetic
  source 1..115: mol_type = other DNA
ctctctgaggccgcccgggcaaagcccgggcgtcgggcgacctttggtcgcccggcctcagtgagcgagcgagcgcgcagagagggagtggccaactccatcactaggggttcct

SEQ ID NO: 22 - DNA, 59 bp, synthetic construct
  misc_feature 1..59: note = Synthetic
  source 1..59: mol_type = other DNA
cactgaggccgcccgggcaaagcccgggcgtcgggcgacctttggtcgcccggcctcag

SEQ ID NO: 23 - DNA, 113 bp, synthetic construct
  misc_feature 1..113: note = Synthetic
  source 1..113: mol_type = other DNA
cactgaggccgcccgggcaaagcccgggcgtcgggcgacctttggtcgcccggcctcagtgagcgagcgagcgcgcagagagggagtggccaactccatcactaggggttcct

SEQ ID NO: 24 - DNA, 61 bp, synthetic construct; identical to SEQ ID NO: 1
  misc_feature 1..61: note = Synthetic
  source 1..61: mol_type = other DNA

SEQ ID NO: 25 - DNA, 115 bp, synthetic construct; identical to SEQ ID NO: 19
  misc_feature 1..115: note = Synthetic
  source 1..115: mol_type = other DNA

SEQ ID NO: 26 - DNA, 61 bp, synthetic construct
  misc_feature 1..61: note = Synthetic
  source 1..61: mol_type = other DNA
ctgaggccgggcgaccaaaggtcgcccgacgcccgggctttgcccgggcggcctcagtgag

SEQ ID NO: 27 - DNA, 115 bp, synthetic construct
  misc_feature 1..115: note = Synthetic
  source 1..115: mol_type = other DNA
aggaacccctagtgatggagttggccactccctctctgcgcgctcgctcgctcactgaggccgggcgaccaaaggtcgcccgacgcccgggctttgcccgggcggcctcagtgag

SEQ ID NO: 28 - DNA, 44 bp, synthetic construct
  misc_feature 1..44: note = Synthetic
  source 1..44: mol_type = other DNA
ctgaggccgggcgaccaaaggtcgcccgacgcccgggcggcctc

SEQ ID NO: 29 - DNA, 98 bp, synthetic construct
  misc_feature 1..98: note = Synthetic
  source 1..98: mol_type = other DNA
aggaacccctagtgatggagttggccactccctctctgcgcgctcgctcgctcactgaggccgggcgaccaaaggtcgcccgacgcccgggcggcctc

SEQ ID NO: 30 - DNA, 61 bp, synthetic construct; identical to SEQ ID NO: 26
  misc_feature 1..61: note = Synthetic
  source 1..61: mol_type = other DNA

SEQ ID NO: 31 - DNA, 115 bp, synthetic construct; identical to SEQ ID NO: 27
  misc_feature 1..115: note = Synthetic
  source 1..115: mol_type = other DNA

SEQ ID NO: 32 - DNA, 42 bp, synthetic construct
  misc_feature 1..42: note = Synthetic
  source 1..42: mol_type = other DNA
tgtagttaatgattaacccgccatgctacttatcgcggccgc

SEQ ID NO: 33 - DNA, 52 bp, synthetic construct
  misc_feature 1..52: note = Synthetic
  source 1..52: mol_type = other DNA
tgtagttaatgattaacccgccatgctacttatctacgtagccatgcatatg

SEQ ID NO: 34 - DNA, 34 bp, synthetic construct
  misc_feature 1..34: note = Synthetic
  source 1..34: mol_type = other DNA
tgtagttaatgattaacccgccatgctacttatc

SEQ ID NO: 35 - DNA, 43 bp, synthetic construct
  misc_feature 1..43: note = Synthetic
  source 1..43: mol_type = other DNA
tgtagttaatgattaacccaccatgctacttatggcctgcagg

SEQ ID NO: 36 - DNA, 42 bp, synthetic construct
  misc_feature 1..42: note = Synthetic
  source 1..42: mol_type = other DNA
tgtagttaatgattaacccaccatgctacttatcaccaattg

SEQ ID NO: 37 - DNA, 61 bp, synthetic construct
  misc_feature 1..61: note = Synthetic
  source 1..61: mol_type = other DNA
tctagagcatggctacgtagataagtagcatggcgggttaatcattaactacacctgcagg

SEQ ID NO: 38 - DNA, 54 bp, synthetic construct
  misc_feature 1..54: note = Synthetic
  source 1..54: mol_type = other DNA
gctagcccacaatctgcctcccagtagtacatgacattagtttattaatagcct

SEQ ID NO: 39 - DNA, 49 bp, synthetic construct
  misc_feature 1..49: note = Synthetic
  source 1..49: mol_type = other DNA
tccacaatctgcctcccagtagtacatgacattagtttattaatagcct

SEQ ID NO: 40 - DNA, 60 bp, synthetic construct
  misc_feature 1..60: note = Synthetic
  source 1..60: mol_type = other DNA
cttaagcatggctacgtagataagtagcatggcgggttaatcattaactacacctgcagg

SEQ ID NO: 41 - DNA, 12 bp, synthetic construct
  misc_feature 1..12: note = Synthetic
  source 1..12: mol_type = other DNA
tgagcgagcgag

SEQ ID NO: 42 - DNA, 20 bp, synthetic construct
  misc_feature 1..20: note = Synthetic
  source 1..20: mol_type = other DNA
ttccgcttcctcgctcactg

SEQ ID NO: 43 - DNA, 21 bp, synthetic construct
  misc_feature 1..21: note = Synthetic
  source 1..21: mol_type = other DNA
agacgtcaggtggcacttttc

SEQ ID NO: 44 - DNA, 35 bp, synthetic construct
  misc_feature 1..35: note = Synthetic
  source 1..35: mol_type = other DNA
gcgcacatttccccgaaaagtgccacctgacgtct

SEQ ID NO: 45 - DNA, 35 bp, synthetic construct
  misc_feature 1..35: note = Synthetic
  source 1..35: mol_type = other DNA
ttccgcttcctcgctcactgactcgctgcgctcgg

SEQ ID NO: 46 - DNA, 54 bp, synthetic construct
  misc_feature 1..54: note = Synthetic
  source 1..54: mol_type = other DNA
tgagcgagcgagcgcgcagagagggagtggccaactccatcactaggggttcct

SEQ ID NO: 47 - DNA, 54 bp, synthetic construct
  misc_feature 1..54: note = Synthetic
  source 1..54: mol_type = other DNA
aggaacccctagtgatggagttggccactccctctctgcgcgctcgctcgctca

SEQ ID NO: 48 - DNA, 24 bp, synthetic construct
  misc_feature 1..24: note = Synthetic
  source 1..24: mol_type = other DNA
tgattaacccgccatgctacttat

SEQ ID NO: 49 - DNA, 16 bp, synthetic construct
  misc_feature 1..16: note = Synthetic
  source 1..16: mol_type = other DNA
tgagcgagcgagcgcg

SEQ ID NO: 50 - DNA, 16 bp, synthetic construct
  misc_feature 1..16: note = Synthetic
  source 1..16: mol_type = other DNA
ggtctcgtgagcgagc

SEQ ID NO: 51 - DNA, 16 bp, synthetic construct
  misc_feature 1..16: note = Synthetic
  source 1..16: mol_type = other DNA
gctcgctcacgagacc

SEQ ID NO: 52 - DNA, 11 bp, synthetic construct
  misc_feature 1..11: note = Synthetic
  source 1..11: mol_type = other DNA
ctcacgagacc

SEQ ID NO: 53 - DNA, 16 bp, synthetic construct
  misc_feature 1..16: note = Synthetic
  source 1..16: mol_type = other DNA
ggtctcgtgcacgagc

SEQ ID NO: 54 - DNA, 16 bp, synthetic construct
  misc_feature 1..16: note = Synthetic
  source 1..16: mol_type = other DNA
gctcgtgcacgagacc

SEQ ID NO: 55 - DNA, 11 bp, synthetic construct
  misc_feature 1..11: note = Synthetic
  source 1..11: mol_type = other DNA
tgcacgagacc

SEQ ID NO: 56 - DNA, 61 bp, synthetic construct
  misc_feature 1..61: note = Synthetic
  source 1..61: mol_type = other DNA
ggtctcgtgagcgagcgagcgcgcagagagggagtggccaactccatcactaggggttcct

SEQ ID NO: 57 - DNA, 61 bp, synthetic construct
  misc_feature 1..61: note = Synthetic
  source 1..61: mol_type = other DNA
aggaacccctagtgatggagttggccactccctctctgcgcgctcgctcgctcacgagacc

SEQ ID NO: 58 - DNA, 54 bp, synthetic construct; identical to SEQ ID NO: 46
  misc_feature 1..54: note = Synthetic
  source 1..54: mol_type = other DNA

SEQ ID NO: 59 - DNA, 50 bp, synthetic construct
  misc_feature 1..50: note = Synthetic
  source 1..50: mol_type = other DNA
aggaacccctagtgatggagttggccactccctctctgcgcgctcgctcg

SEQ ID NO: 60 - DNA, 63 bp, synthetic construct
  misc_feature 1..63: note = Synthetic
  source 1..63: mol_type = other DNA
aggaacccctagtgatggagttggccactccctctctgcgcgctcgctcgctcactggagacc

SEQ ID NO: 61 - DNA, 63 bp, synthetic construct
  misc_feature 1..63: note = Synthetic
  source 1..63: mol_type = other DNA
ggtctccagtgagcgagcgagcgcgcagagagggagtggccaactccatcactaggggttcct

SEQ ID NO: 62 - DNA, 161 bp, synthetic construct
  misc_feature 1..161: note = Synthetic
  source 1..161: mol_type = other DNA
aggaacccctagatggagttggccactccctctctgcgcgctcgctcgctcactgaggccgcccgggcaaagcccgggcgtcgggcgacctttggtcgcccggcctcagtgagcgagcgagcgcgcagagagggagtggccaactccatctaggggttcct

SEQ ID NO: 63 - DNA, 58 bp, synthetic construct
  misc_feature 1..58: note = Synthetic
  source 1..58: mol_type = other DNA
actacacctgcaggaggaacccctagtgatggagttggccactccctctctgcgcgct

SEQ ID NO: 64 - DNA, 93 bp, synthetic construct
  misc_feature 1..93: note = Synthetic
  source 1..93: mol_type = other DNA
actacacctgcaggaggaacccctagtgatggagttggccactccctctctgcgcgctcgctcgctcactgaggccgggcgaccaaaggtcgc

SEQ ID NO: 65 - DNA, 74 bp, synthetic construct
  misc_feature 1..74: note = Synthetic
  source 1..74: mol_type = other DNA
ccggcctcagtgagcgagcgagcgcgcagagagggagtggccaactccatcactaggggttccttgtagttaat

SEQ ID NO: 66 - DNA, 47 bp, synthetic construct
  misc_feature 1..47: note = Synthetic
  source 1..47: mol_type = other DNA
cagagaggagtggccaactccatcactaggggttccttgtagttaat

SEQ ID NO: 67 - DNA, 52 bp, synthetic construct
  misc_feature 1..52: note = Synthetic
  source 1..52: mol_type = other DNA
cgcgcagagagggagtggccaactccatcactaggggttccttgtagttaat